SEQUENCE LISTING (268 sequences)

SEQ ID NO: 1 - protein, 14 aa, synthetic construct
  REGION 1..14: note = Synthetic
CAASDSSASKIIFG

SEQ ID NO: 2 - protein, 13 aa, synthetic construct
  REGION 1..13: note = Synthetic
CASSVGKETQYFG

SEQ ID NO: 3 - protein, 17 aa, synthetic construct
  REGION 1..17: note = Synthetic
CAMREPYYNQGGKLIFG

SEQ ID NO: 4 - protein, 16 aa, synthetic construct
  REGION 1..16: note = Synthetic
CASSQDRLARDTQYFG

SEQ ID NO: 5 - protein, 11 aa, synthetic construct
  REGION 1..11: note = Synthetic
CILRAVYVRFG

SEQ ID NO: 6 - protein, 16 aa, synthetic construct
  REGION 1..16: note = Synthetic
CASSWKGVYNQPQHFG

SEQ ID NO: 7 - protein, 16 aa, synthetic construct
  REGION 1..16: note = Synthetic
CAVQATSGSARQLTFG

SEQ ID NO: 8 - protein, 17 aa, synthetic construct
  REGION 1..17: note = Synthetic
CASSLSKTGRYNEQFFG

SEQ ID NO: 9 - protein, 14 aa, synthetic construct
  REGION 1..14: note = Synthetic
CATVFYGNNRLAFG

SEQ ID NO: 10 - protein, 16 aa, synthetic construct
  REGION 1..16: note = Synthetic
CASSFFPDSNQPQHFG

SEQ ID NO: 11 - protein, 13 aa, synthetic construct
  REGION 1..13: note = Synthetic
CVHMEYGNKLVFG

SEQ ID NO: 12 - protein, 14 aa, synthetic construct
  REGION 1..14: note = Synthetic
ASTLAGRAYEQYFG

SEQ ID NO: 13 - protein, 14 aa, synthetic construct
  REGION 1..14: note = Synthetic
CAVGVLRDYKLSFG

SEQ ID NO: 14 - protein, 14 aa, synthetic construct
  REGION 1..14: note = Synthetic
CASIQGTGLAYTFG

SEQ ID NO: 15 - protein, 14 aa, synthetic construct
  REGION 1..14: note = Synthetic
CAVAVFPGNQFYFG

SEQ ID NO: 16 - protein, 14 aa, synthetic construct
  REGION 1..14: note = Synthetic
CASNLGQAIKYTFG

SEQ ID NO: 17 - protein, 19 aa, synthetic construct
  REGION 1..19: note = Synthetic
CAYRSAMYSGGGADGLTFG

SEQ ID NO: 18 - protein, 14 aa, synthetic construct
  REGION 1..14: note = Synthetic
ASSLGGSFQPQHFG

SEQ ID NO: 19 - protein, 14 aa, synthetic construct
  REGION 1..14: note = Synthetic
CATDAYNFNKFYFG

SEQ ID NO: 20 - protein, 13 aa, synthetic construct
  REGION 1..13: note = Synthetic
CASRANTGELFFG

SEQ ID NO: 21 - protein, 17 aa, synthetic construct
  REGION 1..17: note = Synthetic
CALSERPGGATNKLIFG

SEQ ID NO: 22 - protein, 14 aa, synthetic construct
  REGION 1..14: note = Synthetic
CASIVGQGNEQFFG

SEQ ID NO: 23 - protein, 13 aa, synthetic construct
  REGION 1..13: note = Synthetic
CAVCDSWGKLQFG

SEQ ID NO: 24 - protein, 16 aa, synthetic construct
  REGION 1..16: note = Synthetic
CASSPLRDYFNEQFFG

SEQ ID NO: 25 - protein, 16 aa, synthetic construct
  REGION 1..16: note = Synthetic
CAVKGSGTSYGKLTFG

SEQ ID NO: 26 - protein, 13 aa, synthetic construct
  REGION 1..13: note = Synthetic
CASSLTGYEQFFG

SEQ ID NO: 27 - protein, 15 aa, synthetic construct
  REGION 1..15: note = Synthetic
CAVNSGGGADGLTFG

SEQ ID NO: 28 - protein, 12 aa, synthetic construct
  REGION 1..12: note = Synthetic
CSATEGTPQFFG

SEQ ID NO: 29 - protein, 17 aa, synthetic construct
  REGION 1..17: note = Synthetic
CAGEAFGGNYQLIWGAG

SEQ ID NO: 30 - protein, 16 aa, synthetic construct
  REGION 1..16: note = Synthetic
CASSPDLAGVNEQFFG

SEQ ID NO: 31 - protein, 17 aa, synthetic construct
  REGION 1..17: note = Synthetic
CAGPNAGGTSYGKLTFG

SEQ ID NO: 32 - protein, 15 aa, synthetic construct
  REGION 1..15: note = Synthetic
CASSQDLAIGEQYFG

SEQ ID NO: 33 - protein, 16 aa, synthetic construct
  REGION 1..16: note = Synthetic
CIVRVPSDNFNKFYFG

SEQ ID NO: 34 - protein, 13 aa, synthetic construct
  REGION 1..13: note = Synthetic
CASSRTFRELFFG

SEQ ID NO: 35 - protein, 16 aa, synthetic construct
  REGION 1..16: note = Synthetic
CALSEARGGADGLTFG

SEQ ID NO: 36 - protein, 17 aa, synthetic construct
  REGION 1..17: note = Synthetic
CASSYSGTSGIYEQYFG

SEQ ID NO: 37 - protein, 15 aa, synthetic construct
  REGION 1..15: note = Synthetic
CVVNGGYNNNDMRFG

SEQ ID NO: 38 - protein, 18 aa, synthetic construct
  REGION 1..18: note = Synthetic
CASSYSLSGTSSYEQYFG

SEQ ID NO: 39 - protein, 12 aa, synthetic construct
  REGION 1..12: note = Synthetic
CAVNAGNQFYFG

SEQ ID NO: 40 - protein, 15 aa, synthetic construct
  REGION 1..15: note = Synthetic
CASSPDRSADTQYFG

SEQ ID NO: 41 - protein, 11 aa, synthetic construct
  REGION 1..11: note = Synthetic
CAVGDYKLSFG

SEQ ID NO: 42 - protein, 16 aa, synthetic construct
  REGION 1..16: note = Synthetic
CSASRDIDSGNTIYFG

SEQ ID NO: 43 - protein, 18 aa, synthetic construct
  REGION 1..18: note = Synthetic
CAVQAPYSGAGSYQLTFG

SEQ ID NO: 44 - protein, 12 aa, synthetic construct
  REGION 1..12: note = Synthetic
CASKFGDTQYFG

SEQ ID NO: 45 - protein, 18 aa, synthetic construct
  REGION 1..18: note = Synthetic
CAVIRGYSGAGSYQLTFG

SEQ ID NO: 46 - protein, 12 aa, synthetic construct
  REGION 1..12: note = Synthetic
CASKFGNELFFG

SEQ ID NO: 47 - protein, 17 aa, synthetic construct
  REGION 1..17: note = Synthetic
CIVRVPSGAGSYQLTFG

SEQ ID NO: 48 - protein, 14 aa, synthetic construct
  REGION 1..14: note = Synthetic
CASSVVGTREQFFG

SEQ ID NO: 49 - protein, 18 aa, synthetic construct
  REGION 1..18: note = Synthetic
CAVRTGYSGAGSYQLTFG

SEQ ID NO: 50 - protein, 12 aa, synthetic construct
  REGION 1..12: note = Synthetic
CASSYGNEQFFG

SEQ ID NO: 51 - protein, 14 aa, synthetic construct
  REGION 1..14: note = Synthetic
CAVSPNYGQNFVFG

SEQ ID NO: 52 - protein, 14 aa, synthetic construct
  REGION 1..14: note = Synthetic
CASSQKQAYGYTFG

SEQ ID NO: 53 - protein, 12 aa, synthetic construct
  REGION 1..12: note = Synthetic
CITTNAGKSTFG

SEQ ID NO: 54 - protein, 15 aa, synthetic construct
  REGION 1..15: note = Synthetic
CASSFRAEAYEQYFG

SEQ ID NO: 55 - protein, 13 aa, synthetic construct
  REGION 1..13: note = Synthetic
CAVRDNNNDMRFG

SEQ ID NO: 56 - protein, 16 aa, synthetic construct
  REGION 1..16: note = Synthetic
CASSLGKGRNTEAFFG

SEQ ID NO: 57 - protein, 13 aa, synthetic construct
  REGION 1..13: note = Synthetic
CAVGGSARQLTFG

SEQ ID NO: 58 - protein, 17 aa, synthetic construct
  REGION 1..17: note = Synthetic
CSAPGLAGDPYNEQFFG

SEQ ID NO: 59 - protein, 15 aa, synthetic construct
  REGION 1..15: note = Synthetic
CAVNPLNNFNKFYFG

SEQ ID NO: 60 - protein, 15 aa, synthetic construct
  REGION 1..15: note = Synthetic
CATSGAENTGELFFG

SEQ ID NO: 61 - protein, 16 aa, synthetic construct
  REGION 1..16: note = Synthetic
CAVRVAGGTSYGKLTF

SEQ ID NO: 62 - protein, 15 aa, synthetic construct
  REGION 1..15: note = Synthetic
CASSGQGGSDTQYFG

SEQ ID NO: 63 - protein, 14 aa, synthetic construct
  REGION 1..14: note = Synthetic
CAVQGGSNYKLTFG

SEQ ID NO: 64 - protein, 14 aa, synthetic construct
  REGION 1..14: note = Synthetic
CASSSSETGELFFG

SEQ ID NO: 65 - protein, 15 aa, synthetic construct
  REGION 1..15: note = Synthetic
CAGAGSNSGYALNFG

SEQ ID NO: 66 - protein, 18 aa, synthetic construct
  REGION 1..18: note = Synthetic
CASSLVTGGLVDYEQYFG

SEQ ID NO: 67 - protein, 10 aa, synthetic construct
  REGION 1..10: note = Synthetic
CVVNEGMRFG

SEQ ID NO: 68 - protein, 15 aa, synthetic construct
  REGION 1..15: note = Synthetic
CASSLLTGTGELFFG

SEQ ID NO: 69 - protein, 15 aa, synthetic construct
  REGION 1..15: note = Synthetic
CAVNSGGGADGLTFG

SEQ ID NO: 70 - protein, 14 aa, synthetic construct
  REGION 1..14: note = Synthetic
CASEGLAGAEQYFG

SEQ ID NO: 71 - protein, 17 aa, synthetic construct
  REGION 1..17: note = Synthetic
CAAPMPMDTGRRALTFG

SEQ ID NO: 72 - protein, 14 aa, synthetic construct
  REGION 1..14: note = Synthetic
CAISGGSVGEQYFG

SEQ ID NO: 73 - protein, 14 aa, synthetic construct
  REGION 1..14: note = Synthetic
CAVSSGSARQLTFG

SEQ ID NO: 74 - protein, 17 aa, synthetic construct
  REGION 1..17: note = Synthetic
CASTLSTGQGIYGYTFG

SEQ ID NO: 75 - protein, 15 aa, synthetic construct
  REGION 1..15: note = Synthetic
CAVGGSGSARQLTFG

SEQ ID NO: 76 - protein, 17 aa, synthetic construct
  REGION 1..17: note = Synthetic
CASSPTTSGRIGELFFG

SEQ ID NO: 77 - protein, 272 aa, synthetic construct
  REGION 1..272: note = Synthetic
MTSIRAVFIFLWLQLDLVNGENVEQHPSTLSVQEGDSAVIKCTYSDSASNYFPWYKQELGKGPQLIIDIRSNVGEKKDQRIAVTLNKTAKHFSLHITETQPEDSAVYFCAASDSSASKIIFGSGTRLSIRPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 78 - protein, 307 aa, synthetic construct
  REGION 1..307: note = Synthetic
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVGKETQYFGPGTRLLVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 79 - protein, 278 aa, synthetic construct
  REGION 1..278: note = Synthetic
MSLSSLLKVVTASLWLGPGIAQKITQTQPGMFVQEKEAVTLDCTYDTSDQSYGLFWYKQPSSGEMIFLIYQGSYDEQNATEGRYSLNFQKARKSANLVISASQLGDSAMYFCAMREPYYNQGGKLIFGQGTELSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 80 - protein, 313 aa, synthetic construct
  REGION 1..313: note = Synthetic
MGCRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQDRLARDTQYFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 81 - protein, 265 aa, synthetic construct
  REGION 1..265: note = Synthetic
MKLVTSITVLLSLGIMGDAKTTQPNSMESNEEEPVHLPCNHSTISGTDYIHWYRQLPSQGPEYVIHGLTSNVNNRMASLAIAEDRKSSTLILHRATLRDAAVYYCILRAVYVRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 82 - protein, 310 aa, synthetic construct
  REGION 1..310: note = Synthetic
MGPGLLCWVLLCLLGAGPVDAGVTQSPTHLIKTRGQQVTLRCSPISGHKSVSWYQQVLGQGPQFIFQYYEKEERGRGNFPDRFSARQFPNYSSELNVNALLLGDSALYLCASSWKGVYNQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 83 - protein, 274 aa, synthetic construct
  REGION 1..274: note = Synthetic
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVQATSGSARQLTFGSGTQLTVLPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 84 - protein, 311 aa, synthetic construct
  REGION 1..311: note = Synthetic
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLSKTGRYNEQFFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 85 - protein, 272 aa, synthetic construct
  REGION 1..272: note = Synthetic
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATVFYGNNRLAFGKGNQVVVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 86 - protein, 310 aa, synthetic construct
  REGION 1..310: note = Synthetic
MGPGLLCWVLLCLLGAGPVDAGVTQSPTHLIKTRGQQVTLRCSPISGHKSVSWYQQVLGQGPQFIFQYYEKEERGRGNFPDRFSARQFPNYSSELNVNALLLGDSALYLCASSFFPDSNQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 87 - protein, 271 aa, synthetic construct
  REGION 1..271: note = Synthetic
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVHMEYGNKLVFGAGTILRVKSYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 88 - protein, 309 aa, synthetic construct
  REGION 1..309: note = Synthetic
MSISLLCCAAFPLLWAGPVNAGVTQTPKFRILKIGQSMTLQCTQDMNHNYMYWYRQDPGMGLKLIYYSVGAGITDKGEVPNGYNVSRSTTEDFPLRLELAAPSQTSVYFCASTLAGRAYEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 89 - protein, 270 aa, synthetic construct
  REGION 1..270: note = Synthetic
MKRILGALLGLLSAQVCCVRGIQVEQSPPDLILQEGANSTLRCNFSDSVNNLQWFHQNPWGQLINLFYIPSGTKQNGRLSATTVATERYSLLYISSSQTTDSGVYFCAVGVLRDYKLSFGAGTTVTVRANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 90 - protein, 309 aa, synthetic construct
  REGION 1..309: note = Synthetic
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASIQGTGLAYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 91 - protein, 272 aa, synthetic construct
  REGION 1..272: note = Synthetic
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVAVFPGNQFYFGTGTSLTVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 92 - protein, 309 aa, synthetic construct
  REGION 1..309: note = Synthetic
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASNLGQAIKYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 93 - protein, 280 aa, synthetic construct
  REGION 1..280: note = Synthetic
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRSAMYSGGGADGLTFGKGTHLIIQPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 94 - protein, 310 aa, synthetic construct
  REGION 1..310: note = Synthetic
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSLGGSFQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 95 - protein, 272 aa, synthetic construct
  REGION 1..272: note = Synthetic
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDAYNFNKFYFGSGTKLNVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 96 - protein, 308 aa, synthetic construct
  REGION 1..308: note = Synthetic
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASRANTGELFFGEGSRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 97 - protein, 278 aa, synthetic construct
  REGION 1..278: note = Synthetic
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSERPGGATNKLIFGTGTLLAVQPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 98 - protein, 309 aa, synthetic construct
  REGION 1..309: note = Synthetic
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASIVGQGNEQFFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 99 - protein, 272 aa, synthetic construct
  REGION 1..272: note = Synthetic
MMKCPQALLAIFWLLLSWVSSEDKVVQSPLSLVVHEGDTVTLNCSYEVTNFRSLLWYKQEKKAPTFLFMLTSSGIEKKSGRLSSILDKKELSSILNITATQTGDSAIYLCAVCDSWGKLQFGAGTQVVVTPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 100 - protein, 310 aa, synthetic construct
  REGION 1..310: note = Synthetic
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASSPLRDYFNEQFFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 101 - protein, 276 aa, synthetic construct
  REGION 1..276: note = Synthetic
MMKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVKGSGTSYGKLTFGQGTILTVHPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 102 - protein, 307 aa, synthetic construct
  REGION 1..307: note = Synthetic
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSLTGYEQFFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 103 - protein, 275 aa, synthetic construct
  REGION 1..275: note = Synthetic
MMKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNSGGGADGLTFGKGTHLIIQPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 104 - protein, 304 aa, synthetic construct
  REGION 1..304: note = Synthetic
MLLLLLLLGPGSGLGAVVSQHPSRVICKSGTSVKIECRSLDFQATTMFWYRQFPKQSLMLMATSNEGSKATYEQGVEKDKFLINHASLTLSTLTVTSAHPEDSSFYICSATEGTPQFFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 105 - protein, 271 aa, synthetic construct
  REGION 1..271: note = Synthetic
MVLKFSVSILWIQLAWVSTQLLEQSPQFLSIQEGENLTVYCNSSSVFSSLQWYRQEPGEGPVLLVTVVTGGEVKKLKRLTFQFGDARKDSSLHITAAQPGDTGLYLCAGEAFGGNYQLIWGAGTKLIIKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 106 - protein, 313 aa, synthetic construct
  REGION 1..313: note = Synthetic
MGCRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSPDLAGVNEQFFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 107 - protein, 273 aa, synthetic construct
  REGION 1..273: note = Synthetic
MLLEHLLIILWMQLTWVSGQQLNQSPQSMFIQEGEDVSMNCTSSSIFNTWLWYKQEPGEGPVLLIALYKAGELTSNGRLTAQFGITRKDSFLNISASIPSDVGIYFCAGPNAGGTSYGKLTFGQGTILTVHPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 108 - protein, 309 aa, synthetic construct
  REGION 1..309: note = Synthetic
MGCRLLCCAVLCLLGAVPMETGVTQTPRHLVMGMTNKKSLKCEQHLGHNAMYWYKQSAKKPLELMFVYNFKEQTENNSVPSRFSPECPNSSHLFLHLHTLQPEDSALYLCASSQDLAIGEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 109 - protein, 270 aa, synthetic construct
  REGION 1..270: note = Synthetic
MRLVARVTVFLTFGTIIDAKTTQPPSMDCAEGRAANLPCNHSTISGNEYVYWYRQIHSQGPQYIIHGLKNNETNEMASLIITEDRKSSTLILPHATLRDTAVYYCIVRVPSDNFNKFYFGSGTKLNVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 110 - protein, 307 aa, synthetic construct
  REGION 1..307: note = Synthetic
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSRTFRELFFGEGSRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 111 - protein, 277 aa, synthetic construct
  REGION 1..277: note = Synthetic
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSEARGGADGLTFGKGTHLIIQPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 112 - protein, 311 aa, synthetic construct
  REGION 1..311: note = Synthetic
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASSYSGTSGIYEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 113 - protein, 273 aa, synthetic construct
  REGION 1..273: note = Synthetic
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVNGGYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 114 - protein, 312 aa, synthetic construct
  REGION 1..312: note = Synthetic
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASSYSLSGTSSYEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 115 - protein, 272 aa, synthetic construct
  REGION 1..272: note = Synthetic
MMKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNAGNQFYFGTGTSLTVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 116 - protein, 309 aa, synthetic construct
  REGION 1..309: note = Synthetic
MGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASSPDRSADTQYFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 117 - protein, 271 aa, synthetic construct
  REGION 1..271: note = Synthetic
MMKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVGDYKLSFGAGTTVTVRANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 118 - protein, 308 aa, synthetic construct
  REGION 1..308: note = Synthetic
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSASRDIDSGNTIYFGEGSWLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 119 - protein, 276 aa, synthetic construct
  REGION 1..276: note = Synthetic
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVQAPYSGAGSYQLTFGKGTKLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 120 - protein, 306 aa, synthetic construct
  REGION 1..306: note = Synthetic
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASKFGDTQYFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 121 - protein, 272 aa, synthetic construct
  REGION 1..272: note = Synthetic
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAVIRGYSGAGSYQLTFGKGTKLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 122 - protein, 306 aa, synthetic construct
  REGION 1..306: note = Synthetic
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASKFGNELFFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 123 - protein, 271 aa, synthetic construct
  REGION 1..271: note = Synthetic
MRLVARVTVFLTFGTIIDAKTTQPPSMDCAEGRAANLPCNHSTISGNEYVYWYRQIHSQGPQYIIHGLKNNETNEMASLIITEDRKSSTLILPHATLRDTAVYYCIVRVPSGAGSYQLTFGKGTKLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 124 - protein, 308 aa, synthetic construct
  REGION 1..308: note = Synthetic
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVVGTREQFFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 125 - protein, 272 aa, synthetic construct
  REGION 1..272: note = Synthetic
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAVRTGYSGAGSYQLTFGKGTKLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 126 - protein, 306 aa, synthetic construct
  REGION 1..306: note = Synthetic
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSYGNEQFFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 127 - protein, 272 aa, synthetic construct
  REGION 1..272: note = Synthetic
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVSPNYGQNFVFGPGTRLSVLPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 128 - protein, 308 aa, synthetic construct
  REGION 1..308: note = Synthetic
MGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASSQKQAYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 129 - protein, 266 aa, synthetic construct
  REGION 1..266: note = Synthetic
MKLVTSITVLLSLGIMGDAKTTQPNSMESNEEEPVHLPCNHSTISGTDYIHWYRQLPSQGPEYVIHGLTSNVNNRMASLAIAEDRKSSTLILHRATLRDAAVYYCITTNAGKSTFGDGTTLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 130 - protein, 309 aa, synthetic construct
  REGION 1..309: note = Synthetic
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSFRAEAYEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 131 - protein, 267 aa, synthetic construct
  REGION 1..267: note = Synthetic
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAVRDNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 132 - protein, 311 aa, synthetic construct
  REGION 1..311: note = Synthetic
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSLGKGRNTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 133 - protein, 273 aa, synthetic construct
  REGION 1..273: note = Synthetic
MMKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVGGSARQLTFGSGTQLTVLPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 134 - protein, 309 aa, synthetic construct
  REGION 1..309: note = Synthetic
MLLLLLLLGPGSGLGAVVSQHPSRVICKSGTSVKIECRSLDFQATTMFWYRQFPKQSLMLMATSNEGSKATYEQGVEKDKFLINHASLTLSTLTVTSAHPEDSSFYICSAPGLAGDPYNEQFFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 135 - protein, 275 aa, synthetic construct
  REGION 1..275: note = Synthetic
MMKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNPLNNFNKFYFGSGTKLNVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 136 - protein, 309 aa, synthetic construct
  REGION 1..309: note = Synthetic
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSGAENTGELFFGEGSRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 137 - protein, 275 aa, synthetic construct
  REGION 1..275: note = Synthetic
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVRVAGGTSYGKLTFGQGTILTVHPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 138 - protein, 319 aa, synthetic construct
  REGION 1..319: note = Synthetic
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSGQGGSDTQYFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 139 - protein, 272 aa, synthetic construct
  REGION 1..272: note = Synthetic
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVQGGSNYKLTFGKGTLLTVNPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 140 - protein, 309 aa, synthetic construct
  REGION 1..309: note = Synthetic
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSSSETGELFFGEGSRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 141 - protein, 271 aa, synthetic construct
  REGION 1..271: note = Synthetic
MVLKFSVSILWIQLAWVSTQLLEQSPQFLSIQEGENLTVYCNSSSVFSSLQWYRQEPGEGPVLLVTVVTGGEVKKLKRLTFQFGDARKDSSLHITAAQPGDTGLYLCAGAGSNSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 142 - protein, 312 aa, synthetic construct
  REGION 1..312: note = Synthetic
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSLVTGGLVDYEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 143 - protein, 268 aa, synthetic construct
  REGION 1..268: note = Synthetic
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVNEGMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 144 - protein, 310 aa, synthetic construct
  REGION 1..310: note = Synthetic
MGTSLLCWVVLGFLGTDHTGAGVSQSPRYKVTKRGQDVALRCDPISGHVSLYWYRQALGQGPEFLTYFNYEAQQDKSGLPNDRFSAERPEGSISTLTIQRTEQRDSAMYRCASSLLTGTGELFFGEGSRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 145 - protein, 274 aa, synthetic construct
  REGION 1..274: note = Synthetic
MLLLLIPVLGMIFALRDARAQSVSQHNHHVILSEAASLELGCNYSYGGTVNLFWYVQYPGQHLQLLLKYFSGDPLVKGIKGFEAEFIKSKFSFNLRKPSVQWSDTAEYFCAVNSGGGADGLTFGKGTHLIIQPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 146 - protein, 309 aa, synthetic construct
  REGION 1..309: note = Synthetic
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASEGLAGAEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 147 - protein, 284 aa, synthetic construct
  REGION 1..284: note = Synthetic
MDKILGASFLVLWLQLCWVSGQQKEKSDQQQVKQSPQSLIVQKGGISIINCAYENTAFDYFPWYQQFPGKGPALLIAIRPDVSEKKEGRFTISFNKSAKQFSLHIMDSQPGDSATYFCAAPMPMDTGRRALTFGSGTRLQVQPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 148 - protein, 308 aa, synthetic construct
  REGION 1..308: note = Synthetic
MGTRLFFYVALCLLWTGHMDAGITQSPRHKVTETGTPVTLRCHQTENHRYMYWYRQDPGHGLRLIHYSYGVKDTDKGEVSDGYSVSRSKTEDFLLTLESATSSQTSVYFCAISGGSVGEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 149 - protein, 272 aa, synthetic construct
  REGION 1..272: note = Synthetic
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVSSGSARQLTFGSGTQLTVLPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 150 - protein, 311 aa, synthetic construct
  REGION 1..311: note = Synthetic
MGPGLLCWVLLCLLGAGSVETGVTQSPTHLIKTRGQQVTLRCSSQSGHNTVSWYQQALGQGPQFIFQYYREEENGRGNFPPRFSGLQFPNYSSELNVNALELDDSALYLCASTLSTGQGIYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 151 - protein, 273 aa, synthetic construct
  REGION 1..273: note = Synthetic
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVGGSGSARQLTFGSGTQLTVLPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 152 - protein, 311 aa, synthetic construct
  REGION 1..311: note = Synthetic
MGPGLLCWVLLCLLGAGSVETGVTQSPTHLIKTRGQQVTLRCSSQSGHNTVSWYQQALGQGPQFIFQYYREEENGRGNFPPRFSGLQFPNYSSELNVNALELDDSALYLCASSPTTSGRIGELFFGEGSRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 153 - protein, 132 aa, synthetic construct
  REGION 1..132: note = Synthetic
MTSIRAVFIFLWLQLDLVNGENVEQHPSTLSVQEGDSAVIKCTYSDSASNYFPWYKQELGKGPQLIIDIRSNVGEKKDQRIAVTLNKTAKHFSLHITETQPEDSAVYFCAASDSSASKIIFGSGTRLSIRPN

SEQ ID NO: 154 - protein, 130 aa, synthetic construct
  REGION 1..130: note = Synthetic
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVGKETQYFGPGTRLLVL

SEQ ID NO: 155 - protein, 138 aa, synthetic construct
  REGION 1..138: note = Synthetic
MSLSSLLKVVTASLWLGPGIAQKITQTQPGMFVQEKEAVTLDCTYDTSDQSYGLFWYKQPSSGEMIFLIYQGSYDEQNATEGRYSLNFQKARKSANLVISASQLGDSAMYFCAMREPYYNQGGKLIFGQGTELSVKPN

SEQ ID NO: 156 - protein, 136 aa, synthetic construct
  REGION 1..136: note = Synthetic
MGCRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQDRLARDTQYFGPGTRLTVL

SEQ ID NO: 157 - protein, 125 aa, synthetic construct
  REGION 1..125: note = Synthetic
MKLVTSITVLLSLGIMGDAKTTQPNSMESNEEEPVHLPCNHSTISGTDYIHWYRQLPSQGPEYVIHGLTSNVNNRMASLAIAEDRKSSTLILHRATLRDAAVYYCILRAVYVRFGAGTRLTVKPN

SEQ ID NO: 158 - protein, 133 aa, synthetic construct
  REGION 1..133: note = Synthetic
MGPGLLCWVLLCLLGAGPVDAGVTQSPTHLIKTRGQQVTLRCSPISGHKSVSWYQQVLGQGPQFIFQYYEKEERGRGNFPDRFSARQFPNYSSELNVNALLLGDSALYLCASSWKGVYNQPQHFGDGTRLSIL

SEQ ID NO: 159 - protein, 134 aa, synthetic construct
  REGION 1..134: note = Synthetic
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVQATSGSARQLTFGSGTQLTVLPD

SEQ ID NO: 160 - protein, 134 aa, synthetic construct
  REGION 1..134: note = Synthetic
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLSKTGRYNEQFFGPGTRLTVL

SEQ ID NO: 161 - protein, 132 aa, synthetic construct
  REGION 1..132: note = Synthetic
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATVFYGNNRLAFGKGNQVVVIPN

SEQ ID NO: 162 - protein, 133 aa, synthetic construct
  REGION 1..133: note = Synthetic
MGPGLLCWVLLCLLGAGPVDAGVTQSPTHLIKTRGQQVTLRCSPISGHKSVSWYQQVLGQGPQFIFQYYEKEERGRGNFPDRFSARQFPNYSSELNVNALLLGDSALYLCASSFFPDSNQPQHFGDGTRLSIL

SEQ ID NO: 163 - protein, 131 aa, synthetic construct
  REGION 1..131: note = Synthetic
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVHMEYGNKLVFGAGTILRVKSY

SEQ ID NO: 164 - protein, 132 aa, synthetic construct
  REGION 1..132: note = Synthetic
MSISLLCCAAFPLLWAGPVNAGVTQTPKFRILKIGQSMTLQCTQDMNHNYMYWYRQDPGMGLKLIYYSVGAGITDKGEVPNGYNVSRSTTEDFPLRLELAAPSQTSVYFCASTLAGRAYEQYFGPGTRLTVT

SEQ ID NO: 165 - protein, 130 aa, synthetic construct
  REGION 1..130: note = Synthetic
MKRILGALLGLLSAQVCCVRGIQVEQSPPDLILQEGANSTLRCNFSDSVNNLQWFHQNPWGQLINLFYIPSGTKQNGRLSATTVATERYSLLYISSSQTTDSGVYFCAVGVLRDYKLSFGAGTTVTVRAN

SEQ ID NO: 166 - protein, 132 aa, synthetic construct
  REGION 1..132: note = Synthetic
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASIQGTGLAYTFGSGTRLTVV

SEQ ID NO: 167 - protein, 132 aa, synthetic construct
  REGION 1..132: note = Synthetic
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVAVFPGNQFYFGTGTSLTVIPN

SEQ ID NO: 168 - protein, 132 aa, synthetic construct
  REGION 1..132: note = Synthetic
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASNLGQAIKYTFGSGTRLTVV

SEQ ID NO: 169 - protein, 140 aa, synthetic construct
  REGION 1..140: note = Synthetic
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRSAMYSGGGADGLTFGKGTHLIIQPY

SEQ ID NO: 170 - protein, 133 aa, synthetic construct
  REGION 1..133: note = Synthetic
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSLGGSFQPQHFGDGTRLSIL

SEQ ID NO: 171 - protein, 132 aa, synthetic construct
  REGION 1..132: note = Synthetic
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDAYNFNKFYFGSGTKLNVKPN

SEQ ID NO: 172 - protein, 131 aa, synthetic construct
  REGION 1..131: note = Synthetic
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASRANTGELFFGEGSRLTVL

SEQ ID NO: 173 - protein, 138 aa, synthetic construct
  REGION 1..138: note = Synthetic
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSERPGGATNKLIFGTGTLLAVQPN

SEQ ID NO: 174 - protein, 132 aa, synthetic construct
  REGION 1..132: note = Synthetic
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASIVGQGNEQFFGPGTRLTVL

SEQ ID NO: 175 - protein, 132 aa, synthetic construct
  REGION 1..132: note = Synthetic
MMKCPQALLAIFWLLLSWVSSEDKVVQSPLSLVVHEGDTVTLNCSYEVTNFRSLLWYKQEKKAPTFLFMLTSSGIEKKSGRLSSILDKKELSSILNITATQTGDSAIYLCAVCDSWGKLQFGAGTQVVVTPD

SEQ ID NO: 176 - protein, 133 aa, synthetic construct
  REGION 1..133: note = Synthetic
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASSPLRDYFNEQFFGPGTRLTVL

SEQ ID NO: 177 - protein, 136 aa, synthetic construct
  REGION 1..136: note = Synthetic
MMKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVKGSGTSYGKLTFGQGTILTVHPN

SEQ ID NO: 178 - protein, 130 aa, synthetic construct
  REGION 1..130: note = Synthetic
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSLTGYEQFFGPGTRLTVL

SEQ ID NO: 179 - protein, 135 aa, synthetic construct
  REGION 1..135: note = Synthetic
MMKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNSGGGADGLTFGKGTHLIIQPY

SEQ ID NO: 180 - protein, 127 aa, synthetic construct
  REGION 1..127: note = Synthetic
MLLLLLLLGPGSGLGAVVSQHPSRVICKSGTSVKIECRSLDFQATTMFWYRQFPKQSLMLMATSNEGSKATYEQGVEKDKFLINHASLTLSTLTVTSAHPEDSSFYICSATEGTPQFFGPGTRLTVL

SEQ ID NO: 181 - protein, 131 aa, synthetic construct
  REGION 1..131: note = Synthetic
MVLKFSVSILWIQLAWVSTQLLEQSPQFLSIQEGENLTVYCNSSSVFSSLQWYRQEPGEGPVLLVTVVTGGEVKKLKRLTFQFGDARKDSSLHITAAQPGDTGLYLCAGEAFGGNYQLIWGAGTKLIIKPD

SEQ ID NO: 182 - protein, 136 aa, synthetic construct
  REGION 1..136: note = Synthetic
MGCRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSPDLAGVNEQFFGPGTRLTVL

SEQ ID NO: 183 - protein, 133 aa, synthetic construct
  REGION 1..133: note = Synthetic
MLLEHLLIILWMQLTWVSGQQLNQSPQSMFIQEGEDVSMNCTSSSIFNTWLWYKQEPGEGPVLLIALYKAGELTSNGRLTAQFGITRKDSFLNISASIPSDVGIYFCAGPNAGGTSYGKLTFGQGTILTVHPN

SEQ ID NO: 184 - protein, 132 aa, synthetic construct
  REGION 1..132: note = Synthetic
MGCRLLCCAVLCLLGAVPMETGVTQTPRHLVMGMTNKKSLKCEQHLGHNAMYWYKQSAKKPLELMFVYNFKEQTENNSVPSRFSPECPNSSHLFLHLHTLQPEDSALYLCASSQDLAIGEQYFGPGTRLTVT

SEQ ID NO: 185 - protein, 130 aa, synthetic construct
  REGION 1..130: note = Synthetic
MRLVARVTVFLTFGTIIDAKTTQPPSMDCAEGRAANLPCNHSTISGNEYVYWYRQIHSQGPQYIIHGLKNNETNEMASLIITEDRKSSTLILPHATLRDTAVYYCIVRVPSDNFNKFYFGSGTKLNVKPN

SEQ ID NO: 186 - protein, 130 aa, synthetic construct
  REGION 1..130: note = Synthetic
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSRTFRELFFGEGSRLTVL

SEQ ID NO: 187 - protein, 137 aa, synthetic construct
  REGION 1..137: note = Synthetic
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSEARGGADGLTFGKGTHLIIQPY

SEQ ID NO: 188 - protein, 134 aa, synthetic construct
  REGION 1..134: note = Synthetic
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASSYSGTSGIYEQYFGPGTRLTVT

SEQ ID NO: 189 - protein, 133 aa, synthetic construct
  REGION 1..133: note = Synthetic
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVNGGYNNNDMRFGAGTRLTVKPN

SEQ ID NO: 190 - protein, 135 aa, synthetic construct
  REGION 1..135: note = Synthetic
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASSYSLSGTSSYEQYFGPGTRLTVT

SEQ ID NO: 191 - protein, 132 aa, synthetic construct
  REGION 1..132: note = Synthetic
MMKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNAGNQFYFGTGTSLTVIPN

SEQ ID NO: 192 - protein, 132 aa, synthetic construct
  REGION 1..132: note = Synthetic
MGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASSPDRSADTQYFGPGTRLTVL

SEQ ID NO: 193 - protein, 131 aa, synthetic construct
  REGION 1..131: note = Synthetic
MMKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVGDYKLSFGAGTTVTVRAN

SEQ ID NO: 194 - protein, 131 aa, synthetic construct
  REGION 1..131: note = Synthetic
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSASRDIDSGNTIYFGEGSWLTVV

SEQ ID NO: 195 - protein, 136 aa, synthetic construct
  REGION 1..136: note = Synthetic
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVQAPYSGAGSYQLTFGKGTKLSVIPN

SEQ ID NO: 196 - protein, 129 aa, synthetic construct
  REGION 1..129: note = Synthetic
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASKFGDTQYFGPGTRLTVL

SEQ ID NO: 197 - protein, 132 aa, synthetic construct
  REGION 1..132: note = Synthetic
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAVIRGYSGAGSYQLTFGKGTKLSVIPN

SEQ ID NO: 198 - protein, 129 aa, synthetic construct
  REGION 1..129: note = Synthetic
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASKFGNELFFGPGTRLTVL

SEQ ID NO: 199 - protein, 131 aa, synthetic construct
  REGION 1..131: note = Synthetic
MRLVARVTVFLTFGTIIDAKTTQPPSMDCAEGRAANLPCNHSTISGNEYVYWYRQIHSQGPQYIIHGLKNNETNEMASLIITEDRKSSTLILPHATLRDTAVYYCIVRVPSGAGSYQLTFGKGTKLSVIPN

SEQ ID NO: 200 - protein, 131 aa, synthetic construct
  REGION 1..131: note = Synthetic
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVVGTREQFFGPGTRLTVL

SEQ ID NO: 201 - protein, 132 aa, synthetic construct
  REGION 1..132: note = Synthetic
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAVRTGYSGAGSYQLTFGKGTKLSVIPN

SEQ ID NO: 202 - protein, 129 aa, synthetic construct
  REGION 1..129: note = Synthetic
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSYGNEQFFGPGTRLTVL

SEQ ID NO: 203 - protein, 132 aa, synthetic construct
  REGION 1..132: note = Synthetic
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVSPNYGQNFVFGPGTRLSVLPY

SEQ ID NO: 204 - protein, 131 aa, synthetic construct
  REGION 1..131: note = Synthetic
MGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASSQKQAYGYTFGSGTRLTVV

SEQ ID NO: 205 - protein, 126 aa, synthetic construct
  REGION 1..126: note = Synthetic
MKLVTSITVLLSLGIMGDAKTTQPNSMESNEEEPVHLPCNHSTISGTDYIHWYRQLPSQGPEYVIHGLTSNVNNRMASLAIAEDRKSSTLILHRATLRDAAVYYCITTNAGKSTFGDGTTLTVKPN

SEQ ID NO: 206 - protein, 132 aa, synthetic construct
  REGION 1..132: note = Synthetic
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSFRAEAYEQYFGPGTRLTVT

SEQ ID NO: 207 - protein, 127 aa, synthetic construct
  REGION 1..127: note = Synthetic
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAVRDNNNDMRFGAGTRLTVKPN

SEQ ID NO: 208 - protein, 134 aa, synthetic construct
  REGION 1..134: note = Synthetic
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSLGKGRNTEAFFGQGTRLTVV

SEQ ID NO: 209 - protein, 133 aa, synthetic construct
  REGION 1..133: note = Synthetic
MMKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVGGSARQLTFGSGTQLTVLPD

SEQ ID NO: 210 - protein, 132 aa, synthetic construct
  REGION 1..132: note = Synthetic
MLLLLLLLGPGSGLGAVVSQHPSRVICKSGTSVKIECRSLDFQATTMFWYRQFPKQSLMLMATSNEGSKATYEQGVEKDKFLINHASLTLSTLTVTSAHPEDSSFYICSAPGLAGDPYNEQFFGPGTRLTVL

SEQ ID NO: 211 - protein, 135 aa, synthetic construct
  REGION 1..135: note = Synthetic
MMKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNPLNNFNKFYFGSGTKLNVKPN

SEQ ID NO: 212 - protein, 132 aa, synthetic construct
  REGION 1..132: note = Synthetic
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSGAENTGELFFGEGSRLTVL

SEQ ID NO: 213 - protein, 135 aa, synthetic construct
  REGION 1..135: note = Synthetic
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVRVAGGTSYGKLTFGQGTILTVHPN

SEQ ID NO: 214 - protein, 142 aa, synthetic construct
  REGION 1..142: note = Synthetic
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSGQGGSDTQYFGPGTRLTVL

SEQ ID NO: 215 - protein, 132 aa, synthetic construct
  REGION 1..132: note = Synthetic
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVQGGSNYKLTFGKGTLLTVNPN

SEQ ID NO: 216 - protein, 132 aa, synthetic construct
  REGION 1..132: note = Synthetic
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSSSETGELFFGEGSRLTVL

SEQ ID NO: 217 - protein, 131 aa, synthetic construct
  REGION 1..131: note = Synthetic
MVLKFSVSILWIQLAWVSTQLLEQSPQFLSIQEGENLTVYCNSSSVFSSLQWYRQEPGEGPVLLVTVVTGGEVKKLKRLTFQFGDARKDSSLHITAAQPGDTGLYLCAGAGSNSGYALNFGKGTSLLVTPH

SEQ ID NO: 218 - protein, 135 aa, synthetic construct
  REGION 1..135: note = Synthetic
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSLVTGGLVDYEQYFGPGTRLTVT

SEQ ID NO: 219 - protein, 128 aa, synthetic construct
  REGION 1..128: note = Synthetic
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVNEGMRFGAGTRLTVKPN

SEQ ID NO: 220 - protein, 133 aa, synthetic construct
  REGION 1..133: note = Synthetic
MGTSLLCWVVLGFLGTDHTGAGVSQSPRYKVTKRGQDVALRCDPISGHVSLYWYRQALGQGPEFLTYFNYEAQQDKSGLPNDRFSAERPEGSISTLTIQRTEQRDSAMYRCASSLLTGTGELFFGEGSRLTVL

SEQ ID NO: 221 - protein, 134 aa, synthetic construct
  REGION 1..134: note = Synthetic
MLLLLIPVLGMIFALRDARAQSVSQHNHHVILSEAASLELGCNYSYGGTVNLFWYVQYPGQHLQLLLKYFSGDPLVKGIKGFEAEFIKSKFSFNLRKPSVQWSDTAEYFCAVNSGGGADGLTFGKGTHLIIQPY

SEQ ID NO: 222 - protein, 132 aa, synthetic construct
  REGION 1..132: note = Synthetic
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASEGLAGAEQYFGPGTRLTVT

SEQ ID NO: 223 - protein, 144 aa, synthetic construct
  REGION 1..144: note = Synthetic
MDKILGASFLVLWLQLCWVSGQQKEKSDQQQVKQSPQSLIVQKGGISIINCAYENTAFDYFPWYQQFPGKGPALLIAIRPDVSEKKEGRFTISFNKSAKQFSLHIMDSQPGDSATYFCAAPMPMDTGRRALTFGSGTRLQVQPN

SEQ ID NO: 224 - protein, 131 aa, synthetic construct
  REGION 1..131: note = Synthetic
MGTRLFFYVALCLLWTGHMDAGITQSPRHKVTETGTPVTLRCHQTENHRYMYWYRQDPGHGLRLIHYSYGVKDTDKGEVSDGYSVSRSKTEDFLLTLESATSSQTSVYFCAISGGSVGEQYFGPGTRLTVT

SEQ ID NO: 225 - protein, 132 aa, synthetic construct
  REGION 1..132: note = Synthetic
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVSSGSARQLTFGSGTQLTVLPD

SEQ ID NO: 226 - protein, 134 aa, synthetic construct
  REGION 1..134: note = Synthetic
MGPGLLCWVLLCLLGAGSVETGVTQSPTHLIKTRGQQVTLRCSSQSGHNTVSWYQQALGQGPQFIFQYYREEENGRGNFPPRFSGLQFPNYSSELNVNALELDDSALYLCASTLSTGQGIYGYTFGSGTRLTVV

SEQ ID NO: 227 - protein, 133 aa, synthetic construct
  REGION 1..133: note = Synthetic
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVGGSGSARQLTFGSGTQLTVLPD

SEQ ID NO: 228 - protein, 134 aa, synthetic construct
  REGION 1..134: note = Synthetic
MGPGLLCWVLLCLLGAGSVETGVTQSPTHLIKTRGQQVTLRCSSQSGHNTVSWYQQALGQGPQFIFQYYREEENGRGNFPPRFSGLQFPNYSSELNVNALELDDSALYLCASSPTTSGRIGELFFGEGSRLTVL

SEQ ID NO: 229 - protein, 9 aa, synthetic construct
  REGION 1..9: note = Synthetic
FAFGEPREL

SEQ ID NO: 230 - protein, 12 aa, synthetic construct
  REGION 1..12: note = Synthetic
RRWDEKAVDKSK

SEQ ID NO: 231 - protein, 10 aa, synthetic construct
  REGION 1..10: note = Synthetic
GTDSDSSRQK

SEQ ID NO: 232 - protein, 9 aa, synthetic construct
  REGION 1..9: note = Synthetic
GLYDGMEHL

SEQ ID NO: 233 - protein, 10 aa, synthetic construct
  REGION 1..10: note = Synthetic
ELAGIGILTV

SEQ ID NO: 234 - protein, 9 aa, synthetic construct
  REGION 1..9: note = Synthetic
TADFDITEL

SEQ ID NO: 235 - protein, 7 aa, synthetic construct
  REGION 1..7: note = Synthetic
LPEEKQP

SEQ ID NO: 236 - protein, 9 aa, synthetic construct
  REGION 1..9: note = Synthetic
VQTFLGISF

SEQ ID NO: 237 - protein, 10 aa, synthetic construct
  REGION 1..10: note = Synthetic
RLPSSADVEF

SEQ ID NO: 238 - protein, 9 aa, synthetic construct
  REGION 1..9: note = Synthetic
ALPGFFPRL

SEQ ID NO: 239 - protein, 9 aa, synthetic construct
  REGION 1..9: note = Synthetic
EPLHTPTIM

SEQ ID NO: 240 - protein, 9 aa, synthetic construct
  REGION 1..9: note = Synthetic
IPNPKAGIM

SEQ ID NO: 241 - protein, 10 aa, synthetic construct
  REGION 1..10: note = Synthetic
QSDNGLDSDY

SEQ ID NO: 242 - protein, 8 aa, synthetic construct
  REGION 1..8: note = Synthetic
LPEEKQPL

SEQ ID NO: 243 - protein, 9 aa, synthetic construct
  REGION 1..9: note = Synthetic
ILAKFLHWL

SEQ ID NO: 244 - protein, 9 aa, synthetic construct
  REGION 1..9: note = Synthetic
LTLGEFLKL

SEQ ID NO: 245 - protein, 8 aa, synthetic construct
  REGION 1..8: note = Synthetic
LLGRNSFE

SEQ ID NO: 246 - protein, 9 aa, synthetic construct
  REGION 1..9: note = Synthetic
VLPLTVAEV

SEQ ID NO: 247 - protein, 9 aa, synthetic construct
  REGION 1..9: note = Synthetic
SLLMWITQC

SEQ ID NO: 248 - protein, 9 aa, synthetic construct
  REGION 1..9: note = Synthetic
IISAVVGIL

SEQ ID NO: 249 - protein, 9 aa, synthetic construct
  REGION 1..9: note = Synthetic
KIFGSLAFL

SEQ ID NO: 250 - protein, 9 aa, synthetic construct
  REGION 1..9: note = Synthetic
RLLQETELV

SEQ ID NO: 251 - protein, 9 aa, synthetic construct
  REGION 1..9: note = Synthetic
RMFPNAPYL

SEQ ID NO: 252 - protein, 9 aa, synthetic construct
  REGION 1..9: note = Synthetic
STAPPVHNV

SEQ ID NO: 253 - protein, 9 aa, synthetic construct
  REGION 1..9: note = Synthetic
SEHESNLLW

SEQ ID NO: 254 - protein, 10 aa, synthetic construct
  REGION 1..10: note = Synthetic
QKLRNSRKTW

SEQ ID NO: 255 - protein, 9 aa, synthetic construct
  REGION 1..9: note = Synthetic
AGYDAPRAV

SEQ ID NO: 256 - protein, 9 aa, synthetic construct
  REGION 1..9: note = Synthetic
MPNPQEFTY

SEQ ID NO: 257 - protein, 10 aa, synthetic construct
  REGION 1..10: note = Synthetic
GEYISCVAWI

SEQ ID NO: 258 - protein, 10 aa, synthetic construct
  REGION 1..10: note = Synthetic
DYMIHIIEKW

SEQ ID NO: 259 - protein, 22 aa, synthetic construct
  REGION 1..22: note = Synthetic
KAKARRWDEKAVDKSKEKKERL

SEQ ID NO: 260 - protein, 18 aa, synthetic construct
  REGION 1..18: note = Synthetic
ISVEKLNLGTDSDSSRQK

SEQ ID NO: 261 - protein, 25 aa, synthetic construct
  REGION 1..25: note = Synthetic
TPSPTFSTADFDITELADEQQDEME

SEQ ID NO: 262 - protein, 21 aa, synthetic construct
  REGION 1..21: note = Synthetic
IKKERQKLRNSRKTWNHFSDN

SEQ ID NO: 263 - protein, 24 aa, synthetic construct
  REGION 1..24: note = Synthetic
WGLDSYDYLQNALPGFFPRLGVIG

SEQ ID NO: 264 - protein, 24 aa, synthetic construct
  REGION 1..24: note = Synthetic
GSGMCKAGFAGYDAPRAVFPSIVG

SEQ ID NO: 265 - protein, 31 aa, synthetic construct
  REGION 1..31: note = Synthetic
HLELALEYWCPTEPLHTPTIMGSYLGVAHQR

SEQ ID NO: 266 - protein, 31 aa, synthetic construct
  REGION 1..31: note = Synthetic
ISGESSATNEPRGASMPNPQEFTYSSPTPDM

SEQ ID NO: 267 - protein, 31 aa, synthetic construct
  REGION 1..31: note = Synthetic
GCQGKPLPKDLRFIPNPKAGIMIKSVKRAYH

SEQ ID NO: 268 - protein, 31 aa, synthetic construct
  REGION 1..31: note = Synthetic
ATFSSNQSDNGLDSDYDQPVEGVITNGSKVE

(19) United States
(12) Patent Application Publication
(10) Pub. No.: US 2024/0327491 A1
(43) Pub. Date: Oct. 3, 2024

(54) T CELL RECEPTORS SPECIFIC FOR TUMOR-ASSOCIATED ANTIGENS AND METHODS OF USE THEREOF

(71) Applicant: Ludwig Institute for Cancer Research Ltd, Zurich (CH)

(72) Inventors: George Coukos, Epalinges (CH); Alexandre Harari, Épalinges (CH); Sara Bobisse, Épalinges (CH); Marion Arnaud, Épalinges (CH); Michal Bassani-Sternberg, Épalinges (CH)

(21) Appl. No.: 18/576,468

(22) PCT Filed: Jul. 11, 2022
(86) PCT No.: PCT/US22/73611

§ 371 Date: Jan. 4, 2024

Related U.S. Application Data
(60) Provisional application No. 63/220,593, filed on Jul. 12, 2021.

(51) Int. Cl. C07K 14/725; A61K 39/00; A61P 35/00; C12N 5/0783; G01N 33/574

(52) U.S. Cl. CPC C07K 14/7051; A61K 39/4611; A61K 39/4632; A61K 39/464401; A61P 35/00; C12N 5/0636; G01N 33/57484; A61K 2239/31; C12N 2510/00; G01N 2333/7051

(57) ABSTRACT

This disclosure describes novel T cell receptors (TCRs) specific for tumor-associated antigens (TAAs) and methods of use thereof. The disclosed TCRs and methods of use expand the applications of adoptive cell therapy and TCR-based cellular immunotherapies.

### CROSS-REFERENCE TO RELATED APPLICATIONS

This application claims priority under 35 U.S.C. § 119(e) to U.S. Provisional Patent Application No. 63/220,593, filed Jul. 12, 2021. The foregoing application is incorporated by reference herein in its entirety.

### FIELD OF THE INVENTION

This invention relates to T cell receptors specific for tumor-associated antigens and methods of use thereof.

### BACKGROUND OF THE INVENTION

Cancer immunotherapies based on therapeutic vaccination or on the transfer of tumor-infiltrating lymphocytes (TILs) targeting tumor neoantigens have shown promising clinical outcomes. Furthermore, engineering of blood T cells with tumor-reactive T cell receptors (TCRs) further expanded the horizons of adoptive T cell therapy. Identification of clinically relevant tumor antigens and their cognate TCRs is a critical foundation for such therapies. To this end, in vitro-expanded autologous TILs and/or peripheral blood lymphocytes (PBLs) are usually interrogated for tumor antigen discovery. However, the frequency of neoantigen-specific T cells in PBLs and TILs is low, and PBL and TIL repertoires are often discordant. Also, antigen discovery in PBLs remains challenging, despite pioneer work improving the detection of neoantigen reactivity in blood. Although use of TILs could be advantageous, traditional culture methods for in vitro TIL expansion have been shown to skew the ex vivo TIL repertoire (Poschke, I. C. et al. Clin. Cancer Res. 26, 4289-4301 (2020)), thus likely underestimating the quantification of tumor-reactive lymphocytes and curtailing the validation of tumor epitopes. This limits identification of new TCRs and the applications of adoptive T cell therapy. Thus, there remains a need for novel TCRs specific for TAAs and methods of use thereof.

### SUMMARY OF THE INVENTION

This disclosure addresses the need mentioned above in a number of aspects. In one aspect, this disclosure provides a T cell receptor (TCR) or antigen-binding fragment thereof that binds specifically to a tumor-associated antigen (TAA). In some embodiments, the TCR or antigen-binding fragment thereof comprises an amino acid sequence having at least 90% sequence identity to an amino acid sequence of SEQ ID NOs: 1-228 or comprises an amino acid sequence of SEQ ID NOs: 1-228.

In some embodiments, the TCR or antigen-binding fragment thereof comprises an α chain amino acid sequence having at least 90% sequence identity to the amino acid sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, or 227; or comprises an α chain amino acid sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, or 227.

In some embodiments, the TCR or antigen-binding fragment thereof comprises a β chain amino acid sequence having at least 90% sequence identity to the amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, or 228; or comprises a β chain amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, or 228.

In some embodiments, the TCR or antigen-binding fragment thereof comprises: an α chain amino acid sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, or 227; and a β chain amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222,224, 226, or 228.

In some embodiments, the TCR or antigen-binding fragment thereof comprises an α chain complementarity-determining region 3 (CDR3) having an amino acid sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, or 75; and a β chain CDR3 having an amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, or 76.

In some embodiments, the TCR or antigen-binding fragment thereof comprises the α chain amino acid sequence and the R chain amino acid sequence having respective amino acid sequences of SEQ ID NOs: 1-2, 3-4, 5-6, 7-8, 9-10, 11-12, 13-14, 15-16, 17-18, 19-20, 21-22, 23-24, 25-26, 27-28, 29-30, 31-32, 33-34, 35-36, 37-38, 39-40, 41-42, 43-44, 45-46, 47-48, 49-50, 51-52, 53-54, 55-56, 57-58, 59-60, 61-62, 63-64, 65-66, 67-68, 69-70, 71-72, 73-74, 75-76, 77-78, 79-80, 81-82, 83-84, 85-86, 87-88, 89-90, 91-92, 93-94, 95-96, 97-98, 99-100, 101-102, 103-104, 105-106, 107-108, 109-110, 111-112, 113-114, 115-116, 117-118, 119-120, 121-122, 123-124, 125-126, 127-128, 129-130, 131-132, 133-134, 135-136, 137-138, 139-140, 141-142, 143-144, 145-146, 147-148, 149-150, 151-152, 153-154, 155-156, 157-158, 159-160, 161-162, 163-164, 165-166, 167-168, 169-170, 171-172, 173-174, 175-176, 177-178, 179-180, 181-182, 183-184, 185-186, 187-188, 189-190, 191-192, 193-194, 195-196, 197-198, 199-200, 201-202, 203-204, 205-206, 207-208, 209-210, 211-212, 213-214, 215-216, 217-218, 219-220, 221-222, 223-224, 225-226, or 227-228.

In some embodiments, the TCR or antigen-binding fragment thereof comprises the α chain CDR3 and the β chain CDR3 having a amino acid sequence pair of SEQ ID NOs: 1-2, 3-4, 5-6, 7-8, 9-10, 11-12, 13-14, 15-16, 17-18, 19-20, 21-22, 23-24, 25-26, 27-28, 29-30, 31-32, 33-34, 35-36, 37-38, 39-40, 41-42, 43-44, 45-46, 47-48, 49-50, 51-52, 53-54, 55-56, 57-58, 59-60, 61-62, 63-64, 65-66, 67-68, 69-70, 71-72, 73-74, or 75-76.

In some embodiments, the TCR is an αβ heterodimeric TCR. In some embodiments, the TCR is an αβ single chain TCR.

In some embodiments, the TAA is selected from the group consisting of: 707-AP, a biotinylated molecule, a-Actinin-4, abl-bcr alb-b3 (b2a2), abl-bcr alb-b4 (b3a2), adipophilin, AFP, AIM-2, Annexin II, ART-4, BAGE, BCMA, b-Catenin, bcr-abl, bcr-abl p190 (e1a2), bcr-abl p210 (b2a2), bcr-abl p210 (b3a2), BING-4, CA-125, CAG-3, CAIX, CAMEL, Caspase-8, CD171, CD19, CD20, CD22, CD4, CD23, CD24, CD30, CD33, CD38, CD44v7/8, CD70, CD123, CD133, CDC27, CDK-4, CEA, CLCA2, CLL-1, CTAG1B, Cyp-B, DAM-10, DAM-6, DEK-CAN, DLL3, EGFR, EGFRvIII, EGP-2, EGP-40, ELF2, Ep-CAM, EphA2, EphA3, erb-B2, erb-B3, erb-B4, ES-ESO-1a, ETV6/AML, FAP, FBP, fetal acetylcholine receptor, FGF-5, FN, FR-α, G250, GAGE-1, GAGE-2, GAGE-3, GAGE-4, GAGE-5, GAGE-6, GAGE-7B, GAGE-8, GD2, GD3, GnT-V, Gp100, gp75, GPC3, GPC-2, Her-2, HLA-A*0201-R170I, HMW-MAA, HSP70-2 M, HST-2 (FGF6), HST-2/neu, hTERT, iCE, IL-11Ra, IL-13Rα2, KDR, KIAA0205, K-RAS, L1-cell adhesion molecule, LAGE-1, LDLR/FUT, Lewis Y, L1-CAM, MAGE-1, MAGE-10, MAGE-12, MAGE-2, MAGE-3, MAGE-4, MAGE-6, MAGE-A1, MAGE-A2, MAGE-A3, MAGE-A6, MAGE-B1, MAGE-B2, Malic enzyme, Mammaglobin-A, MART-1/Melan-A, MART-2, MC1R, M-CSF, mesothelin, MUC1, MUC16, MUC2, MUM-1, MUM-2, MUM-3, Myosin, NA88-A, Neo-PAP, NKG2D, NPM/ALK, N-RAS, NY-ESO-1, OA1, OGT, oncofetal antigen (h5T4), OS-9, P polypeptide, P15, P53, PRAME, PSA, PSCA, PSMA, PTPRK, RAGE, ROR1, RU1, RU2, SART-1, SART-2, SART-3, SOX10, SSX-2, Survivin, Survivin-2B, SYT/SSX, TAG-72, TEL/AML1, TGFαRII, TGFβRII, TP1, TRAG-3, TRG, TRP-1, TRP-2, TRP-2/INT2, TRP-2-6b, Tyrosinase, VEGF-R2, WT1, α-folate receptor, and κ-light chain.

In some embodiments, the TAA comprises an amino acid sequence of SEQ ID NOs: 229-268.

In another aspect, this disclosure also provides a polypeptide comprising an amino acid sequence having at least 90% sequence identity to an amino acid sequence of SEQ ID NOs: 1-228 or comprising an amino acid sequence of SEQ ID NOs: 1-228.

In some embodiments, the polypeptide comprises a first polypeptide chain that comprises an amino acid sequence having at least 90% sequence identity to the amino acid sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, or 227; or comprises an amino acid sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, or 227.

In some embodiments, the polypeptide comprises a second polypeptide chain that comprises an amino acid sequence having at least 90% sequence identity to the amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, or 228; or comprises an amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, or 228.

In some embodiments, the polypeptide comprises the first polypeptide chain and the second polypeptide chain having respective amino acid sequences of SEQ ID NOs: 1-2, 3-4, 5-6, 7-8, 9-10, 11-12, 13-14, 15-16, 17-18, 19-20, 21-22, 23-24, 25-26, 27-28, 29-30, 31-32, 33-34, 35-36, 37-38, 39-40, 41-42, 43-44, 45-46, 47-48, 49-50, 51-52, 53-54, 55-56, 57-58, 59-60, 61-62, 63-64, 65-66, 67-68, 69-70, 71-72, 73-74, 75-76, 77-78, 79-80, 81-82, 83-84, 85-86, 87-88, 89-90, 91-92, 93-94, 95-96, 97-98, 99-100, 101-102, 103-104, 105-106, 107-108, 109-110, 111-112, 113-114, 115-116, 117-118, 119-120, 121-122, 123-124, 125-126, 127-128, 129-130, 131-132, 133-134, 135-136, 137-138, 139-140, 141-142, 143-144, 145-146, 147-148, 149-150, 151-152, 153-154, 155-156, 157-158, 159-160, 161-162, 163-164, 165-166, 167-168, 169-170, 171-172, 173-174, 175-176, 177-178, 179-180, 181-182, 183-184, 185-186, 187-188, 189-190, 191-192, 193-194, 195-196, 197-198, 199-200, 201-202, 203-204, 205-206, 207-208, 209-210, 211-212, 213-214, 215-216, 217-218, 219-220, 221-222, 223-224, 225-226, or 227-228.

In another aspect, this disclosure additionally provides a nucleic acid comprising a polynucleotide sequence that encodes the TCR or antigen-binding fragment thereof or the polypeptide, as described herein.

Also within the scope of this disclosure is a vector comprising the nucleic acid. In some embodiments, the vector comprises a retroviral vector or a lentiviral vector.

In another aspect, this disclosure further provides a cell comprising the nucleic acid or the vector, as described herein. In some embodiments, the cell comprises an immune cell.

In some embodiments, the immune cell comprises a lymphocyte. In some embodiments, the lymphocyte comprises a T cell or a natural killer (NK) cell. In some embodiments, the T cell comprises a CD8+ T cell or a CD4+ T cell. In some embodiments, the T cell comprises a human T cell.

In another aspect, this disclosure further provides a composition comprising the TCR or antigen-binding fragment thereof, the polypeptide, the nucleic acid, the, or the cell, as described herein.

In some embodiments, the composition further comprises a therapeutic agent. In some embodiments, the therapeutic agent comprises an anti-tumor or anti-cancer agent.

In some embodiments, the anti-tumor or anti-cancer agent is selected from the group consisting of taxotere, carboplatin, trastuzumab, epirubicin, cyclophosphamide, cisplatin, docetaxel, doxorubicin, etoposide, 5-FU, gemcitabine, methotrexate, and paclitaxel, mitoxantrone, epothilone B, epidermal-growth factor receptor (EGFR)-targeting monoclonal antibody 7A7.27, vorinostat, romidepsin, docosahexaenoic acid, bortezomib, shikonin, an oncolytic virus, and combinations thereof.

In some embodiments, the therapeutic agent comprises a chemotherapeutic agent selected from the group consisting of asparaginase, busulfan, carboplatin, cisplatin, daunorubicin, doxorubicin, fluorouracil, gemcitabine, hydroxyurea, methotrexate, paclitaxel, rituximab, vinblastine, and vincristine.

Also provided in this disclosure is a kit comprising the TCR or antigen-binding fragment thereof, the polypeptide, the nucleic acid, the vector, the cell, or the composition, as described herein.

In another aspect, this disclosure also provides a method of preparing a population of cells expressing a TCR specific for a target cell in a subject. The method comprises: (a) isolating a plurality of cells from a subject; (b) transfecting the plurality of cells with the vector described herein; and (c) optionally expanding the transfected cells.

In some embodiments, the subject is a patient. In some embodiments, the subject is a healthy donor. In some embodiments, the target cell comprises a tumor cell.

In another aspect, this disclosure additionally provides a method of directing immune cells to a target cell in a subject. The method comprises administering to the subject the composition described herein.

In another aspect, this disclosure also provides a method for an adoptive T cell therapy in a subject. The method comprises administering to the subject a therapeutically effective amount of the composition described herein.

In another aspect, this disclosure also provides a method for stimulating or enhancing an immune response in a subject in need thereof. The method comprises administering to the subject the composition described herein.

In another aspect, this disclosure also provides a method of preventing or treating cancer in a subject in need thereof. The method comprises administering to the subject a therapeutically effective amount of the composition described herein.

In some embodiments, the cancer is selected from adrenal gland tumors, biliary cancer, bladder cancer, brain cancer, breast cancer, carcinoma, central or peripheral nervous system tissue cancer, cervical cancer, colon cancer, endocrine or neuroendocrine cancer or hematopoietic cancer, esophageal cancer, fibroma, gastrointestinal cancer, glioma, head and neck cancer, Li-Fraumeni tumors, liver cancer, lung cancer, lymphoma, melanoma, meningioma, multiple neuroendocrine type I and type II tumors, nasopharyngeal cancer, oral cancer, oropharyngeal cancer, osteogenic sarcoma tumors, ovarian cancer, pancreatic cancer, pancreatic islet cell cancer, parathyroid cancer, pheochromocytoma, pituitary tumors, prostate cancer, rectal cancer, renal cancer, respiratory cancer, sarcoma, skin cancer, stomach cancer, testicular cancer, thyroid cancer, tracheal cancer, urogenital cancer, and uterine cancer.

In some embodiments, the tumor comprises a solid tumor.

In some embodiments, the method further comprises administering to the subject a second agent or therapy. In some embodiments, the second agent comprises an anti-tumor or anti-cancer agent. In some embodiments, the second agent or therapy is administered before or after the composition. In some embodiments, the second agent or therapy is administered concurrently with the composition. In some embodiments, the composition is administered by intravenous infusion.

In another aspect, this disclosure additionally provides a method of detecting cancer in a biological sample. The method comprises: (a) contacting the biological sample with the TCR or antigen-binding fragment thereof described herein, and (b) detecting binding of the TCR or antigen-binding fragment thereof to the biological sample, wherein detection of binding is indicative of cancer. In some embodiments, the TCR or antigen-binding fragment thereof comprises a detectable label. In some embodiments, the detectable label is selected from the group consisting of a radionuclide, a fluorophore, and biotin.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIGS. 1A, 1B, 1C, 1D, 1E, 1F, and 1G are a set of diagrams showing the process of sensitive tumor antigen discovery. FIG. 1A shows the NeoScreen pipeline. FIGS. 1B, 1C, 1D, and 1E show antigen discovery with NeoScreen (n=7 patients). FIGS. 1B and 1C show representative examples of flow cytometry data (FIG. 1B) and cumulative frequencies (FIG. 1C) of tumor antigen-specific CD8 T cells (n=19 epitopes) in conventional (x-axis) and NeoScreen (y-axis) TIL cultures, by pMHC-multimers or 4-1BB up-regulation. FIG. 1D shows proportions of neoepitope versus TAA-specific among enriched versus newly-detected T cell reactivities. FIG. 1E shows the number of tumor epitopes per patient identified with conventional and NeoScreen strategies (histograms report median values). FIG. 1F shows frequencies of tumor antigen-specific CD8 T cells (n=23 epitopes from nine patients) in conventional (x-axis) and NeoScreen (y-axis) cultures. FIG. 1G shows frequencies of antigen-specific CD8 T cells (n=23) in in vitro-expanded TIL cultures (×2: re-stimulated). Box plots represent median (line), 25% and 75% confidence limit (box limits), and min to max (whiskers). In FIGS. 1C, 1F, and 1G, the background levels of 4-1BB expressed by cognate negative controls were subtracted. In FIGS. 1C and 1F, the highest values between 1×NeoScreen and 2×NeoScreen are considered, and data are displayed in a logarithmic scale. In FIGS. 1C, 1E, 1F, and 1G, P-values were determined with one-tailed paired t-tests.

FIGS. 2A, 2B, 2C, 2D, 2E, 2F, 2G, and 2H are a set of diagrams showing tumor-reactive TCRs identification and validation. FIG. 2A shows a representative example of neoepitope-specific CD8 T cell sorting by pMHC-multimer. The Manhattan plot depicts TCRβ chains V-J recombination of PHLPP2N1186Y-specific clonotypes A, B, and C. FIG. 2B shows validation of antigen-specificity after TCR cloning. FIG. 2C shows a superimposition of the modeled TCR-pMHC complexes for TCR-A, -B, and -C. The location of CDR3a, and CDR3P loops is shown by arrows. FIG. 2D shows violin plots displaying frequencies of TCRβ-A, -B, and -C in bulk TCR repertoires of the different TIL cultures and of the original tumor. NeoScreen TILs from patient 7 were generated with long peptides. FIG. 2E shows the results of a cumulative analysis of the frequency of tumor antigen-specific TCRβ detected in conventional (x-axis) and NeoScreen (y-axis) cultures. The highest values between 1×NeoScreen and 2×NeoScreen are considered, and data are displayed in a logarithmic scale. P-value was determined with a one-tailed paired t-test.

FIG. 2F shows proportions of neoepitope versus TAA-specific TCRβ among enriched versus newly-detected clonotypes. FIG. 2G shows adoptive cell transfer (ACT) of TCR-transduced T cells in an autologous patient-derived xenograft tumor model. FIG. 2H shows in vivo efficacy of adoptively-transferred tyrosinase₅₀₈₋₅₁₄ TCR-transduced T cells against autologous patient-derived tumor xenografts. The graph shows tumor size (mean±SEM of replicates) over time. P-value was determined with a one-tailed unpaired t-test.

FIGS. 3A, 3B, and 3C are a set of graphs showing the phenotype and potency of APCs. FIG. 3A shows representative profiling of viable CD40-act B. Ex vivo peripheral CD19 cells from a healthy donor (HD) were used as control. FIG. 3B shows a comparison of the level of neoepitope-specific T cell stimulation obtained with CD40-act B cells loaded with different sources of antigen. Autologous CD40-act B cells were either pulsed with the minimal epitope, electroporated with RNA-encoding tandem minigene (TMG) or loaded with the 31mer. B cells were co-cultured with ×1NeoScreen TILs from patient 7 (Table 4). T cell reactivity to PHLPP2_{N1186Y} was assessed by IFNγ ELISpot assay (mean±SD of triplicate). MOCK: B cells transfected with PBS. FIG. 3C shows CD40-act B cells electroporated with RNA encoding immune stimulatory molecules OX40L, 4-1BBL, and IL-12. FIG. 3C (left panel) shows the results of a flow cytometry analysis of 4-1BBL and OX40L expression after electroporation of B cells from a representative patient. FIG. 3C (right panel) shows the results of an MSD measurement of IL-12p70 production by electroporated B cells. MOCK: B cells transfected with PBS, NT: non-transfected, stim: stimulatory.

FIGS. 4A, 4B, 4C, 4D, 4E, 4F, 4G, 4H, and 4I are a set of diagrams showing increased detection of tumor antigen-specific CD8 T cells with NeoScreen. FIG. 4A shows the frequency of neoepitope-specific CD8 T cells from patients 6 and 7 measured with pMHC multimers (CTRL: control, NA: not available, LP: long peptide, TMG: tandem minigene). FIG. 4B shows the results of a cumulative analysis of the frequency of tumor antigen-specific T cells (n=4 epitopes, Table 4) in conventional (x-axis) and NeoScreen (y-axis) cultures of patients 6 and 7. FIG. 4C shows a representative example of the frequency of neoepitope and TAA-specific CD8 T cells from patient 1 measured with pMHC multimers. FIG. 4D shows the results of a cumulative analysis of the frequency of tumor antigen-specific T cells (n=9 enriched epitopes from seven patients dedicated to antigen discovery, Table 2) in conventional (x-axis) and NeoScreen (y-axis) cultures by pMHC multimers. FIGS. 4E, 4F, and 4G show the magnitude of tumor antigen-specific CD8 T cells (determined by IFNγ Spot Forming Unit per 10⁵ cells (FIG. 4E, n=22 epitopes), pMHC-multimers staining (FIG. 4F, n=13) or upregulation of 4-1BB (FIG. 4G, n=20)) obtained with NeoScreen or conventional cultures. Box plots represent median (line), 25% and 75% confidence limit (box limits), and min to max (whiskers). FIG. 4H shows cumulative frequencies of tumor antigen-specific T cells, for enriched epitopes only (n=13 epitopes from all nine patients) in conventional (x-axis) and NeoScreen (y-axis) cultures, by pMHC multimers or 4-1BB up-regulation. FIG. 4I shows cumulative frequencies of tumor antigen-specific T cells (n=20 epitopes) in ×1NeoScreen (x-axis) and ×2NeoScreen (y-axis) cultures, by pMHC multimers or 4-1BB up-regulation. In FIGS. 4D, 4E, 4G, 4H, and 4I, the background levels of IFNγ Spot Forming Unit (FIG. 4E) or 4-1BB expression (FIGS. 4D, 4G, 4H, and 4I) by cognate negative controls (TILs alone) were subtracted. In FIGS. 4B, 4D, 4H, and 4I, the highest values between 1×NeoScreen and 2×NeoScreen are considered, and data are displayed in a logarithmic scale. In FIGS. 4B, 4D, 4E, 4F, 4G, 4H, and 4I, P-values were determined with one-tailed paired t-tests.

FIGS. 5A, 5B, 5C, 5D, 5E, 5F, 5G, 5H, 5I, and 5J are a set of diagrams showing improved identification of neoantigen-specific CD8 T cells with NeoScreen. FIGS. 5A and 5B show neoantigen discovery with NeoScreen (n=6 patients, Table 2). FIG. 5A shows the results of a cumulative analysis of the frequency of neoepitope-specific T cells (n=11 neoepitopes) in conventional (x-axis) and NeoScreen (y-axis) cultures of patients 1, 2, 4, 5, 8, and 9 (Table 4) by pMHC multimers staining or 4-1BB up-regulation. FIG. 5B shows the proportion of neoepitopes among enriched versus newly-detected T cell reactivities. FIG. 5C shows cumulative frequencies of neoepitope-specific CD8 T cells (n=15 neoepitopes from 8 patients) in in vitro-expanded TIL cultures (×2: re-stimulated). Box plots represent median (line), 25% and 75% confidence limit (box limits), and min to max (whiskers). FIG. 5D shows frequencies of neoepitope-specific CD8 T cells (n=15) in conventional (x-axis) and NeoScreen (y-axis) cultures. FIG. 5E shows cumulative frequencies of neoepitope-specific T cells (n=13 neoepitopes) in ×1NeoScreen (x-axis) and ×2NeoScreen (y-axis) cultures. In FIGS. 5A, 5C, 5D, and 5E, the background levels of 4-1BB expression by cognate negative controls were subtracted. In FIGS. 5A and 5D, the highest values between 1×NeoScreen and 2×NeoScreen are considered. In FIGS. 5A, 5C, 5D, and 5E, P-values were determined with one-tailed paired t-tests, and data are displayed in a logarithmic scale.

FIGS. 6A, 6B, 6C, 6D, and 6E are a set of diagrams showing improved identification of neoantigen-specific CD8 T cells with NeoScreen. FIGS. 6A and 6B show neoantigen discovery with NeoScreen (n=6 patients). FIG. 6A shows a cumulative analysis of the frequency of neoepitope-specific T cells (n=11 neoepitopes) in conventional (x-axis) and NeoScreen (y-axis) cultures of patients 1, 2, 4, 5, 8, and 9 by pMHC multimers staining or 4-1BB up-regulation. FIG. 6B shows the proportion of neoepitopes among enriched versus newly-detected T cell reactivities. FIG. 6C shows cumulative frequencies of neoepitope-specific CD8 T cells (n=15 neoepitopes from 8 patients) in vitro-expanded TIL cultures (×2: re-stimulated). Box plots represent median (line), 25% and 75% confidence limit (box limits) and min to max (whiskers). FIG. 6D shows frequencies of neoepitope-specific CD8 T cells (n=15) in conventional (x-axis) and NeoScreen (y-axis) cultures. FIG. 6E shows a cumulative frequencies of neoepitope-specific T cells (n=13 neoepitopes) in ×1NeoScreen (x-axis) and ×2NeoScreen (y-axis) cultures. In FIGS. 6A, 6C, 6D, and 6E, the background levels of 4-1BB expression by cognate negative controls were subtracted. In FIGS. 6A and 6D, the highest values between 1×NeoScreen and 2×NeoScreen are considered. In FIGS. 6A, 6C, 6D, and 6E, P-values were determined with one-tailed paired t-tests, and data are displayed in a logarithmic scale.

FIGS. 7A, 7B, 7C, and 7D are a set of diagrams showing the added value of the presence of engineered B cells in NeoScreen (Increased sensitivity of NeoScreen over peptides alone (Primed) for antigen discovery). FIG. 7A shows a comparison of NeoScreen to Primed (peptides alone), based on the addition of peptide pools (in the absence of APC) at the initiation of TIL cultures. FIG. 7B shows the potency of re-stimulation of TILs by Primed versus NeoScreen for patient 7. The frequency of neoantigen-specific T cells was determined by IFNγ Spot Forming Unit per 10⁵ cells following re-challenge with PHLPP2_{N1186Y} peptide. FIG. 7C shows the magnitude of tumor antigen-specific T cells determined by IFNγ Spot Forming Unit per 10⁵ cells (n=9 epitopes) obtained with NeoScreen, Primed or conventional cultures. The box plots represent median (line), 25% and 75% confidence limit (box limits), and min to max (whiskers). FIG. 7D shows the results of a cumulative analysis of the frequency of tumor epitope-specific T cells (n=9 neoepitopes, Table 4) in Primed (x-axis) and NeoScreen (y-axis) cultures of patients 1, 6, 7, 8, and 9 (Table 2), by IFNγ Spot Forming Unit per 10⁵ cells. For FIGS. 7C and 7D, the background levels of IFNγ Spot Forming Unit by cognate negative controls (TILs alone) were subtracted, and the highest values between 1×NeoScreen and 2×NeoScreen are considered. P-values were determined with one-tailed paired t-tests, and data are displayed in a logarithmic scale.

FIGS. 8A and 8B are a set of diagrams showing identification of tumor antigen-specific TCRs. FIGS. 8A and 8B show the representative examples of TCR repertoire analyses upon isolation of antigen-specific T cells by FACS. Tumor antigen-specific T cells from patients 1 and 2×2NeoScreen-stimulated TILs were FACS sorted using pMHC multimers and immediately processed for TCR bulk sequencing analysis (TCRα and TCRβ chains). Tumor antigens and cognate TCRs are described in Tables 4 and 5. The Manhattan plots report TCRα and TCRβ V/J recombinations of tumor antigen-specific T cells: V and J segments are represented according to chromosomal locations on x and y-axes, respectively.

FIGS. 9A, 9B, 9C, and 9D are a set of diagrams showing validation of antigen-specific TCRs upon TCR cloning. FIGS. 9A, 9B, and 9C show the representative examples of validation of TAA and neoepitope-specific TCRαβ pairs from patients 1, 2, and 4. FIGS. 9A, 9B, and 9C show validation of tumor antigen-specificity after labeling with cognate pMHC multimers of Jurkat cells co-electroporated with TCRα and TCRβ chain RNAs. The dot plots report the concomitant expression of the transgenic TCR and of the mouse TCRβ constant region used as a marker of transfection efficiency. In FIG. 9D, for patient 3, tyrosinase₅₀₈₋₅₁₄-specific TILs were FACS-sorted based on 4-1BB upregulation. Autologous activated primary T cells cloned with TCRαβ pair were co-cultured with autologous CD40-act B cells pulsed with peptide LPEEKQPL (SEQ ID NO: 242). Reactivity was assessed by 4-1BB upregulation. MOCK: control of transfection, neg pair: irrelevant TCRα/β pair, UNP: unpulsed no antigen.

FIGS. 10A, 10B, and 10C are a set of diagrams showing molecular modeling of PHLPP2N1186Y-specific pMHC-TCRs interactions. FIGS. 10A, 10B, and 10C show the modeled structures of the three PHLPP2N1186Y-specific TCRs (TCR-A (FIG. 10A), TCR-B (FIG. 10), and TCR-C (FIG. 10C; Table 6) depicting detailed predicted interactions with a cognate pMHC complex. The relevant interacting residues are displayed as sticks, the peptide is shown in grey sticks, and residues are labeled in black.

FIGS. 11A, 11B, 11C, 11D, and 11E are a set of diagrams showing tracking of antigen-specific TCRs in ex vivo and in vitro-expanded TIL samples. TCRβ repertoire analysis was performed on ex vivo tumors, bulk conventional TILs, and NeoScreen expanded TILs. The frequency of each tumor antigen-specific TCRβ within the bulk TIL populations and within ex vivo tumors, validated as shown in FIG. 8. Representative examples of TCR tracking from patients 1 (FIG. 11A), 2 (FIG. 11B), 3 (FIG. 11C), 6 (FIG. 11D), and 7 (FIG. 11E) are displayed. For patient 7, NeoScreen TILs generated with tandem minigene are shown. The violin plots report, in each sample, the bulk TCR repertoire distribution, as well as the frequency of tumor antigen-specific TCRβ clonotypes. (×2: re-stimulated).

FIGS. 12A and 12B are a set of diagrams showing the frequency, reactivity, and efficacy of tumor-reactive TCRs. FIG. 12A shows a representative example of flow cytometry data showing in vitro tumor recognition (4-1in upregulation) of antigen-specific TCRs (MAGEC1 TCRs A and B and SCM1A_{L674S} TCR C) from patient 1. MOCK: control of transfection, neg pair: irrelevant TCRα/P pair. FIG. 12B shows in vivo efficacy of adoptively-transferred tyrosinase₅₀₈₋₅₁₄ TCR-transduced T cells against autologous patient-derived tumor xenografts. The graph shows the tumor size of individual hIL-2 NOG mice adoptively-transferred with TCR-transduced (in orange; n=7) and untransduced (in blue; n=5) cells. ACT was performed on Day 14.

FIGS. 13A and 13B are a set of graphs showing evaluation of the frequency of tumor antigen-specific T cells by IFNγ ELISpot and 4-1in upregulation. FIGS. 13A and 13B show an example of T cell reactivity to neoepitope PHLPP2_{N1186Y}, assessed by IFNγ ELISpot assay (FIG. 13A) and 4-1in upregulation (FIG. 13n). FIG. 13A shows IFNγ Spot Forming Unit per 10⁵ cells (mean±SD of duplicate) of TILs alone (negative control) or following stimulation with neoepitope PHLPP2_{N1186Y}. FIG. 13B shows the frequency of neoepitope-specific CD8 T cells assessed by 4-1BB staining following cell recovery from ELISpot plates.

FIGS. 14A and 14B are a set of diagrams showing evaluation of the frequency of tumor antigen-specific T cells by flow cytometry. FIG. 14A shows the gating strategy and assessment of the frequency of tumor antigen-specific CD8 T cells by 4-1BB (CD137) staining following cell recovery from ELISpot plates and an example of 4-1BB upregulation following stimulation with neoepitope FCLR2_{R440M} of ×2NeoScreen TILs from patient 6. FIG. 14B shows gating strategy and assessment of the frequency of tumor antigen-specific CD8 T cells by pMHC multimer staining and an example of MAGEC1 and SMC1A_{L674S}-multimer staining of ×2NeoScreen TILs from patient 1.

FIGS. 15A and 15B are a set of diagrams showing validation of antigen-specific TCRs and assessment of in vitro tumor recognition. FIG. 15A shows validation of antigen-specificity of MAGEC1 TCR A from patient 1 by transfection of Jurkat cells and pMHC multimer staining. FIG. 15B shows interrogation of in vitro tumor recognition of MAGEC1-specific TCR A by co-culture of TCR-transfected primary activated T cells with autologous tumor cells and evaluation of 4-1BB (CD137)-upregulation. TRAN: control of transfection by evaluation of the expression of the mouse β constant chain of transfected TCR.

FIGS. 16A, 16B, 16C, 16D, 16E, and 16F are a set of diagrams showing validation of tumor reactivity of identified tumor antigen-specific TCRs. FIGS. 16A, 16B, 16C, 16D, and 16E show an overview of tumor reactivity of TCR-transfected CD8 T cells for patients 1-5. To assess antitumor reactivity of validated tumor antigen-specific TCRs (Table 5), autologous (patients 1-3 & 5) or HLA-matched (patient 4) TCR-transfected primary activated T cells were co-cultured with autologous tumor cells, and 4-1BB up-regulation was measured. MOCK-T cells (transfected with PBS), T cells transfected with a control TCR (irrelevant crossmatch of a TCRα and β chain), and T cells transfected with a viral TCR (when available) were included as controls. The proportion of TCR-T cells expressing 4-1BB is depicted in the presence or absence of tumor cells. Statistics were applied when ≥2 unique TCRs were targeting the same antigen, and P-values were then determined with one-tailed paired t-tests. FIG. 16F shows the results of cumulative statistics of all identified tumor antigen-specific TCRs (n=31). P-value was determined with a one-tailed paired t-test.

### DETAILED DESCRIPTION OF THE INVENTION

This disclosure describes novel T cell receptors (TCRs) specific for tumor-associated antigens (TAAs) and methods of use thereof. The disclosed TCRs and methods of use expand the applications of adoptive cell therapy and TCR-based cellular immunotherapies.

### A. T CELL RECEPTORS

a. T Cell Receptors

In one aspect, this disclosure provides a TCR or antigen-binding fragment thereof that binds specifically to a tumor-associated antigen (TAA), e.g., a neoantigen. In some embodiments, the TCR or antigen-binding fragment thereof comprises an amino acid sequence having at least 90% sequence identity to an amino acid sequence of SEQ ID NOs: 1-228 or comprises an amino acid sequence of SEQ ID NOs: 1-228.

In some embodiments, the TCR or antigen-binding fragment thereof comprises an α chain amino acid sequence having at least 80% (e.g., 80%, 85%, 90%, 95%, 96%, 97%, 98%, 99%) sequence identity to the amino acid sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, or 227; or comprises an α chain amino acid sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, or 227.

In some embodiments, the TCR or antigen-binding fragment thereof comprises a β chain amino acid sequence having at least 80% (e.g., 80%, 85%, 90%, 95%, 96%, 97%, 98%, 99%) sequence identity to the amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, or 228; or comprises a β chain amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, or 228.

In some embodiments, the TCR or antigen-binding fragment thereof comprises: (i) an α chain amino acid sequence having at least 80% (e.g., 80%, 85%, 90%, 95%, 96%, 97%, 98%, 99%) sequence identity to the amino acid sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, or 227; or comprises an α chain amino acid sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, or 227; and (ii) a R chain amino acid sequence having at least 80% (e.g., 80%, 85%, 90%, 95%, 96%, 97%, 98%, 99%) sequence identity to the amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, or 228; or comprises a β chain amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, or 228.

In some embodiments, the TCR or antigen-binding fragment thereof comprises: an α chain amino acid sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, or 227; and a β chain amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222,224, 226, or 228.

In some embodiments, the TCR or antigen-binding fragment thereof comprises an α chain complementarity-determining region 3 (CDR3) having an amino acid sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, or 75; and a β chain CDR3 having an amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, or 76.

In some embodiments, the TCR or antigen-binding fragment thereof comprises the α chain amino acid sequence and the β chain amino acid sequence having respective amino acid sequences of SEQ ID NOs: 1-2, 3-4, 5-6, 7-8, 9-10, 11-12, 13-14, 15-16, 17-18, 19-20, 21-22, 23-24, 25-26, 27-28, 29-30, 31-32, 33-34, 35-36, 37-38, 39-40, 41-42, 43-44, 45-46, 47-48, 49-50, 51-52, 53-54, 55-56, 57-58, 59-60, 61-62, 63-64, 65-66, 67-68, 69-70, 71-72, 73-74, 75-76, 77-78, 79-80, 81-82, 83-84, 85-86, 87-88, 89-90, 91-92, 93-94, 95-96, 97-98, 99-100, 101-102, 103-104, 105-106, 107-108, 109-110, 111-112, 113-114, 115-116, 117-118, 119-120, 121-122, 123-124, 125-126, 127-128, 129-130, 131-132, 133-134, 135-136, 137-138, 139-140, 141-142, 143-144, 145-146, 147-148, 149-150, 151-152, 153-154, 155-156, 157-158, 159-160, 161-162, 163-164, 165-166, 167-168, 169-170, 171-172, 173-174, 175-176, 177-178, 179-180, 181-182, 183-184, 185-186, 187-188, 189-190, 191-192, 193-194, 195-196, 197-198, 199-200, 201-202, 203-204, 205-206, 207-208, 209-210, 211-212, 213-214, 215-216, 217-218, 219-220, 221-222, 223-224, 225-226, or 227-228.

In some embodiments, the TCR or antigen-binding fragment thereof comprises the α chain CDR3 and the β chain CDR3 having a amino acid sequence pair of SEQ ID NOs: 1-2, 3-4, 5-6, 7-8, 9-10, 11-12, 13-14, 15-16, 17-18, 19-20, 21-22, 23-24, 25-26, 27-28, 29-30, 31-32, 33-34, 35-36, 37-38, 39-40, 41-42, 43-44, 45-46, 47-48, 49-50, 51-52, 53-54, 55-56, 57-58, 59-60, 61-62, 63-64, 65-66, 67-68, 69-70, 71-72, 73-74, or 75-76.

As used herein, the term “T cell receptor” or “TCR” refers to a surface protein of a T cell that allows the T cell to recognize an antigen and/or an epitope thereof, typically bound to one or more major histocompatibility complex (MHC) molecules. A TCR functions to recognize an antigenic determinant and to initiate an immune response. Typically, TCRs are heterodimers comprising two different protein chains. In the vast majority of T cells, the TCR comprises an α chain and a β chain. Approximately 5% of T cells have TCRs made up of 7/6 chains. TCRs are membrane-anchored heterodimers that are found as part of a complex with a CD3 chain molecule. Each chain comprises two extracellular domains: a variable (V) region and a constant (C) region, the latter of which is membrane-proximal. The variable domains of α chains and β chains consist of three hypervariable regions that are also referred to as the complementarity determining regions (CDRs). The CDRs, in particular CDR3, are primarily responsible for contacting antigens and thus define the specificity of the TCR, although CDR1 of the α chain can interact with the N-terminal part of the antigen. CDR1 of the β chain interacts with the C-terminal part of the peptide. TCRs are also characterized by a series of highly conserved disulfide bonds that link the two chains.

In some embodiments, the TCR α chains may further comprise a TCR α transmembrane domain and/or a TCR α intracellular domain. Similarly, the TCR R chains may further comprise a TCR β transmembrane domain and/or a TCR β intracellular domain. The TCRs may further comprise a constant region derived from any suitable species, such as any mammal, e.g., human, rat, monkey, rabbit, donkey, or mouse. In some embodiments, the TCRs further comprise a human constant region. In some embodiments, the TCR constant region may be modified, for example, by the introduction of heterologous sequences, which may increase TCR expression and stability. In some embodiments, the TCR sequences are murinized or humanized.

In some embodiments, the TCR is an αβ heterodimeric TCR. In some embodiments, the TCR is an αβ single chain TCR (scTCR) or a TCR-like polypeptide. In some embodiments, the TCR as disclosed herein may be provided as a scTCR. A scTCR may comprise in one polypeptide chain a full or partial α chain sequence and a full or partial β chain sequence, which may be connected via a peptide linker. A scTCR can comprise a polypeptide of a variable region of a first TCR chain (e.g., an α chain) and a polypeptide of an entire (full-length) second TCR chain (e.g., a β chain), or vice versa. Furthermore, the scTCR can optionally comprise one or more linkers that join the two or more polypeptides together. The linker can be, for example, a peptide, which joins together two single chains, as described herein.

As used herein, the phrase “TCR-like polypeptide” refers to a polypeptide that behaves similarly to a TCR in that it specifically binds to an MHC-bound peptide, optionally an MHC-bound phosphopeptide. A “TCR-like antibody” refers to an antibody, optionally a monoclonal antibody, which specifically recognizes an MHC-bound phosphopeptide of the presently disclosed subject matter. In some embodiments, such polypeptides are members of the Ig superfamily. In some embodiments, a TCR-like polypeptide is a single chain TCR (see, e.g., U.S. Patent Application Publication No. 2012/0252742; PCT International Patent Application Publication Nos. WO 1996/013593, WO 1999/018129, and WO 2004/056845; U.S. Pat. No. 7,569,664).

As used herein, a “fragment” or “portion” of a TCR or TCR-like polypeptide is a subsequence of a TCR or TCR-like polypeptide that retains a desired function of the TCR or TCR-like polypeptide. In some embodiments, a fragment or portion of a TCR or TCR-like polypeptide comprises the domain of the TCR or TCR-like polypeptide that binds to a phosphopeptide/MHC complex (e.g., a phosphopeptide/HLA-A2 complex). Thus, in some embodiments, the phrase “TCR, TCR-like molecule, or portion thereof” refers to TCRs, TCR-like molecules, and portions thereof that bind to phosphopeptide/MHC complexes, including but not limited to phosphopeptide/HLA-A2 complexes.

b. Polypeptides

In another aspect, this disclosure also provides a polypeptide comprising an amino acid sequence having at least 80% (e.g., 80%, 85%, 90%, 95%, 96%, 97%, 98%, 99%) sequence identity to an amino acid sequence of SEQ ID NOs: 1-228 or comprising an amino acid sequence of SEQ ID NOs: 1-228.

In some embodiments, the polypeptide comprises a first polypeptide chain that comprises an amino acid sequence having at least 80% (e.g., 80%, 85%, 90%, 95%, 96%, 97%, 98%, 99%) sequence identity to the amino acid sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, or 227; or comprises an amino acid sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, or 227.

In some embodiments, the polypeptide comprises a second polypeptide chain that comprises an amino acid sequence having at least 80% (e.g., 80%, 85%, 90%, 95%, 96%, 97%, 98%, 99%) sequence identity to the amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, or 228; or comprises an amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, or 228.

In some embodiments, the polypeptide comprises: (i) a first polypeptide chain that comprises an amino acid sequence having at least 80% (e.g., 80%, 85%, 90%, 95%, 96%, 97%, 98%, 99%) sequence identity to the amino acid sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, or 227; or comprises an amino acid sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, or 227; and (ii) a second polypeptide chain that comprises an amino acid sequence having at least 80% (e.g., 80%, 85%, 90%, 95%, 96%, 97%, 98%, 99%) sequence identity to the amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, or 228; or comprises an amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, or 228.

In some embodiments, the polypeptide comprises the first polypeptide chain and the second polypeptide chain having respective amino acid sequences of SEQ ID NOs: 1-2, 3-4, 5-6, 7-8, 9-10, 11-12, 13-14, 15-16, 17-18, 19-20, 21-22, 23-24, 25-26, 27-28, 29-30, 31-32, 33-34, 35-36, 37-38, 39-40, 41-42, 43-44, 45-46, 47-48, 49-50, 51-52, 53-54, 55-56, 57-58, 59-60, 61-62, 63-64, 65-66, 67-68, 69-70, 71-72, 73-74, 75-76, 77-78, 79-80, 81-82, 83-84, 85-86, 87-88, 89-90, 91-92, 93-94, 95-96, 97-98, 99-100, 101-102, 103-104, 105-106, 107-108, 109-110, 111-112, 113-114, 115-116, 117-118, 119-120, 121-122, 123-124, 125-126, 127-128, 129-130, 131-132, 133-134, 135-136, 137-138, 139-140, 141-142, 143-144, 145-146, 147-148, 149-150, 151-152, 153-154, 155-156, 157-158, 159-160, 161-162, 163-164, 165-166, 167-168, 169-170, 171-172, 173-174, 175-176, 177-178, 179-180, 181-182, 183-184, 185-186, 187-188, 189-190, 191-192, 193-194, 195-196, 197-198, 199-200, 201-202, 203-204, 205-206, 207-208, 209-210, 211-212, 213-214, 215-216, 217-218, 219-220, 221-222, 223-224, 225-226, or 227-228.

Also within the scope of this disclosure are the variants of the TCR or the polypeptide, as described above. As used herein, the term “variant” refers to a first molecule that is related to a second molecule (also termed a “parent” molecule). The variant molecule can be derived from, isolated from, based on or homologous to the parent molecule. A “functional variant” of a protein as used herein refers to a variant of such protein that retains at least partially the activity of that protein. Functional variants may include mutants (which may be insertion, deletion, or replacement mutants), including polymorphs, etc. Also included within functional variants are fusion products of such protein with another, usually unrelated, nucleic acid, protein, polypeptide, or peptide. Functional variants may be naturally occurring or may be man-made.

In some embodiments, a variant of the TCR or the polypeptide may include one or more conservative modifications. The variant with one or more conservative modifications may retain the desired functional properties, which can be tested using the functional assays known in the art.

As used herein, the term “conservative sequence modifications” refers to amino acid modifications that do not significantly affect or alter the binding characteristics of the protein containing the amino acid sequence. Such conservative modifications include amino acid substitutions, additions, and deletions. Modifications can be introduced by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include: amino acids with basic side chains (e.g., lysine, arginine, histidine); acidic side chains (e.g., aspartic acid, glutamic acid); uncharged polar side chains (e.g., glycine, asparagine, glutamine, serine, threonine, tyrosine, cysteine, tryptophan); nonpolar side chains (e.g., alanine, valine, leucine, isoleucine, proline, phenylalanine, methionine); beta-branched side chains (e.g., threonine, valine, isoleucine); and aromatic side chains (e.g., tyrosine, phenylalanine, tryptophan, histidine) includes one or more conservative modifications. The variant of the TCR or the polypeptide with one or more conservative modifications may retain the desired functional properties, which can be tested using the functional assays known in the art.

As used herein, the percent homology between two amino acid sequences is equivalent to the percent identity between the two sequences. The percent identity between the two sequences is a function of the number of identical positions shared by the sequences (i.e., % homology=#of identical positions/total #of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program, using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum62 matrix or a PAM250 matrix, and a gap weight of 16, 14, 12, 10, 8, 6, or 4 and a length weight of 1, 2, 3, 4, 5, or 6.

The term “homolog” or “homologous,” when used in reference to a polypeptide, refers to a high degree of sequence identity between two polypeptides, or to a high degree of similarity between the three-dimensional structure or to a high degree of similarity between the active site and the mechanism of action. In some embodiments, a homolog has a greater than 60% sequence identity, and more preferably greater than 75% sequence identity, and still more preferably greater than 90% sequence identity, with a reference sequence. The term “substantial identity,” as applied to polypeptides, means that two peptide sequences, when optimally aligned, such as by the programs GAP or BESTFIT using default gap weights, share at least 75% sequence identity.

Also within the scope of this disclosure are the variants, mutants, and homologs with significant identity to the disclosed TCRs or polypeptides. For example, such variants and homologs may have sequences with at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the sequences of TCRs or polypeptides described herein.

As used herein, the term “antigen” is a molecule and/or substance that can generate peptide fragments that are recognized by a TCR and/or induces an immune response. An antigen may contain one or more “epitopes.” In some embodiments, the antigen has several epitopes. An epitope is recognized by a TCR, an antibody or a lymphocyte in the context of an MHC molecule.

The terms “tumor-associated antigen” and “cancer antigen,” as used herein, refer to any molecule (e.g., protein, peptide, lipid, carbohydrate, etc.) solely or predominantly expressed or over-expressed by a tumor cell and/or a cancer cell, such that the antigen is associated with the tumor and/or the cancer. The TAA/cancer antigen can also be expressed by normal, non-tumor, or non-cancerous cells. However, in such a situation, the expression of the TAA/cancer antigen by normal, non-tumor, or non-cancerous cells is, in some embodiments, not as robust as the expression of the TAA/cancer antigen by tumor and/or cancer cells. Thus, in some embodiments, the tumor and/or cancer cells overexpress the TAA and/or express the TAA at a significantly higher level as compared to the expression of the TAA by normal, non-tumor, and/or non-cancerous cells. In some embodiments, the phosphopeptides are fragments of TAAs or TAAs themselves.

The TAA can be an antigen expressed by any cell of any cancer or tumor, including the cancers and tumors described herein. The TAA can be a TAA of only one type of cancer or tumor, such that the TAA is associated with or characteristic of only one type of cancer or tumor. Alternatively, the TAA can be characteristic of more than one type of cancer or tumor. For example, the TAA can be expressed by both breast and prostate cancer cells and not expressed at all by normal, non-tumor, or non-cancer cells.

Non-limiting examples of tumor-associated proteins from which tumor antigens (including neoantigens) can be identified include, e.g., 13HCG, 43-9F, 5T4, 791Tgp72, adipophilin, AIM-2, ALDH1A1, alpha-actinin-4, alpha-fetoprotein (“AFP”), ARTC1, B-RAF, BAGE-1, BCA225, BCLX (L), BCR-ABL fusion protein b3a2, beta-catenin, BING-4, brain glycogen phosphorylase, BTAA, c-met, CA-125, CA-15-3 (CA 27.29BCAA), CA-19-9, CA-242, CA-50, CA-72-4, CALCA, CAM 17.1, CAM43, carcinoembryonic antigen (“CEA”), CASP-5, CASP-8, CD274, CD45, CD68\KP1, Cdc27, CDK12, CDK4, CDK 2A, CEA, CLPP, CO-029, COA-1, CPSF, CSNK1A1, CT-7, CT9/BRDT, CTAG1, CTAG2, CTpl l, cyclin Dl, Cyclin-A1, dek-can fusion protein, DK 1, E2A-PRL, EBNA, EF2, EFTUD2, Elongation factor 2, ENAH (hMena), Ep-CAM, EpCAM, EphA3, epithelial tumor antigen (“ETA”), Epstein Barr virus antigens, ETV6-AML1 fusion protein, EZH2, FGF5, FLT3-ITD, FN1, G250, G250/MN/CALX, Ga733 (EpCAM), GAGE-1,2,8, GAGE-3,4,5,6,7, GAS7, glypican-3, GnTV, gplOO, gplOO/Pmel 17, GPNMB, H-ras, H4-RET, HAUS3, Hepsin, HER-2/neu, HERV-K-MEL, HLA-A11, HLA-A2, HLA-DOB, HOM-MD-21, HOM-MD-397, Horn/Me 1-40, Horn/Mel-55, HPV E2, HPV E6, HPV E7, hsp70-2, HTgp-175, IDOl, IGF2B3, IGH-IGK, IL13Ralpha2, Intestinal carboxyl esterase, K-ras, Kallikrein 4, KIAAO205, KIF20A, KK-LC-1, KKLC1, KM-HN-1, KMHN1 also known as CCDC110, LAGE-1, LAGE-2, LB33/MUM-1, LDLR-fucosyltransferaseAS fusion protein, Lengsin, M-CSF, M344, MA-50, MAGE-1, MAGE-2, MAGE-3, MAGE-4, MAGE-5, MAGE-6, MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-A13, MAGE-B (MAGE-B 1-MAGE-B24), MAGE-C (MAGE-C1/CT7, CT10), MAGE-C1, MAGE-C2, MAGE-Xp2 (MAGE-B2), MAGE-Xp3 (MAGE-B3), MAGE-Xp4 (MAGE-B4), malic enzyme, mammaglobin-A, MAPE, MART-I, MART-2, MATN, MC1R, MCSP, mdm-2, ME1, Melan-A/MART-1, Meloe, MG7-Ag, Midkine, MMP-2, MMP-7, MOV 18, MUC1, MUCSAC, mucin, MUM-1, MUM-2, MUM-3, MYL-RAR, Myosin, Myosin class I, N-ras, N-raw, NA88-A, NAG, NBU70K, neo-PAP, NFYC, nm-23Hl, NuMa, NY-BR-1, NY-CO-1, NY-CO-2, NY-ESOl, NY-ESO-1/LAGE-2, OA1, OGT, OS-9, P polypeptide, pl5(58), pl6, pl85erbB2, pl80erbB-3, p53, PAP, PAX5, PBF, pml-RARalpha fusion protein, polymorphic epithelial mucin (“PEM”), PPP1R3B, PRAME, PRDX5, PSA, PSCA, PSMA, PTPRK, RAB38/NY-MEL-1, RAGE-1, RBAF600, RCAS1, RGS5, R oC, RNF43, RU2AS, SAGE, SART-1, SART-3, SCP-1, SDCCAG16, secernin 1, SIRT2, SNRPD1, SOX10, Spl7, SPA17, SSX-1, SSX-2, SSX-4, SSX-5, STEAP1, survivin, SYT-SSX1 or -SSX2 fusion protein, TA-90 (Mac-2 binding protein\cyclophilin C-associated protein), TAAL6, TAG-1, TAG-2, TAG-72-4, TAGE, Telomerase, TERT, TGF-betaRII, TLP, TPBG, TPS TRAG-3, Triosephosphate isomerase, TRP-1, TRP-2, TRP-1/gp75, TRP-2, TRP2-INT2, TSP-180, TSP50, tyrosinase, tyrosinase (“TYR”), VEGF, WT1, XAGE-lb/GAGED2a, Kras, WT-1 antigen (in lymphoma and other solid tumors), ErbB receptors, Melan A (MARTI), gp 100, tyrosinase, TRP-l/gp 75, and TRP-2 (in melanoma); MAGE-1 and MAGE-3 (in bladder, head and neck, and non-small cell carcinoma); HPV EG and E7 proteins (in cervical cancer); Mucin (MUC-1) (in breast, pancreas, colon, and prostate cancers); prostate-specific antigen (PSA) (in prostate cancer); carcinoembryonic antigen (CEA) (in colon, breast, and gastrointestinal cancers), and such shared tumor-specific antigens as MAGE-2, MAGE-4, MAGE-6, MAGE-10, MAGE-12, BAGE-1, CAGE-1,2,8, CAGE-3 TO 7, LAGE-1, NY-ESO-1/LAGE-2, NA-88, GnTV, TRP2-INT2. For example, antigenic peptides characteristic of tumors include those listed in Cancer Vaccines and Immunotherapy (2000) Eds Stem, Beverley and Carroll, Cambridge University Press, Cambridge, Cancer Immunology (2001), Kluwer Academic Publishers, The Netherlands, International Patent Application Publication No. WO 20000/020581 and U.S. Patent Application Publication No. 2010/0284965, and www.cancerimmunity.org/peptidedatabase/Tcellepitopes, which are each incorporated herein by reference in their entirety.

In some embodiments, the TAA comprises an amino acid sequence having at least 80% (e.g., 80%, 85%, 90%, 95%, 96%, 97%, 98%, 99%) sequence identity to the amino acid sequence of SEQ ID NOs: 229-268 or an amino acid sequence of SEQ ID NOs: 229-268.

As used herein, the term “neoantigen” refers to a newly formed antigenic determinant that arises from a somatic mutation(s) and is recognized as “non-self” A neoantigen can include a polypeptide sequence or a nucleotide sequence. A mutation can include a frameshift or non-frameshift indel, missense or nonsense substitution, splice site alteration (e.g., alternatively spliced transcripts), genomic rearrangement or gene fusion, or any genomic or expression alteration giving rise to a neoORF. A mutation can also include a splice variant. Post-translational modifications specific to a tumor cell can include aberrant phosphorylation. Post-translational modifications specific to a tumor cell can also include a proteasome-generated spliced antigen (see, e.g., Liepe et al., Science; 354(6310):354-358 (2006), incorporated herein by reference in its entirety). A neoantigen can include a canonical antigen. A neoantigen can also include non-canonical antigen.

Neoantigen can be tumor-specific.

As used herein, the phrase “specific binding” refers to binding between a TCR, TCR-like molecule, or antigen-binding fragment thereof and an antigen and/or an epitope thereof (including but not limited to a peptide, optionally in complex with an MHC molecule) that is indicative of the presence of the antigen and/or the epitope thereof. As such, a TCR, TCR-like molecule, or antigen-binding fragment thereof is said to “specifically” bind an antigen and/or an epitope thereof when the dissociation constant (Kd) is less than about 1 μM, less than about 100 nM, or less than about nM. Interactions between a TCR, TCR-like molecule, or antigen-binding fragment thereof and an epitope can also be characterized by an affinity constant (Kₐ). In some embodiments, a Kₐ of less than about 10⁷/M is considered “high affinity.”

c. Bifunctional Molecules

One approach to overcome the lack of potent anti-tumor T-cell immunity is the ex vivo genetic modification of T cells to target tumors through the use of affinity-enhanced receptors generated from either T cell receptors as described above or antibody-derived receptors. A complementary approach that does not require ex vivo manipulation of T cells involves the use of fusion proteins that combine tumor recognition and T cell engaging domains to redirect T cells to target tumors. Specificity and anti-tumor activity of such fusion proteins are described in, for example, Cancer Immunol Immunother (2013) 62:773-785, Nat Med. 2012 June; 18(6):980-7), and U.S. Pat. Nos. 7,763,718; and 10,130,721, each of which is incorporated herein by reference in its entirety for all purposes.

In one aspect, provided herein is a bifunctional molecule comprising the TCR or the polypeptide as disclosed herein, or a functional fragment thereof, and a second polypeptide that specifically binds to a cell surface protein on a T-cell. Examples of cell surface proteins on T-cells include, but are not limited to, CD2, CD3, CD4, CD8, CD44, CD45RA, CD45RB, CD45RO, CD49a, CD49b, CD49c, CD49d, CD49e, CD49f, CD16, CD28, and IL-2R.

In some embodiments, the second polypeptide comprises an immune effector polypeptide. As used herein, the term “immune effector polypeptide” generally refers to any molecule which induces or stimulates an immune response through direct or indirect activation of the humoral or cellular arm of the immune system, such as by activation of T-cells. Examples of immune effector polypeptides include, but are not limited to, IL-1, IL-1α, IL-3, IL-4, IL-5, IL-6, IL-7, IL-10, IL-11, IL-12, IL-13, IL-15, IL-21, IL-23, TGF-β, IFN-γ, TNFα, anti-CD2 antibody, anti-CD3 antibody, anti-CD4 antibody, anti-CD8 antibody, anti-CD44 antibody, anti-CD45RA antibody, anti-CD45RB antibody, anti-CD45RO antibody, anti-CD49a antibody, anti-CD49b antibody, anti-CD49c antibody, anti-CD49d antibody, anti-CD49e antibody, anti-CD49f antibody, anti-CD16 antibody, anti-CD28 antibody, anti-IL-2R antibodies, viral proteins and peptides, and bacterial proteins or peptides.

In some embodiments, the second polypeptide comprises an antibody or an antibody fragment. Antibody fragments may include, but are not limited to, single chain antibodies, Fab fragments, Fv fragments, single-chain Fv fragments (scFv), a divalent antibody fragment such as an (Fab)2′-fragment, F(ab′) fragments, disulfide-linked Fvs (sdFv), intrabodies, minibodies, diabodies, triabodies, and decabodies. In some embodiments, the polypeptide comprises an scFv.

In some embodiment, the second polypeptide specifically binds to CD3. In some embodiment, the second polypeptide comprises an anti-CD3 antibody. Examples of anti-CD3 antibodies include but are not limited to OKT3, UCHT-1, BMA031, 12F6, and an scFv derived therefrom. In some embodiment, the second polypeptide comprises an scFv derived from an anti-CD3 antibody. In some embodiments, the second polypeptide comprises an scFv derived from OKT3, UCHT-1, BMA031 or 12F6. In some embodiments, the second polypeptide that specifically binds to CD3 is an immune effector polypeptide.

In some embodiments, the N-terminus of the TCR or the polypeptide is linked to the C-terminus of the second polypeptide that specifically binds to an antigen presented by a T cell. In some embodiments, the C-terminus of the TCR or the polypeptide is linked to the N-terminus of the second polypeptide that specifically binds to an antigen presented by a T cell. In some embodiments, the TCR is a heterodimeric αβ TCR polypeptide pair, or a single chain αβ TCR (scTCR) polypeptide, and the N-terminus of the α or β chain of the heterodimeric TCR polypeptide pair, or the N-terminus of the scTCR polypeptide, is linked to a C-terminal amino acid of the polypeptide that specifically binds to an antigen presented by a T cell. In some embodiments, the TCR is a heterodimeric αβ TCR polypeptide pair, or a single chain αβ TCR polypeptide, and the C-terminus of the α or β chain of the heterodimeric TCR polypeptide pair, or the C-terminus of the scTCR polypeptide, is linked to a N-terminal amino acid of the polypeptide that specifically binds to an antigen presented by a T cell.

Linkage of the TCR and the second polypeptide that specifically binds to a cell surface protein on a T-cell may be direct or indirect via a linker sequence. Linker sequences are usually flexible, in that they are made up of amino acids such as glycine, alanine, and serine, which do not have bulky side chains likely to restrict flexibility. Usable or optimum lengths of linker sequences are easily determined in the case of any given TCR bifunctional molecule. In some instances, the linker will be less than about 12, such as less than about 10, or from 5-10 amino acids in length.

d. Nucleic Acids

In another aspect, the disclosure provides nucleic acids encoding a TCR or antigen-binding fragment thereof or a polypeptide of the disclosure. In some embodiments, the TCR or antigen-binding fragment thereof or the polypeptide is encoded by a single nucleic acid. In other embodiments, for example, in the case of a heterodimeric molecule or a polypeptide composed of more than one polypeptide chain. In some embodiments, the TCR or antigen-binding fragment thereof or the polypeptide can be encoded by a plurality (e.g., two, three, four or more) nucleic acids.

In some embodiments, a single nucleic acid can encode a TCR or antigen-binding fragment thereof that comprises a single polypeptide chain, a TCR or antigen-binding fragment thereof that comprises two or more polypeptide chains, or a TCR or antigen-binding fragment thereof that comprises more than two polypeptide chains. For example, a single nucleic acid can encode two polypeptide chains of a TCR or antigen-binding fragment thereof comprising three, four or more polypeptide chains, or three polypeptide chains of a TCR or antigen-binding fragment thereof comprising four or more polypeptide chains. For separate control of expression, the open reading frames encoding two or more polypeptide chains can be under the control of separate transcriptional regulatory elements (e.g., promoters and/or enhancers). The open reading frames encoding two or more polypeptides can also be controlled by the same transcriptional regulatory elements and separated by internal ribosome entry site (IRES) sequences allowing for translation into separate polypeptides.

In some embodiments, a TCR or antigen-binding fragment thereof comprising two or more polypeptide chains is encoded by two or more nucleic acids. The number of nucleic acids encoding a TCR or antigen-binding fragment thereof can be equal to or less than the number of polypeptide chains in the TCR or antigen-binding fragment thereof (for example, when two or more polypeptide chains are encoded by a single nucleic acid).

In some embodiments, the nucleic acids of the disclosure can be DNA or RNA (e.g., mRNA).

e. Vectors

In another aspect, the disclosure provides vectors comprising the nucleic acids encoding the TCRs or antigen-binding fragment thereof or the polypeptides as described above. The nucleic acids may be present in a single vector or separate vectors that are present in the same host cell or separate host cell.

In some embodiments, vectors can be derived from retroviruses, including avian reticuloendotheliosis virus (duck infectious anaemia virus, spleen necrosis virus, Twiehaus-strain reticuloendotheliosis virus, C-type retrovirus, reticuloendotheliosis virus Hungary-2 (REV-H-2)), and feline leukemia virus (FeLV)). Retroviral genomes have been modified for use as a vector (Cone & Mulligan, Proc. Natl. Acad. Sci., USA, 81:6349-6353, (1984)). Non-limiting examples of retroviruses include lentiviruses, such as human immunodeficiency viruses (HIV-1 and HIV-2), feline immunodeficiency virus (FIV), simian immunodeficiency virus (SIV), Maedi/Visna virus, caprine arthritis/encephalitis virus, equine infectious anaemia virus (EIAV), and bovine immunodeficiency virus (BIV); avian type C retroviruses, such as the avian leukosis virus (ALV); HTLV-BLV retroviruses, such as bovine leukaemia virus (BLV), human T cell lymphotropic virus (HTLV), and simian T cell lymphotropic virus; mammalian type B retroviruses, such as the mouse mammary tumor virus (MMTV); mammalian type C retroviruses, such as the murine leukaemia virus (MLV), feline sarcoma virus (FeSV), murine sarcoma virus, Gibbon ape leukemia virus, guinea pig type C virus, porcine type C virus, wooly monkey sarcoma virus, and viper retrovirus; spumavirus (foamy virus group), such as human spumavirus (HSRV), feline synctium-forming virus (FeSFV), human foamy virus, simian foamy virus, and bovine syncytial virus; and type D retroviruses, such as Mason-Pfizer monkey virus (MPMV), squirrel monkey retrovirus, and langur monkey virus.

In some embodiments, the vector comprises a retroviral vector or a lentiviral vector. In some embodiments, lentiviral and retroviral vectors may be packaged using their native envelope proteins or may be modified to be encapsulated with heterologous envelope proteins. Examples of envelope proteins include, but are not limited to, an amphotropic envelope, an ecotropic envelope, or a xenotropic envelope, or may be an envelope including amphotropic and ecotropic portions. The protein also may be that of any of the above-mentioned retroviruses and lentiviruses. Alternatively, the env proteins may be modified, synthetic or chimeric env constructs, or may be obtained from non-retro viruses, such as vesicular stomatitis virus and HVJ virus. Specific non-limiting examples include the envelope of Moloney Murine Leukemia Virus (MMLV), Rous Sarcoma Virus, Baculovirus, Jaagsiekte Sheep Retrovirus (JSRV) envelope protein, and the feline endogenous virus RD 114; gibbon ape leukemia virus (GALV) envelope; baboon endogenous virus (BaEV) envelope; simian sarcoma-associated virus (SSAV) envelope; amphotropic murine leukemia virus (MLV-A) envelope; human immunodeficiency virus envelope; avian leukosis virus envelope; the endogenous xenotropic NZB viral envelopes; and envelopes of the paramyxoviridiae family such as, but not limited to, the HVJ virus envelope.

f. Cells

In another aspect, this disclosure further provides a cell (e.g., antigen-specific lymphocyte) comprising the nucleic acid or the vector, as described above. In some embodiments, the cell comprises an immune cell.

In some embodiments, the immune cell comprises a lymphocyte. In some embodiments, the lymphocyte comprises a T cell or a natural killer (NK) cell. In some embodiments, the T cell comprises a CD8+ T cell or a CD4+ T cell. In some embodiments, the T cell comprises a human T cell.

Lymphocytes are one subtype of white blood cells in the immune system. In some embodiments, lymphocytes may include tumor-infiltrating immune cells. Tumor-infiltrating immune cells consist of both mononuclear and polymorphonuclear immune cells (i.e., T cells, B cells, natural killer cells, macrophages, neutrophils, dendritic cells, mast cells, eosinophils, basophils, etc.) in variable proportions. In some embodiments, lymphocytes may include tumor-infiltrating lymphocytes (TILs). TILs are white blood cells that have left the bloodstream and migrated toward a tumor. TILs can often be found in the tumor stroma and within the tumor itself. In some embodiments, TILs are “young” T cells or minimally cultured T cells. In some embodiments, the young cells have a reduced culturing time (e.g., between about 22 to about 32 days in total). In some embodiments, the lymphocytes express CD27.

In some embodiments, lymphocytes may include peripheral blood lymphocytes (PBLs). In some embodiments, lymphocytes include T lymphocytes (T cells) and/or natural killer cells (NK cells).

In some embodiments, the lymphocytes may be autologous, allogeneic, syngeneic, or xenogeneic with respect to the subject. In some embodiments, the lymphocytes are autologous in order to reduce an immunoreactive response against the lymphocyte when reintroduced into the subject for immunotherapy treatment.

In some embodiments, the T cells are CD8+ T cells. In some embodiments, the T cells are CD4+ cells. In some embodiments, the NK cells are CD 16+ CD56+ and/or CD57+ NK cells. NKs are characterized by their ability to bind to and kill cells that fail to express “self MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

In another aspect, this disclosure also provides a method of genetically engineering cells (e.g., lymphoid cells) that express a TCR or a polypeptide described herein. The method includes preparing a population of cells (e.g., antigen-specific lymphocytes) expressing a TCR specific for a target cell in a subject. In some embodiments, the method comprises: (a) isolating a plurality of cells from a subject; (b) transfecting the plurality of cells with the vector described above; and (c) optionally expanding the transfected cells.

In some embodiments, the subject is a patient. In some embodiments, the subject is a healthy donor. In some embodiments, the target cell comprises a tumor cell.

In some embodiments, the genetically engineered cells are lymphoid cells. The lymphoid cells may be obtained from PBMC, tumor draining lymph nodes or tumor infiltrates. In some embodiments, the genetically engineered lymphoid cells are further engineered to express additional receptor(s). In some embodiments, the genetically engineered lymphoid cells are further engineered to express soluble protein(s). Soluble proteins include, but are not limited to, cytokines, chemokines, growth factors, soluble receptors, ligands, antibodies, antibody fragments, and antigen binding domains. In some embodiments, the genetically engineered lymphoid cells are further engineered to express additional receptor(s) and soluble protein(s).

Lymphoid cells are cells of the immune system that react specifically with antigens and elaborate specific cell products. The sample containing the lymphoid cells can be obtained from numerous sources in the subject, including but not limited to such as but not limited to, a tissue (including tumor tissue viral infected tissue, tissue at the site of inflammation, site of lymphocyte infiltration, and site of leukocyte infiltration), thymus, tumor tissue (e.g., samples, fragments), or enzymatically digested tissue, dissociated/suspended cells, a lymph node sample, or a bodily fluid sample (e.g., blood, ascites, lymph). Exemplary tissues include skin, adipose tissue, cardiovascular tissue such as veins, arteries, capillaries, and valves; neural tissue, bone marrow, breast, gastrointestinal, pulmonary tissue, ocular tissue such as corneas and lens, cartilage, bone, and mucosal tissue.

The sample can be an untreated, enzymatically treated, and/or dissociated/suspended to form a cell suspension. When the sample is enzymatically treated, non-limited examples of enzymes that can be used include collagenase, dispase, hyaluronidase, liberase, and deoxyribonuclease (DNase).

In some embodiments, lymphoid cells for use in this disclosure include tumor-infiltrating immune cells. Tumor-infiltrating immune cells consist of both mononuclear and polymorphonuclear immune cells (i.e., T cells, B cells, natural killer cells, macrophages, neutrophils, dendritic cells, mast cells, eosinophils, basophils, etc.) in variable proportions. In some embodiments, lymphocytes include tumor-infiltrating lymphocytes (TILs). TILs are white blood cells that have left the bloodstream and migrated toward a tumor. TILs can often be found in the tumor stroma and within the tumor itself.

In some embodiments, lymphoid cells include peripheral blood lymphocytes (PBLs). In some embodiments, lymphoid cells include T lymphocytes (T cells) and/or natural killer cells (NK cells). In some embodiments, lymphoid cells are T cells. In some embodiments, the T cells are CD8⁺ T cells. In some embodiments, the T cells are CD4⁺ cells. In some embodiments, the T cells are regulatory T cells.

In some embodiments, lymphoid cells are NK cells. In some embodiments, the NK cells are CD16⁺ CD56⁺ and/or CD57⁺ NK cells. NKs are characterized by their ability to bind to and kill cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

In some embodiments, the method includes culturing or expanding the genetically engineered lymphoid cells, e.g., to allow for increased immunogenic activity (e.g., greater and/or longer activity). The term “culturing” or “expanding” refers to maintaining or cultivating cells under conditions in which they can proliferate and avoid senescence. For example, cells may be cultured in media optionally containing one or more growth factors, i.e., a growth factor cocktail. In some embodiments, the cell culture medium is a defined cell culture medium. The cell culture medium may include neoantigen peptides. Stable cell lines may be established to allow for the continued propagation of cells.

Conditions appropriate for lymphocyte culture include an appropriate media (e.g., Minimal Essential Media (MEM), RPMI Media 1640, Lonza RPMI 1640, Advanced RPMI, Clicks, AIM-V, DMEM, a-MEM, F-12, TexMACS, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion).

Examples of other additives for lymphocyte expansion include, but are not limited to, surfactant, piasmanate, pH buffers such as HEPES, and reducing agents such as N-acetyl-cysteine and 2-mercaptoethanol, Antibiotics (e.g., penicillin and streptomycin), are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO2).

Expansion of the lymphoid cells may be carried out using the methods and conditions known in the art. In some embodiments, expansion of the lymphoid cells is carried out according to the methods described in International Application No. PCT/EP2018/080343.

In some embodiments, additional receptor(s) may be engineered into the genetically engineered lymphoid cells expressing a modified TCR. Having additional receptor(s) on the genetically engineered lymphoid cell may enhance the lymphoid cell function (e.g., anti-tumor function).

In some embodiments, the additional receptor is a chimeric antigen receptor (CAR). Chimeric antigen receptors (CARs) typically have an antigen-binding domain that is fused to an intracellular signaling domain which is capable of activating or stimulating an immune cell. The CAR's extracellular binding domain may be composed of a single chain variable fragment (scFv) derived from fusing the variable heavy and light regions of a murine or humanized monoclonal antibody. Alternatively, scFvs may be used that are derived from Fab's (instead of from an antibody, e.g., obtained from Fab libraries). The scFv may be fused to a transmembrane domain and then to an intracellular signaling domain. The CAR can be a first-generation, second generation or third-generation CAR. “First-generation” CARs include those that solely provide CD3z signals upon antigen binding. “Second-generation” CARs include those that provide both costimulation (e.g., CD28 or CD137) and activation (E\ddot{υ}3z). “Third-generation” CARs include those that provide multiple costimulation (e.g., CD28 and CD137) and activation (Eb3z). The CAR may specifically recognize a cancer antigen.

In some embodiments, the cancer antigen may be selected from CD7, CD74, CDS, CEA, EGP-2, EGP-40, EpCAM, erb-B2,3,4, FBP, Fetal acetylcholine receptor, folate receptor-a, GD2, GD3, HER2, hTERT, IL-13R-a2, KDR, K-light chain, LeY, LI cell, MAGE-A1, Mesothelin, MUC1, MUC16, NKG2D ligands, NY-ESO-1, oncofetal antigen (h5T4), PSCA, PSMA, ROR1, TAG-72, VEGF-R2, and WT-1.

Genetically engineered lymphoid cells expressing a modified TCR may be engineered to express and secrete a soluble protein or multiple soluble proteins. Soluble proteins for use in the present invention include, but are not limited to, cytokines, chemokines, growth factors, soluble receptors, ligands, antibodies, antibody fragments, and antigen binding domains and functional variants thereof.

Cytokines that may be expressed and/or secreted by the genetically engineered lymphoid cells described herein include, but are not limited to, interleukin-2 (IL-2), interleukin-3 (IL-3), interleukin-6 (IL-6), interleukin-7 (IL-7), interleukin-9 (IL-9), interleukin-11 (IL-11), interleukin-12 (IL-12), interleukin-15 (IL-15), interleukin-17 (IL-17), interleukin-18 (IL-18), interleukin-21 (IL-21), interleukin-33 (IL-33), granulocyte macrophage colony stimulating factor (GM-CSF), interferon alpha (IFN-alpha or IFN-a), interferon beta (IFN-beta or IFN-b), interferon gamma (IFN-gamma or IFN-g), transforming growth factor-beta (TGF-b), CCL19 and erythropoietin. In some embodiments, the cytokine expressed by the genetically engineered lymphoid cell is IL-2. In some embodiments, the cytokine expressed by the genetically engineered lymphoid cell is IFN-gamma. In some embodiments, the cytokine expressed by the genetically engineered lymphoid cell is GM-CSF.

Chemokines that may be expressed and/or secreted by the genetically engineered lymphoid cells described herein include, but are not limited to, CXC-chemokines such as interleukin-8 (IL-8), neutrophil-activating protein-1 (NAP-1), neutrophil-activating protein-2 (NAP-2), GRO, GROp, GROγ, ENA-78, GCP-2, IP-10, MIG, CXCL1, CXCL12, CXCL16, CXCL19 and PF4; and CC chemokines, RANTES, MIR-Ia, MIR-2b, monocyte chemotactic protein-1 (MCP-1), MCP-2, MCP-3, CCL5, and eotaxin. Suitable chemokines described in the International Publication No. WO2000078334A1 (e.g., Table 1), which is incorporated herein by reference in its entirety, are also contemplated by the present invention.

Growth factors that may be expressed and/or secreted by the genetically engineered lymphoid cells described herein include, but are not limited to, granulocyte macrophage-colony stimulating factor (GM-CSF), granulocyte-colony stimulating factor, macrophage-colony stimulating factor, tumor necrosis factor, transforming growth factors, epidermal growth factors, stem cell factor, platelet-derived growth factors, nerve growth factors, fibroblast growth factors, insulin-like growth factor, growth hormone, interleukin-1 (IL-1), interleukin-2 (IL-2), keratinocyte growth factor, ciliary neurotrophic growth factor, Schwann cell-derived growth factor, vaccinia virus growth factor, bombyxin, neu differentiation factor, v-Sis and glial growth factor.

Soluble receptors that may be expressed and/or secreted by the genetically engineered lymphoid cells described herein include, but are not limited to, soluble cytokine receptors such as IL-1RI, IL-IRII, TNFRI, TNFRII, IFN-α/pR, IL-4 receptor, IL-6 receptor, IL-10 receptor, IL-11 receptor, IL-13 receptor, IL-18 binding protein, and TGF-b receptor; and soluble growth factor receptors such as soluble epidermal growth factor receptors (sEGFRs), soluble vascular endothelial growth factor receptors and PD-1 ectodomain, and soluble VEGFR-i and SIRP-alpha molecules. Soluble receptors that may be expressed and/or secreted by the genetically engineered lymphoid cells described herein may further be fused to CD28 endodomain or 4 IBB endodomain or any other co-stimulatory endodomains known in the art.

The cells can be engineered to express the peptide(s) and/or immunomodulators by any means known in the art, including, but not limited to, transfection, viral delivery (i.e., transduction), liposomal delivery, electroporation, cell squeeze (e.g., cells are first disrupted (e.g., squeezed, deformed, or compressed) followed by exposure to an applied energy field, e.g., an electric, magnetic, or acoustic field), injection, cationic polymer, a cationic lipid, calcium phosphate, and endocytosis.

In some embodiments, genetic engineering of lymphoid cells may be accomplished by at least one of transfection, transduction, or temporary cell membrane disruption (i.e., cell squeeze) to introduce at least one polynucleotide encoding the TCR or the polypeptide disclosed herein into the lymphoid cell. In some embodiments, the polynucleotide(s) are introduced into the lymphoid cells by transducing a substantially homogeneous cell population with a recombinant expression vector. Such vectors may be a viral vector or non-viral vector. Exemplary viral vectors for use in the invention include, but are not limited to, a retroviral vector (including lentiviral vectors), an adenoviral vector, an adeno-associated viral (AAV) vector, a herpes viral vector, or a baculoviral vector. In one embodiment, the viral vector for use in the invention is a lentiviral vector.

In some embodiments, electroporation can be used to permeabilize the cells by the application of electrostatic potential to the cell of interest. Cells subjected to an external electric field in this manner are subsequently predisposed to the uptake of exogenous nucleic acids. Electroporation of mammalian cells is described in detail, e.g., in Chu et al., Nucleic Acids Research 15: 131 1 (1987), the disclosure of which is incorporated herein by reference. A similar technique, Nucleofection™, utilizes an applied electric field in order to stimulate the uptake of exogenous polynucleotides into the nucleus of a eukaryotic cell. Nucleofection™ and protocols useful for performing this technique are described in detail, e.g., in Distler et al., Experimental Dermatology 14:315 (2005), as well as in US 2010/031714, the disclosures of each of which are incorporated herein by reference in their entirety.

Additional techniques useful for the transfection of cells include the cell squeeze-poration methodology. This technique induces the rapid mechanical deformation of cells in order to stimulate the uptake of exogenous DNA through membranous pores that form in response to the applied stress. This technology is advantageous in that a vector is not required for delivery of nucleic acids into a cell, such as a human target cell. Cell squeeze-poration is described in detail, e.g., in Sharei et al., Journal of Visualized Experiments 81:e50980 (2013), the disclosure of which is incorporated herein by reference in its entirety.

Lipofection represents another technique useful for transfection of target cells. This method involves the loading of nucleic acids into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous nucleic acids, for instance, by direct fusion of the liposome with the cell membrane or by endocytosis of the complex. Lipofection is described in detail, for example, in U.S. Pat. No. 7,442,386, the disclosure of which is incorporated herein by reference. Similar techniques that exploit ionic interactions with the cell membrane to provoke the uptake of foreign nucleic acids include contacting a cell with a cationic polymer-nucleic acid complex. Exemplary cationic molecules that associate with polynucleotides so as to impart a positive charge favorable for interaction with the cell membrane include activated dendrimers (described, e.g., in Dennig J., Topics in Current Chemistry 228:227 (2003), the disclosure of which is incorporated herein by reference) and diethylamino ethyl (DEAE)-dextran, the use of which as a transfection agent is described in detail, for instance, in Gulick et al., Current Protocols in Molecular Biology 40:1:9.2:9.2.1 (1997), the disclosure of which is incorporated herein by reference. Magnetic beads are another tool that can be used to transfect target cells in a mild and efficient manner, as this methodology utilizes an applied magnetic field in order to direct the uptake of nucleic acids. This technology is described in detail, for instance, in US 2010/0227406. The disclosure of this reference discussed above is incorporated herein by reference in its entirety.

Another useful tool for inducing the uptake of exogenous nucleic acids by the cell is laserfection, a technique that involves exposing a cell to electromagnetic radiation of a particular wavelength in order to gently permeabilize the cells and allow polynucleotides to penetrate the cell membrane. This technique is described in detail, e.g., in Rhodes et al., Methods in Cell Biology 82:309 (2007), the disclosure of which is incorporated herein by reference in its entirety.

Microvesicles represent another potential vehicle that can be used to modify the genome of a cell according to the methods described herein. For instance, microvesicles that have been induced by the co-overexpression of the glycoprotein VSV-G with, e.g., a genome-modifying protein, such as a nuclease, can be used to efficiently deliver proteins into a cell that subsequently catalyze the site-specific cleavage of an endogenous polynucleotide sequence so as to prepare the genome of the cell for the covalent incorporation of a polynucleotide of interest, such as a gene or regulatory sequence. The use of such vesicles, also referred to as gesicles, for the genetic modification of eukaryotic cells is described in detail, e.g., in Quinn et al., Proceedings of the 18th Annual Meeting of the American Society of Gene and Cell Therapy; 2015 May 13, Abstract No. 122, the disclosure of which is incorporated herein by reference in its entirety.

Various methods may be used to transduce cells. In some embodiments, a cell is transduced with a vector or plasmid, i.e., a nucleic acid molecule capable of transporting a nucleic acid sequence between different cellular or genetic environments. Different cellular environments include different cell types of the same organism, while different genetic environments include cells of different organisms or other situations of cells with different genetic material and/or genomes. Non-limiting vectors of this disclosure include those capable of autonomous replication and expression of nucleic acid sequences (for delivery into the cell) present therein. Vectors may also be inducible for expression in a way that is responsive to factors specific for a cell type. Non-limiting examples include inducible by addition of an exogenous modulator in vitro or systemic delivery of vector inducing drugs in vivo. Vectors may also optionally comprise selectable markers that are compatible with the cellular system used. One type of vector for use in this disclosure is maintained as an episome, which is a nucleic acid capable of extra-chromosomal replication. Another type is a vector which is stably integrated into the genome of the cell in which it is introduced.

g. Compositions and Kits

In another aspect, the above-described TCRs, polypeptides, nucleic acids, vectors, or cells can be incorporated into compositions, e.g., pharmaceutical compositions suitable for administration.

In some embodiments, the pharmaceutical compositions may include a population of antigen-specific (e.g., neoantigen-specific) lymphocytes produced by the methods described herein and a pharmaceutically acceptable carrier and/or excipient. In some embodiments, the pharmaceutical compositions may comprise substantially isolated/purified lymphocytes and a pharmaceutically acceptable carrier in a form suitable for administration to a subject. Pharmaceutically acceptable carriers are determined in part by the particular composition being administered, as well as by the particular method used to administer the composition. The pharmaceutical compositions are generally formulated in full compliance with all Good Manufacturing Practice (GMP) regulations of the U.S. Food and Drug Administration.

The terms “pharmaceutically acceptable” and “physiologically tolerable,” as referred to compositions, carriers, diluents, and reagents, are used interchangeably and include materials that are capable of administration to or upon a subject without the production of undesirable physiological effects to the degree that would prohibit administration of the composition. For example, “pharmaceutically acceptable excipient” includes an excipient that is useful in preparing a pharmaceutical composition that is generally safe, non-toxic, and desirable, and includes excipients that are acceptable for veterinary use as well as for human pharmaceutical use.

Examples of such carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the disclosed composition, use thereof in the compositions is contemplated. In some embodiments, a second therapeutic agent, such as an anti-cancer or anti-tumor, can also be incorporated into pharmaceutical compositions.

Pharmaceutical compositions suitable for injectable use include sterile aqueous solutions (where water-soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate-buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion, and by the use of surfactants.

In some embodiments, the pharmaceutical composition further comprises a therapeutic agent. In some embodiments, the therapeutic agent comprises an anti-tumor or anti-cancer agent.

In some embodiments, the anti-tumor or anti-cancer agent is selected from the group consisting of taxotere, carboplatin, trastuzumab, epirubicin, cyclophosphamide, cisplatin, docetaxel, doxorubicin, etoposide, 5-FU, gemcitabine, methotrexate, and paclitaxel, mitoxantrone, epothilone B, epidermal-growth factor receptor (EGFR)-targeting monoclonal antibody 7A7.27, vorinostat, romidepsin, docosahexaenoic acid, bortezomib, shikonin, an oncolytic virus, and combinations thereof.

In some embodiments, the therapeutic agent comprises a chemotherapeutic agent selected from the group consisting of asparaginase, busulfan, carboplatin, cisplatin, daunorubicin, doxorubicin, fluorouracil, gemcitabine, hydroxyurea, methotrexate, paclitaxel, rituximab, vinblastine, and vincristine.

In some embodiments, the disclosed pharmaceutical compositions can also include adjuvants such as aluminum salts and other mineral adjuvants, tensioactive agents, bacterial derivatives, vehicles, and cytokines. Adjuvants can also have antagonizing immunomodulating properties. For example, adjuvants can stimulate Th1 or Th2 immunity. Compositions and methods as disclosed herein can also include adjuvant therapy.

In some embodiments, the pharmaceutical compositions can be formulated in any conventional manner using one or more physiologically acceptable carriers and/or excipients. The lymphocytes may be formulated for administration by, for example, injection, parenteral, vaginal, rectal administration, or by administration directly to a tumor.

In some embodiments, the pharmaceutical compositions can be formulated for parenteral administration by injection, e.g., by bolus injection or continuous infusion. Formulations for injection can be presented in a unit dosage form, e.g., in ampoules or in multi-dose containers, with an optionally added preservative. In some embodiments, the pharmaceutical compositions can further be formulated as suspensions, solutions or emulsions in oily or aqueous vehicles and may contain other agents, including suspending, stabilizing and/or dispersing agents.

In some embodiments, the pharmaceutical forms suitable for injectable use can include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid. It must be stable under the conditions of manufacture and certain storage parameters (e.g., refrigeration and freezing) and must be preserved against the contaminating action of microorganisms, such as bacteria and fungi.

If formulations disclosed herein are used as a therapeutic to boost immune response in a subject, a therapeutic agent can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine, and the like.

A carrier can also be a solvent or dispersion medium containing, for example, water, saline, polyol (for example, glycerol, propylene glycol, and liquid polyethylene glycol, and the like), suitable mixtures thereof, and vegetable oils. The prevention of the action of microorganisms can be brought about by various antibacterial and antifungal agents known in the art. In many cases, it will be preferable to include isotonic agents, for example, sugars or sodium chloride.

In some embodiments, the above-described TCRs, polypeptides, nucleic acids, vectors, cells or the composition (e.g., the pharmaceutical composition) can be provided in a kit. In one embodiment, the kit includes a container that contains an agent comprising the above-described TCRs, polypeptides, nucleic acids, vectors, cells or the composition, and optionally informational material. The informational material can be descriptive, instructional, marketing or other material that relates to the methods described herein and/or the use of the agents for therapeutic benefit. For example, kits may include instruction for the manufacturing, for the therapeutic regimen to be used, and periods of administration. In an embodiment, the kit includes also includes an additional therapeutic agent (e.g., a checkpoint modulator, a chemotherapeutic compound). The kit may comprise one or more containers, each with a different reagent. For example, the kit includes a first container that contains the above-described TCRs, polypeptides, nucleic acids, vectors, cells or the composition and a second container for the additional therapeutic agent.

In some embodiments, the containers can include a unit dosage of the pharmaceutical composition. In addition to the composition, the kit can include other ingredients, such as a solvent or buffer, an adjuvant, a stabilizer, or a preservative.

In some embodiments, the kit optionally includes a device suitable for administration of the composition, e.g., a syringe or other suitable delivery device. The device can be provided pre-loaded with one or both of the agents or can be empty but suitable for loading.

### B. METHODS OF USE

a. Methods of Treatment and Adoptive T Cell Therapy

In another aspect, this disclosure additionally provides a method of directing immune cells to a target cell (e.g., a tumor cell) in a subject. The method comprises administering to the subject the composition described above. In some embodiments, the immune cell comprises a lymphocyte. In some embodiments, the lymphocyte comprises a T cell or a natural killer (NK) cell. In some embodiments, the T cell comprises a CD8+ T cell or a CD4+ T cell. In some embodiments, the T cell comprises a human T cell.

In another aspect, this disclosure also provides a method for stimulating or enhancing an immune response in a subject in need thereof. The method comprises administering to the subject the composition described herein.

In yet another aspect, the TCR or antigen-binding fragment thereof and T cells comprising the TCRs or antigen-binding fragments thereof can be employed for use in adoptive T cell therapy. In some embodiments, the method for an adoptive T cell therapy in a subject comprises administering to the subject a therapeutically effective amount of the composition described above.

Generally, adoptive T cell therapy relies on the in vitro expansion of endogenous, cancer-reactive T cells. These T cells can be harvested from cancer patients, manipulated, and then reintroduced into the same or a different patient as a mechanism for generating productive tumor immunity. In some embodiments, CD8+ cytotoxic T lymphocytes can be used in adoptive T cell therapy. CD4+ T cells can also play an important role in maintaining CD8+ cytotoxic function, and transplantation of tumor-reactive CD4+ T cells has been associated with some efficacy in metastatic melanoma.

T cells used in adoptive therapy can be harvested from a variety of sites, including peripheral blood, malignant effusions, resected lymph nodes, and tumor biopsies. Although T cells harvested from the peripheral blood are easier to obtain technically, TILs obtained from biopsies may contain a higher frequency of tumor-reactive cells. Once harvested, T cells can be transfected with a vector as described above.

In some embodiments, a TCR or antigen-binding fragment as disclosed has antigen specificity for an antigen that is characteristic of a disease or disorder. The disease or disorder can be any disease or disorder involving an antigen, such as but not limited to a tumor and/or a cancer an infectious disease, or an autoimmune disease.

Accordingly, in one aspect, this disclosure further provides a method of preventing or treating a cancer or a tumor in a subject. The method comprises administering to the subject a therapeutically effective amount of a composition or a pharmaceutical composition, as described above, to a subject in need thereof.

As used herein, the terms “subject” and “patient” are used interchangeably irrespective of whether the subject has or is currently undergoing any form of treatment. As used herein, the terms “subject” and “subjects” may refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgus monkey, chimpanzee, etc.) and a human). The subject may be a human or a non-human.

In some embodiments, the subject is a human. In some embodiments, the subject has a cancer. In some embodiments, the subject is immune-depleted.

As used herein, “cancer,” “tumor,” and “malignancy” all relate equivalently to hyperplasia of a tissue or organ. If the tissue is a part of the lymphatic or immune system, malignant cells may include non-solid tumors of circulating cells. Malignancies of other tissues or organs may produce solid tumors. The methods described herein can be used in the treatment of lymphatic cells, circulating immune cells, and solid tumors.

Cancers that can be treated include tumors that are not vascularized or are not substantially vascularized, as well as vascularized tumors. Cancers may comprise non-solid tumors (such as hematologic tumors, e.g., leukemias and lymphomas) or may comprise solid tumors. The types of cancers to be treated with the disclosed compositions include, but are not limited to, carcinoma, blastoma and sarcoma, and certain leukemias or malignant lymphoid tumors, benign and malignant tumors and malignancies, e.g., sarcomas, carcinomas, and melanomas. Also included are adult tumors/cancers and pediatric tumors/cancers.

Hematologic cancers are cancers of the blood or bone marrow. Examples of hematologic (or hematogenous) cancers include leukemias, including acute leukemias (such as acute lymphocytic leukemia, acute myelocytic leukemia, acute myelogenous leukemia, promyelocytic, myelomonocytic, monocytic, and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high-grade forms), myeloma Multiple, Waldenstrom's macroglobulinemia, heavy chain disease, myelodysplastic syndrome, hairy cell leukemia, and myelodysplasia.

Solid tumors are abnormal masses of tissue that usually do not contain cysts or liquid areas. Solid tumors can be benign or malignant. The different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma and other sarcomas, synovium, mesothelioma, Ewing tumor, leiomyosarcoma, rhabdomyosarcoma, colon carcinoma, lymphoid malignancy, pancreatic cancer, breast cancer, lung cancer, ovarian cancer, prostate cancer, hepatocellular carcinoma, squamous cell carcinoma, basal cell carcinoma, adenocarcinoma, carcinoma of the sweat gland, medullary thyroid carcinoma, papillary thyroid carcinoma, sebaceous gland carcinoma of pheochromocytomas, carcinoma papillary, papillary adenocarcinomas, medullary carcinoma, bronchogenic carcinoma, renal cell carcinoma, hepatoma, bile duct carcinoma, choriocarcinoma, Wilms tumor, cervical cancer, testicular tumor, seminoma, bladder carcinoma, melanoma, and CNS tumors (such as glioma) (such as brainstem glioma and mixed gliomas), glioblastoma (also astrocytoma, CNS lymphoma, germinoma, medulloblastoma, Schwannoma craniopharyngioma, ependymoma, pinealoma, hemangioblastoma, acoustic neuroma, oligodendroglioma, meningioma, neuroblastoma, retinoblastoma, and brain metastasis).

Non-limiting examples of tumors that can be treated by the methods described herein include, for example, carcinomas, lymphomas, sarcomas, blastomas, and leukemias. Non-limiting specific examples, include, for example, breast cancer, pancreatic cancer, liver cancer, lung cancer, prostate cancer, colon cancer, renal cancer, bladder cancer, head and neck carcinoma, thyroid carcinoma, soft tissue sarcoma, ovarian cancer, primary or metastatic melanoma, squamous cell carcinoma, basal cell carcinoma, brain cancers of all histopathologic types, angiosarcoma, hemangiosarcoma, bone sarcoma, fibrosarcoma, myxosarcoma, liposarcoma, chondrosarcoma, osteogenic sarcoma, chordoma, angiosarcoma, endothelio sarcoma, lymphangiosarcoma, lymphangioendotheliosarcoma, synovioma, testicular cancer, uterine cancer, cervical cancer, gastrointestinal cancer, mesothelioma, cancers associated with viral infection (such as but not limited to human papilloma virus (HPV) associated tumors (e.g., cancer cervix, vagina, vulva, head and neck, anal, and penile carcinomas)), Ewing's tumor, leiomyosarcoma, Ewing's sarcoma, rhabdomyosarcoma, carcinoma of unknown primary (CUP), squamous cell carcinoma, basal cell carcinoma, adenocarcinoma, sweat gland carcinoma, sebaceous gland carcinoma, papillary carcinoma, Waldenstrom's macroglobulinemia, papillary adenocarcinomas, cystadenocarcinoma, bronchogenic carcinoma, bile duct carcinoma, choriocarcinoma, seminoma, embryonal carcinoma, Wilms' tumor, lung carcinoma, epithelial carcinoma, cervical cancer, testicular tumor, glioma, glioblastoma, astrocytoma, medulloblastoma, craniopharyngioma, ependymoma, pinealoma, hemangioblastoma, acoustic neuroma, oligodendroglioma, meningioma, retinoblastoma, leukemia, neuroblastoma, small cell lung carcinoma, bladder carcinoma, lymphoma, multiple myeloma, medullary carcinoma, B cell lymphoma, T cell lymphoma, NK cell lymphoma, large granular lymphocytic lymphoma or leukemia, gamma-delta T cell lymphoma or gamma-delta T cell leukemia, mantle cell lymphoma, myeloma, leukemia, chronic myeloid leukemia, acute myeloid leukemia, chronic lymphocytic leukemia, acute lymphocytic leukemia, hairy cell leukemia, hematopoietic neoplasias, thymoma, sarcoma, non-Hodgkin's lymphoma, Hodgkin's lymphoma, Epstein-Barr virus (EBV) induced malignancies of all types including but not limited to EBV-associated Hodgkin's and non-Hodgkin's lymphoma, all forms of post-transplant lymphomas including post-transplant lymphoproliferative disorder (PTLD), uterine cancer, renal cell carcinoma, hepatoma, hepatoblastoma.

Cancers that may be treated by methods and compositions described herein include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus.

In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lympho epithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangio sarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglio neuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythro leukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

The anti-tumor responses after treatment by the methods disclosed herein may be determined in xenograft tumor models. Tumors may be established using any human cancer cell line expressing the TAAs presented by the viral particles. In order to establish xenograft tumor models, about 5×10⁶ viable cells, may be injected, e.g., s.c, into nude athymic mice using, for example, Matrigel (Becton Dickinson). The endpoint of the xenograft tumor models can be determined based on the size of the tumors, weight of animals, survival time, and histochemical and histopathological examination of the cancer, using methods known to one skilled in the art.

In another aspect, disclosed herein is a method for treating infectious and/or zoonotic diseases in a subject in need thereof comprising administering to the subject the effective amount of the composition, e.g., a population of antigen-specific lymphocytes produced by the methods disclosed herein. Infectious diseases are caused by pathogenic microorganisms, such as bacteria, viruses, parasites or fungi; the diseases can be spread, directly or indirectly, from one person to another. Zoonotic diseases are infectious diseases of animals that can cause disease when transmitted to humans. Examples of infectious and/or zoonotic diseases include, but are not limited to, acute and chronic infectious processes that can result in obstruction of body passageways including, for example, obstructions of the male reproductive tract (e.g., strictures due to urethritis, epididymitis, prostatitis); obstructions of the female reproductive tract (e.g., vaginitis, cervicitis, pelvic inflammatory disease (e.g., tuberculosis, gonococcus, chlamydia, enterococcus, and syphilis); urinary tract obstructions (e.g., cystitis, urethritis); respiratory tract obstructions (e.g., chronic bronchitis, tuberculosis, other mycobacterial infections (MAI, etc.), anaerobic infections, fungal infections, and parasitic infections) and cardiovascular obstructions (e.g., mycotic aneurysms and infective endocarditis).

In some embodiments, administration of the lymphocytes generated by the methods as disclosed herein can be used to treat viral infections and/or tumors resulting from viral infection. Exemplary viruses include, but are not limited herpesviruses such as the simplexviruses (e.g., human herpesvirus-1 (HHV-1), human herpesvirus-2 (HHV-2)), the varicelloviruses (e.g., human herpesvirus-3 (HHV-3, also known as varicella zoster virus)), the lymphocryptoviruses (e.g., human herpesvirus-4 (HHV-4, also known as Epstein Barr virus (EBV))), the cytomegaloviruses (e.g., human herpesvirus-5 (HHV-5), also known as human cytomegalovirus (HCMV)), the roseoloviruses (e.g., human herpesvirus 6 (HHV-6), human herpesvirus 7 (HHV-7)), the rhadinovirues (e.g., human herpesvirus 8 (HHV-8, also known as Kaposi's Sarcoma associated herpesvirus (KSHV)); poxviruses such as orthopoxviruses (e.g., cowpoxvirus, monkeypoxvirus, vaccinia virus, variola virus), parapoxviruses (e.g., bovine popular stomatitis virus, orf virus, pseudocowpox virus), moUuscipoxviruses (e.g., molluscum contagiosum virus), yatapoxviruses (e.g., tanapox virus, yaba monkey tumor virus); adenoviruses (e.g., Human adenovirus A (HAdV-A), Human adenovirus B (HAdV-B), Human adenovirus C (HAdV-C), Human adenovirus D (HAdV-D), Human adenovirus E (HAdV-E), Human adenovirus F (HAdV-F)); papillomaviruses (e.g., human papillomavirus (HPV); parvoviruses (e.g., B19 virus); hepadnoviruses (e.g., Hepatitis B virus (HBV)); retroviruses such as deltaretroviruses (e.g., primate T-lymphotrophic virus 1 (HTLV-1) and primate T-lymphotrophic virus 2 (HTLV-2)) and lentiviruses (e.g., Human Immunodeficiency Virus 1 (HIV-1) and Human Immunodeficiency Virus 2 (HIV-2); reoviruses such the orthoreo viruses (e.g., mammalian orthoreovirus (MRV)), the orbviruses (e.g., African horse sickness virus (AHSV), Changuinola virus (CORV), Orungo virus (ORUV), and the rotaviruses (e.g., rotavirus A (RV-A) and rotavirus B (RV-B)); filoviruses such as the “Marburg-like viruses” (e.g., MARV), the “Ebola-like viruses” (e.g., CIEBOV, REBOV, SEBOV, ZEBOV); paramyxoviruses such as respiroviruses (e.g., human parainfluenza virus 1 (HPIV-1), human parainfluenza virus 3 (HPIV-3), rubulaviruses (e.g., human parainfluenza virus 2 (HPIV-2), human parainfluenza virus 4 (HPIV-4)), mumps virus (MuV)), and morbilliviruses (e.g., measles virus); pneumoviruses (e.g., human respiratory syncitial virus (HSCV); rhabdoviruses such as the vesiculoviruses (e.g., vesicular stomatitis virus), the lyssaviruses (e.g., rabies virus); orthomyxoviruses (e.g., Influenza A virus, Influenza B virus, Influenza C virus); bunyaviruses (e.g., California encephalitis virus (CEV)); hantaviruses (e.g., Black Creek Canal virus (BCCV), New York virus (NYV), Sin Nombre virus (SNV)); picomaviruses including the enteroviruses (e.g., human enterovirus A (HEV-A), human enterovirus B (HEV-B), human enterovirus C (HEV-C), human enterovirus D (HEV-D), poliovirus (PV)), the rhinoviruses (e.g., human rhino virus A (HRV-A), human rhino virus B (HRV-B)), the hepatoviruses (e.g., Hepatitis A virus (HAV)); caliciviruses including the “Norwalk-like viruses” (e.g., Norwalk Virus (NV), and the “Sapporo-like viruses” (e.g., Sapporo virus (SV)); togaviruses including alphaviruses (e.g., Western equine encephalitis virus (WEEV) and Eastern equine encephalitis virus (EEEV)) and rubiviruses (e.g., Rubella virus); flaviviruses (e.g., Dengue virus (DENV), Japanese encephalitis (JEV), St. Louis encephalitis virus (SLEV), West Nile virus (WNV), Yellow fever virus (YFV); arenaviruses (e.g., lassa virus); coronaviruses (e.g., the severe acute respiratory syndrome (SARS)-associated virus); and hepaciviruses (e.g., Hepatitis C virus (HCV)).

In some embodiments, the condition treatable by the disclosed methods includes autoimmune disease. The term “autoimmune disease” as used herein is defined as a disorder that results from an autoimmune response. An autoimmune disease can be the result of an inappropriate and excessive response to a self-antigen. Non-limiting examples of autoimmune disease of Acquired Immunodeficiency Syndrome (AIDS), alopecia areata, ankylosing spondylitis, antiphospholipid syndrome, autoimmune Addison's disease, autoimmune hemolytic anemia, autoimmune hepatitis, autoimmune inner ear disease (AIED), autoimmune lymphoproliferative syndrome (ALPS), autoimmune thrombocytopenic purpura (ATP), Behcet's disease, cardiomyopathy, celiac sprue-dermatitis hepetiformis; chronic fatigue immune dysfunction syndrome (CFIDS), chronic inflammatory demyelinating polyneuropathy (CIPD), cicatricial pemphigoid, cold agglutinin disease, crest syndrome, Crohn's disease, Degos' disease, dermatomyositis-juvenile, discoid lupus, essential mixed cryoglobulinemia, fibromyalgia-fibromyositis, Graves' disease, Guillain-Barre syndrome, Hashimoto's thyroiditis, idiopathic pulmonary fibrosis, idiopathic thrombocytopenia purpura (ITP), IgA nephropathy, insulin-dependent diabetes mellitus, juvenile chronic arthritis (Still's disease), juvenile rheumatoid arthritis, Meniere's disease, mixed connective tissue disease, multiple sclerosis, myasthenia gravis, pernacious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, Reiter's syndrome, rheumatic fever, rheumatoid arthritis, sarcoidosis, scleroderma (progressive systemic sclerosis (PSS), also known as systemic sclerosis (SS)), Sjogren's syndrome, stiff-man syndrome, systemic lupus erythematosus, Takayasu arteritis, temporal arteritis/giant cell arteritis, ulcerative colitis, uveitis, vitiligo, Wegener's granulomatosis, and any combination thereof.

In some embodiments, lymphodepletion prior to adoptive transfer of antigen-specific lymphocytes can play a role in enhancing treatment efficacy by eliminating regulatory T cells and competing elements of the immune system. Accordingly, in some embodiments, a lymphodepletion step (also referred to as “immunosuppressive conditioning”) is utilized on the subject prior to the introduction of the antigen-specific lymphocytes. Lymphodepletion can be achieved by administering compounds such as, but not limited to, fludarabine or cyclophosphamide (the active form being referred to as mafosfamide) and combinations thereof. Such methods are described in Gassner, et al., Cancer Immunol. Immunother. 2011, 60, 75-85; Muranski, et al., Nat. Clin. Pract. Oncol, 2006, 3, 668-681; Dudley, et al., J. Clin. Oncol. 2008, 26, 5233-5239; and Dudley, et al., J. Clin. Oncol. 2005, 23, 2346-2357, the disclosures of which are incorporated by reference herein in their entireties.

In some embodiments, the subject is immunodepleted prior to treatment with the composition, e.g., antigen-specific lymphocytes. For example, the subject can be pre-treated with non-myeloablative chemotherapy prior to an infusion of lymphocytes generated by the methods described herein. In one embodiment, a population of antigen-specific lymphocytes can be administered by infusion. In one embodiment, the non-myeloablative chemotherapy can be cyclophosphamide 60 mg/kg/d for 2 days (days 27 and 26 prior to antigen-specific lymphocyte infusion) and fludarabine 25 mg/m2/d for 5 days (days 27 to 23 prior to antigen-specific lymphocyte infusion). In one embodiment, after non-myeloablative chemotherapy and antigen-specific lymphocyte infusion (at day 0), according to the present disclosure, the subject can receive an intravenous infusion of IL-2 intravenously at 720,000 IU/kg every 8 hours to physiologic tolerance. In some embodiments, the population of antigen-specific lymphocytes can be used for treating cancer in combination with IL-2, wherein the IL-2 is administered after the population of antigen-specific lymphocytes.

In some embodiments, the composition, e.g., a population of antigen-specific lymphocytes, is administered with an additional therapeutic agent or therapy. In some embodiments, the composition can be administered to a subject either simultaneously with, before (e.g., 1-30 days before) or after (e.g., 1-30 days after) the additional therapeutic (including but not limited to small molecules, antibodies, or cellular reagents) that acts to elicit an immune response (e.g., to treat cancer) in the subject. When co-administered with an additional therapeutic, the composition and the additional therapeutic agent may be administered simultaneously or sequentially (in any order). Suitable therapeutically effective dosages for each agent may be lowered due to the additive action or synergy.

In some embodiments, the methods described herein can be combined with additional immunotherapies and therapies. For example, when used for treating cancer, the composition can be used in combination with conventional cancer therapies, such as, e.g., surgery, radiotherapy, chemotherapy or combinations thereof, depending on type of the tumor, patient condition, other health issues, and a variety of factors. In some embodiments, other therapeutic agents useful for combination cancer therapy with the inhibitors described herein include anti-angiogenic agents. Many anti-angiogenic agents have been identified and are known in the art, including, e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by Carmeliet and Jain (2000). In some embodiments, the inhibitors described herein can be used in combination with a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof (e.g., anti-hVEGF antibody A4.6.1, bevacizumab or ranibizumab).

Non-limiting examples of chemotherapeutic compounds which can be used in combination treatments include, for example, aminoglutethimide, amsacrine, anastrozole, asparaginase, beg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramnustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic compounds may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, Cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP 16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

In some embodiments, the composition can be combined with other immunomodulatory treatments such as, e.g., therapeutic vaccines (including but not limited to GVAX, DC-based vaccines, etc.), checkpoint inhibitors (including but not limited to agents that block CTLA4, PD1, LAG3, TIM3, etc.) or activators (including but not limited to agents that enhance 41BB, OX40, etc.). The inhibitory treatments described herein can also be combined with other treatments that possess the ability to modulate NKT function or stability, including but not limited to CD Id, CD Id-fusion proteins, CD Id dimers or larger polymers of CD Id, either unloaded or loaded with antigens, CDld-chimeric antigen receptors (CDld-CAR), or any other of the five known CD1 isomers existing in humans (CD la, CD lb, CDlc, CDle), in any of the aforementioned forms or formulations, alone or in combination with each other or other agents.

The pharmaceutical compositions, as described, can be administered in a manner appropriate to the disease to be treated or prevented. The amount and frequency of administration will be determined by factors such as the condition of the patient, and the type and severity of the patient's disease, although appropriate dosages can be determined by clinical trials.

When “a therapeutically effective amount,” “an immunologically effective amount,” “an effective antitumor quantity,” or “an effective tumor-inhibiting amount” is indicated, the precise amount of the compositions of the present disclosure to be administered can be determined by a physician having account for individual differences in age, weight, tumor size, extent of infection or metastasis, and patient's condition. It can generally be stated that a pharmaceutical composition comprising the lymphocytes described herein can be administered at a dose of 10⁴ to 10⁹ cells/kg body weight, e.g., 10⁵ to 10⁶ cells/kg body weight, including all values integers within these intervals. The lymphocyte compositions can also be administered several times at these dosages. The cells can be administered using infusion techniques that are commonly known in immunotherapy (see, for example, Rosenberg et al., New Eng. J. of Med. 319: 1676, 1988). The optimal dose and treatment regimen for a particular patient can be readily determined by one skilled in the art of medicine by monitoring the patient for signs of the disease and adjusting the treatment accordingly.

In some embodiments, the composition can be administered to the subject in a manner compatible with the dosage formulation and in such amount as is therapeutically effective. Dose ranges and frequency of administration can vary depending on, e.g., the nature of the population of cells (e.g., antigen-specific lymphocytes) produced by the methods described herein and the medical condition as well as parameters of a specific patient and the route of administration used.

In some embodiments, the population of cells, e.g., antigen-specific lymphocytes (e.g., neoantigen-specific lymphocytes), produced by the methods described herein can be administered to a subject at a dose ranging from about 10⁷ to about 10¹². A more accurate dose can also depend on the subject in which it is being administered. For example, a lower dose may be required if the subject is juvenile, and a higher dose may be required if the subject is an adult human subject. In some embodiments, a more accurate dose can depend on the weight of the subject.

The administration of the compositions as disclosed can be carried out in any convenient way, including infusion or injection (i.e., intravenous, intrathecal, intramuscular, intraluminal, intratracheal, intraperitoneal, or subcutaneous), transdermal administration, or other methods known in the art. Administration can be once every two weeks, once a week, or more often, but the frequency may be decreased during a maintenance phase of the disease or disorder. In some embodiments, the composition is administered by intravenous infusion.

In some embodiments, the cells, e.g., antigen-specific lymphocytes, are activated and expanded using the methods described herein or other methods known in the art, wherein the cells are expanded to therapeutic levels, before administering to a patient together with (e.g., before, simultaneously or after) any number of relevant treatment modalities.

Also described herein, the compositions can be used in combination with chemotherapy, radiation, immunosuppressive agents, such as cyclosporin, azathioprine, methotrexate, mycophenolate, and FK506, antibodies, or other immunoablating agents such as CAMPATH, anti-cancer antibodies. CD3 or other antibody therapies, cytoxine, fludarabine, cyclosporine, FK506, rapamycin, mycophenolic acid, steroids, FR901228, cytokines, and irradiation.

In some embodiments, the compositions can also be administered to a patient together with (e.g., before, simultaneously or after) bone marrow transplantation, therapy with T lymphocyte ablation using chemotherapy agents such as fludarabine, radiation therapy external beam (XRT), cyclophosphamide, or antibodies such as OKT3 or CAMPATH. Also described herein, the compositions can be administered after ablative therapy of B lymphocytes, such as agents that react with CD20, for example, Rituxan. For example, subjects may undergo standard treatment with high-dose chemotherapy, followed by transplantation of peripheral blood stem cells. In some embodiments, after transplantation, the subjects receive an infusion of the expanded lymphocytes, or the expanded lymphocytes are administered before or after surgery.

b. Methods of Detection

In another aspect, this disclosure additionally provides a method of detecting cancer in a biological sample. The method comprises: (a) contacting the biological sample with the TCR or antigen-binding fragment thereof described above, and (b) detecting binding of the TCR or antigen-binding fragment thereof to the biological sample, wherein detection of binding is indicative of cancer.

In some embodiments, the method of detecting cancer is carried out in vitro, in vivo or in situ.

In some embodiments, the biological sample can be a sample comprising whole cells, lysates thereof, or a fraction of the whole cell lysates, e.g., a nuclear or cytoplasmic fraction, a whole protein fraction, or a nucleic acid fraction. In some embodiments, the biological sample can be untreated, enzymatically treated, and/or dissociated/suspended to form a cell suspension. When the sample is enzymatically treated, non-limited examples of enzymes that can be used include collagenase, dispase, hyaluronidase, liberase, and DNase.

In some embodiments, the TCR or antigen-binding fragment thereof comprises a detectable label. In some embodiments, the detectable label is selected from the group consisting of a radionuclide, a fluorophore, and biotin.

In some embodiments, the TCR or antigen-binding fragment thereof may be immobilized, either directly or indirectly onto a variety of supports, such as magnetic or chromatographic matrix particles, the surface of an assay plate (such as microtiter wells), pieces of a solid substrate material, and the like. An assay plate or strip can be prepared by coating the TCR or antigen-binding fragment thereof in an array on a solid support. Any solid support known in the art can be used, including but not limited to, solid supports made out of polymeric materials in the forms of wells, tubes or beads. The TCR or antigen-binding fragment thereof can be bound to the solid support by adsorption, by covalent bonding using a chemical coupling agent, by binding to a capture antibody, or by other means known in the art, provided that such binding does not interfere with the binding ability of the capture proteins. Moreover, if necessary, the solid support can be derivatized to allow reactivity with various functional groups on the proteins. Such derivatization requires the use of certain coupling agents such as, but not limited to, maleic anhydride, N-hydroxysuccinimide, and 1-ethyl-3-(3-dimethylaminopropyl)carbodiimide. In some embodiments, the solid phase substrate may include microparticles, microbeads, magnetic beads, membrane, and an affinity purification column.

### C. DEFINITIONS

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the phrases “nucleic acid,” “polynucleotide,” “oligonucleotide,” and “nucleic acid molecule” are used interchangeably to refer to a polymer of DNA and/or RNA, which can be single-stranded, double-stranded, or multi-stranded, synthesized or obtained (e.g., isolated and/or purified) from natural sources, which can contain natural, non-natural, and/or altered nucleotides, and which can contain natural, non-natural, and/or altered internucleotide linkages including, but not limited to phosphoroamidate linkages and/or phosphorothioate linkages instead of the phosphodiester found between the nucleotides of an unmodified oligonucleotide.

As used herein, “expression” refers to the process by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as “gene products.” If the polynucleotide is derived from genomic DNA, expression may include splicing of the mRNA in a eukaryotic cell.

As used herein, the term “recombinant” refers to a cell, microorganism, nucleic acid molecule or vector that has been modified by the introduction of an exogenous nucleic acid molecule or has controlled expression of an endogenous nucleic acid molecule or gene., Deregulated or altered to be constitutively altered, such alterations or modifications can be introduced by genetic engineering. Genetic alteration includes, for example, modification by introducing a nucleic acid molecule encoding one or more proteins or enzymes (which may include an expression control element such as a promoter), or addition, deletion, substitution of another nucleic acid molecule. Or other functional disruption of, or functional addition to, the genetic material of the cell. Exemplary modifications include modifications in the coding region of a heterologous or homologous polypeptide derived from the reference or parent molecule or a functional fragment thereof.

The terms “T cell” and “T lymphocyte” are interchangeable and used synonymously herein. As used herein, T-cell includes thymocytes, naive T lymphocytes, immature T lymphocytes, mature T lymphocytes, resting T lymphocytes, or activated T lymphocytes. A T-cell can be a T helper (Th) cell, for example, a T helper 1 (Thl) or a T helper 2 (Th2) cell. The T-cell can be a helper T-cell (HTL; CD4⁺ T-cell) CD4⁺ T-cell, a cytotoxic T-cell (CTL; CD8⁺ T-cell), a tumor-infiltrating cytotoxic T-cell (TIL; CD8⁺ T-cell), CD4⁺ CD8⁺ T-cell, or any other subset of T-cells. Other illustrative populations of T-cells suitable for use in particular embodiments include naive T-cells and memory T-cells. Also included are “NKT cells,” which refer to a specialized population of T-cells that express a semi-invariant ab T-cell receptor, but also express a variety of molecular markers that are typically associated with NK cells, such as NK1.1. NKT cells include NK1.1′ and NK1. G, as well as CD4⁺, CD4, CD8⁺, and CD8 cells. The TCR on NKT cells is unique in that it recognizes glycolipid antigens presented by the MHC I-like molecule CD Id. NKT cells can have either protective or deleterious effects due to their abilities to produce cytokines that promote either inflammation or immune tolerance. Also included are “gamma-delta T-cells (T6 T-cells),” which refer to a specialized population that to a small subset of T-cells possessing a distinct TCR on their surface, and unlike the majority of T-cells in which the TCR is composed of two glycoprotein chains designated a- and b-TCR chains, the TCR in 76 T-cells is made up of a g-chain and a d-chain. γδ T-cells can play a role in immunosurveillance and immunoregulation and were found to be an important source of IL-17 and to induce robust CD8⁺ cytotoxic T-cell response. Also included are “regulatory T-cells” or “Tregs” which refer to T-cells that suppress an abnormal or excessive immune response and play a role in immune tolerance. Tregs cells are typically transcription factor Foxp3-positive CD4⁺ T cells and can also include transcription factor Foxp3-negative regulatory T-cells that are IL-10-producing CD4+ T cells.

The terms “natural killer cell” and “NK cell” are used interchangeably and used synonymously herein. As used herein, NK cell refers to a differentiated lymphocyte with a CD 16+ CD56+ and/or CD57+ TCR− phenotype. NKs are characterized by their ability to bind to and kill cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition, but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof, or (3) relieving the disease, i.e., causing regression of the state, disorder or condition or at least one of its clinical or sub-clinical symptoms. The benefit to a subject to be treated is either statistically significant or at least perceptible to the patient or to the physician. Thus, the term “treatment” includes preventing a condition from occurring in a patient, particularly when the patient is predisposed to acquiring the condition; reducing and/or inhibiting the condition and/or its development and/or progression; and/or ameliorating and/or reversing the condition. Insofar as some embodiments of the methods of the presently disclosed subject matter are directed to preventing conditions, it is understood that the term “prevent” does not require that the condition be completely thwarted. Rather, as used herein, the term “preventing” refers to the ability of one of ordinary skill in the art to identify a population that is susceptible to the condition, such that administration of the compositions of the presently disclosed subject matter might occur prior to the onset of the condition. The term does not imply that the condition must be completely avoided.

The term “inhibiting cell growth” or “inhibiting proliferation of cells” refers to reducing or halting the growth rate of cells. For example, by inhibiting the growth of tumor cells, the rate of increase in size of the tumor may slow. In other embodiments, the tumor may stay the same size or decrease in size, i.e., regress. In particular embodiments, the rate of cell growth or cell proliferation is inhibited by at least 20%, at least 30%, at least 40%, at least 50%, at least 60%, at least 70%, at least 80%, or at least 90%.

The terms “transformation” and “transfection” refer to the directed modification of the genome of a cell by the external application of purified recombinant DNA from another cell of different genotype, leading to its uptake and integration into the subject cell's genome. In bacteria, the recombinant DNA is not typically integrated into the bacterial chromosome, but instead replicates autonomously as a plasmid. The terms “transformed” and “transfected” are used interchangeably herein. For example, a T cell may be transfected with a DNA sequence encoding a modified or high affinity TCR described herein prior to adoptive T cell treatment.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments f(ab′)2, and fab. F(ab′)2, and fab fragments that lack the Fe fragment of intact antibody, clear more rapidly from the circulation and may have less non-specific tissue binding of an intact antibody (Wahl et al., J. Nucl. Med. 24:316-325 (1983). The antibodies of this disclosure comprise whole native antibodies, bispecific antibodies; chimeric antibodies; fab, fab′, single-chain v region fragments (scFv), fusion polypeptides, and unconventional antibodies.

As used herein, the term “single-chain variable fragment” or “scFv” is a fusion protein of the variable regions of the heavy (VH) and light chains (VL) of an immunoglobulin covalently linked to form a VH::VL heterodimer. The heavy (VH) and light chains (VL) are either joined directly or joined by a peptide-encoding linker (e.g., 10, 15, 20, 25 amino acids), which connects the N-terminus of the VH with the C-terminus of the VL, or the C-terminus of the VH with the N-terminus of the VL. The linker is usually rich in glycine for flexibility, as well as serine or threonine for solubility. Despite removal of the constant regions and the introduction of a linker, scFv proteins retain the specificity of the original immunoglobulin. Single-chain Fv polypeptide antibodies can be expressed from a nucleic acid including VH- and VL-encoding sequences as described by Huston, et al. (Proc. Nat. Acad. Sci., 85:5879-5883, 1988). See also, U.S. Pat. Nos. 5,091,513, 5,132,405 and 4,956,778; and US patent publication nos. 20050196754 and 20050196754. Antagonistic scFvs having inhibitory activity have been described (see, e.g., Zhao et al., Hybridoma (Larchmont) 2008 27(6):455-51; Peter et al., J cachexia sarcopenia muscle 2012 Aug. 12; Shieh et al., J Immunol 2009 183(4):2277-85; Giomarelli et al., Thromb Haemost 2007 97(6):955-63; Fife etal., J Clin Invst 2006 116(8):2252-61; Brocks etal., Immunotechnology 1997 3(3):173-84; Moosmayer et al., Ther Immunol 1995 2(10:31-40). Agonistic scFvs having stimulatory activity have been described (see, e.g., Peter et al., J Bioi Chem 2003 25278(38):36740-7; Xie etal., Nat Biotech 1997 15(8):768-71; Ledbetter et al., Crit Rev Immunol 1997 17(5-6):427-55; Ho et al., Biochim Biophys Acta 2003 1638(3):257-66).

The term “eliciting” or “enhancing” in the context of an immune response refers to triggering or increasing an immune response, such as an increase in the ability of immune cells to target and/or kill cancer cells or to target and/or kill pathogens and pathogen-infected cells (e.g., EBV-positive cancer cells).

The term “immune response,” as used herein, refers to any type of immune response, including, but not limited to, innate immune responses (e.g., activation of Toll receptor signaling cascade), cell-mediated immune responses (e.g., responses mediated by T cells (e.g., antigen-specific T cells) and non-specific cells of the immune system) and humoral immune responses (e.g., responses mediated by B cells (e.g., via generation and secretion of antibodies into the plasma, lymph, and/or tissue fluids). The term “immune response” is meant to encompass all aspects of the capability of a subject's immune system to respond to antigens and/or immunogens (e.g., both the initial response to an immunogen (e.g., a pathogen) as well as acquired (e.g., memory) responses that are a result of an adaptive immune response).

The term “disease” as used herein is intended to be generally synonymous and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “effective amount,” “effective dose,” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A “prophylactically effective amount” or a “prophylactically effective dosage” of a drug is an amount of the drug that, when administered alone or in combination with another therapeutic agent to a subject at risk of developing a disease or of suffering a recurrence of disease, inhibits the development or recurrence of the disease. The ability of a therapeutic or prophylactic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

Doses are often expressed in relation to bodyweight. Thus, a dose which is expressed as [g, mg, or other unit]/kg (or g, mg etc.) usually refers to [g, mg, or other unit] “per kg (or g, mg etc.) bodyweight,” even if the term “bodyweight” is not explicitly mentioned.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

The terms “therapeutic agent,” “therapeutic capable agent,” or “treatment agent” are used interchangeably and refer to a molecule or compound that confers some beneficial effect upon administration to a subject. The beneficial effect includes enablement of diagnostic determinations; amelioration of a disease, symptom, disorder or pathological condition; reducing or preventing the onset of a disease, symptom, disorder or condition; and generally counteracting a disease, symptom, disorder or pathological condition.

“Combination” therapy, as used herein, unless otherwise clear from the context, is meant to encompass administration of two or more therapeutic agents in a coordinated fashion and includes, but is not limited to, concurrent dosing. Specifically, combination therapy encompasses both co-administration (e.g., administration of a co-formulation or simultaneous administration of separate therapeutic compositions) and serial or sequential administration, provided that administration of one therapeutic agent is conditioned in some way on administration of another therapeutic agent. For example, one therapeutic agent may be administered only after a different therapeutic agent has been administered and allowed to act for a prescribed period of time. See, e.g., Kohrt et al. (2011) Blood 117:2423.

As used herein, the term “pharmaceutical composition” refers to a mixture of at least one compound useful within this disclosure with other chemical components, such as carriers, stabilizers, diluents, dispersing agents, suspending agents, thickening agents, and/or excipients.

The pharmaceutical composition facilitates administration of the compound to an organism.

As used herein, the term “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained.

The term “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting an agent within or to the subject such that it may perform its intended function. Typically, such agents are carried or transported from one organ, or portion of the body, to another organ, or portion of the body. Each salt or carrier must be “acceptable” in the sense of being compatible with the other ingredients of the formulation and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, absorption delaying agents, and the like that are compatible with the activity of the compound, and are physiologically acceptable to the subject. Supplementary active compounds may also be incorporated into the compositions.

“Parenteral” administration of a composition includes, e.g., subcutaneous (s.c.), intravenous (i.v.), intramuscular (i.m.), or intrasternal injection, or infusion techniques.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

The word “substantially” does not exclude “completely,” e.g., a composition which is “substantially free” from Y may be completely free from Y. Where necessary, the word “substantially” may be omitted from the definition of this disclosure.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%1, 1%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

It is to be understood that wherever values and ranges are provided herein, all values and ranges encompassed by these values and ranges are meant to be encompassed within the scope of the present disclosure. Moreover, all values that fall within these ranges, as well as the upper or lower limits of a range of values, are also contemplated by the present application.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of this disclosure unless otherwise claimed. No language in the specification should be construed as indicating any non-claimed element as essential to the practice of this disclosure.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present disclosure. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### D. EXAMPLES

### Example 1

This example describes the materials and methods used in the subsequent EXAMPLES below.

### Patient

Patients included stage III/IV metastatic melanoma, ovarian, non-small cell lung cancer, and colorectal cancer patients and had received several lines of chemotherapy (Table 1). Patients were enrolled under protocols approved by the respective institutional regulatory committees at the University of Pennsylvania, USA, and Lausanne University Hospital (Ethics Committee, University Hospital of Lausanne-CHUV, Switzerland). Also, samples from four melanoma patients enrolled in a phase I clinical trial of TIL ACT were collected at baseline (NCT03475134). All patients signed informed consent.

### Tumors and Blood Processing

Resected tumors were minced into 1-2 mm² pieces or enzymatically digested and cryopreserved in 90% human serum+10% dimethyl sulfoxide (DMSO) as described (Bobisse, S. et al. Nat. Commun. 9, 1092 (2018); Gannon, P. O. et al. Cytotherapy 000, 1-12 (2020)). Both enzymatically digested tumor cells and tumor fragments were used as starting material for TIL generation. PBMCs were isolated from leukapheresis upon thawing and washing using the LoVo spinning membrane filtration system (Frenesius Kabi AG). PBMCs were cryopreserved in 90% human serum+10% DMSO.

### Generation of Tumor Cell Lines

Tumor cell lines were established from tumor fragments and cultured in R10 medium (RPMI 1640 complemented with 10% fetal bovine serum, 100 mM HEPES (Gibco), 100IU/mL Peninicillin and 100 μg/mL Streptomycin (Bioconcept)) at 37° C. at 5% CO₂. Culture medium was replenished every 2-3 days, and cultures were split when confluent. To this end, tumor cells were gently detached with Accutase (Thermo Fisher Scientific) and split, and R10 medium was fully replenished. The day before any co-culture assay (screening assay described below), tumor cells were incubated 24 hrs in R10 medium supplemented with 200 ng/mL IFNγ (Miltenyi).

### Generation and Electroporation of Antigen-Presenting Cells (APCs)

B cells were isolated from autologous cryopreserved PBMCs or apheresis samples by positive selection of CD19 cells with microbeads (Miltenyi). CD19 cells were then cultured at 37° C. at 5% CO₂ for 7 to −20 days in R8 medium (RPMI 1640 (Gibco) with 8% Human AB serum (Biowest), non-essential amino acids, 100 mM HEPES, 1 mM Sodium Pyruvate, 50 μM 2-mercaptoethanol (Gibco), 100IU/mL Penicillin, 100 μg/mL Streptomycin (Bioconcept), 2 mM L-Glutamine Solution (Bioconcept), supplemented with 0.5-1 μg/mL multimeric CD40L (Adipogen), with 40 ng/mL IL-4 (Miltenyi) and 50 ng/mL IL-21 (Miltenyi). Between day 7 and 14, B cells were harvested and either used for screening or TIL generation or frozen for future use. For flow cytometry phenotyping analysis, day 9-12 B cells were stained with anti-human CD19, -CD80, -OX40L, -CD70 (BD Biosciences), -HLA-ABC, -HLA-DR, -CD40, -CD83, -CD86 (Biolegend), -4-1BBL (Miltenyi) and Aqua viability dye (Thermo Fisher Scientific) in two distinct FACS panels, acquired on a four-lasers Fortessa (BD biosciences) and analyzed with FlowJo X (TreeStar).

The secretion of IL-12 by B cells was assessed by MSD immunoassay (Human Cytokine 30-Plex, Meso Scale Discovery) according to the manufacturer's instructions and was analyzed with the MESO QuickPlex SQ 120 instrument (Meso Scale Discovery).

Before electroporation, B cells were rested overnight in their culture medium including cytokines and without CD40L. Cells were electroporated using both the Neon transfection 10 μL and 100 μL kits (Thermo Fisher Scientific). Briefly, B cells were harvested, washed twice, and resuspended at 10-20e6 cells/mL in buffer T. B cells were mixed with 100 μg/mL IVT TMG RNA and/or with 33 μg/mL of each immune-stimulatory IVT RNA. Cells were then electroporated in 10 μl (0.1-0.2e6 cells) or 100 μl (1-2e6 cells) tips with the following parameters: 1400V, 10 msec, 3 pulses. After transfection, cells were added to a pre-warmed medium and either incubated for 2 to 17 hrs (overnight) at 37° C. or used immediately.

### Identification of Non-Synonymous Tumor Mutations and Prediction of Neoantigens

Non-synonymous point tumor mutations arising from single nucleotide variants (SNVs) were identified from tumor tissues and matched blood cells. Samples from patients 4, 6, 7, 8, and 9 were analyzed as previously described (Bobisse, S. et al. Nat. Commun. 9, 1092 (2018)). Samples from patients 1, 2, 3, and 5 were analyzed with NeoDisc V1.2 pipeline (Bassani-Sternberg, M. et al. Front. Immunol. 10, 1-17 (2019)) that includes the GATK (der Auwera, G. A. et al. Curr. Protoc. Bioinforma. 43, 11.10.1-11.10.33 (2013)) variant calling algorithm Mutect2, Mutect1, HaplotypeCaller and VarScan 2. NeoDisc v1.2 also determines the presence of each mutation and quantifies the expression of each mutant gene and mutation from RNAseq data. Predictions for binding to HLA class-I of all candidate peptides of samples from patients 4, 6, 7, 8, and 9 were performed using the netMHC v3.4, netMHCpan-3.0 (Nielsen, M. et al. Genome Med. 8, 1-9 (2016)) algorithms. Predictions for binding and immunogenicity on HLA class-I and HLA-class II candidate peptides of samples from patients 1, 2, 3, and 5 were performed using the PRIME (Schmidt, J. et al. Cell Reports Med. (2021)) and MixMHCpred2 algorithms (Gfeller, D. et al. J. Immunol. 201, 3705-3716 (2018); assani-Sternberg, M. et al. PLoS Comput. Biol. 13, e1005725 (2017)). Long peptides consisted of 31mers with the mutation at the center position for samples from patients 4 and 7 and for samples from patients 1, 2, 3, and 5 were optimally designed, as described (Bassani-Sternberg, M. et al. Front. Immunol. 10, 1-17 (2019)). Long and short peptides analyzed with NeoDisc v1.2 were selected based on their binding and immunogenicity predictions, the expression of the mutant genes, the expression of the mutations, and the presentation of the peptides in IpMSDB (a database of hotspots of antigens presentation) (Müller, M., et al. Front. Immunol. 8, 1-14 (2017)).

For HLA typing, genomic DNA was extracted from the sample using a DNeasy kit from Qiagen. HLA typing was performed with the TruSight HLA v2 Sequencing Panel from CareDx. Briefly, 400ng of gDNA was used to amplify HLA genes by polymerase chain reaction (PCR). Nextera adapters were added by tagmentation, and the resulting libraries were sequenced on the MiniSeq instrument of Illumina. Sequencing data were then analyzed with the Assign TruSight HLA v2.1 software provided by CareDx.

Identification of TAAs by immunopeptidomics

Immunoaffinity purification of HLA-I complexes from tissues was performed as previously described (Chong, C. et al. Mol. Cell. Proteomics 17, 533-548 (2018)) with the anti-HLA-I W6/32 antibody. HLA-I binding peptides were eluted with 1% TFA and concentrated. Peptides were measured with LC-MS/MS system consisting of an Easy-nLC 1200 and the Q Exactive HF-X mass spectrometer (Thermo Fisher Scientific). The immunopeptidomics MS data against the patient-specific customized reference database as previously described (Bassani-Sternberg, M. et al. Nat. Commun. 7, 1-16 (2016)) were searched with the MaxQuant computational environment (Cox, J. et al. Nat. Biotechnol. 26, 1367-1372 (2008)). The enzyme specificity was set as unspecific, and peptides with a length between 8 and 25 amino acids were allowed. A false discovery rate (FDR) of 5% was required for peptides, and no protein FDR was set. Peptides derived from known TAAs were selected for further analysis.

### Design of DNA Constructs and In Vitro Transcription of RNA

Tandem minigenes (TMGs) were in silico designed as previously described (Sahin, U. et al. Nature 547, 222-226 (2017); Holtkamp, S. et al. Blood 108, 4009-4018 (2006), codon-optimized, and synthesized by gene synthesis at GeneArt (Thermo Fisher Scientific). Briefly, five minigenes by 31mer each were centered on identified mutated amino acids and spaced by non-immunogenic glycine/serine linkers. The resulting TMGs were flanked by a signaling peptide (SP) and by MHC-class I trafficking signals (MITD) (Kreiter, S. etal. J. Immunol. 180, 309-318 (2007)).

To obtain OX40L, 4-1BBL, and IL-12 (alpha/beta) expressing vectors, full-length sequences coding for each immune stimulatory molecule were cloned into pcDNA™6/myc-His-C for OX40L and 4-1BBL (Thermo Fisher Scientific) and pGEM®-T (Promega) for IL-12, downstream of a T7 promoter. Plasmids encoding OX40L, 4-1BBL, and IL-12 were linearized respectively with Eco RV., Sma I. (NEB), and Xba I (Thermo Fisher Scientific).

For the TCR cloning methodology, DNA sequences coding for full-length TCR chains were codon-optimized and synthesized by GeneArt (Thermo Fisher Scientific) as strings. Each DNA sequence included a T7 promoter upstream of the ATG codon while human constant regions of alpha and beta chains were replaced by corresponding homologous murine constant regions.

Linearized plasmid DNA and purified PCR products served as templates for the in vitro transcription (IVT) and polyadenylation of RNA molecules as per manufacturer's instructions (Thermo Fisher Scientific). Polyadenylation and integrity were assessed by gel electrophoresis in denaturing conditions, and RNA was quantified with a Qbit fluorometer (Thermo Fisher Scientific). Purified RNA was resuspended in H₂O at 1-10 μg/mL and stored at −80° C. until used.

### Peptide Loading

Peptides (purity >70%) were synthesized and lyophilized by the Peptide and Tetramer Core Facility of the Department of Oncology UNIL-CHUV (Lausanne, Switzerland) or by Covalab (Lyon, France).

For minimal epitope loading (i.e., 9-10mer), cells were harvested, washed twice with RPMI medium, and resuspended at 1e6 cells/mL in RPMI complemented with 1% Human serum and with individual peptides or peptide pools at 1 μg/mL. APCs were incubated at 37° C. for 1-2 hrs and washed twice with RPMI medium before use in co-culture assays.

For long peptide (i.e., 31mer) pulsing, APCs were harvested, washed twice with RPMI medium, and resuspended at 1e6 cells/mL in R8 medium complemented only with cytokines. Peptides were added at 1 μg/mL. APCs were then incubated at 37° C. for 17-20 hrs and washed twice with RPMI medium before use in co-culture assays.

### TIL Cultures

Conventional TILs were grown in R8 medium supplemented with 60001U/mL IL-2 (Proleukin). 2-6 tumor fragments (1-3 mm³) or a total of 1e6 dissociated tumor cells were plated per well of p24-well plate. Besides tumor samples and high-dose of IL-2, NeoScreen TILs were generated by addition of engineered B cells presenting tumor antigen candidates at day 0 of culture. Antigens were in form of minigenes or pools of predicted peptides (<139) at 1 μg/mL each. For patient 4, a total of 191 peptides were split into two pools, noted as follows: NeoScreen (1) and NeoScreen (2) (Table 2 and 4). 1e6 and 2e6 of B cells were added per well of the p24-well plate with dissociated tumor cells and tumor fragments, respectively. Cells were cultured at 37° C. at 5% CO₂ and maintained at a concentration of 1e6 cells/mL. On day 7-10, TILs were harvested, counted, and washed, and a fraction of NeoScreen TILs underwent a second round of stimulation with B cells (i.e., identical setting than at day 0). After 16-22 days, TILs were collected, screened, TCR sequenced, and cryopreserved.

### Antigen Screening of TIL Cultures

IFNγ Enzyme-Linked ImmunoSpot (ELISpot) and pNMC-multimer complexes staining were performed at the end of cultures, and antigens were validated by ≥3 independent experiments. For patient 4, NeoScreen (1) and NeoScreen (2) were interrogated, each with corresponding antigen candidates, added at the initiation of TIL generation. For patient 7, NeoScreen TILs were generated (×1) and re-stimulated (×2) in parallel with TMG and LP loaded engineered CD40-act B cells, so the frequency of antigen-specific TILs obtained was averaged between the two antigen sources, unless specified.

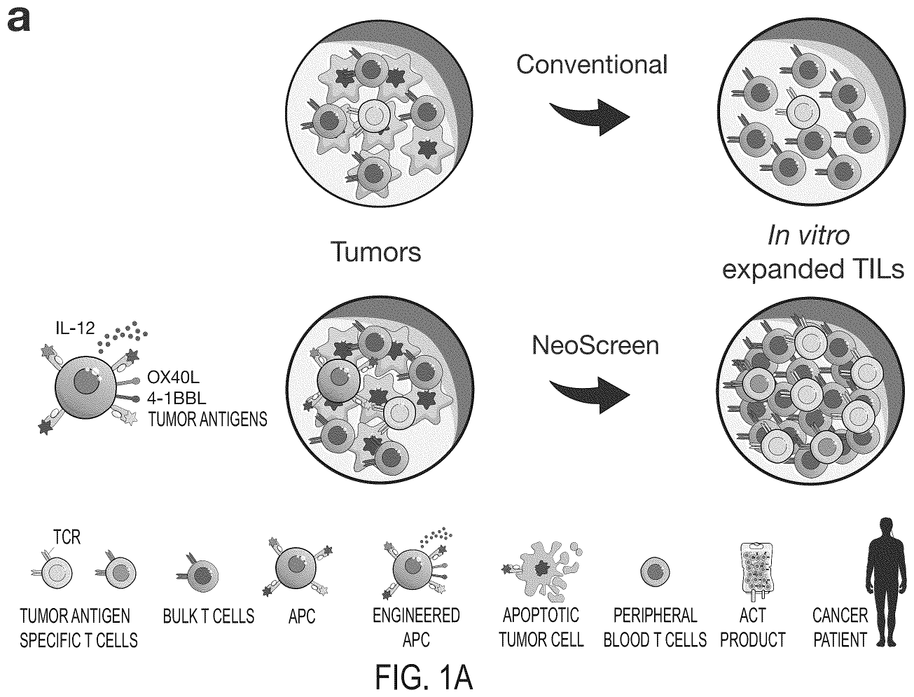

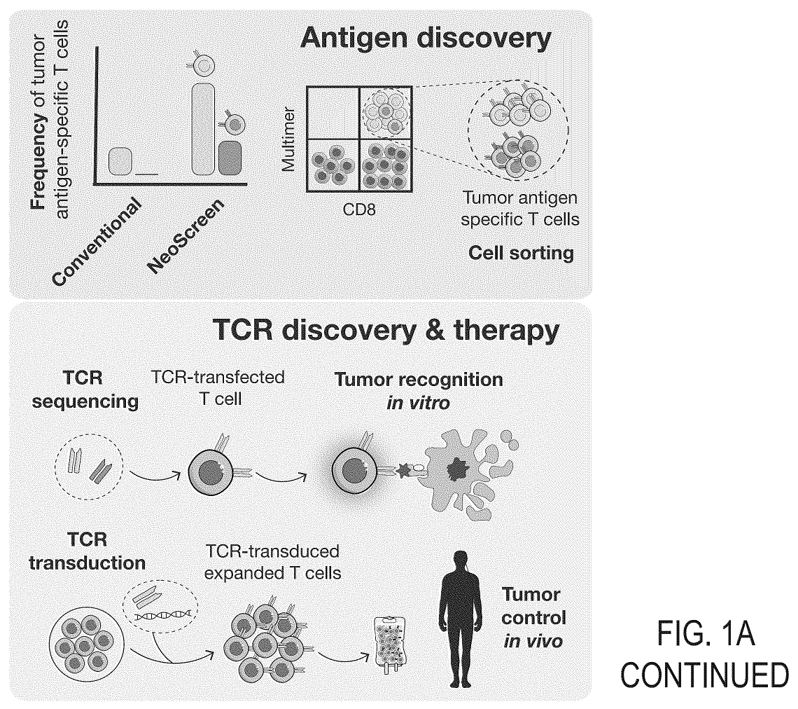

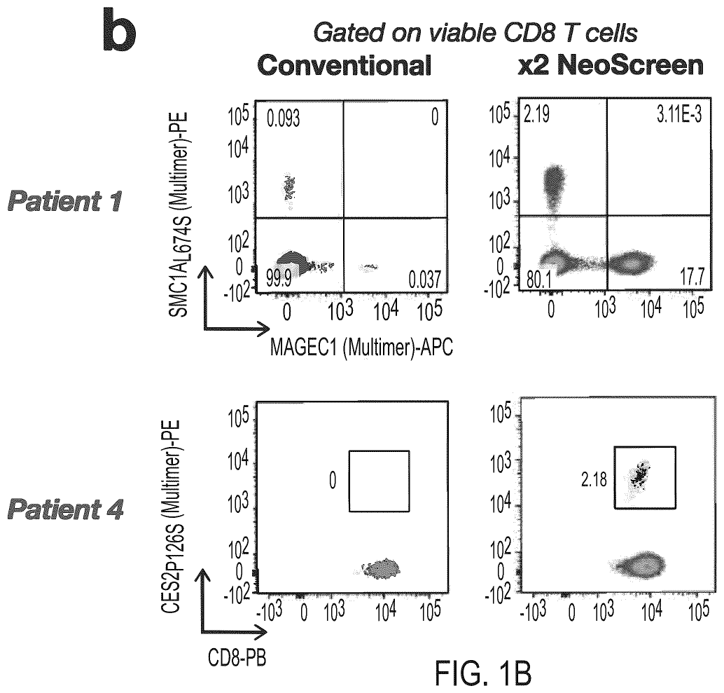

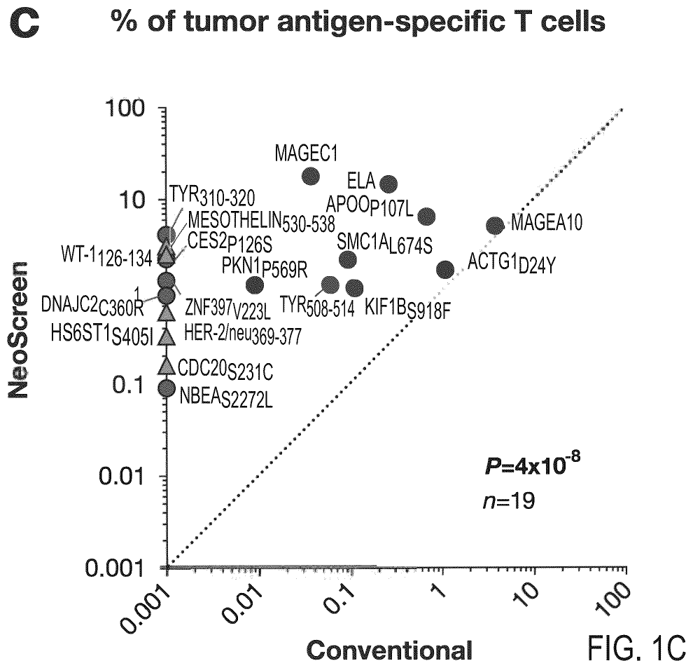

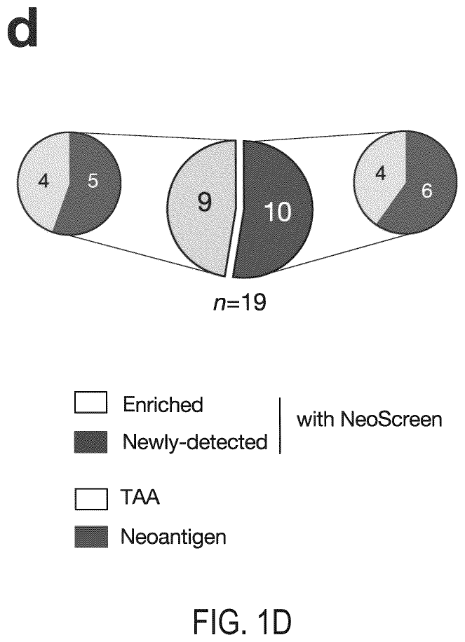

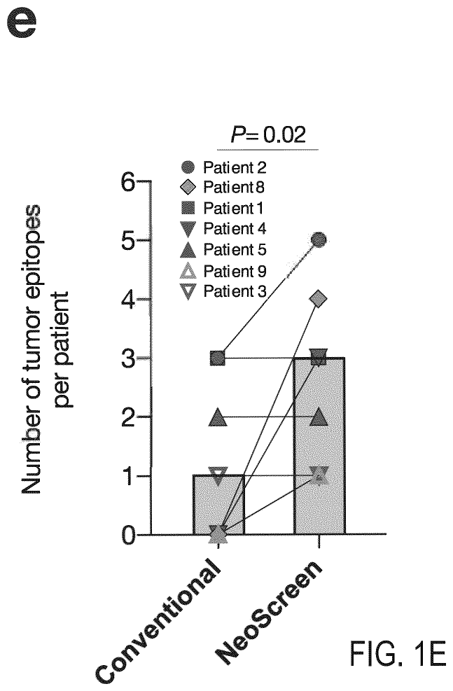

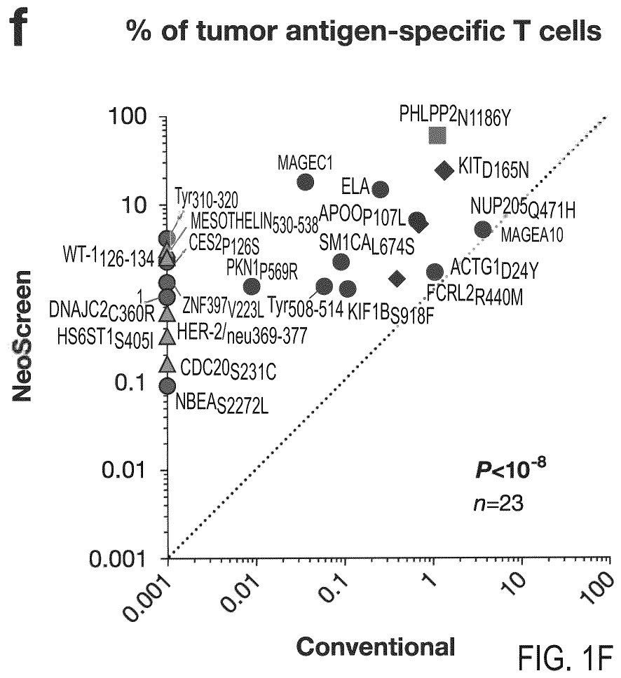

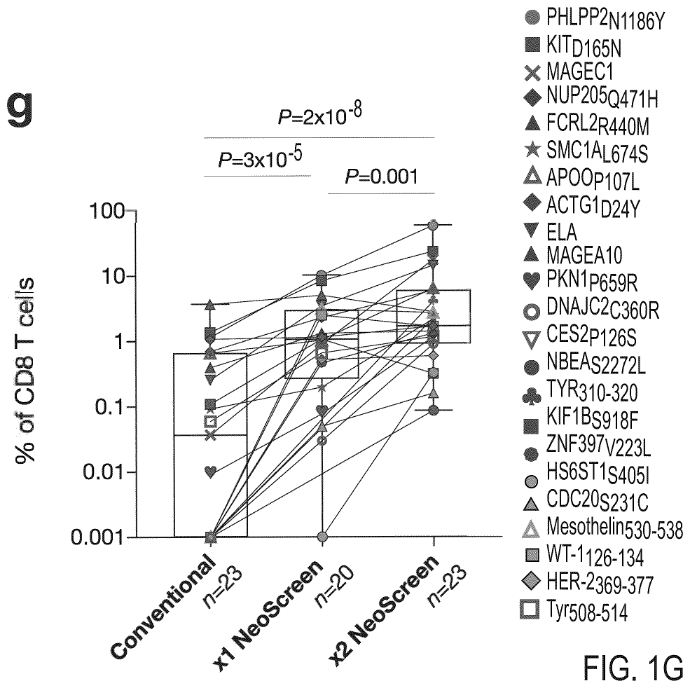

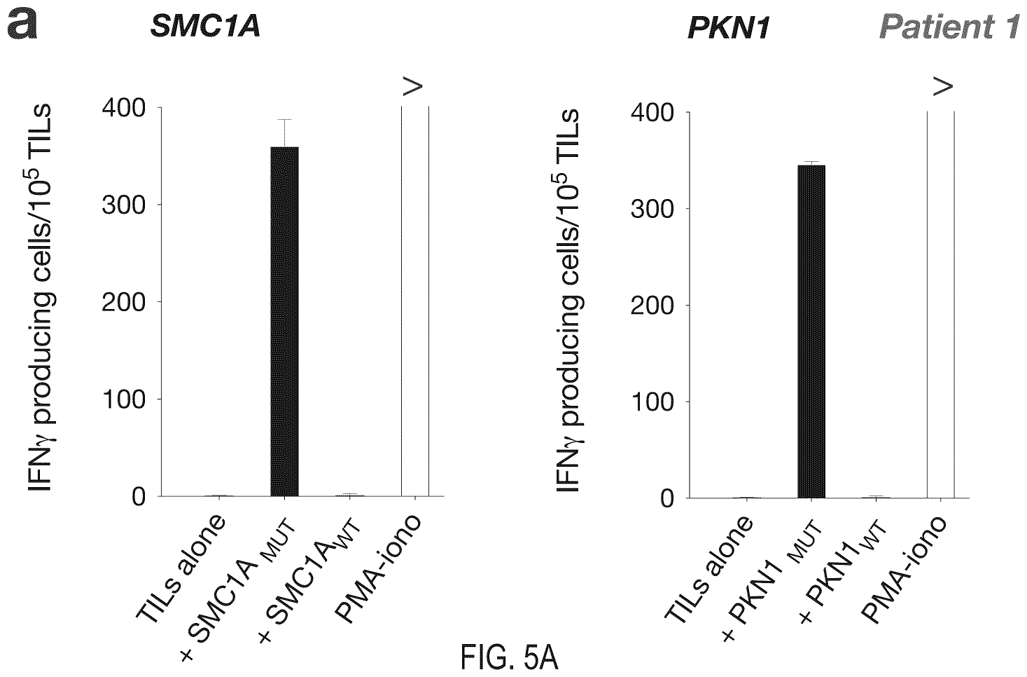

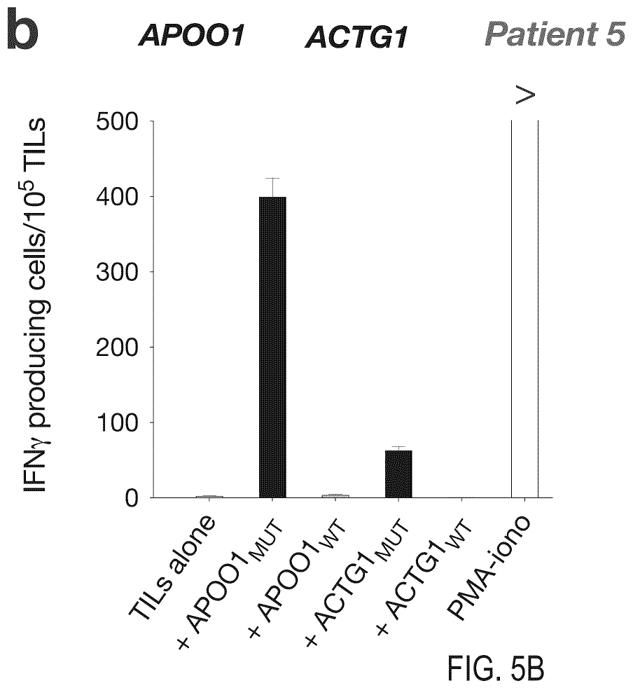

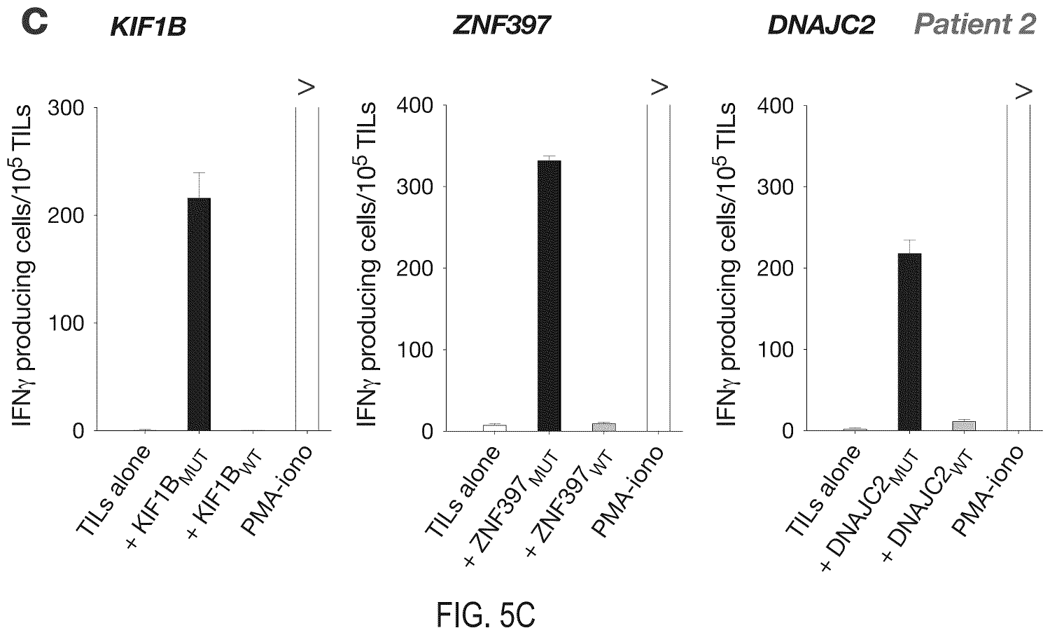

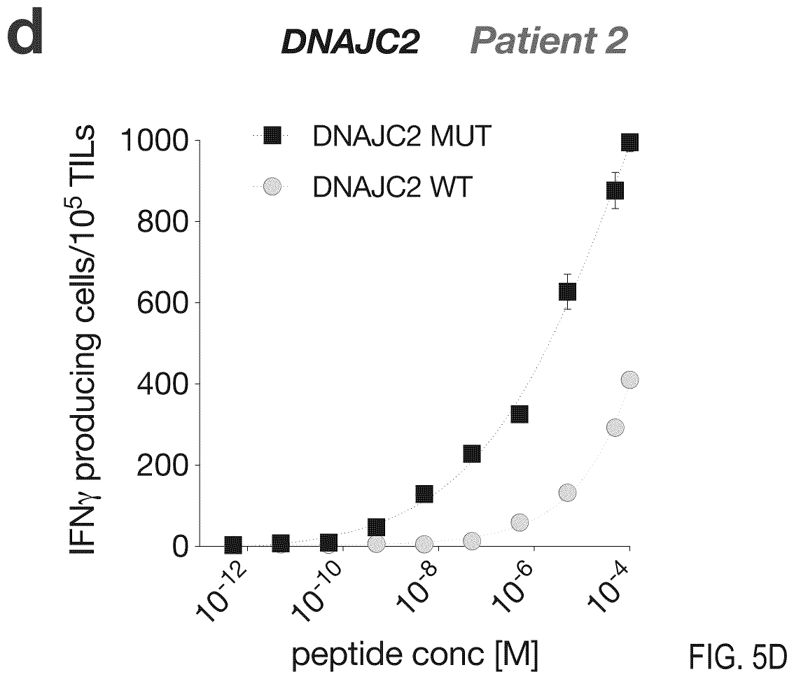

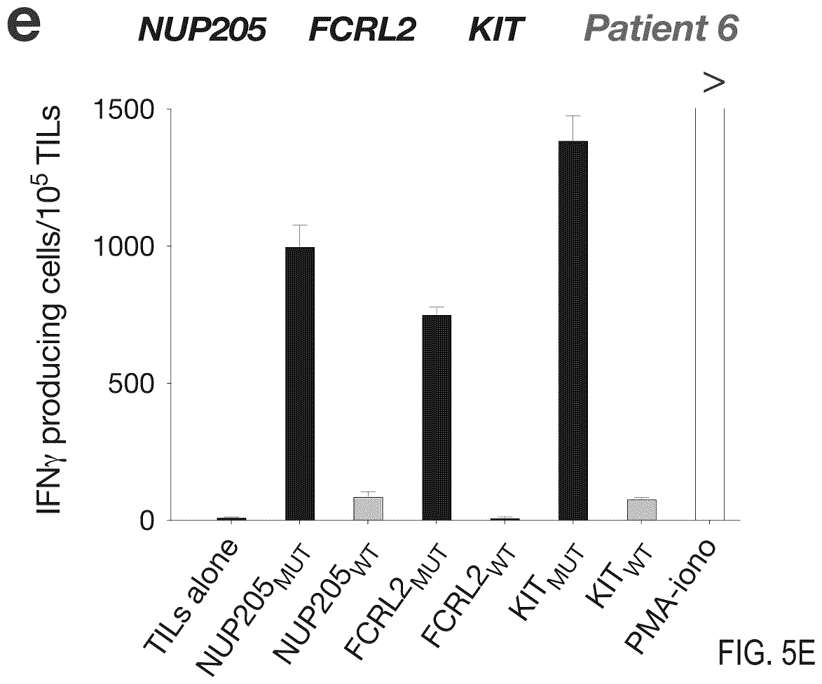

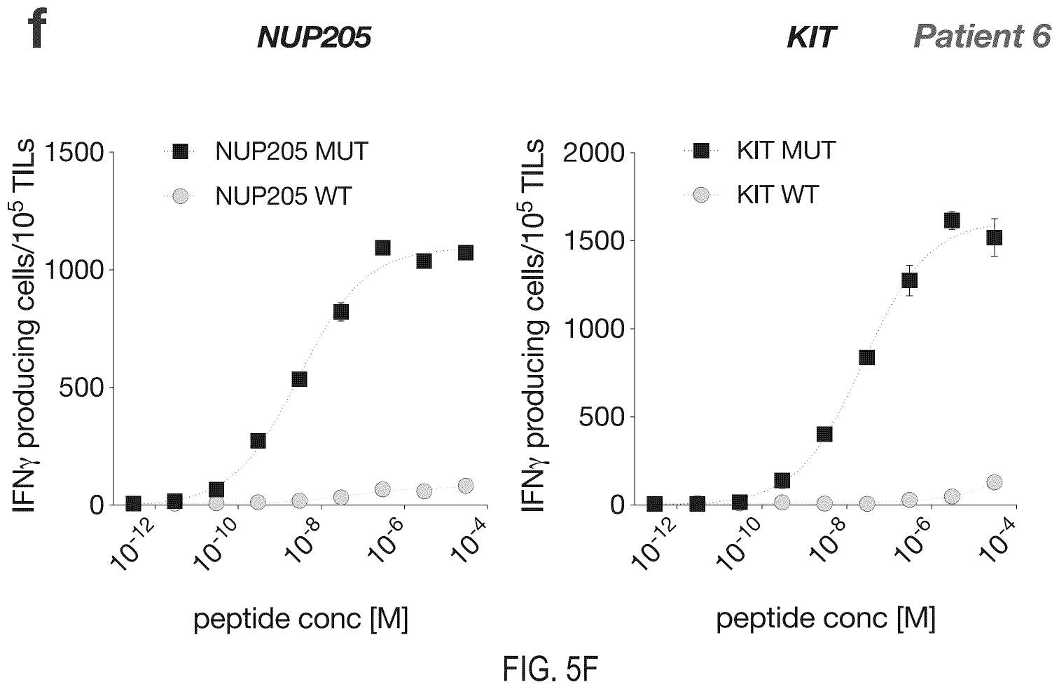

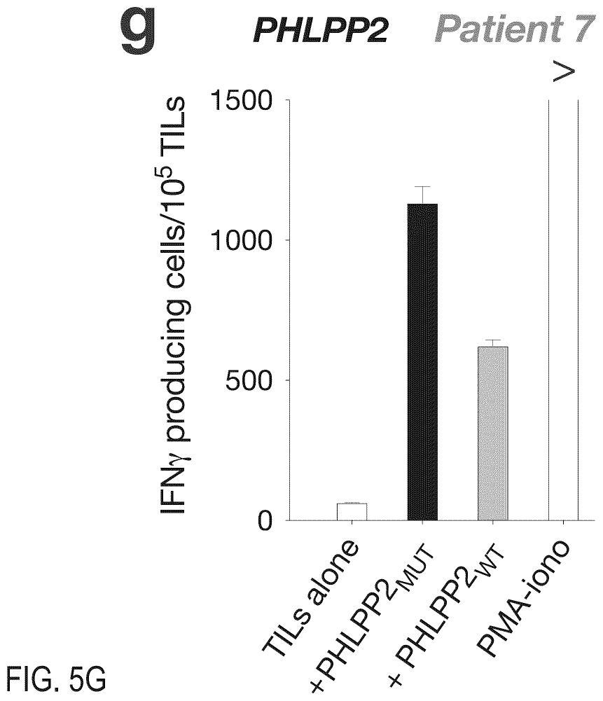

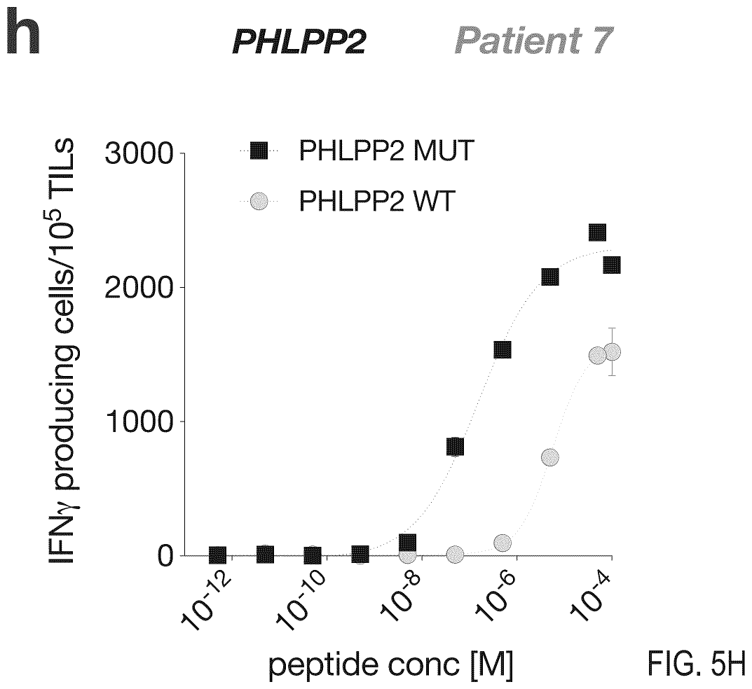

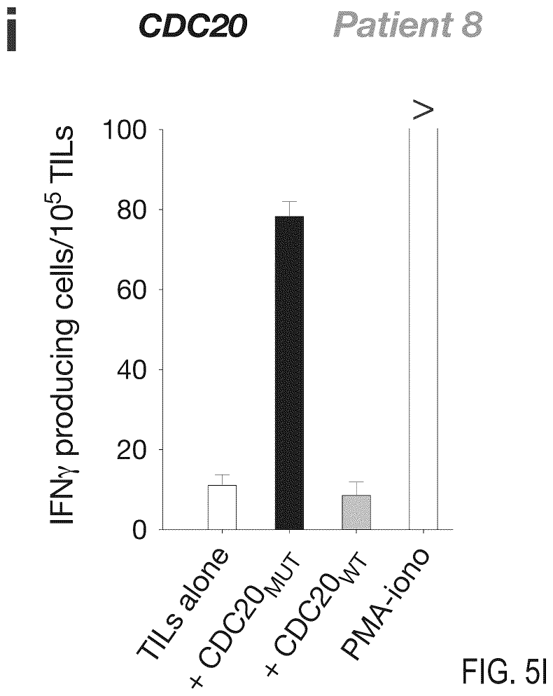

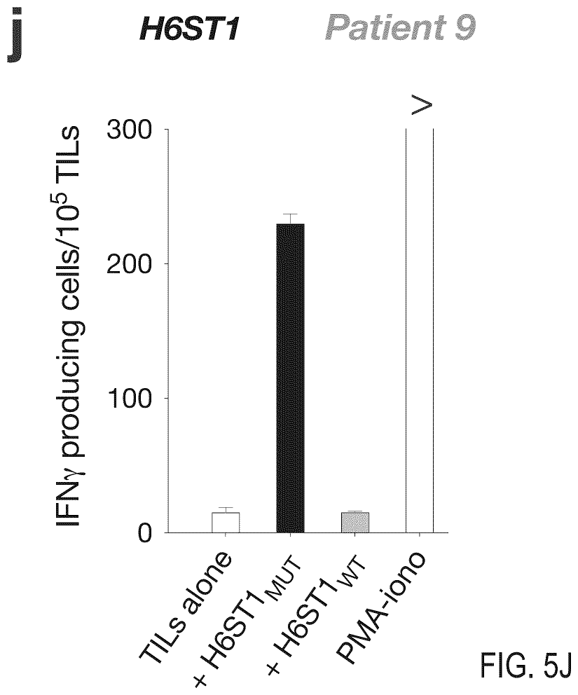

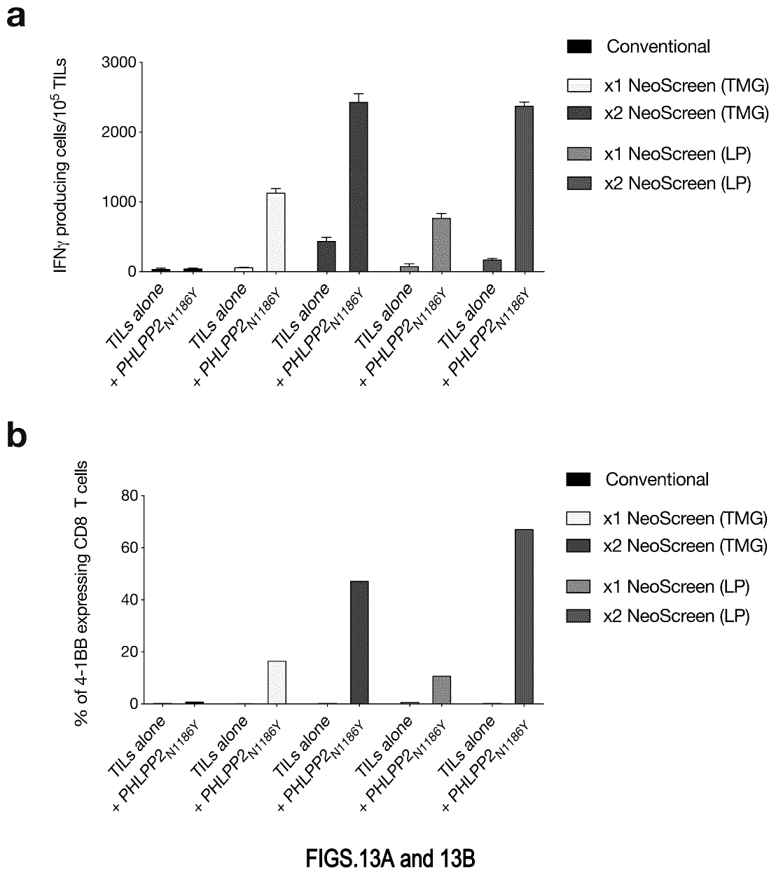

ELISpot assays were performed using pre-coated 96-well ELISpot plates (Mabtech), as previously described (Bobisse, S. et al. Nat. Commun. 9, 1092 (2018)). Briefly, 5e4 to 2e5 TILs were plated per well and challenged with tumor-specific peptides at 1 μg/mL (single peptides or peptide pools of ≤100 peptides) (see example on FIG. 13A). The background level of IFNγ Spot Forming Unit per 10⁵ cells by the negative control (TILs alone) was subtracted to that of antigen re-challenged TILs in all cumulative figures. The cross-reactivity of neoepitope-specific T cell responses was assessed by challenging TILs with the wild-type peptide at 1 μg/mL. Cross-reactivity was then further evaluated by performing limiting peptide dilutions (ranging from 100 μg/mL to 0.1 μg/mL) (FIG. 5). When autologous B cells were used in ELISpot assay, a ratio of 2:1, TILs: APCs was applied (see FIG. 1i). Before the assay, TILs were rested for 48 hrs in a culture medium from which IL-2 was removed in two steps. Phorbol 12-myristate 13-acetate ionomycin (PMA-iono) (Thermo Fisher Scientific) was used to stimulate TILs as positive control, and 1e3 TILs were plated per ELISpot well.

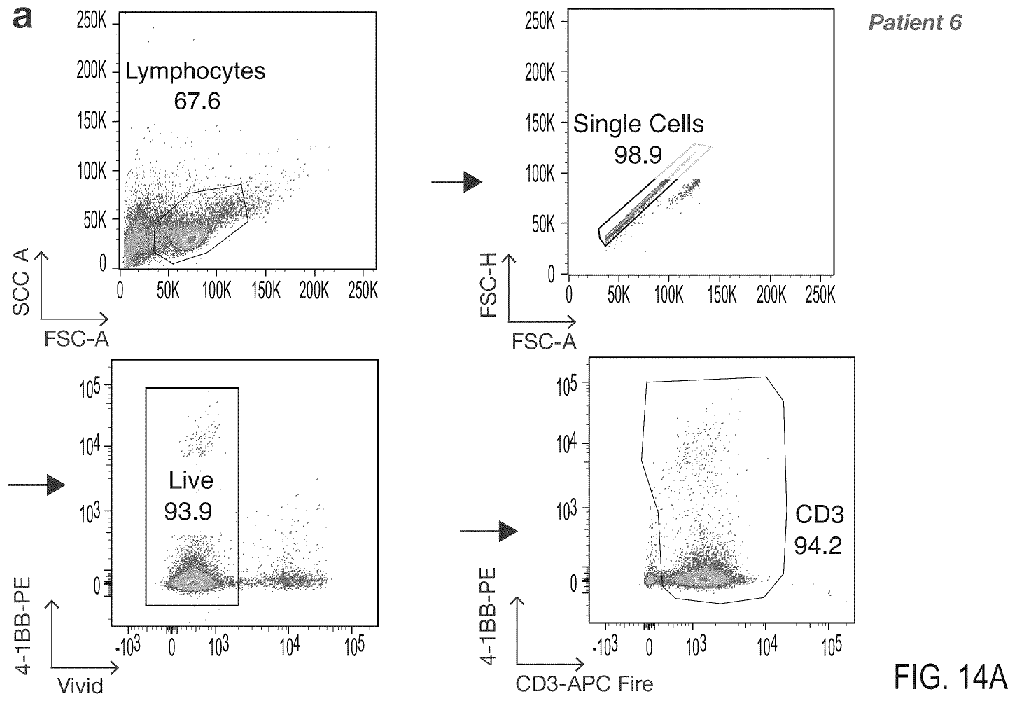

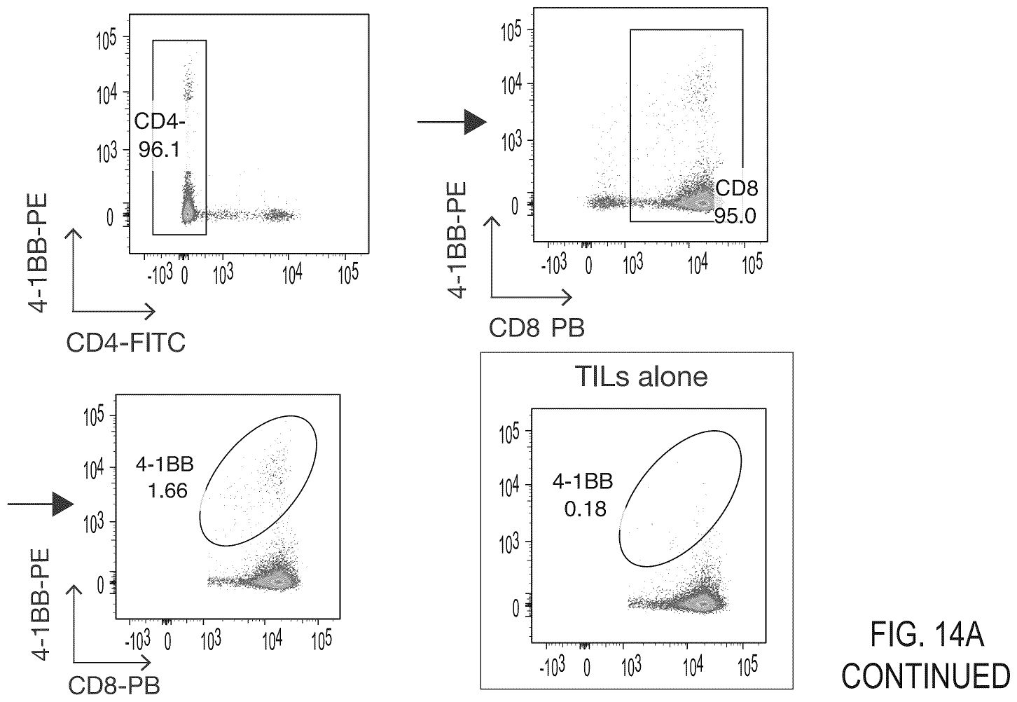

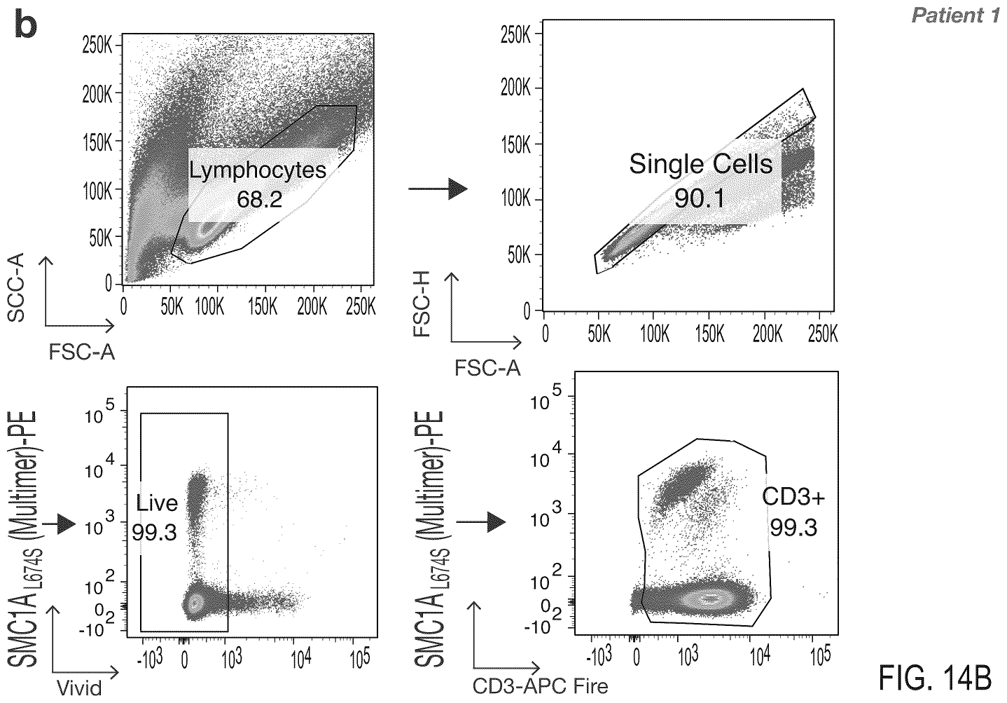

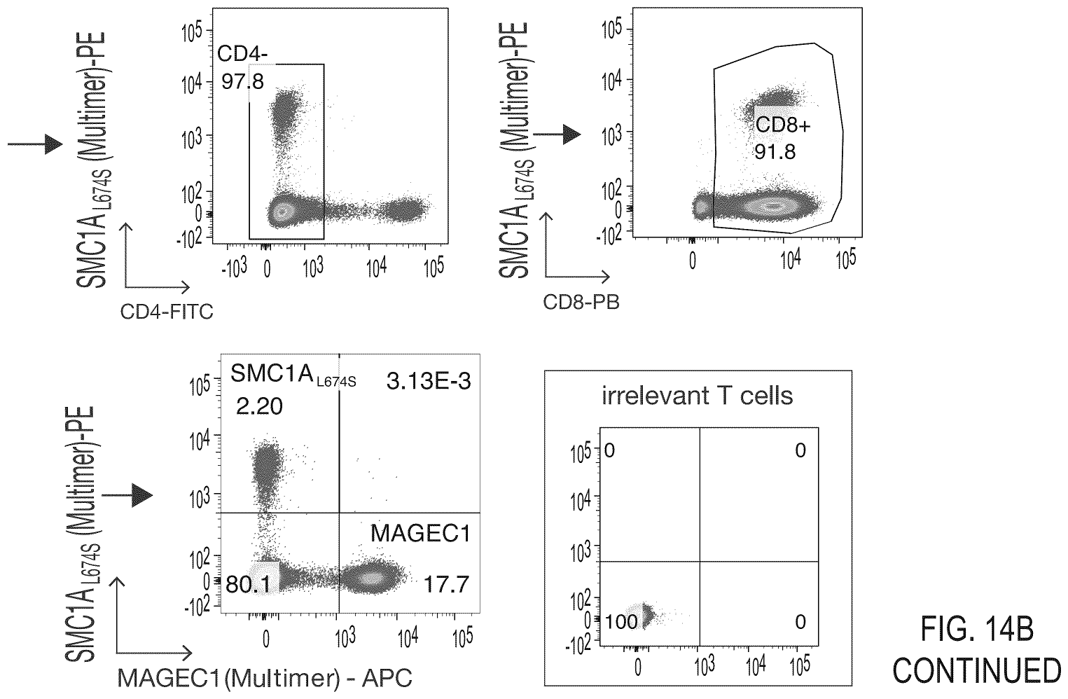

After 16 to 20 hrs, cells were gently harvested from ELISpot plates to assess 4-1BB upregulation, and plates were developed according to the manufacturer's instructions and counted with a Bioreader-6000-E (BioSys). Positive conditions were defined as those with an average number of spots higher than the counts of the negative control (TILs alone) plus 3 times the standard deviation of the negative. Cells retrieved from plates were centrifuged and stained with anti-human CD3, -CD4 (Biolegend), -CD8 (BD Biosciences), -4-1BB (Miltenyi), and Aqua viability dye (Thermo Fisher Scientific) (see example on FIG. 13B and gating strategy on FIG. 14A). The background levels of 4-1BB expression by the negative controls (TILs alone) were subtracted to that of antigen re-challenged TILs in all cumulative figures.

For pNMC-multimer staining, TILs were labeled with cognate in-house pNMC-multimers (produced by the Peptide and Tetramer Core Facility of the Department of Oncology UNIL-CHUV, Lausanne, Switzerland) and anti-CD3, -CD4 (Biolegend), -CD8 (BD Biosciences) and Aqua viability dye (Thermo Fisher Scientific) (see gating strategy on FIG. 14B).

### Isolation of Tumor Antigen-Specific T Cells

Antigen-specific CD8 TILs were FACS sorted using either pNMC-multimers or based on 4-1BB upregulation (Seliktar-Ofir, S. et al. Front. Immunol. 8, 1211 (2017)). For pNMC-multimer sorting, cells were stained with the Aqua viability marker (Thermo Fisher Scientific) and anti-CD4 (Biolegend) and anti-CD8 (BD Biosciences). For activation marker sorting, anti-human 4-1BB (Miltenyi) was used instead of the multimer. Cell sorting experiments were performed using either a BD FACS ARIA II or a BD FACS Melody (BD Biosciences). Purified cells were used for TCR sequencing (see below).

Plots reporting cumulative frequencies of CD8 antigen-specific T cells in the different TIL cultures are based on pMHC-multimer data (Table 4) or 4-1BB upregulation.

### TCR α and β Sequencing and Analysis

mRNA was isolated using the Dynabeads mRNA DIRECT purification kit (Life Technologies) and was amplified using the MessageAmp II aRNA Amplification Kit (Ambion) with the following modifications: IVT was performed at 37° C. for 16h. First, strand cDNA was synthesized using the Superscript III (Thermo Fisher Scientific) and a collection of TRAV/TRBV specific primers. TCRs were then amplified by PCR (20 cycles with the Phusion from NEB) with a single primer pair binding to the constant region and the adapter linked to the TRAV/TRBV primers added during the reverse transcription. A second round of PCR cycle (25 cycles with the Phusion from NEB) was performed to add the Illumina adapters containing the different indexes. The TCR products were purified with AMPure XP beads (Beckman Coulter), quantified, and loaded on the MiniSeq instrument (Illumina) for deep sequencing of the TCRα/TCRβ chain. The TCR sequences were further processed using ad hoc Perl scripts to: (i) pool all TCR sequences coding for the same protein sequence; (ii) filter out all out-frame sequences; (iii) determine the abundance of each distinct TCR sequence. TCR sequences with a single read were not considered for analysis.

### Single-Cell TCR Sequencing

The tumor samples were thawed on the day of the assay, and fragments were dissociated in RPMI complemented with 2% Gelatin (Sigma-Aldrich), 2001U/mL Collagenase I (Thermo Fisher Scientific), 4001U/mL Collagenase IV (Thermo Fisher Scientific), 51U/mL Deoxyribonuclease I (Sigma-Aldrich), and 0.1% RNasin Plus RNase Inhibitor (Promega) for 30 min at 37° C. Digested cells were then filtered and resuspended in PBS+1% Gelatin+0.1% RNasin. Cells were stained first with 50 mM/mL of Calcein AM (ThermoFisher Scientific) and FcR blocked (Miltenyi Biotec) for 15 min at RT and next with anti-CD45 (BioLegend). Dissociated cells were resuspended in PBS complemented with 0.04% BSA+0.1% RNasin, and DAPI (Invitrogen) staining was performed. CD45 live cells were sorted with a FACS Astrios (Beckman Coulter). Sorted cells were then resuspended at 0.6-1.2e4 cells/μL with viability of >90% and subject to a 10× Chromium instrument for the single-cell analysis (10× Genomics). 1.7e4 cells were loaded per sample, with the targeted cell recovery of 1e4 cells. Using a microfluidic technology, single cells were captured and lysed, mRNA was reverse transcribed to barcoded cDNA (10× Genomics). 14 PCR cycles were performed for cDNA amplification, and a targeted enrichment for TCRs was performed. V(D)J libraries were obtained following the manufacturer's instructions (10× Genomics). Barcoded VDJ libraries were then pooled and sequenced by an HiSeq 2500 sequencer (Illumina). Single-cell TCR sequencing data were processed by the Cell Ranger software pipeline (version 3.1.0, 1OX Genomics).

### TCR Validation

To validate antigen specificity and interrogate antitumor reactivity, TCRαβ pairs were cloned into recipient-activated T cells or Jurkat cell line (TCR/CD3 Jurkat-luc cells (NFAT), Promega). Paired a and β chains were annotated based on bulk (i.e., top TCR clonotypes obtained by TCR sequencing of tumor-antigen FACS sorted TILs) or single-cell TCR sequencing data.

Autologous or HLA-matched allogeneic PBMCs were plated at 1e6 cells/mL in p48-well plates in R8 medium supplemented with 50IU/mL IL-2 (Proleukin). T cells were activated with Dynabeads Human T Activator CD3/CD28 beads (Thermo Fisher Scientific) at a ratio of 0.75 beads: 1 total PBMCs. After 3 days of incubation at 37° C. and 5% CO₂, beads were removed and activated T cells cultured for four extra days before electroporation or freezing.

For the transfection of TCRαβ pairs into T cells and Jurkat cells, the Neon electroporation system (Thermo Fisher Scientific) was used. Briefly, T cells and Jurkat cells were resuspended at 15-20e6 cells/ml in buffer R (buffer from the Neon kit), mixed with 25-50 μg/mL of TCRα chain RNA together with 25-50 μg/mL of TCRβ chain RNA and electroporated with the following parameters: 1600V, 10 ms, 3 pulses and 1325V, 10 msec, 3 pulses, respectively. Electroporated cells were either incubated for 17-20 hrs at 37° C. or used immediately.

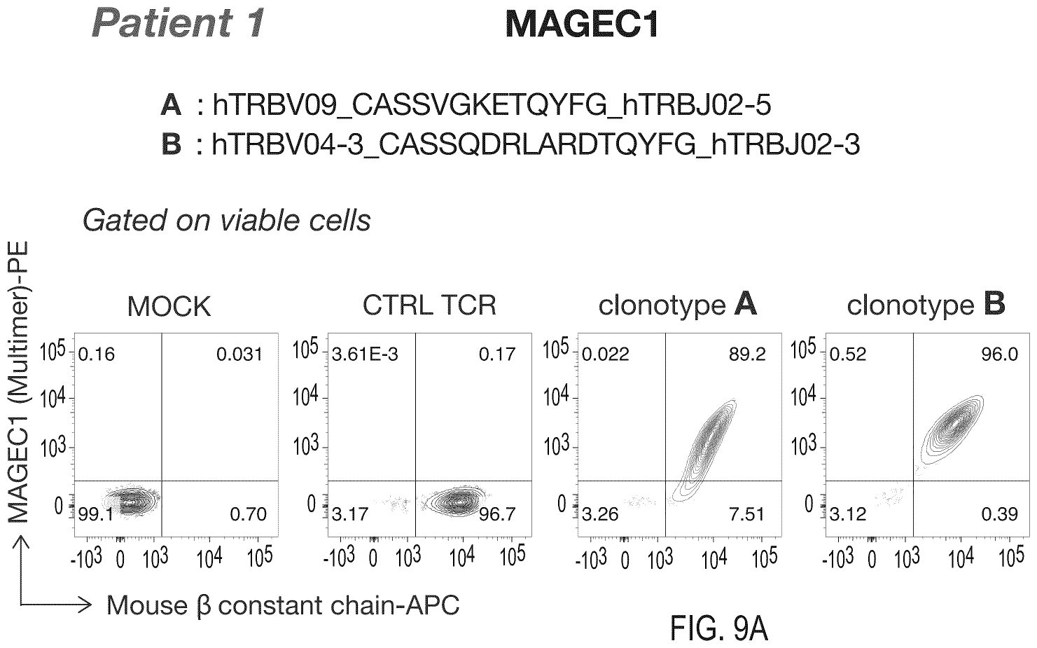

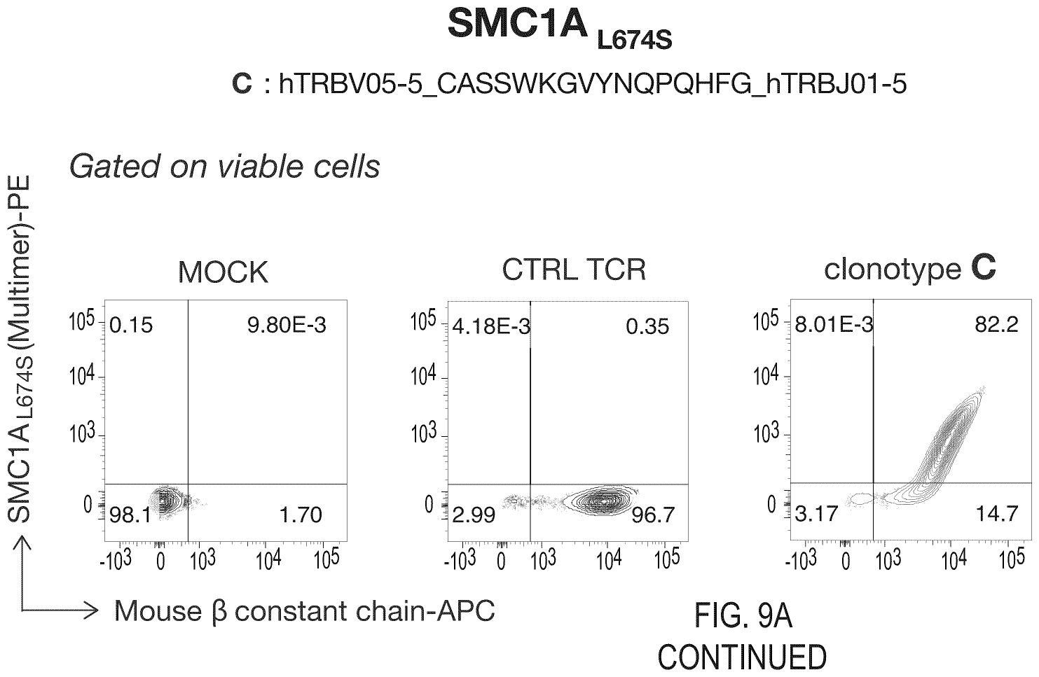

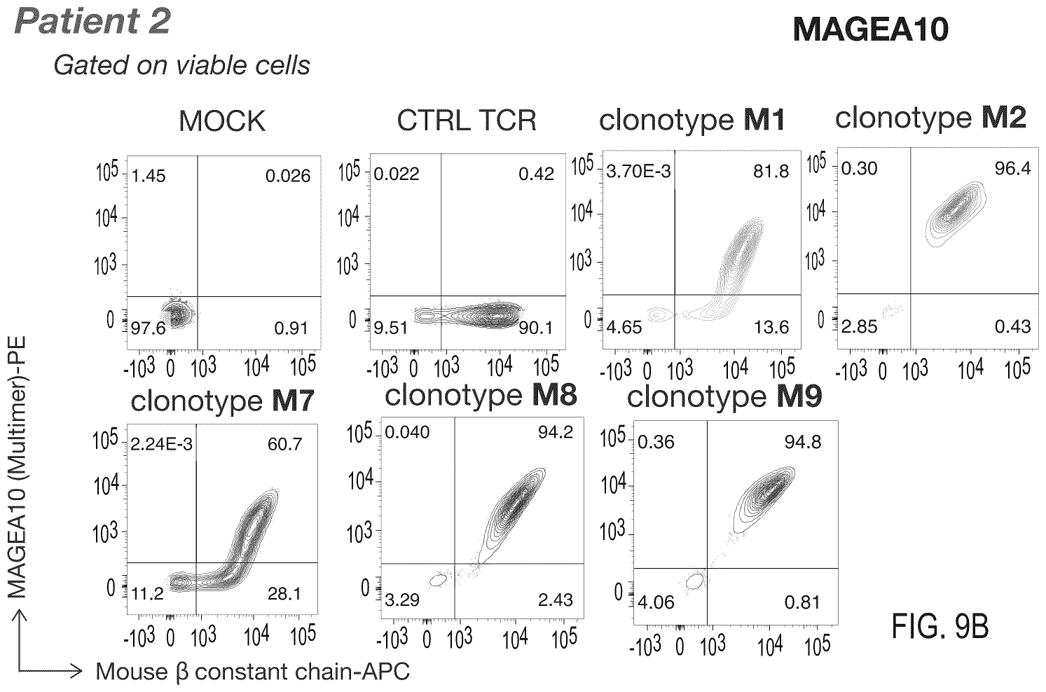

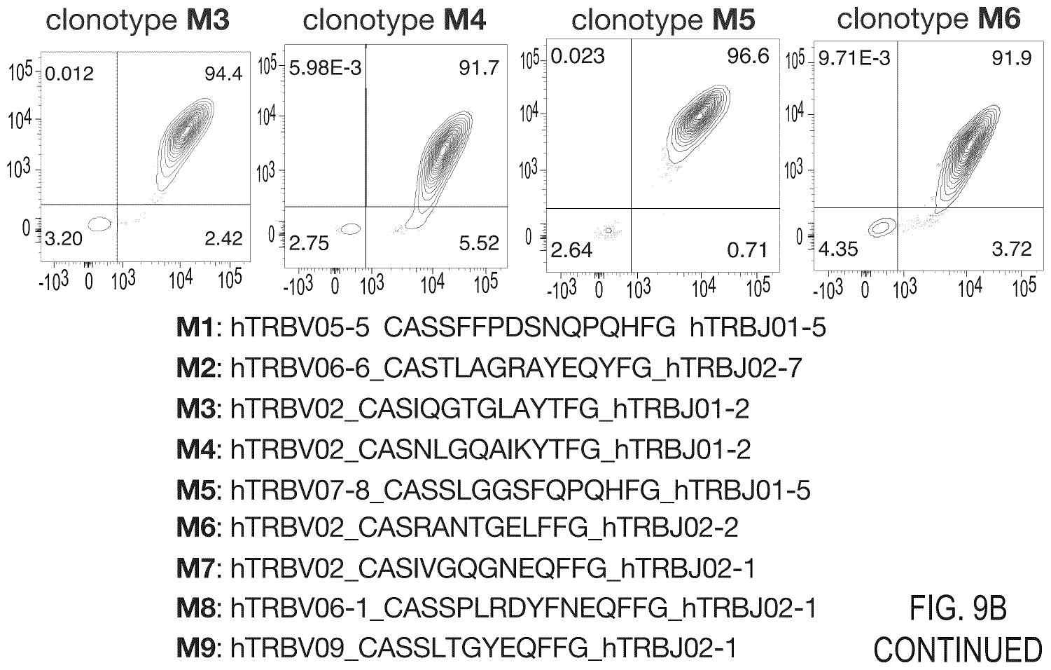

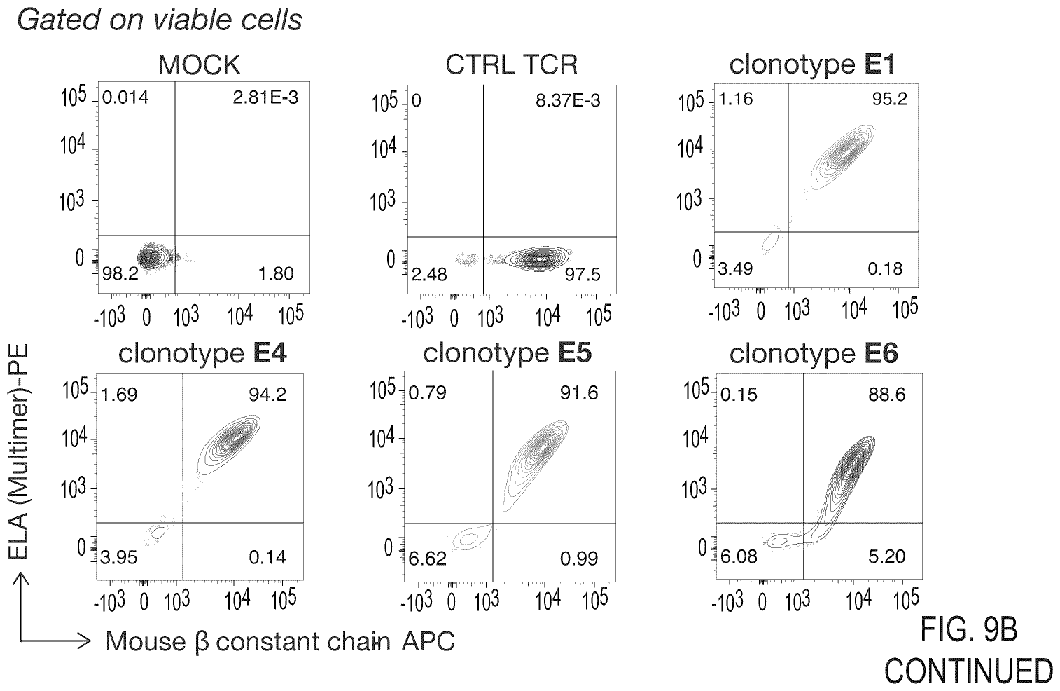

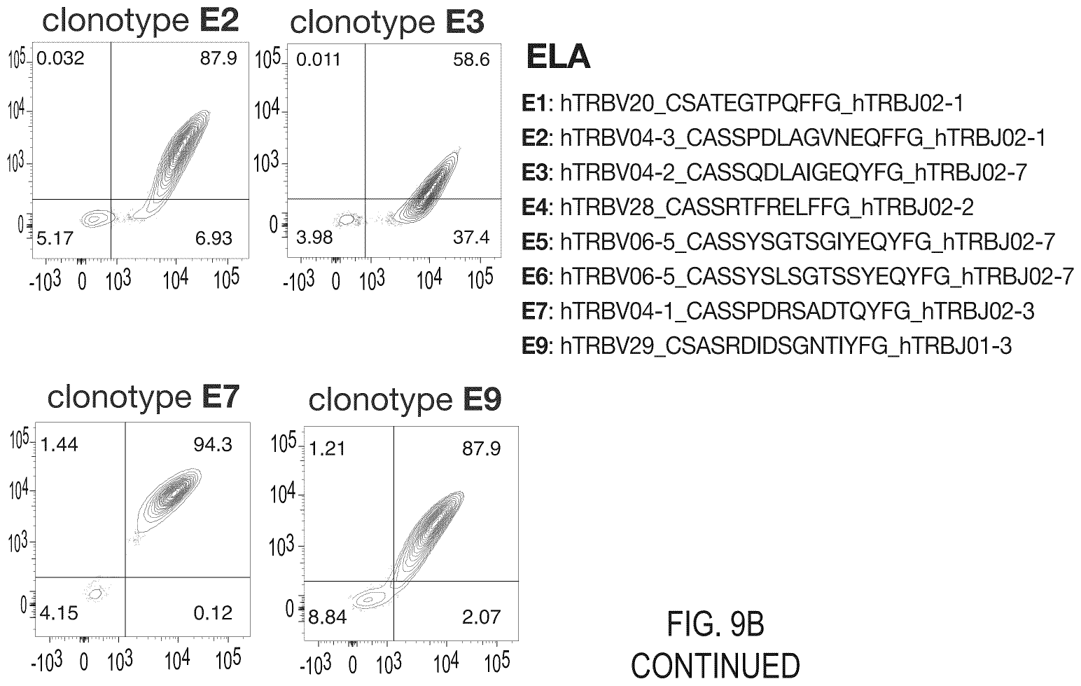

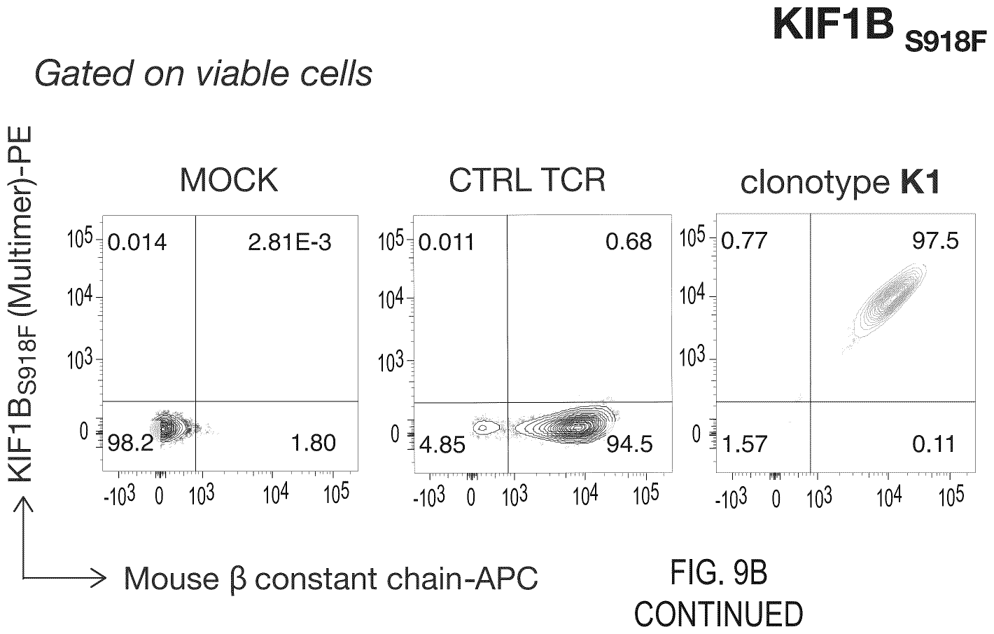

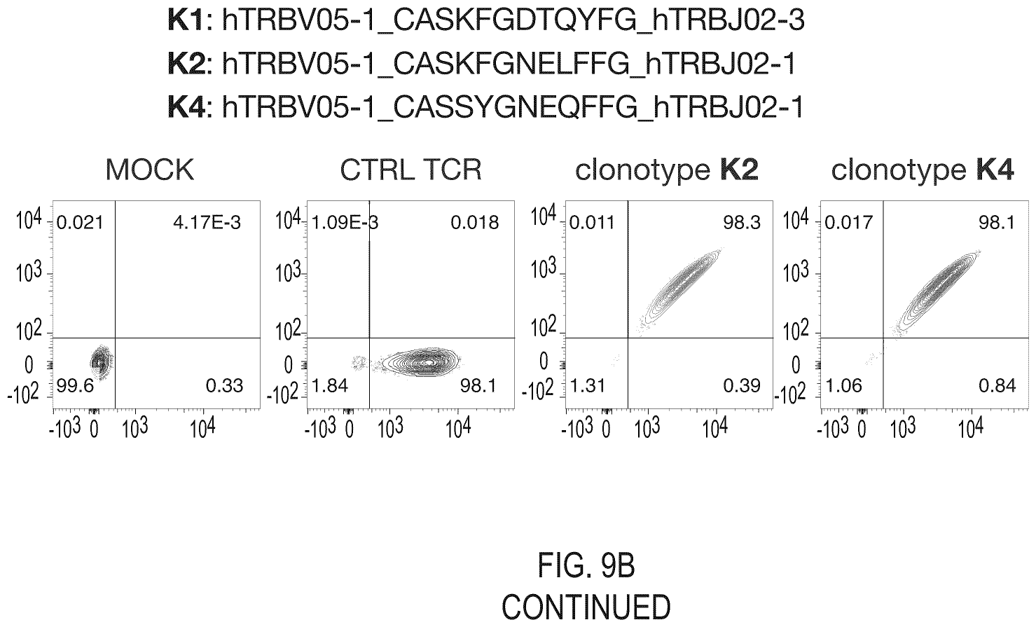

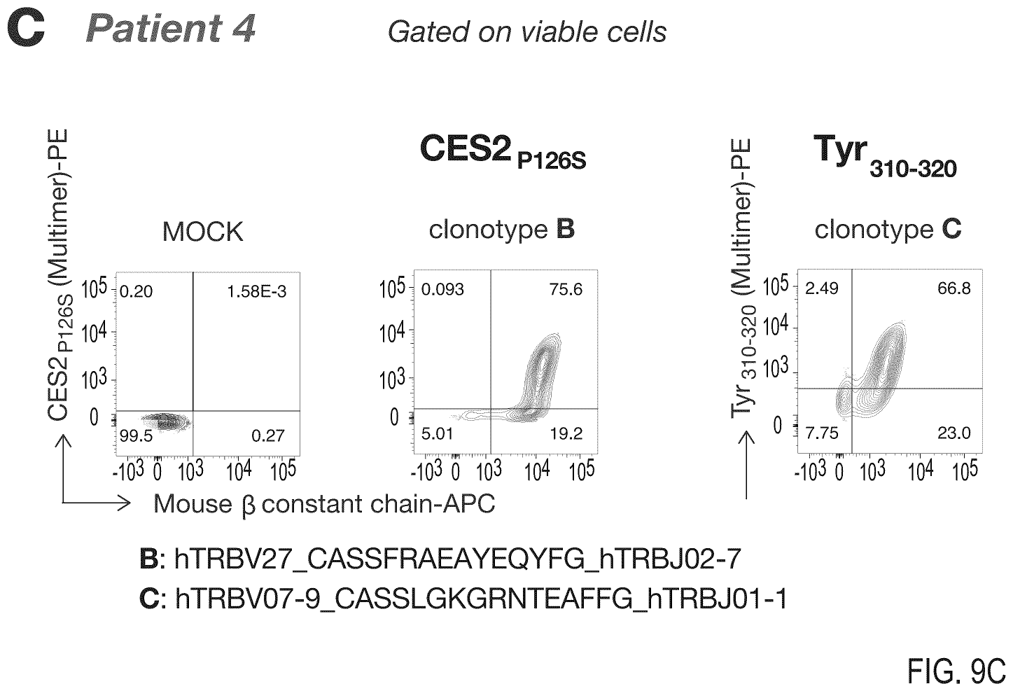

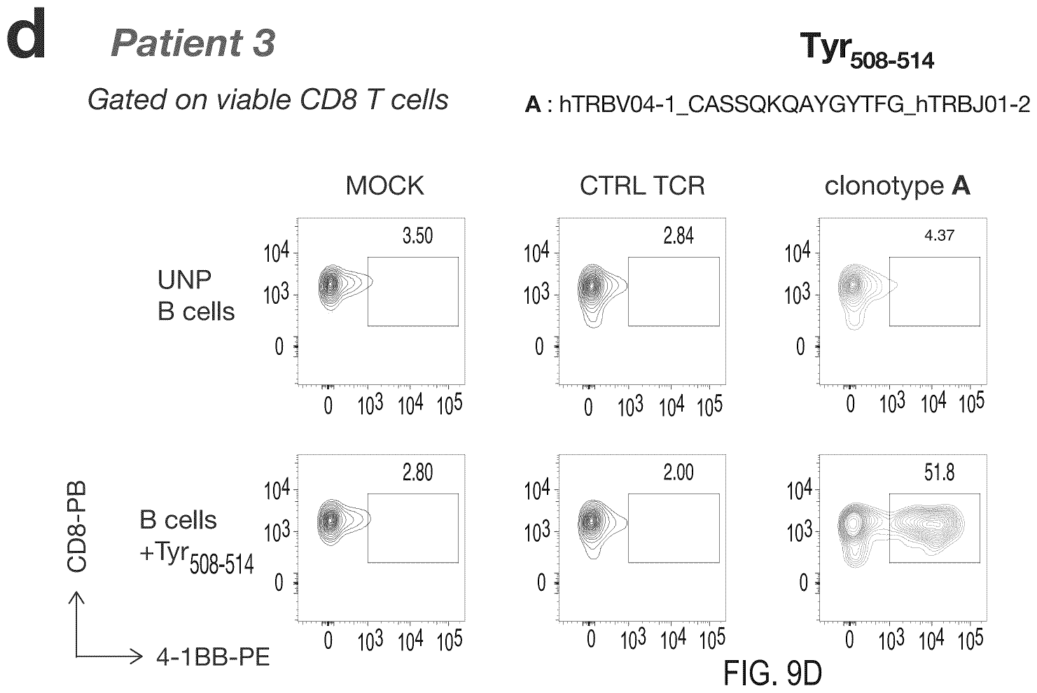

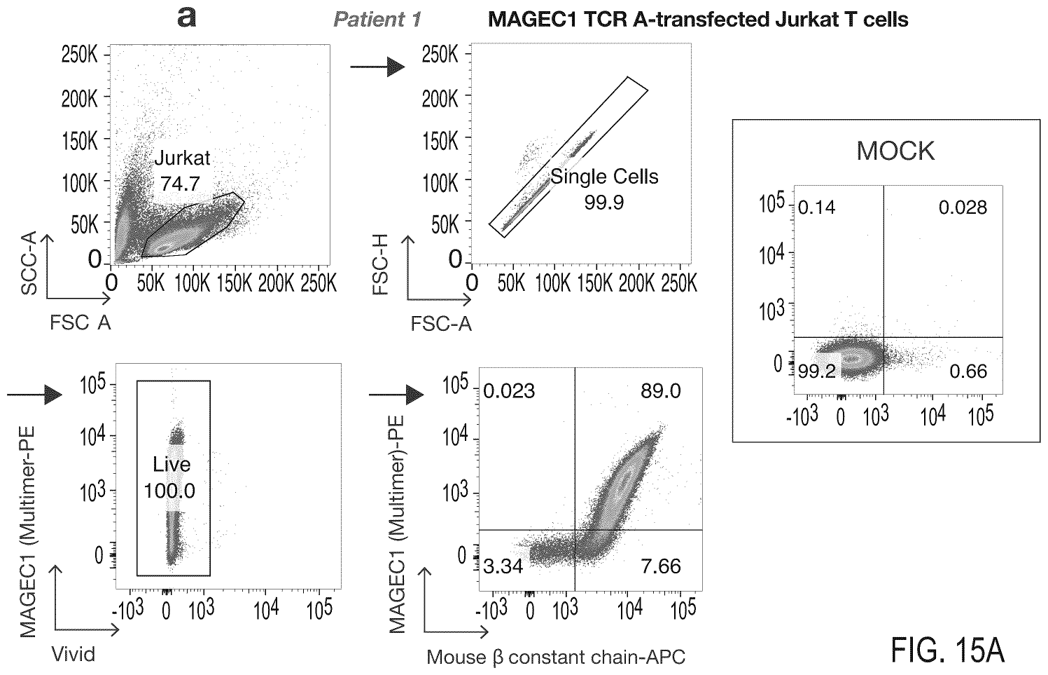

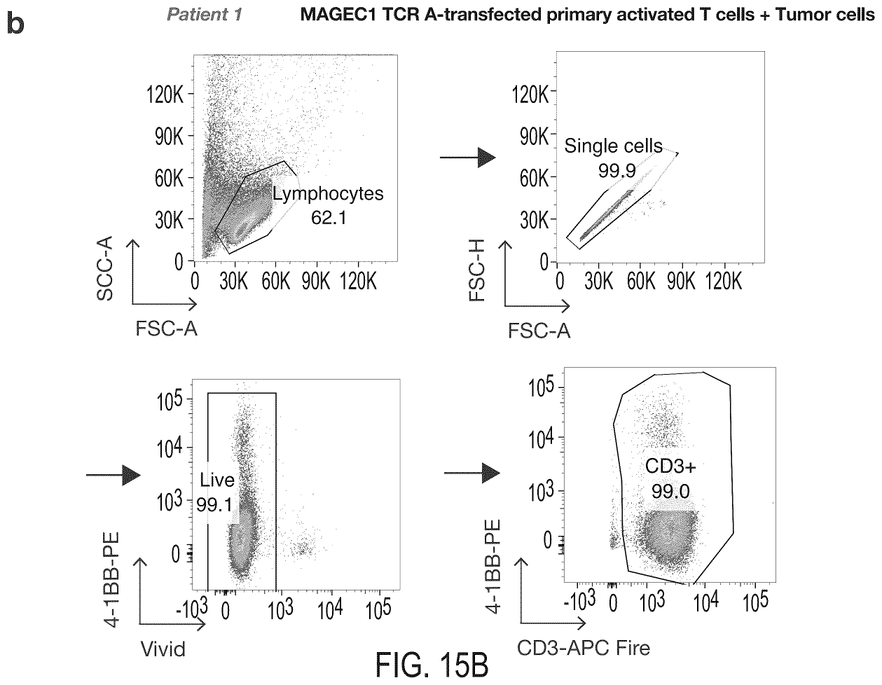

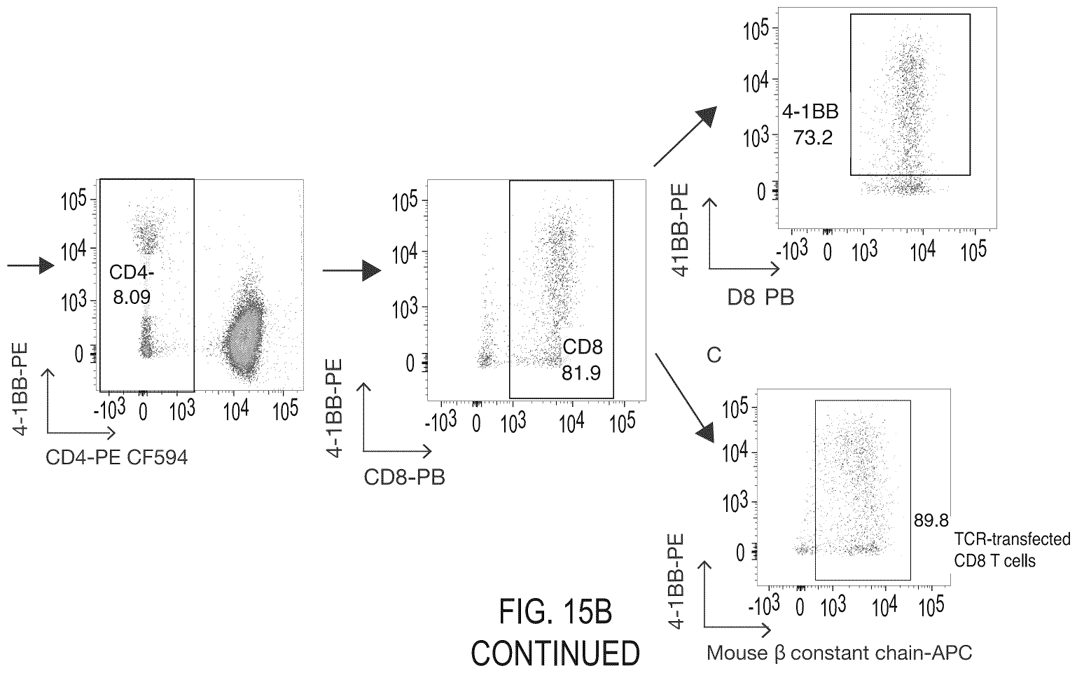

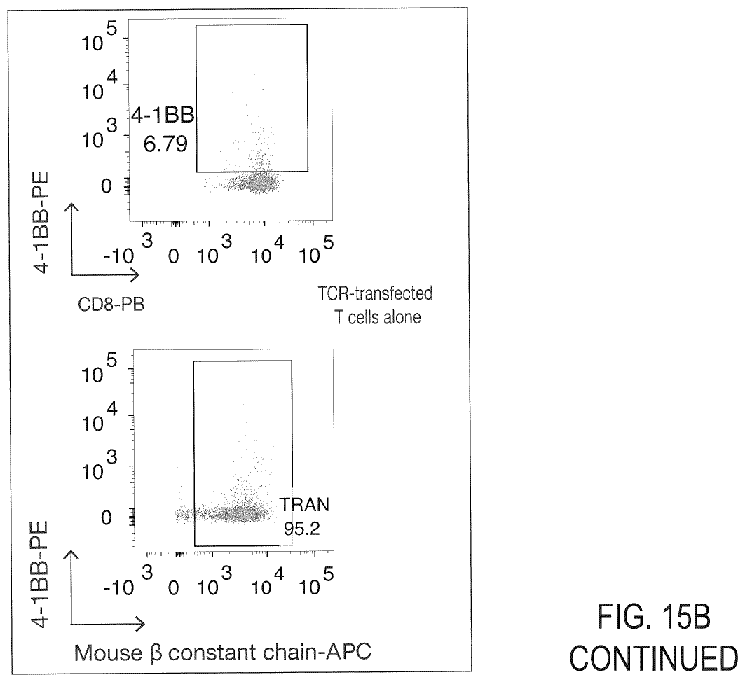

For the validation of antigen specificity, electroporated Jurkat cells were interrogated by pMHC-multimer staining with the following surface panel: anti-CD3, -CD4 (Biolegend), -CD8 (BD Biosciences), anti-mouse TCRβ-constant (Thermo Fisher Scientific), and Aqua viability dye (Thermo Fisher Scientific) (see gating strategy on FIG. 15A). The following experimental controls were included: MOCK (transfection with PBS) and a control TCR (irrelevant crossmatch of a TCRα and β chain) (FIG. 9).

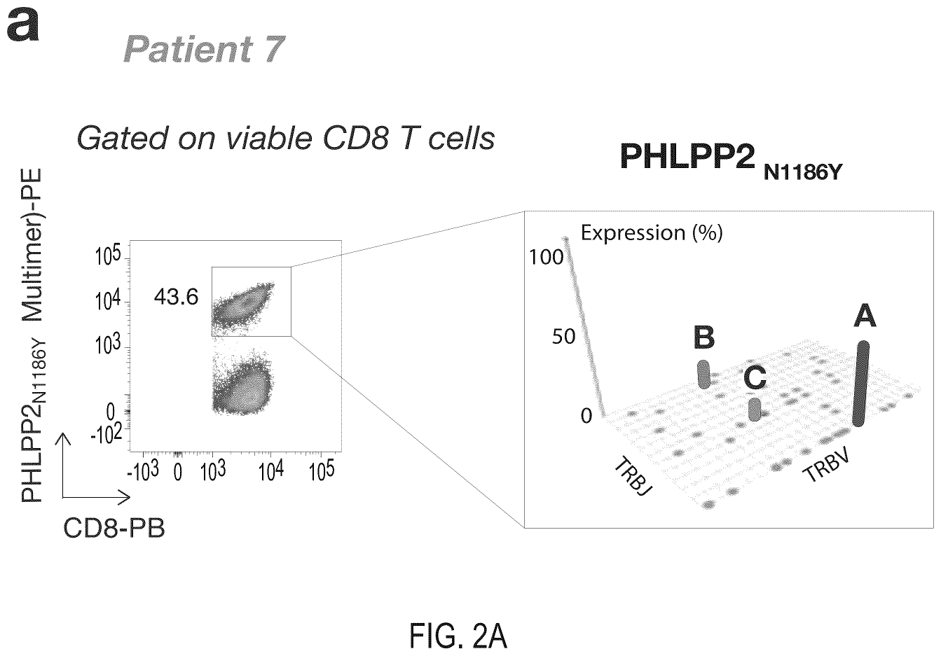

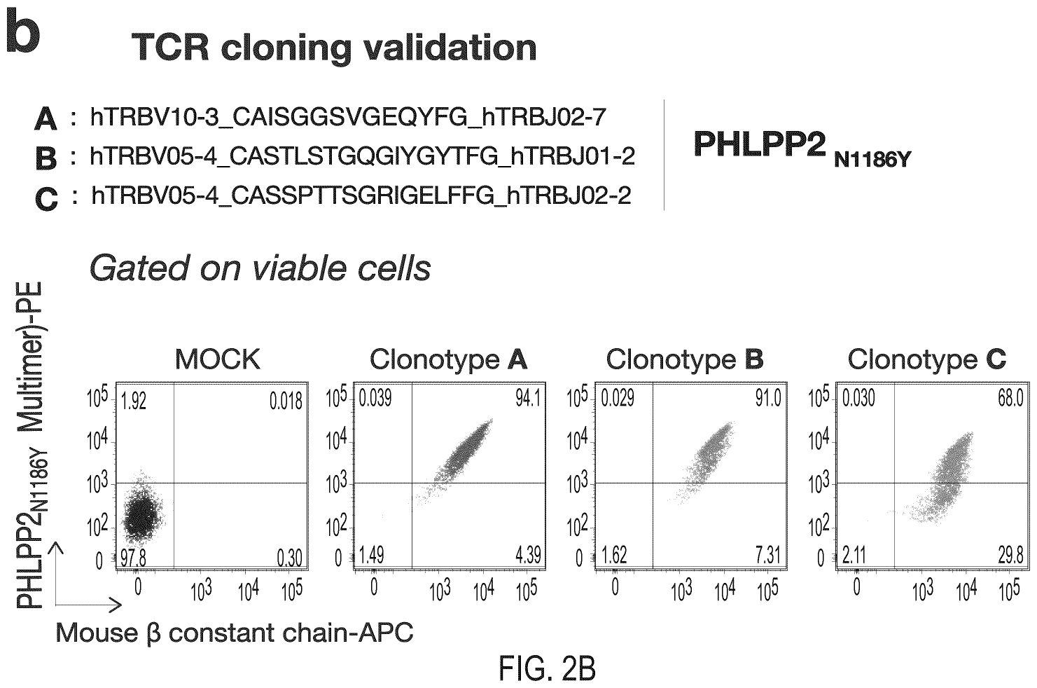

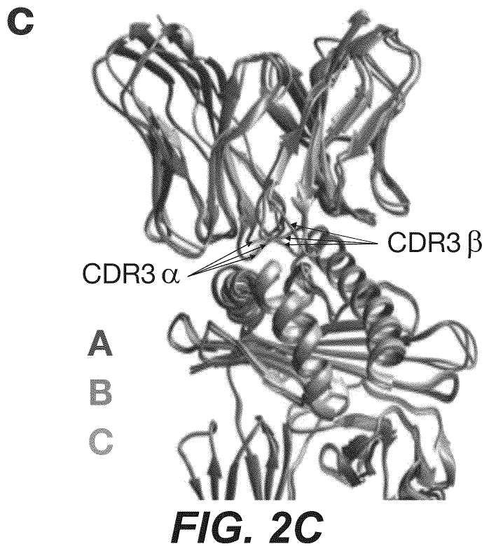

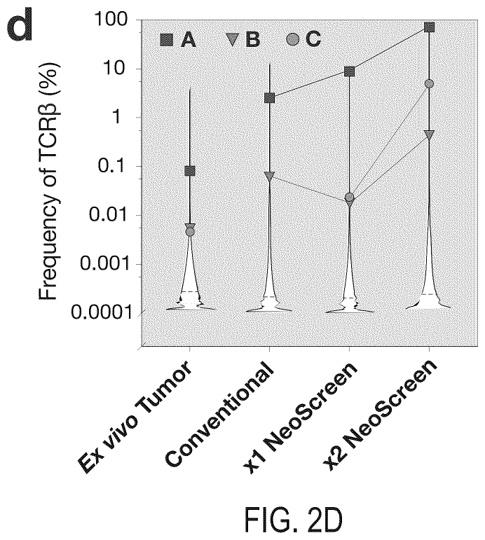

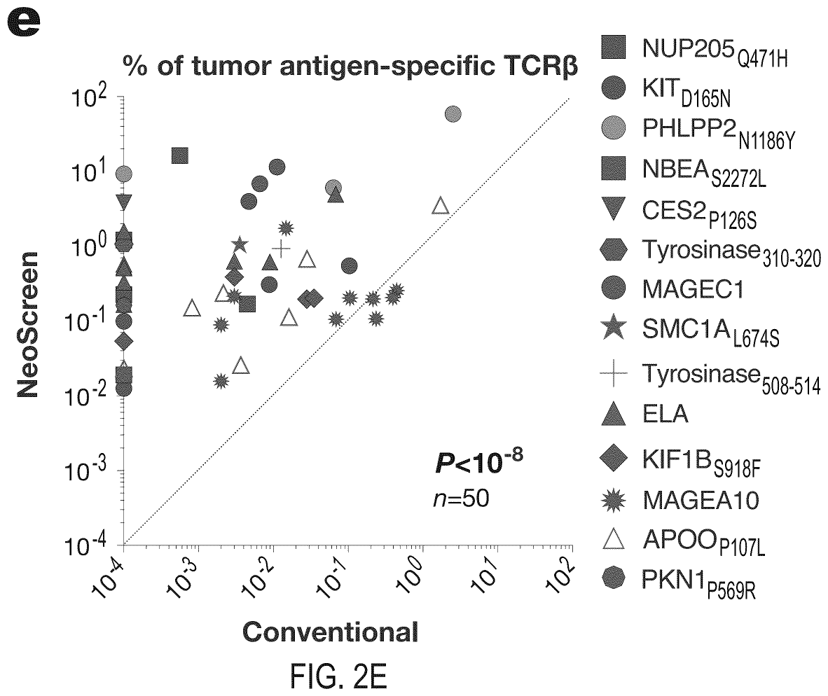

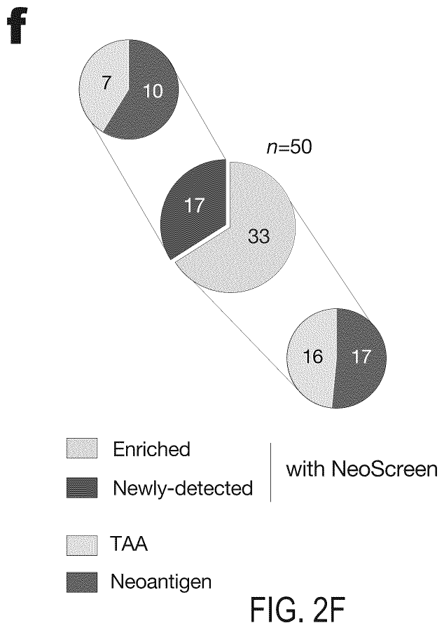

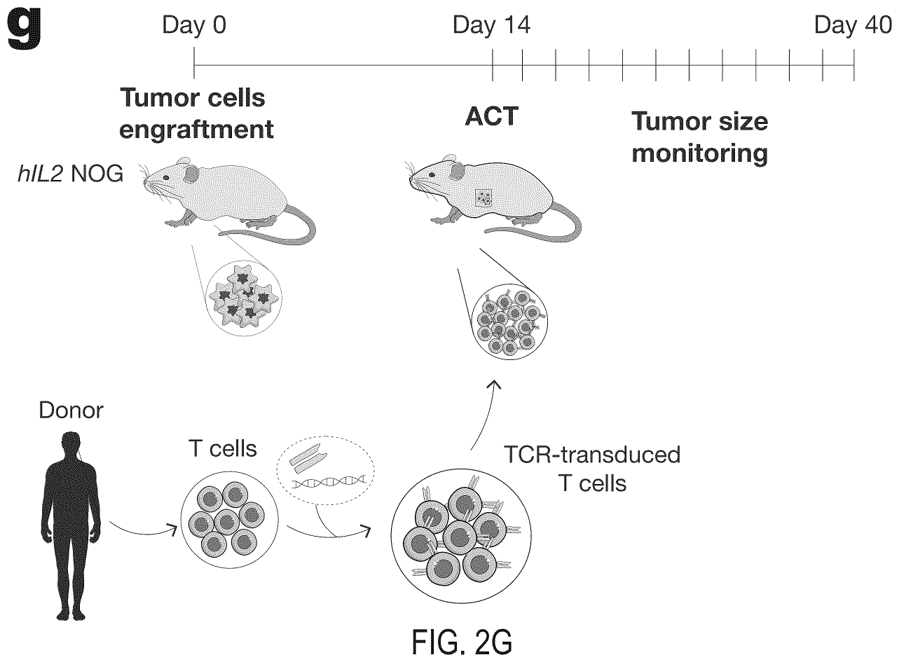

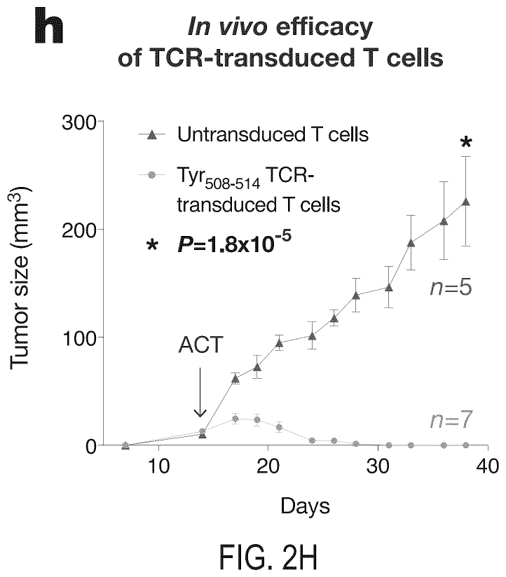

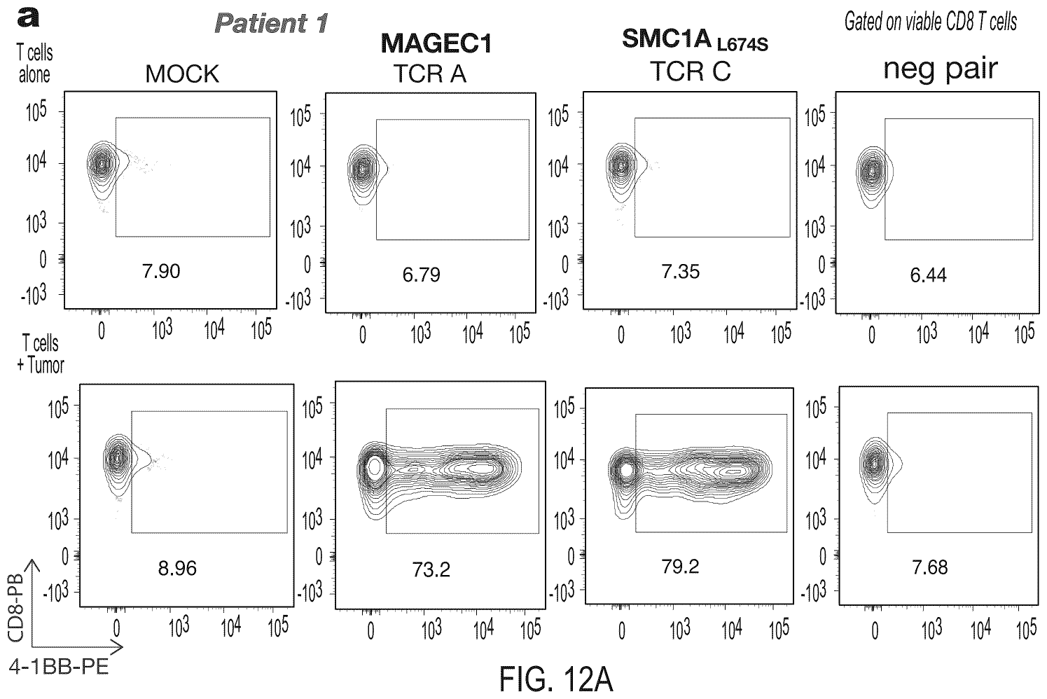

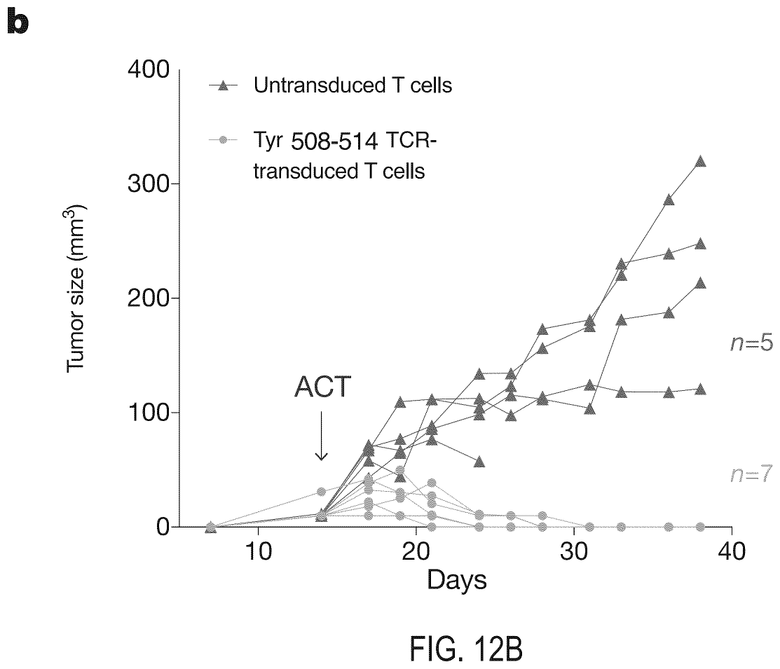

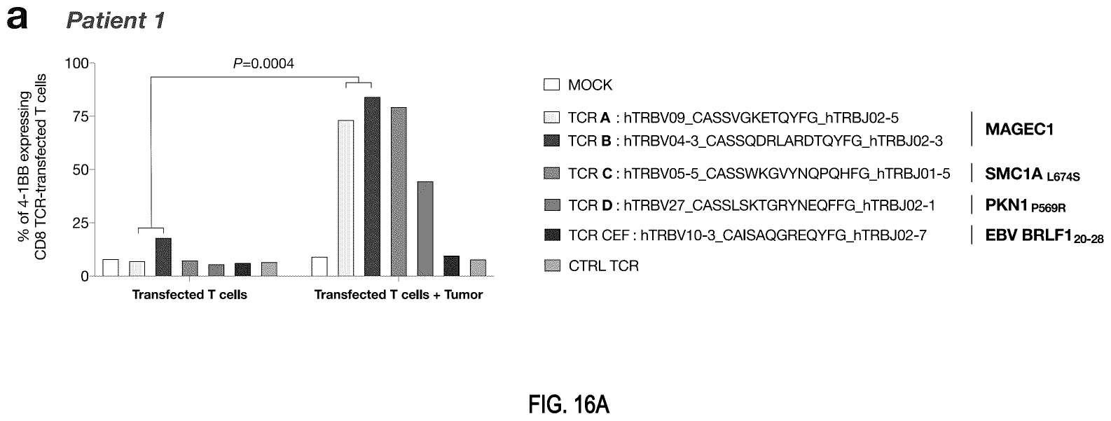

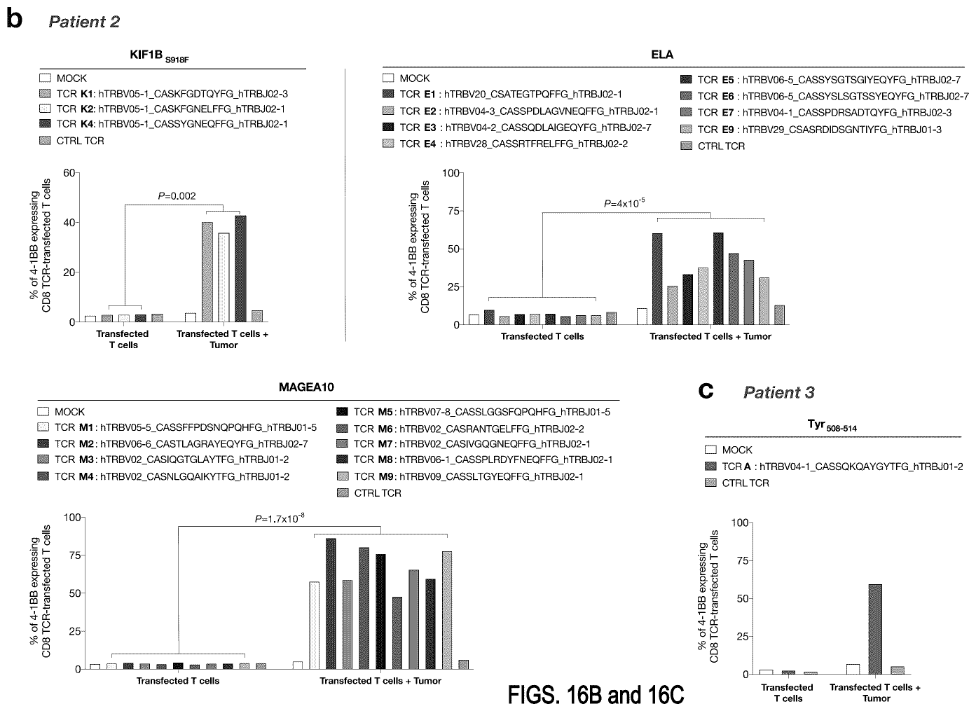

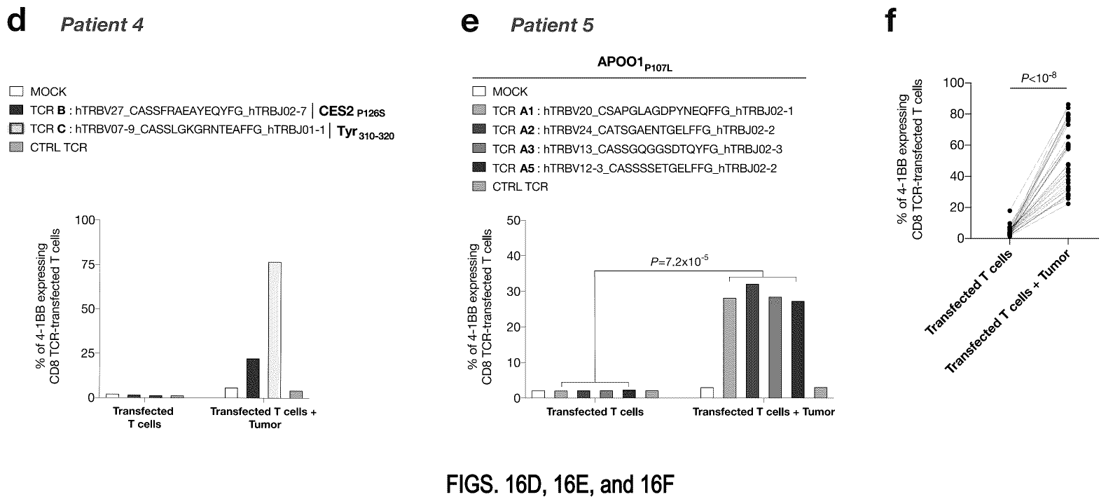

To assess antitumor reactivity of validated TCRs, 1e5 TCR RNA-electroporated T cells and 3e4 IFNγ-treated autologous tumor cells were co-cultured in IFNγ ELISpot assay. After 20-24 hrs incubation, cells were recovered, and the upregulation of 4-1BB (CD137) was evaluated by staining with anti-4-1BB (Miltenyi), anti-CD3 (Biolegend), anti-CD4 and anti-CD8 (BD Biosciences), anti-mouse TCRβ-constant (Thermo Fisher Scientific) and with viability dye Aqua (Thermo Fisher Scientific) (see gating strategy on FIG. 15B). The following experimental controls of TCR transfection were included: MOCK (transfection with PBS), a control TCR (irrelevant crossmatch of a TCRα and β chain) and, when available, a virus-specific TCR (see FIGS. 16A-F). Validation of tumor reactivity of TCRαβ pairs required: 1) the background level of 4-1BB expression to be <20% in all control conditions, 2) the fold expansion of 4-1BB expression between transfected T cells exposed to autologous tumors and TCR-T cells alone (background) to be >10 and 3) the percentage of 4-1BB expression after tumor challenge of transfected T cells and subtraction of the 4-1BB background obtained with transfected T cells alone to be >20% (FIGS. 16A-F and FIG. 12B). Displayed data show the percentage of 4-1BB expression after tumor challenge of transfected T cells and subtraction of the 4-1BB background obtained with transfected T cells alone (FIGS. 2E and 12A).

### Adoptive T Cell Transfer in Immunodeficient IL-2 NOG Mice

Tyr₅₀₈₋₅₁₄-TCRα and β chains, divided by a Furin/GS linker/T2A element were cloned into a pCRRL-pGK lentiviral plasmid to produce high-titer replication-defective lentiviral particles, as previously described (Giordano-Attianese, G. et al. Nat. Biotechnol. 38, 426-432 (2020)). For primary human T cell transduction, CD8 T cells were negatively selected with beads (Miltenyi) from PBMCs of a healthy donor (apheresis filter), activated, and transduced with minor modifications. Briefly, CD8 T cells were activated with anti-CD3/CD28 beads (Thermo Fisher Scientific) and added with lentiviral particles after overnight activation. Activation beads were removed after 5 days of T cell culture in R8 medium supplemented with IL-2 at 50IU/mL. On day 6, transduced T cells expressing the mouse TCRβ-constant region were sorted with a FACS ARIA III. Isolated Tyr₅₀₈₋₅₁₄ TCR-transduced CD8 T cells were then expanded for 10 days in R8 medium and 50IU/mL IL-2 before mouse injection.

IL-2 NOG mice (Taconic) were maintained in a conventional animal facility at the University of Lausanne under specific pathogen-free status. Six- to nine-week-old female mice were anesthetized with isoflurane and subcutaneously injected with 1e6 tumor cells from melanoma patient 3. Once the tumors became palpable (at day 14), 5e6 human Tyr₅₀₈₋₅₁₄ TCR-transduced T cells were injected intravenously into the tail vein. Tumor volumes were measured by caliper twice a week and calculated as follows: volume=length×width×width/2. Mice were sacrificed by CO₂ inhalation before the tumor volume exceeded 1000 mm³ or when necrotic skin lesions were observed at the tumor site. When sacrificed, tumors were harvested and processed at the Tumor Processing Facility of the University of Lausanne. This study was approved by the Veterinary Authority of the Canton de Vaud (under license 3387) and performed in accordance with Swiss ethical guidelines.

### TCR-pMHC Structure Modeling

The 3D structures of the three PHLPP2_{N1186Y}-specific TCRs bound to peptide QSDNGLDSDY (SEQ ID NO: 241) in complex with HLA-A*01:01 were modeled. Starting from V and J segment identifiers and from the CDR3 sequences, the full sequence of the constant and variable domains of TCRα, and TCRβ were reconstituted based on IMGT/GENE-DB reference sequences (Giudicelli, V., et al. Nucleic Acids Res. 33, 256-261 (2005)). Homology models of the TCR-pMHC complexes were generated using Rosetta 3.10 (Leaver-Fay, A. et al. Methods Enzym. 487, 545-574 (2011)) and Modeller 9.21 (Webb, B. & Sali, A. Curr. Protoc. Bioinforma. 54, 5.6.1-5.6.37 (2016)). Template libraries include TCR, TCR-pMHC, and pMHC structures retrieved from the Protein Data Bank (Rose, P. W. et al. Nucleic Acids Res. 45, D271-D281 (2017)). The Rosetta ‘TCRmodel’ protocol (Gowthaman, R. & Pierce, B. G. Nucleic Acids Res. 46, W396-W401 (2018)) was adapted to the present approach and applied to find the respective templates and model TCR (Table 6). The orientation of modeled Vα and Vβ structure was performed based on Vα/Vβ templates, while the orientation of the TCR relative to the pMHC was performed based on TCR-pMHC templates, identified using sequence similarity (Table 6). Side chains and backbones of the TCR-pMHC models were refined using Modeller. A total of 1500 models were produced for each TCR-pMHC. These models were subsequently ranked based on the Discrete Optimized Potential Energy as implemented in Modeller. For each TCR-pMHC, the best model according to the score was selected for CDR loop refinement. The latter was performed by creating 100 alternative loop conformations using the kinematic closure loop modeling of Rosetta and subsequent refinement using the fast ‘relax’ protocol. Molecular interactions were analyzed in the top5 ranked models over 1600. The final TCR-pMHIC structural model is the one with the highest number of favorable interactions within the top five high-score models. In these structure files, TCRα, is chain D, TCRβ is chain E, the peptide is chain C, MHC is chain A and β2-macroglobulin chain B. Residue numbers start from 1 for each chain. Molecular graphics and analyses of the molecular interactions are presented, making use of the UCSF Chimera package (Pettersen, E. F. et al. J. Comput. Chem. 25, 1605-1612 (2004)).

### Statistical Analyses

Differences between averages of variables were compared using a one-tailed t-test for variables with normal distribution, as specified. Some variables underwent a logarithmic transformation to obtain normality, as reported in figure legends. Statistical analyses were performed using Graphpad PRISM 8.3.0.

### Example 2

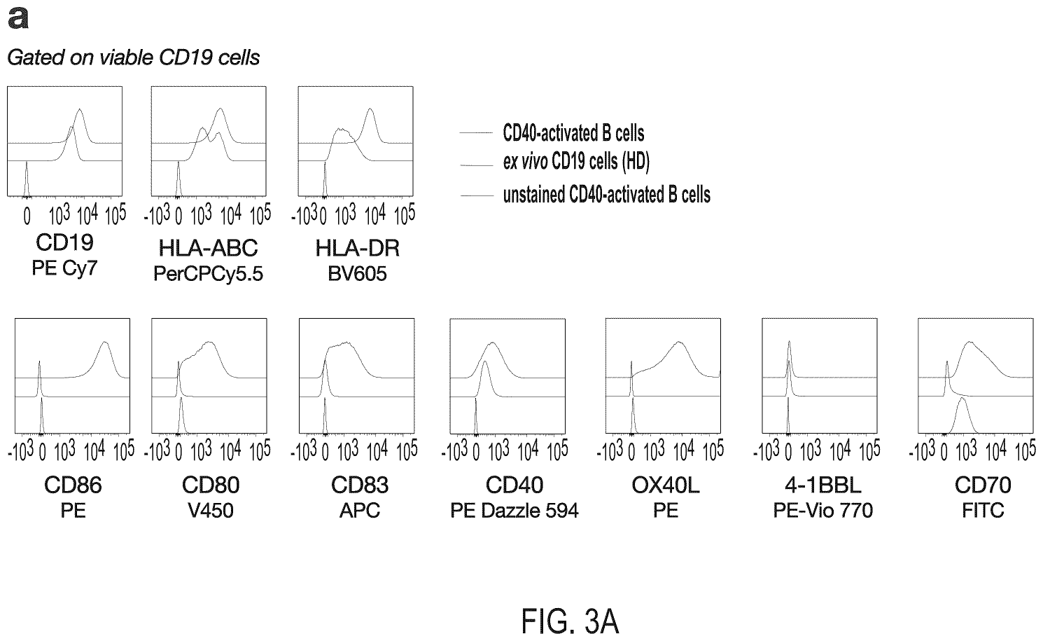

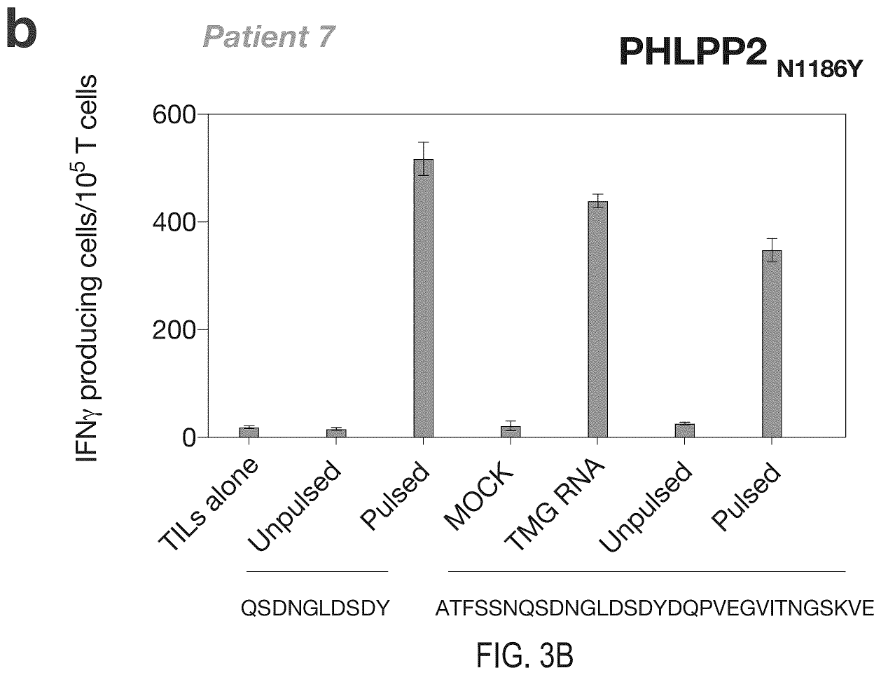

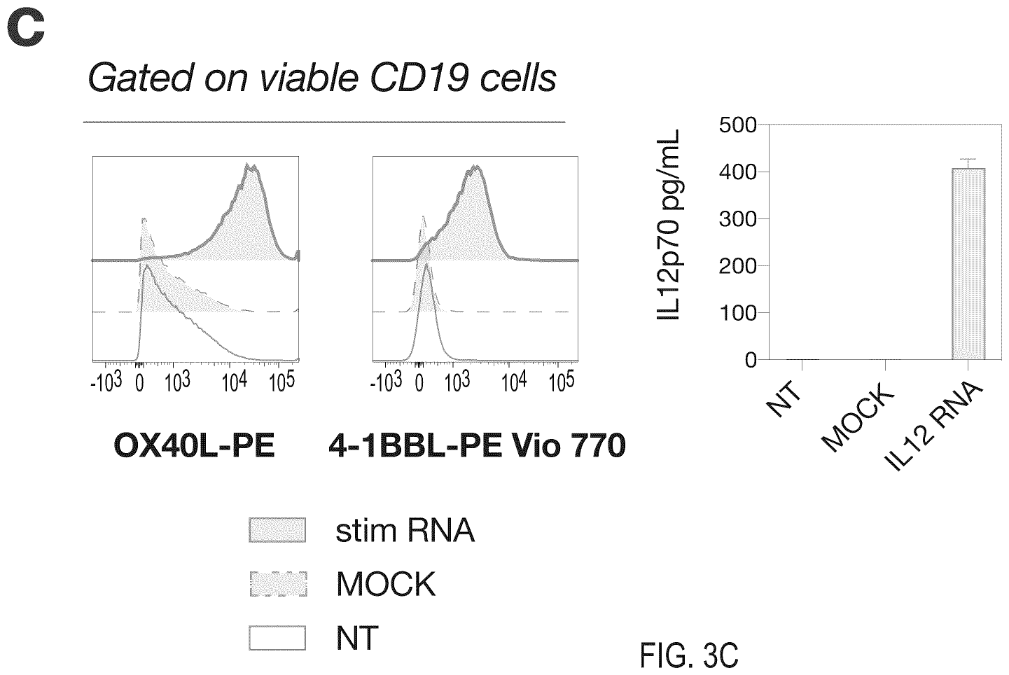

This disclosure describes NeoScreen, an in vitro TIL expansion and screening methodology, aiming at optimizing the sensitivity of antigen validation, but also of isolation of rare tumor antigen-specific CD8 T cells for cloning of cognate TCRs from highly enriched tumor antigen-specific CD8 T cells. Unlike conventional culture methods that rely solely on the growth factor IL-2, NeoScreen is based on the early exposure of TILs grown from whole tumor fragments or from dissociated tumor cells to antigens of choice loaded on competent autologous antigen-presenting cells (APCs) (FIG. 1A). CD40-activated (CD40-act) B cells were used as APCs since they are easily procurable and expandable from low amounts of blood relative to dendritic cells, and easy to engineer by electroporation. As shown in FIG. 3A, CD40-act B cells expressed key molecules required for antigen presentation and T cell activation. Accordingly, CD40-act B cells loaded with diverse sources of neoantigen (i.e., transfected with minigenes or pulsed with synthetic peptides) ensured efficient stimulation of neoepitope-specific CD8 TILs ex vivo (FIG. 3B). To optimize APC potency, CD40-act B cells were engineered by co-electroporation of RNA encoding immune stimulatory 4-1BB ligand (4-1BBL/CD137), OX40 ligand (OX40L/CD252), and IL-12 (FIG. 3C).

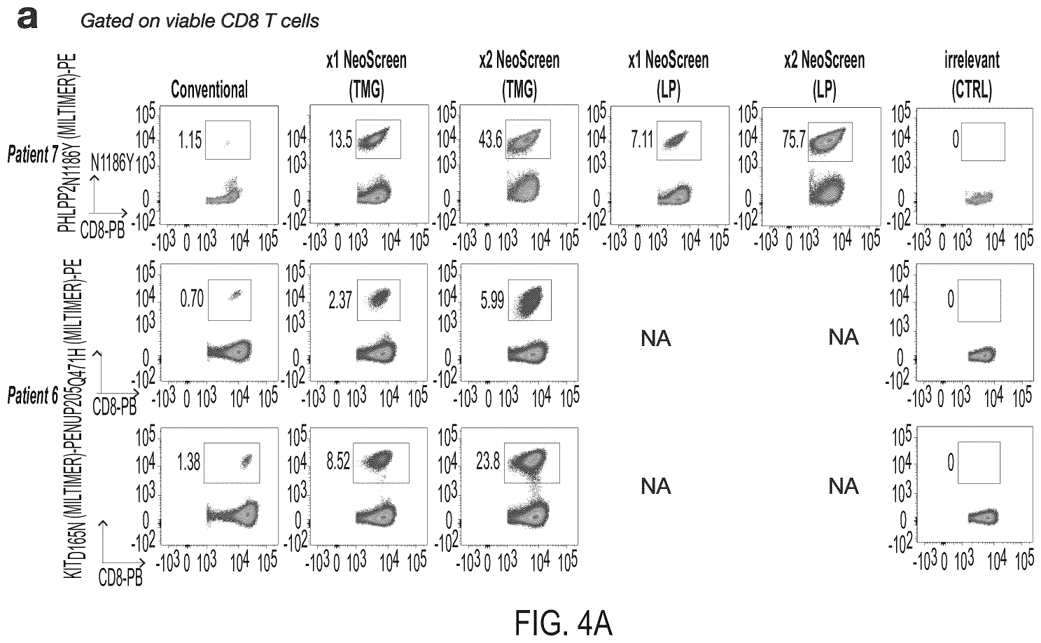

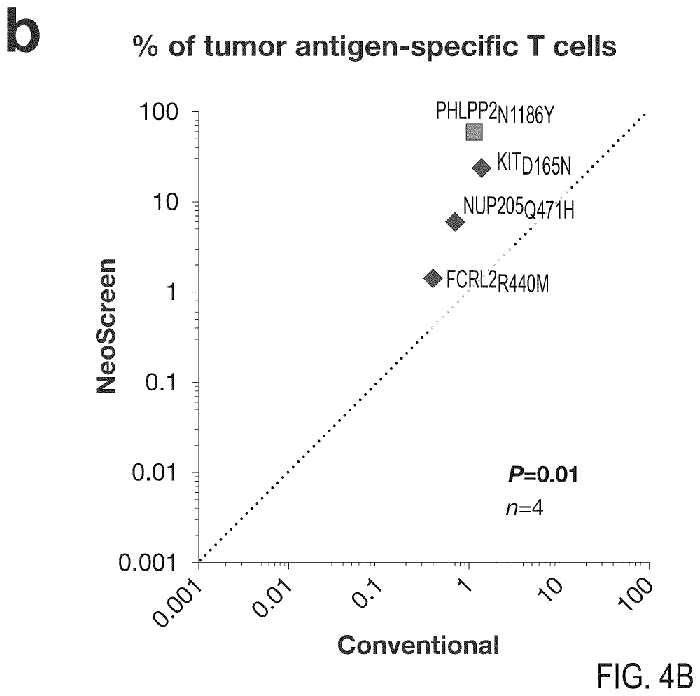

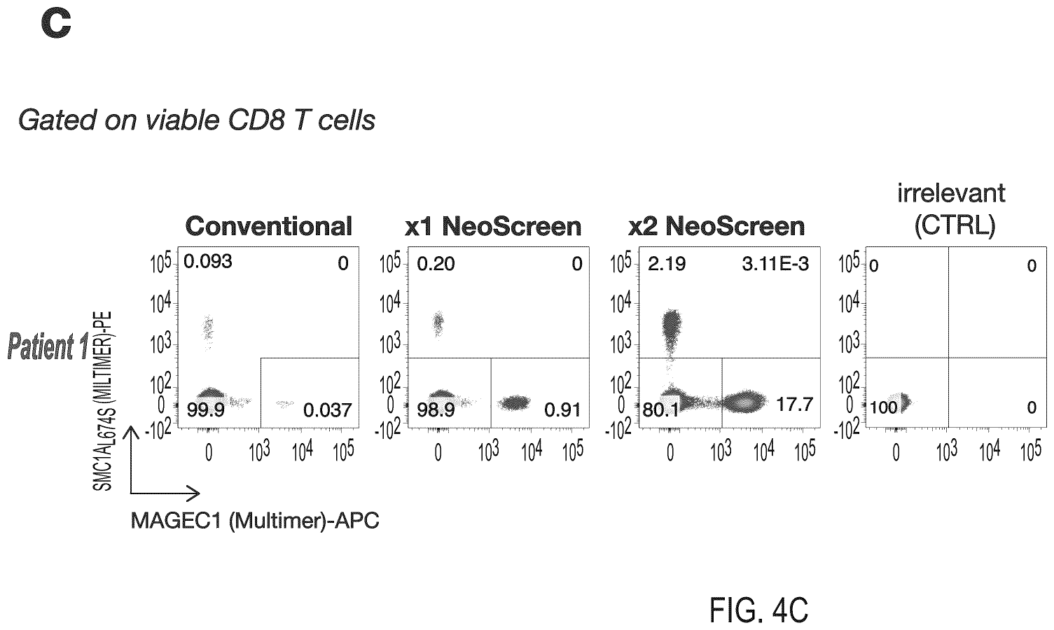

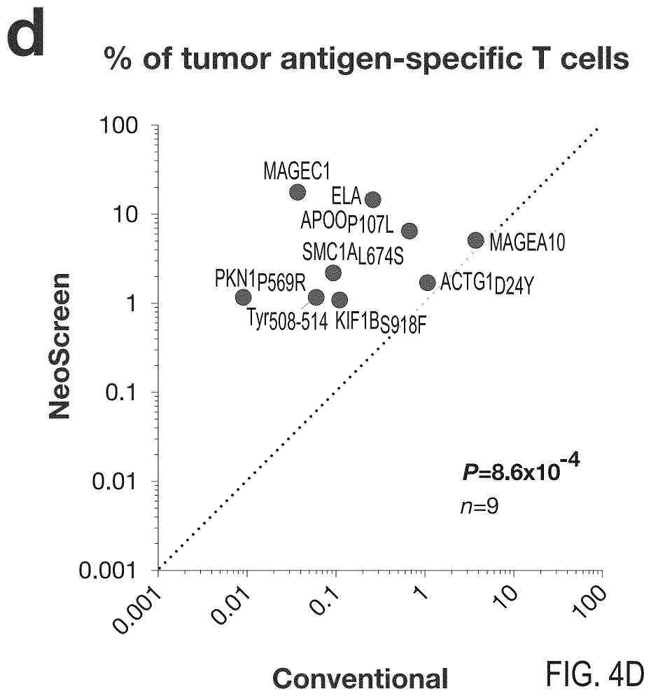

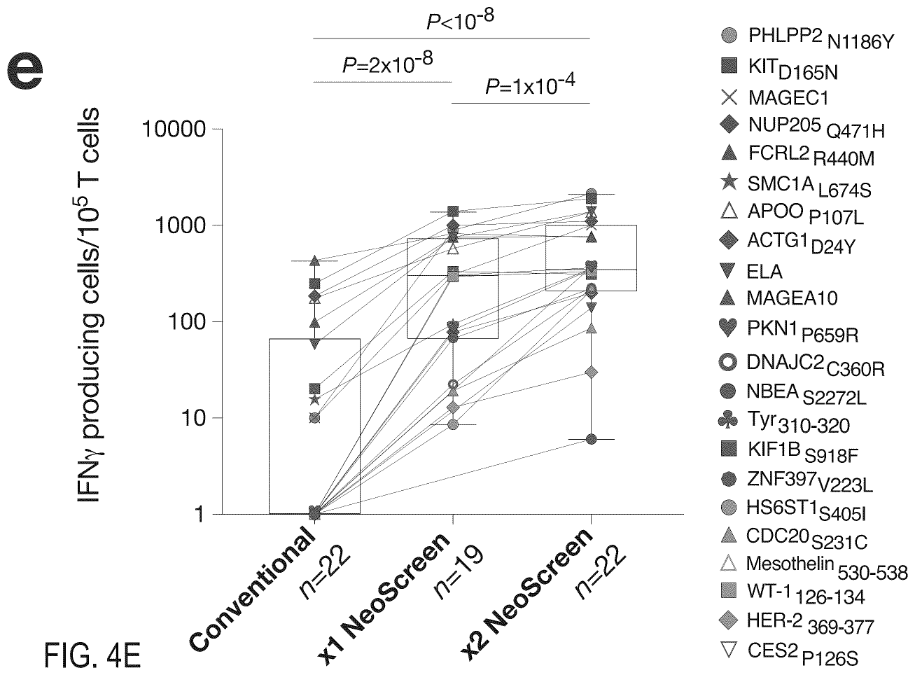

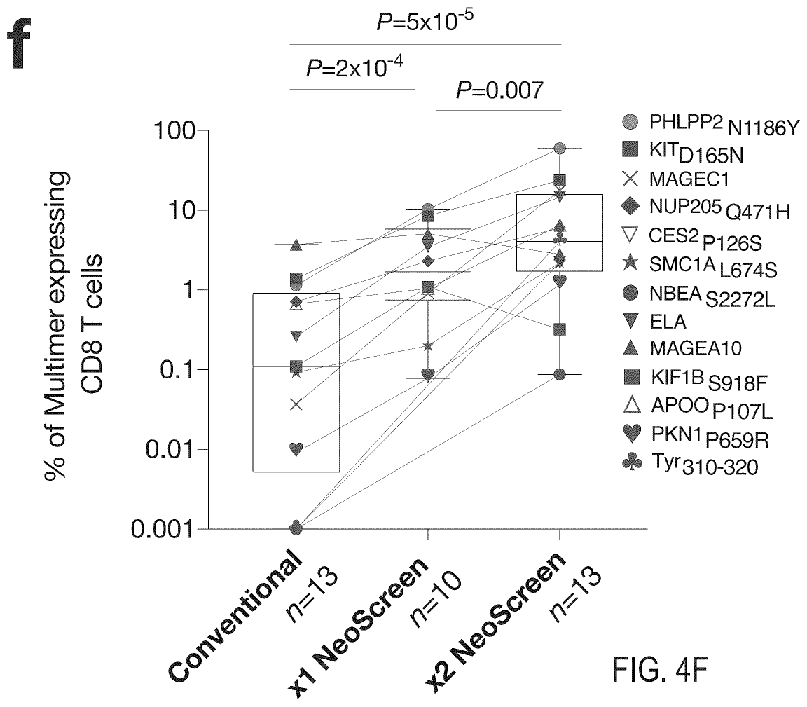

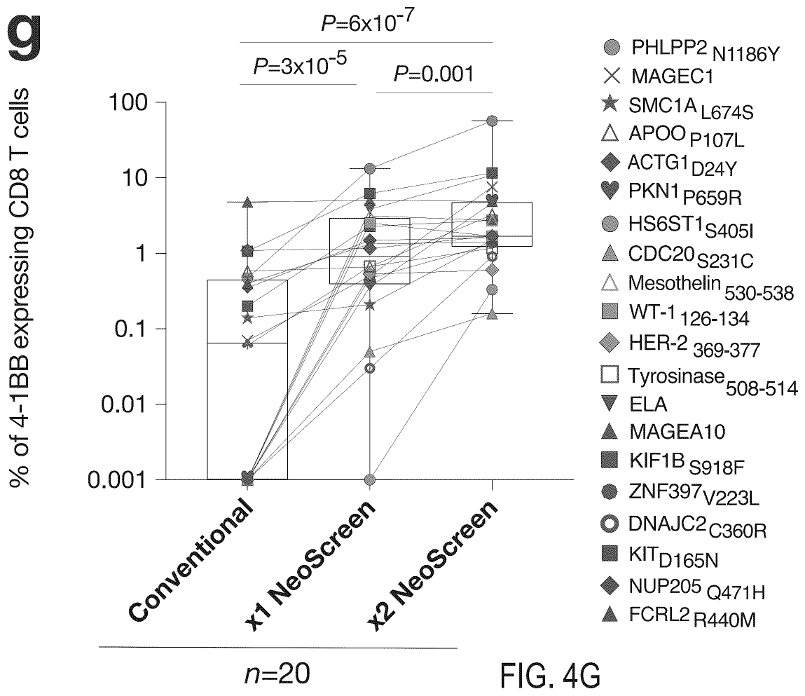

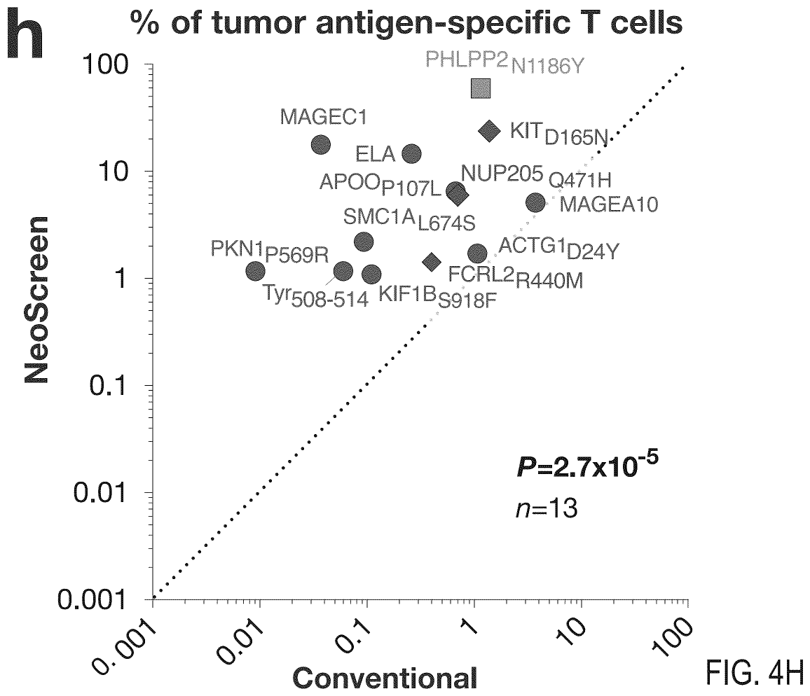

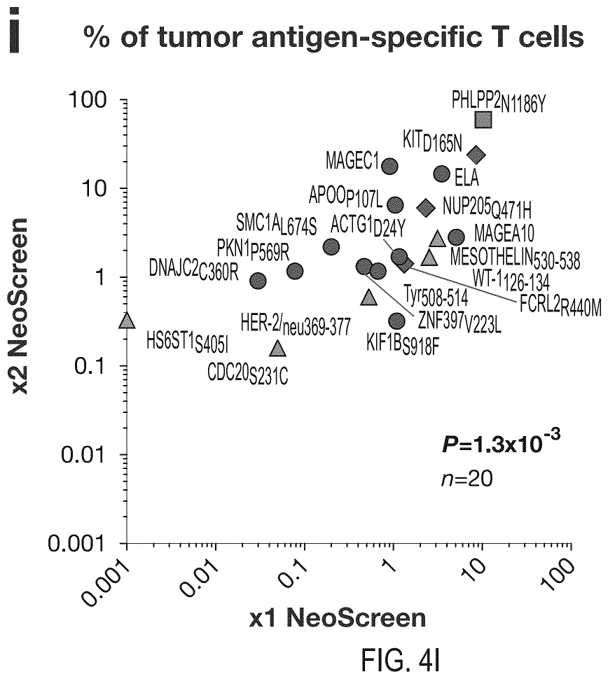

Next, the contribution of the Neoscreen approach was first validated by interrogating TILs from two tumor specimens (patient 6 and 7; Tables 1 and 4), where four neoepitope reactivities among (conventional) TILs expanded with IL-2 were readily identified. As compared to conventional TILs, markedly increased frequencies of neoepitope-specific CD8 T cells among TILs exposed to autologous engineered APCs were detected (P=0.01, n=4, FIG. 4A-B).

The ability of NeoScreen to reveal novel tumor antigens in seven additional patients was then tested (Tables 1-4). The proteogenomics NeoDisc pipeline was applied for prediction, immunopeptidomics-based identification, and prioritization of neoantigen, with a focus on non-synonymous somatic point mutations and tumor-associated antigens (TAA) candidates. Engineered autologous APCs loaded with neoantigens and/or TAAs candidates were added once (×1) or twice (×2) during TIL stimulation, and NeoScreen-expanded TILs were compared to conventional TIL cultures for the presence of antigen-specific cells (FIG. 1A). NeoScreen enabled the identification of 19 tumor epitopes in the seven patients (FIG. 1B-E). For 9/19 epitopes, a significantly higher frequency of specific TILs was observed in NeoScreen relative to conventional cultures (P=8.6×10⁻⁴, n=9, FIGS. 2C-D, and FIGS. 1B-D), while for 10/19 epitopes, tumor antigen-specific TILs were exclusively found in NeoScreen TILs (FIGS. 1B-D). Taken together, the average number of tumor epitopes per patient was three with Neoscreen against one using the conventional strategy (P=0.02, FIG. 1E).

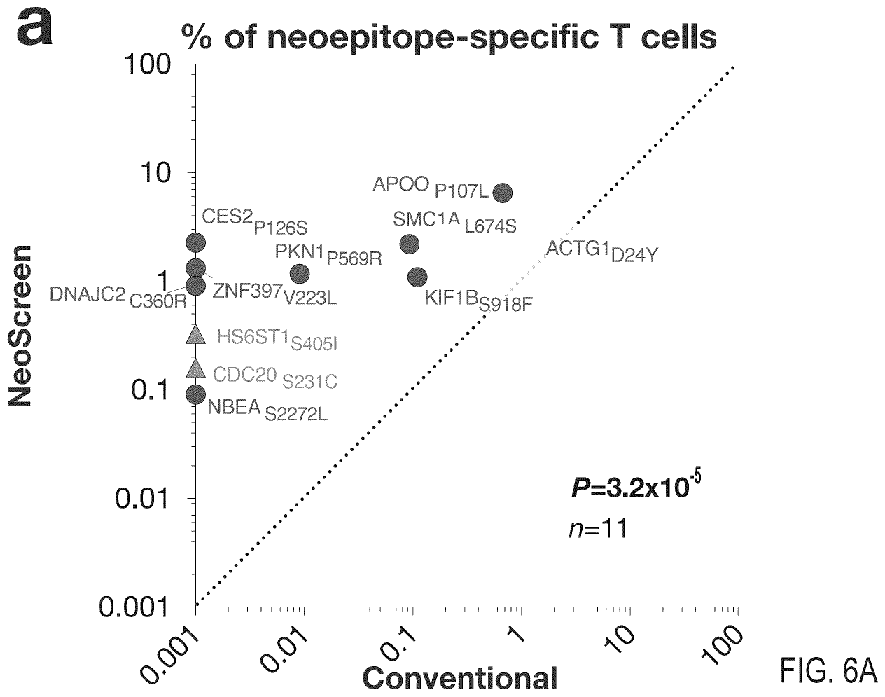

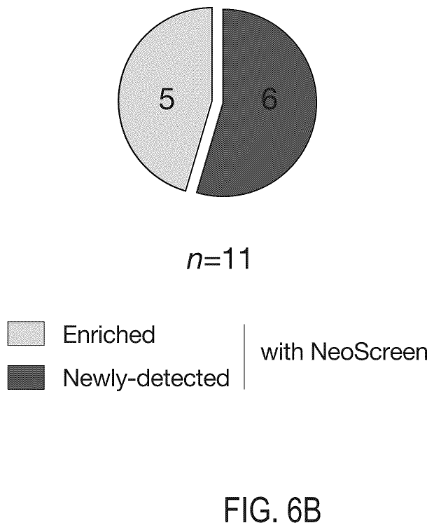

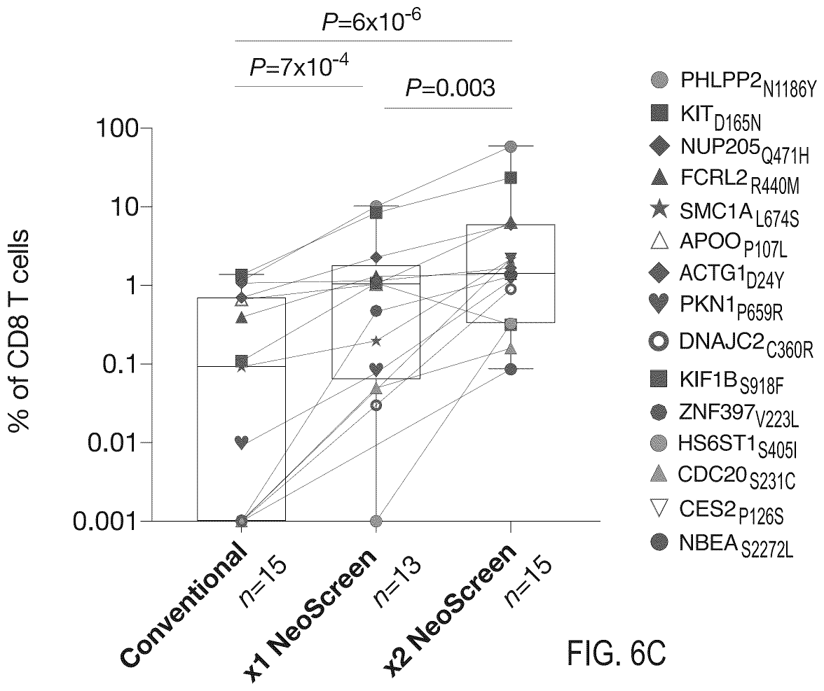

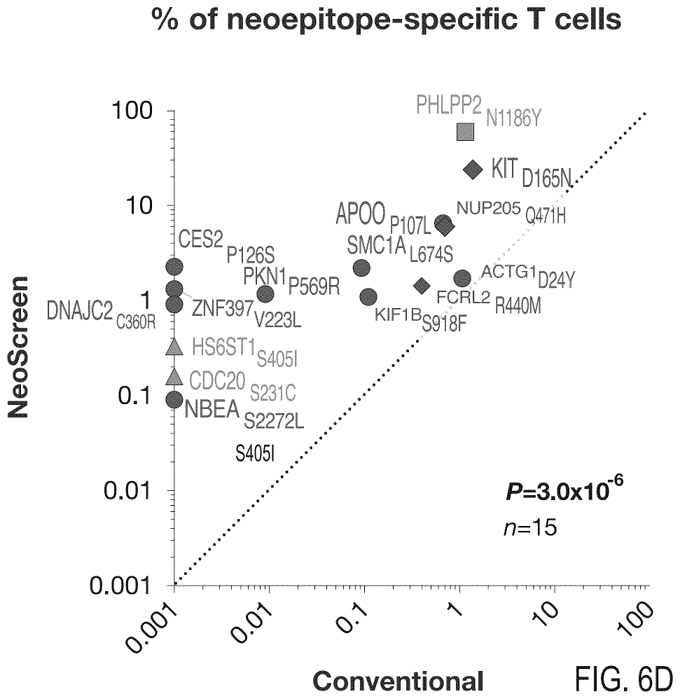

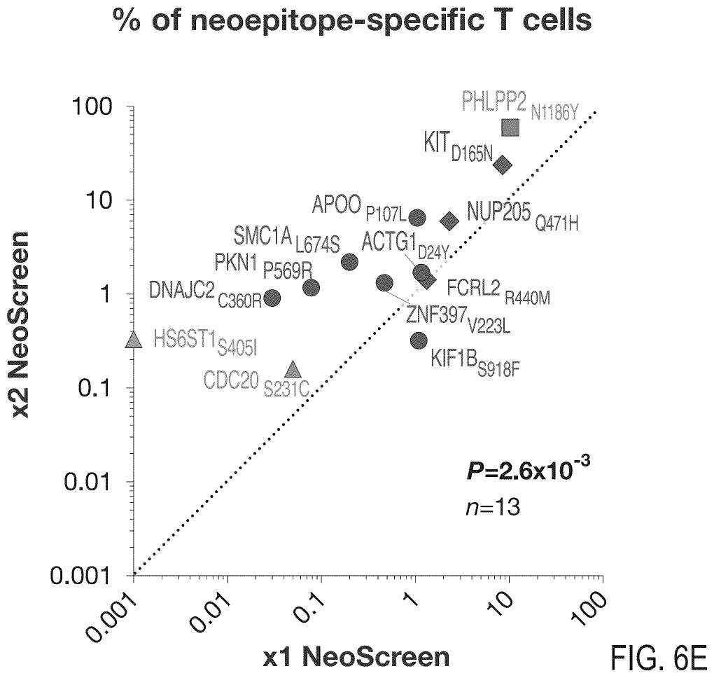

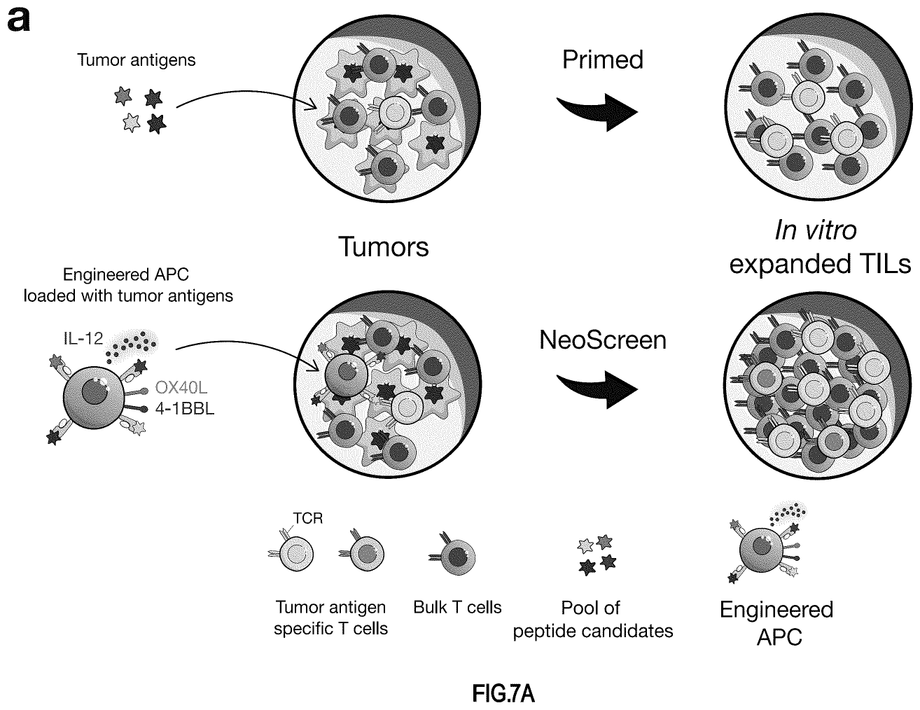

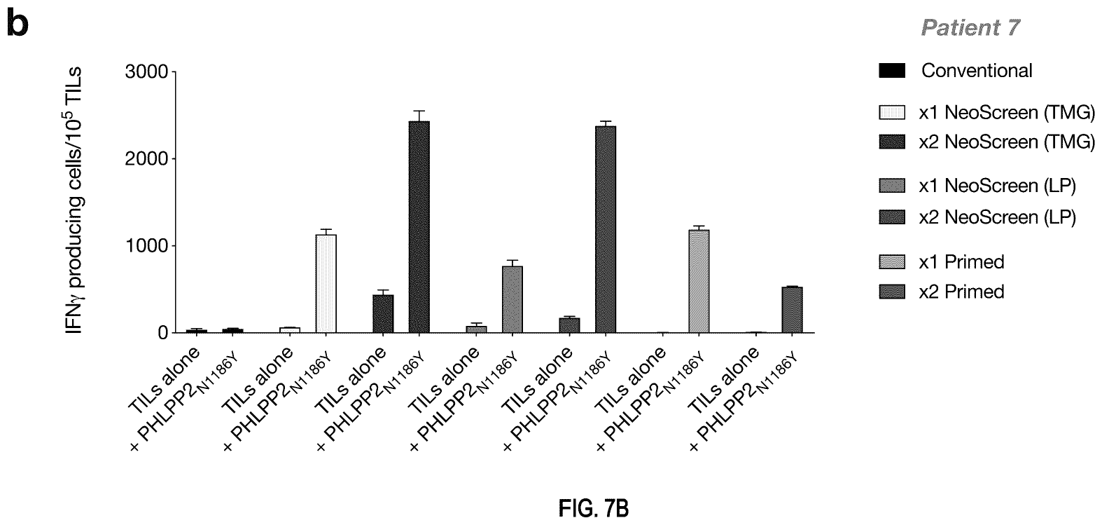

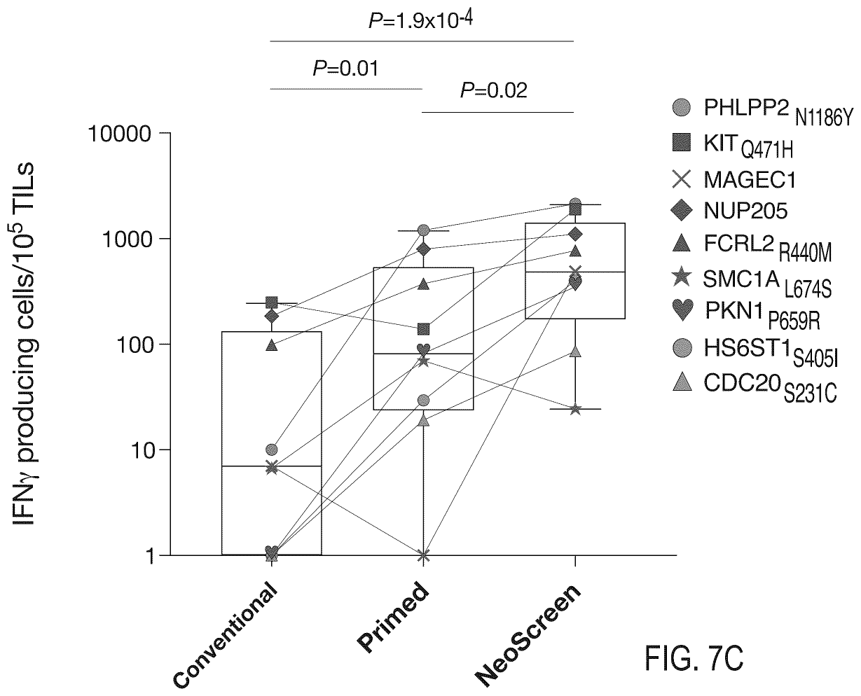

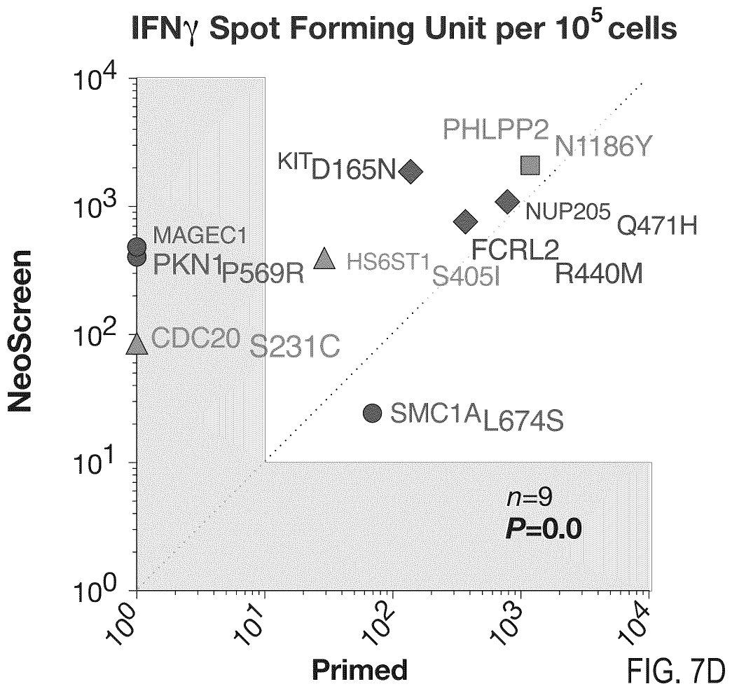

Cumulatively, through NeoScreen, using IFNγ ELISpot, pMHC-multimer, and 4-1BB staining, a total of 23 tumor antigens (Table 4) were validated, including 15 neoepitopes (FIG. 4E-G). In addition, neoantigen-specific TILs exhibited no or limited cross-reactivity against cognate wild-type peptides (FIG. 5). Relative to conventional TIL cultures, NeoScreen TILs were significantly enriched by several orders of magnitude for cells reactive to neoepitopes or TAAs (P<10⁻⁸, n=23, FIG. 1F). The frequency of TILs targeting epitopes identified in both NeoScreen and conventional conditions was increased by ˜67-fold (P=2.7×10⁻⁵, n=13 epitopes, FIG. 4H). Of interest, a second round of TIL stimulation further increased their frequency (FIG. 1G and FIG. 4I). Of note, NeoScreen remains significantly superior to the conventional strategy when exclusively neoantigens are considered (FIG. 6). Also, NeoScreen was found to be significantly improved relative to the previous study using peptides alone (FIG. 7) (Bobisse, S. et al. Nat. Commun. 9, 1092 (2018)). Overall, engineered APCs in the presence of tumor antigens enabled the significant expansion of neoantigen- (and TAA)-specific CD8 T cells in melanoma, ovarian, lung, and colon cancer, thus establishing a highly sensitive and reproducible methodology to identifying tumor antigens.

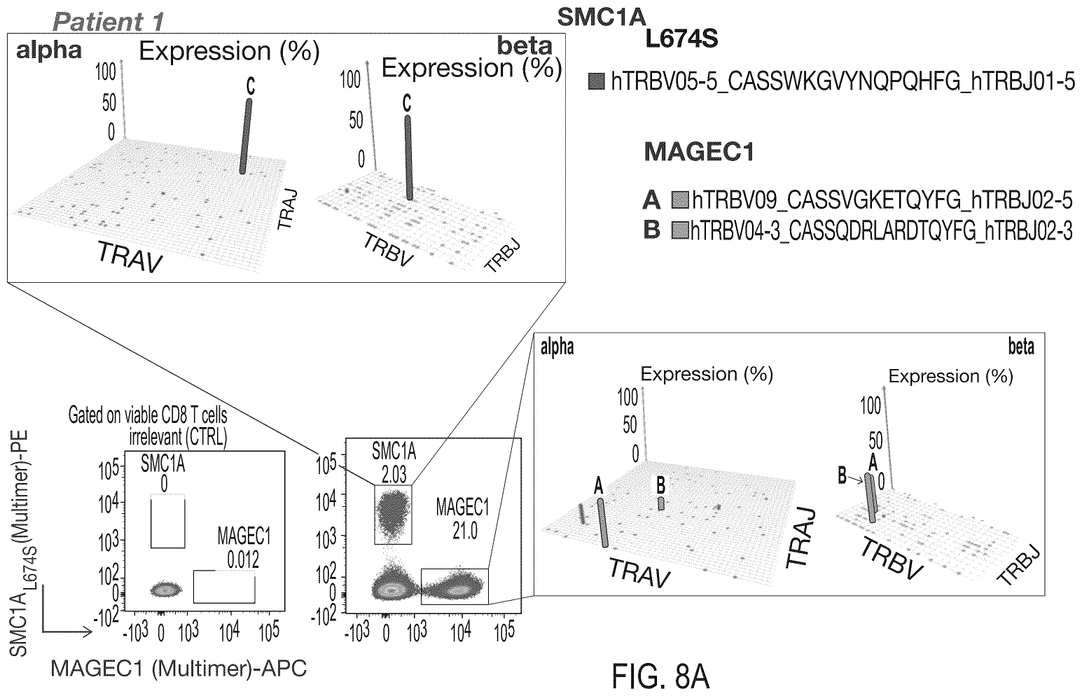

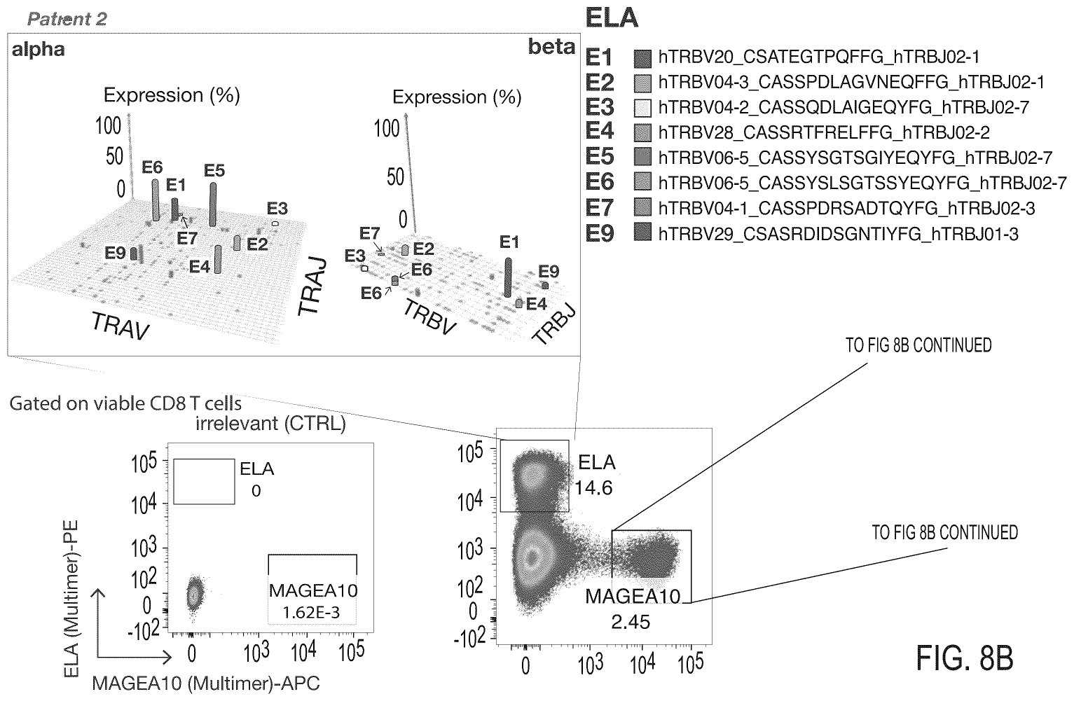

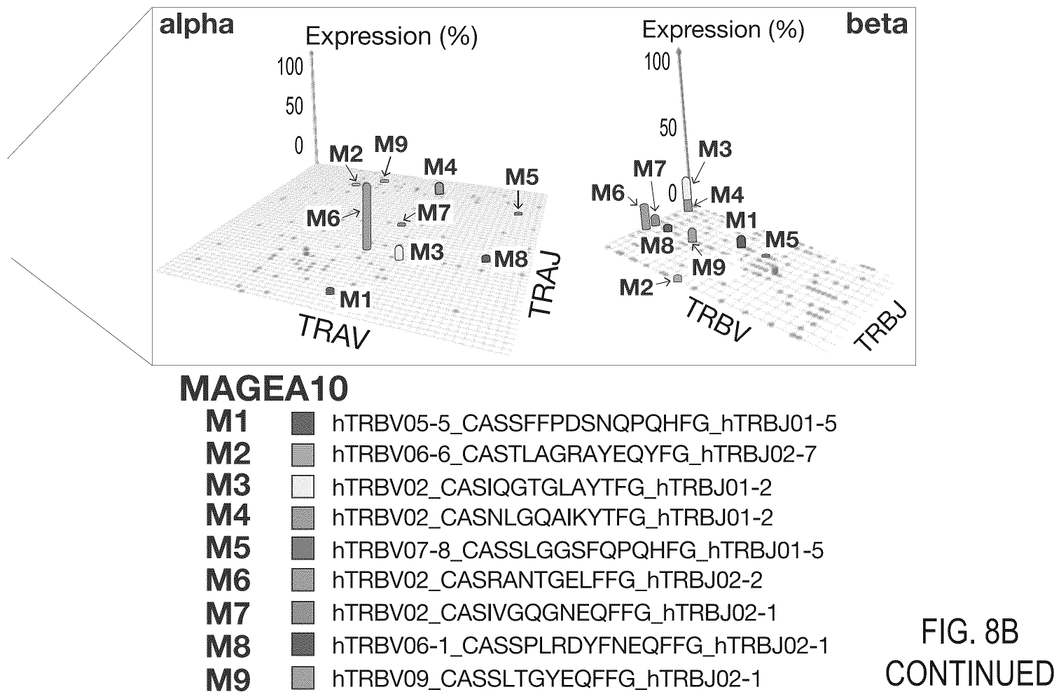

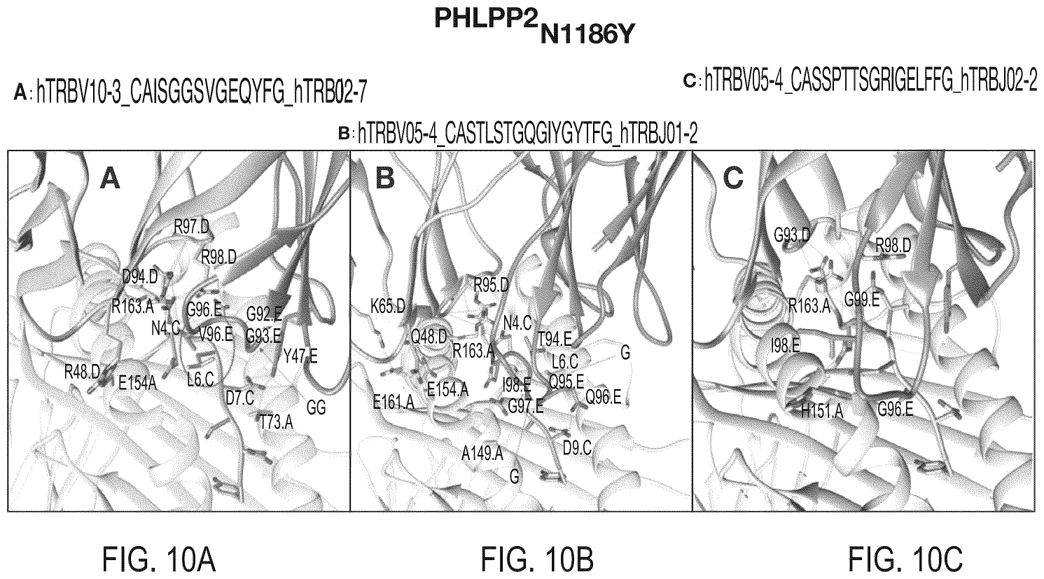

To test whether this novel platform would enable sensitive isolation of relevant TCRs directed against private tumor antigens (FIG. 1A), tumor antigen-specific NeoScreen TILs were purified using pMHC-multimers or 4-1BB upregulation and bulk TCRα and TCRβ sequencing of isolated T cells was performed (FIGS. 2A and 8; and Table 5). Individual tumor epitopes were recognized by >1 clonotypes, occurring at different frequencies among NeoScreen TILs. To confirm the specific recognition of tumor antigens, TCRαβ pairs were cloned into recipient Jurkat cells or primary T cells, which were then interrogated for expression of functional TCRs by pMHC-multimers (FIG. 2B and FIG. 9A-C) or 4-1BB upregulation (FIG. 9D). FIG. 2B shows an example of functional validation of three distinct TCRs (A, B, and C) cloned from sorted PHLPP2N1186Y-specific NeoScreen TILs. In addition, analysis of the three-dimensional TCR-pMHC structures obtained by homology modeling indicates that all three PHLPP2_{N1186Y}-specific TCRs could establish interactions with the cognate pMHC (FIGS. 2C and 10; and Table 6).

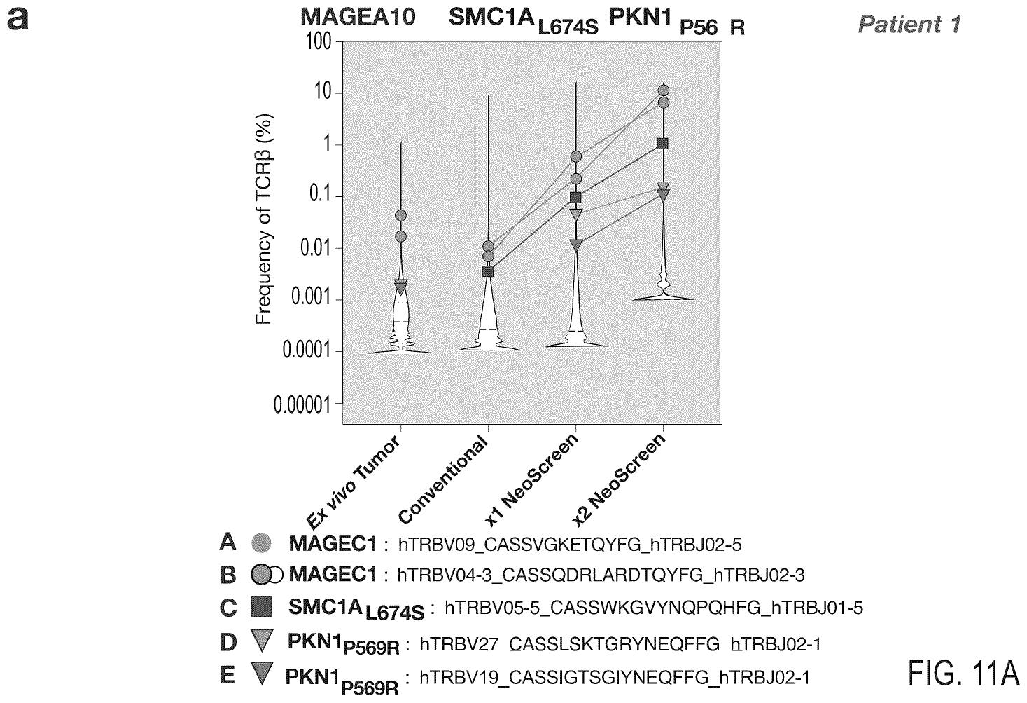

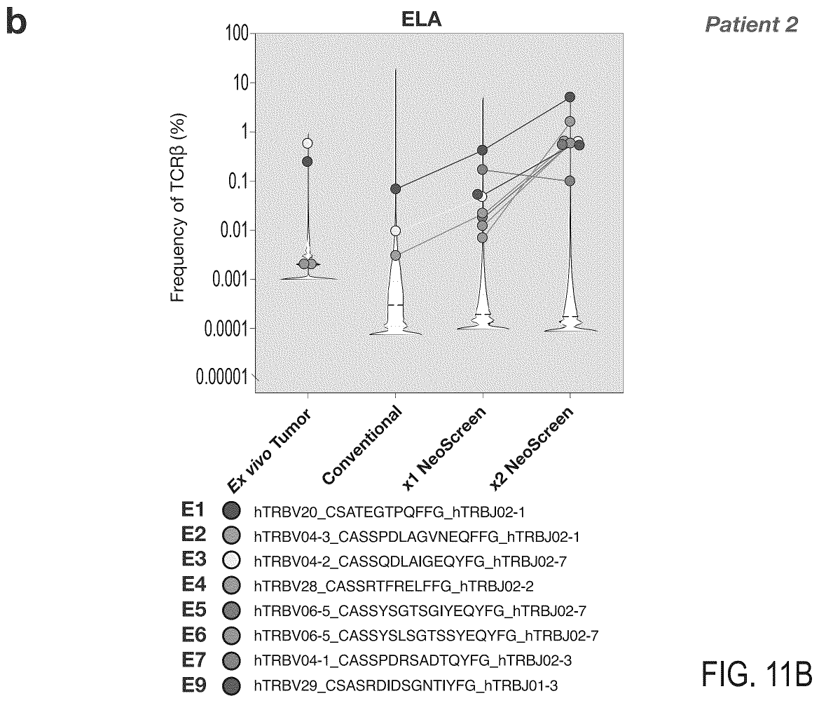

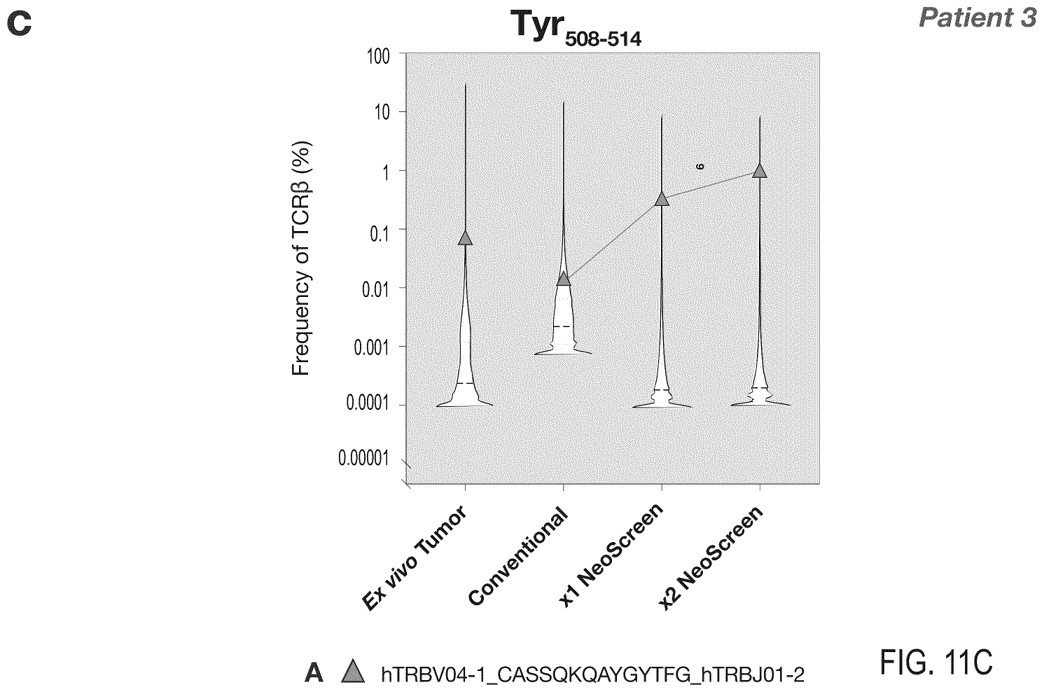

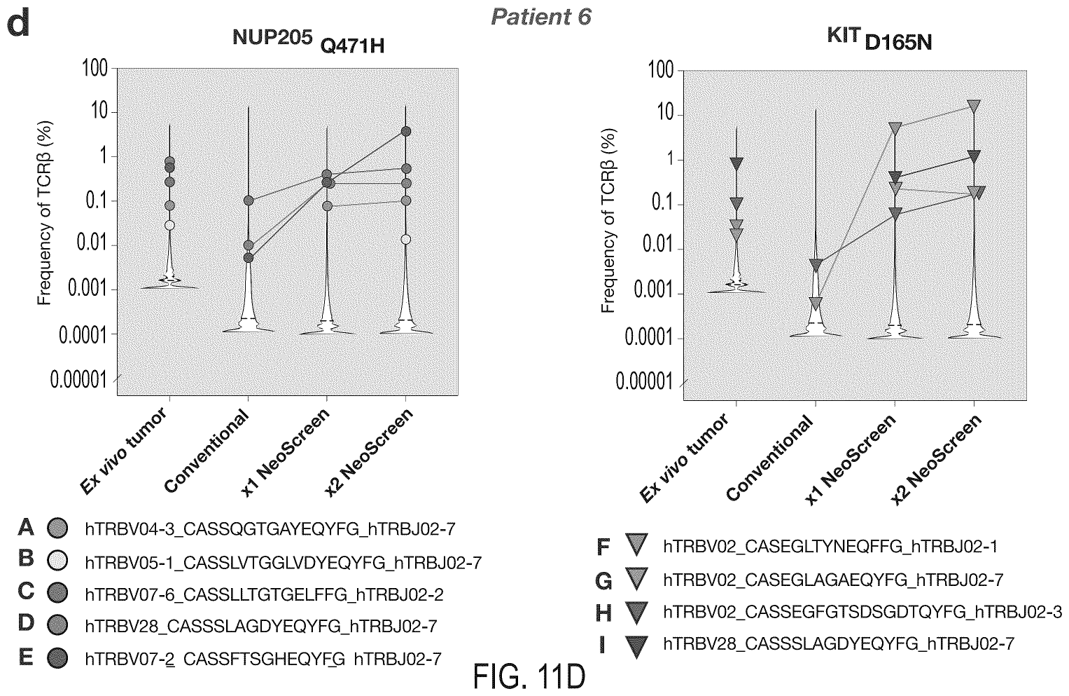

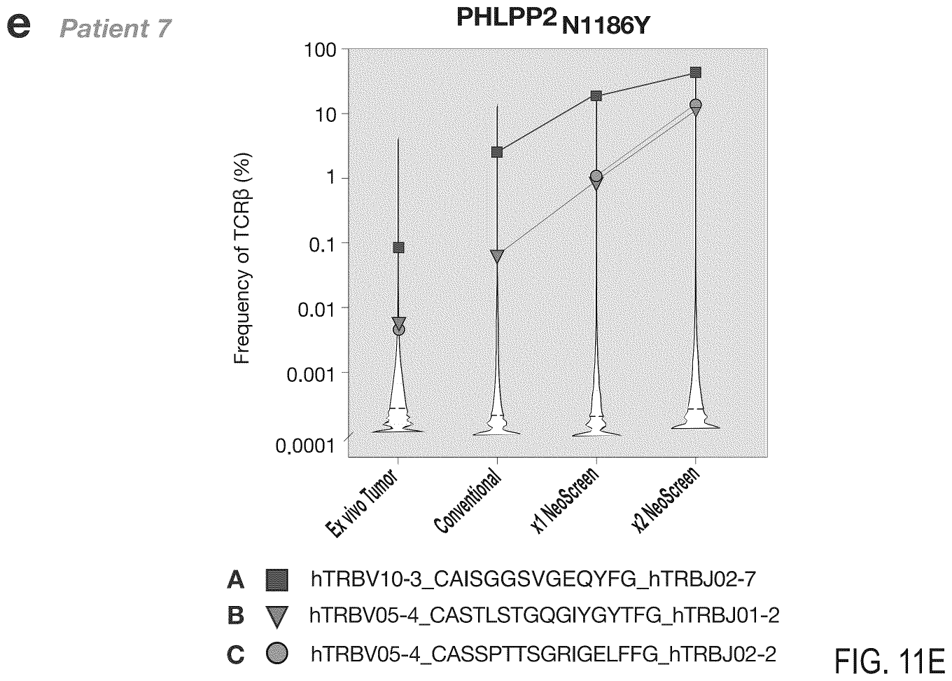

Next, TCRβ sequencing of bulk TIL cultures and ex vivo tumors (when available) was performed. The NeoScreen process indeed led to a marked expansion of tumor antigen-specific TILs through tracking validated TCRβ sequences in the original tumor and in NeoScreen expanded TILs (FIG. 2D and FIG. 11). As shown for representative PHLPP2_{N1186Y}-specific TCRs, all three TCRs were detected in the original tumor, and their respective frequencies considerably increased with NeoScreen (FIG. 2D and FIG. 11A). Although TCR-B and -C were detected at comparable frequencies (˜0.005%) in the original tumor, only TCR-B was found in conventional TILs, TCR-C being likely diluted under conventional culture conditions or only mobilized under NeoScreen conditions (FIG. 2D). Cumulative data of 50 clonotypes confirmed the potential of NeoScreen to identify novel TCRs specific to neoantigens or TAAs that were not detected in conventional TILs (n=17/50, FIG. 2F). Overall, a considerable enrichment of tumor antigen-specific TCRs by several orders of magnitude in NeoScreen-TILs over conventional TILs was demonstrated (P<10⁻⁸, n=50, FIG. 2E and FIG. 11).

Although neoantigen-specific TILs have been associated with clinical responses to immune checkpoint blockade and TIL-ACT, the recognition of autologous tumors by neoantigen-specific TCRs has not been consistently investigated. The antitumor reactivity of validated tumor antigen-specific TCRs revealed by NeoScreen was thus interrogated, when autologous tumor cell lines were available (FIG. 12A). Upon TCR cloning in primary activated T cells, all NeoScreen derived TCRs (n=31) specific to neoepitopes or TAAs were found to be tumor-reactive (FIGS. 16A-F). This is the first extensive demonstration that neoantigen-specific TCRs consistently target autologous tumors.

Finally, the hypothesis that TCRs identified with NeoScreen could be used for individualized TCR-based ACT was tested. Using patient-derived xenograft (PDX) tumors in the human IL-2 transgenic (hIL-2) NOG mouse model, it was shown that adoptively-transferred peripheral blood T cells transduced with tumor antigen-specific TCR cloned from NeoScreen-TILs mediated specific regression of established tumors in vivo (FIGS. 2G-H and FIG. 12B). Taken together, the data demonstrate in vitro and in vivo antitumor reactivity of antigen-specific TCRs identified through NeoScreen. This supports the feasibility of using NeoScreen for TCR-gene transfer therapy.

This disclosure demonstrates that NeoScreen, a method that enables highly sensitive screening of tumor (neo)antigens, yielded an unexpectedly broader repertoire of tumor antigen-reactive TCRs. NeoScreen acts not only by increasing the frequency of antigen-specific TCRs found with conventional methods, but also by recruiting additional TCR clonotypes which can be newly detected with markedly enhanced sensitivity. This is also the first time that engineered B cells were used at the initiation of TIL growth to enrich the sensitivity of antigen discovery. The RNA electroporation technology makes the disclosed approach easily applicable and offers the possibility to further engineer APCs for future improvements. Of note, although the requirement to generate autologous B cells and to predict and synthesize antigens delays the initiation of NeoScreen in vitro cultures by a couple of weeks, timelines remain in the same overall range as compared to alternative strategies with limited sensitivity.

The disclosed approach is also applicable for identification of CD4 T cell responses, given their emerging clinical relevance. Further, NeoScreen enables the highly efficient identification of tumor-specific antigens in melanoma, as well as in ovarian, colorectal, and lung cancer, and the highly-sensitive isolation of cognate tumor-reactive TCRs. Thus, NeoScreen represents a valuable pipeline to select relevant private target antigens for cancer vaccines and isolate tumor-reactive TCRs for personalized engineered T cell therapy of solid tumors.

TABLE 1
Description of patients.

| Patient | | | Tumor | Stage at | Samples' | Tumor | Other |
|---|---|---|---|---|---|---|---|
| Id | Gender | Age | type | diagnosis | origin | sample | remarks |
| Patient 1 | Male | 44 | Mucosal | pT4b pN0 | Lymph node | Tumor | BRAF |
| | | | melanoma | M0, IIC | metastasis | fragments | mutation |
| Patient 2 | Male | 60 | Skin | pT3 pN1b | Adrenal | Tumor | BRAF |
| | | | melanoma | M0, IIIB | metastasis | fragments | mutation |
| Patient 3 | Male | 53 | Melanoma | cT0 cN2b | Subcutaneous | Tumor | BRAF |
| | | | of unknown | cM0, IIIB | metastasis | fragments | mutation |
| | | | origin | | | | |
| Patient 4 | Male | 40 | Skin | pT3b pN0 | subcutaneous | Tumor | BRAF |
| | | | melanoma | M0, IIC | metastasis | fragments | mutation |
| Patient 5 | Female | 65 | Skin | pT3a | Lymph node | Tumor | BRAF |
| | | | melanoma | pN1a | metastasis | fragments | mutation |
| | | | | cM0, IIIA | | | |
| Patient 6 | Male | 69 | Lung | pT2b pN0 | Lung | Tumor | |
| | | | squamous cell | M0, IIA | | fragments | |
| | | | carcinoma | | | | |
| Patient 7 | Male | 73 | Colon | pT3 pN0 | caecum | Tumor | MSI |
| | | | adenocarcinoma | M0, II | | fragments | |
| Patient 8 | Female | 63 | Epithelial | IV | | Dissociated | |
| | | | ovarian | | | tumor | |
| | | | adenocarcinoma | | | cells | |
| Patient 9 | Female | 60 | Epithelial | IV | | Dissociated | |
| | | | ovarian | | | tumor | |
| | | | adenocarcinoma | | | cells | |

Patient identification number, gender, age, tumor type, stage at diagnosis, origin of sample, tumor sample type (tumor fragments or dissociated tumor cells) and further details (oncogene mutations, microsatellite instability (MSI)).

TABLE 2
Number of SNVs, HLA class-I haplotype and number of tumor
antigen candidates used for NeoScreen discovery.

| | Non synonymous | | | | | | | Tumor antigen |
|---|---|---|---|---|---|---|---|---|
| Patient Id | mutations SNV* | HLA-A | | HLA-B | | HLA-C | | candidates^{‡} |
| Patient 1 | 46 | 01:01 | 03:01 | 27:05 | 57:01 | 01:02 | 06:02 | 83 |
| Patient 2 | 95 | 02:05 | 32:01 | 07:02 | 44:03 | 04:01 | 07:02 | 124 |
| Patient 3 | 71 | 02:01 | 01:01 | 08:01 | 40:01 | 07:01 | 03:04 | 139 |
| Patient 4 | 128 | 01:01 | 23:01 | 07:02 | 15:01 | 14:02 | 12:03 | 191 |
| Patient 5 | 70 | 02:01 | 26:01 | 44:02 | 51:08 | 05:01 | 05:01 | 70 |
| Patient 8 | 46 | 02:01 | 29:02 | 44:02 | 44:04 | 05:01 | 16:01 | 19 |
| Patient 9 | 10 | 23:01 | 23:01 | 14:02 | 50:01 | 06:02 | 08:02 | 1 |

*single nucleotide variants (SNVs) related to tumor antigen candidates used for NeoScreen of patients 1 to 5;
† total number of tumor antigens used in NeoScreen; for patient 8, ten HLA-A*02:01-restricted epitopes detailed in Table 3 were also included.

TABLE 3
HLA-class I restriction and epitope sequences of shared HLA-A*02:01-restricted
epitopes used for NeoScreen for patient 8.

| Epitopes | HLA restriction | Peptide sequence | SEQ ID NO |
|---|---|---|---|
| A2/hTERT (689-697) | A02:01 | ILAKFLHWL | 243 |
| Survivin (96-104) (T98M) | A02:01 | LTLGEFLKL | 244 |
| p53 | A02:01 | LLGRNSFE | 245 |
| Mesothelin (530-538) | A02:01 | VLPLTVAEV | 246 |
| NY-ESO1 (457-165) | A02:01 | SLLMWITQC | 247 |
| HER-2/neu (654-662) | A02:01 | IISAVVGIL | 248 |
| HER-2/neu (369-377) | A02:01 | KIFGSLAFL | 249 |
| HER-2/neu (689-697) | A02:01 | RLLQETELV | 250 |
| WT-1 (126-134) | A02:01 | RMFPNAPYL | 251 |
| MUC-1 (950-958) | A02:01 | STAPPVHNV | 252 |

TABLE 4
Validated tumor-associated antigens and neoepitopes.

| | Non | | | | | |
|---|---|---|---|---|---|---|
| | synonymous | | | SEQ | | SEQ |
| | mutations | HLA re- | Minimal | ID | Long epitope | ID |
| Gene | SNVs | striction | epitope | NO | (for LP or TMG) | NO |
| SMC1A | L674S | B27:05 | RRWDEKA | 230 | KAKARRWDEKAVDKSKEK | 259 |
| | | | VDKSK | | KERL | |
| MAGEC10 | NA | C01:02 | FAFGEPRE | 229 | NA | |
| | | | L | | | |
| PKN1 | P569R | A03:01 | GTDSDSSR | 231 | ISVEKLNLGTDSDSSRQK | 260 |
| | | | QK | | | |
| ZNF397 | V223L | B44:03* | SEHESNLL | 253 | NA | |
| | | | W | | | |
| KIF1B | S918F | A02:05 | TADFDITE | 234 | TPSPTFSTADFDITELADEQQ | 261 |
| | | | L | | DEME | |
| DNAJC2 | C360R | B44:03* | QKLRNSRK | 254 | IKKERQKLRNSRKTWNHFS | 262 |
| | | | TW | | DN | |
| MAGEA10_{□} | NA | A02:05 | GLYDGME | 232 | NA | |
| | | | HL | | | |
| ELA_{□} | NA | A02:05 | ELAGIGILT | 233 | NA | |
| | | | V | | | |
| Tyro- | NA | B08:01* | LPEEKQPL | 242 | NA | |
| sinase | | | | | | |
| (508- | | | | | | |
| 504)0 | | | | | | |
| NBEA• | S2272L | B07:02 | LPQARRIL | 244 | NA | |
| | | | L | | | |
| Tyro- | NA | B15:01 | RLPSSADV | 237 | NA | |
| sinase | | | EF | | | |
| (310- | | | | | | |
| 320)0• | | | | | | |
| CES2• | P126S | B15:01 | VQTFLGISF | 236 | NA | |
| APOO | P107L | A02:01 | ALPGFFPR | 238 | WGLDSYDYLQNALPGFFPR | 263 |
| | | | L | | LGVIG | |
| ACTG1 | D24Y | B51:08* | AGYDAPR | 255 | GSGMCKAGFAGYDAPRAV | 264 |
| | | | AV | | FPSIVG | |
| NUP205 | Q471H | B35:03 | EPLHTPTI | 239 | HLELALEYWCPTEPLHTPTI | 265 |
| | | | M | | MGSYLGVAHQR | |
| FCRL2 | R440M | B35:03* | MPNPQEFT | 256 | ISGESSATNEPRGASMPNPQ | 266 |
| | | | Y | | EFTYSSPTPDM | |
| KIT | D165N | B35:03 | IPNPKAGI | 240 | GCQGKPLPKDLRFIPNPKAG | 267 |
| | | | M | | IMIKSVKRAYH | |
| PHLPP2 | N1186Y | A01:01 | QSDNGLDS | 241 | ATFSSNQSDNGLDSDYDQP | 268 |
| | | | DY | | VEGVITNGSKVE | |
| CDC20 | S231C | B44:02* | GEYISCVA | 257 | NA | |
| | | | WI | | | |
| Meso- | NA | A02:01* | VLPLTVAE | 246 | NA | |
| thelin | | | V | | | |
| (530- | | | | | | |
| 538)0 | | | | | | |
| WT-1 | NA | A02:01* | RMFPNAPY | 25 | NA | |
| (126- | | | L | | | |
| 134)0 | | | | | | |
| HER- | NA | A02:01* | KIFGSLAFL | 249 | NA | |
| 2/neu | | | | | | |
| (369- | | | | | | |
| 377)0 | | | | | | |
| HS6ST1 | S405I | A23:01* | DYMIHIIEK | 258 | NA | |
| | | | W | | | |

Patient identification number; gene († MAGEA10 & ELA were considered since patient 2 was previously vaccinated with these TAAs; ^{▴} TAAs were identified as described in the Methods section; • patient 4 had a total number of 191 antigen candidates and thus they were split into two pools for in vitro TIL expansion, CES2P126s was in a first pool, noted NeoScreen (1), and NBEA_{S2272L} and tyrosinase310-320 were in the second pool NeoScreen (2) (Methods); ★ patient 8 was interrogated with a list of TAA, as described in Table 3; single nucleotide variants (SNVs) (NA: not applicable); HLA restriction (predicted HLA-restrictions not confirmed by pMHC multimers are shown with an *); minimal peptide sequence; long peptide sequence (when tandem minigenes (TMG) and/or long peptides (LP) were used at the initiation of TIL cultures).

TABLE 5
Description of tumor antigen-specific TCRs.

| | | | | | | | | TCR | | | |
|---|---|---|---|---|---|---|---|---|---|---|---|
| | | Non | | | | | Add- | pMHC | | | |
| | | synonymus | | | | | itional | beta | SEQ | | SEQ |
| Patient | | mutations | HLA | Minimal | | TCR | Id in- | chain | ID | | ID |
| ID | Gene | SNVs | restriction | epitope | | Id | formation | CDR3 | NO | TCR pMHC beta chain | NO |
| Patient | MAGEC1 | | C01:02 | FA | 229 | A | hTRBV09_ | CAS | 2 | MGFRLLCCVAFCLLGAGPVDSGVTQTPKH | 78 |
| 1 | | | | FG | | | CASS | SVG | | LITATGQRVTLRCSPRSGDLSVYWYQQSL | |
| | | | | EP | | | VGKET | KET | | DQGLQFLIQYYNGEERAKGNILERFSAQQF | |
| | | | | RE | | | QYFG_ | QYF | | PDLHSELNLSSLELGDSALYFCASSVGKET | |
| | | | | L | | | hTRBJ02-5 | G | | QYFGPGTRLLVLEDLNKVFPPEVAVFEPSE | |
| | | | | | | | | | | AEISHTQKATLVCLATGFFPDHVELSWWV | |
| | | | | | | | | | | NGKEVHSGVSTDPQPLKEQPALNDSRYC | |
| | | | | | | | | | | LSSRLRVSATFWQNPRNHFRCQVQFYGL | |
| | | | | | | | | | | SENDEWTQDRAKPVTQIVSAEAWGRADC | |
| | | | | | | | | | | GFTSVSYQQGVLSATILYEILLGKATLYAV | |
| | | | | | | | | | | LVSALVLMAMVKRKDF | |
| | | | | | | B | hTRBV04- | CAS | 4 | MGCRLLCCAVLCLLGAGELVPMETGVTQT | 80 |
| | | | | | | | 3_CASS | SQD | | PRHLVMGMTNKKSLKCEQHLGHNAMYWY | |
| | | | | | | | QDRL | RLA | | KQSAKKPLELMFVYSLEERVENNSVPSRFS | |
| | | | | | | | ARDT | RDT | | PECPNSSHLFLHLHTLQPEDSALYLCASSQ | |
| | | | | | | | QYFG_ | QYF | | DRLARDTQYFGPGTRLTVLEDLNKVFPPE | |
| | | | | | | | hTRBJ02-3 | G | | VAVFEPSEAEISHTQKATLVCLATGFFPDH | |
| | | | | | | | | | | VELSWWVNGKEVHSGVSTDPQPLKEQPA | |
| | | | | | | | | | | LNDSRYCLSSRLRVSATFWQNPRNHFRC | |
| | | | | | | | | | | QVQFYGLSENDEWTQDRAKPVTQIVSAEA | |
| | | | | | | | | | | WGRADCGFTSVSYQQGVLSATILYEILLG | |
| | | | | | | | | | | KATLYAVLVSALVLMAMVKRKDF | |
| | SMC1A | L674S | B27:05 | RRW | 230 | C | hTRBV05- | CAS | 6 | MGPGLLCWVLLCLLGAGPVDAGVTQSPTH | 82 |
| | | | | DEK | | | 5_ | SWK | | LIKTRGQQVTLRCSPISGHKSVSWYQQVLG | |
| | | | | AVD | | | CASS | GVY | | QGPQFIFQYYEKEERGRGNFPDRFSARQF | |
| | | | | KSK | | | WKGV | NQP | | PNYSSELNVNALLLGDSALYLCASSWKGV | |
| | | | | | | | YNQP | QHF | | YNQPQHFGDGTRLSILEDLNKVFPPEVAVF | |
| | | | | | | | QHFG_ | G | | EPSEAEISHTQKATLVCLATGFFPDHVELS | |
| | | | | | | | hTRBJ01-5 | | | WWVNGKEVHSGVSTDPQPLKEQPALNDS | |
| | | | | | | | | | | RYCLSSRLRVSATFWQNPRNHFRCQVQF | |
| | | | | | | | | | | YGLSENDEWTQDRAKPVTQIVSAEAWGR | |
| | | | | | | | | | | ADCGFTSVSYQQGVLSATILYEILLGKATL | |
| | | | | | | | | | | YAVLVSALVLMAMVKRKDF | |
| | PKN1 | P569R | A03:01 | GTD | 231 | D | hTRBV27_ | CAS | 8 | MGPQLLGYVVLCLLGAGPLEAQVTQNPRY | 84 |
| | | | | SDS | | | CASS | SLS | | LITVTGKKLTVTCSQNMNHEYMSWYRQDP | |
| | | | | SRQ | | | LSK | KTG | | GLGLRQIYYSMNVEVTDKGDVPEGYKVSR | |
| | | | | K | | | TG | RYN | | KEKRNFPLILESPSPNQTSLYFCASSLSK | |
| | | | | | | | RY | EQF | | TGRYNEQFFGPGTRLTVLEDLNKVFPPEVA | |
| | | | | | | | NE | FG | | VFEPSEAEISHTQKATLVCLATGFFPDHVE | |
| | | | | | | | QFFG_ | | | LSWWVNGKEVHSGVSTDPQPLKEQPALND | |
| | | | | | | | hTRBJ02-1 | | | SRYCLSSRLRVSATFWQNPRNHFRCQVQ | |
| | | | | | | | | | | FYGLSENDEWTQDRAKPVTQIVSAEAWG | |
| | | | | | | | | | | RADCGFTSVSYQQGVLSATILYEILLGKAT | |
| | | | | | | | | | | LYAVLVSALVLMAMVKRKDF | |

| | TCR pMHC beta | | | TCR | | | | TCR pMHC alpha | |
|---|---|---|---|---|---|---|---|---|---|
| | chain | | | pMHC | | | | chain | |
| | (excluding | SEQ | Additional | alpha | SEQ | | SEQ | (excluding | SEQ |
| Patient | the constant | ID | Id | chain | ID | | ID | the constant | ID |
| Id | region) | NO | information | CDR3 | NO | TCR pMHC alpha chain | NO | region) | NO |
| Patient | MGFRLLCCVAFCL | 154 | hTRAV13-1_ | CAA | 1 | MTSIRAVFIFLWLQLDLVNGENVEQHP | 77 | MTSIRAVFIFLWLQ | 153 |
| 1 | LGAGPVDSGVTQT | | CAA | SDS | | STLSVQEGDSAVIKCTYSDSASNYFP | | LDLVNGENVEQHP | |
| | PKHLITATGQRVTL | | SDS | SAS | | WYKQELGKGPQLIIDIRSNVGEKKDQR | | STLSVQEGDSAVI | |
| | RCSPRSGDLSVY | | SAS | KIF | | IAVTLNKTAKHFSLHITETQPEDSAVYF | | KCTYSDSASNYFP | |
| | WYQQSLDQGLQF | | KII | G | | CAASDSSASKIIFGSGTRLSIRPNIQNP | | WYKQELGKGPQLI | |
| | LIQYYNGEERAKG | | FG_ | | | DPAVYQLRDSKSSDKSVCLFTDFDSQ | | IDIRSNVGEKKDQ | |
| | NILERFSAQQFPD | | hTRAJ03 | | | TNVSQSKDSDVYITDKTVLDMRSMDF | | RIAVTLNKTAKHFS | |
| | LHSELNLSSLELG | | | | | KSNSAVAWSNKSDFACANAFNNSIIP | | LHITETQPEDSAVY | |
| | DSALYFCASSVGK | | | | | EDTFFPSPESSCDVKLVEKSFETDTN | | FCAASDSSASKIIF | |
| | ETQYFGPGTRLLV | | | | | LNFQNLSVIGFRILLLKVAGFNLLMTL | | GSGTRLSIRPN | |
| | L | | | | | RLWSS | | | |
| | MGCRLLCCAVLCL | 156 | hTRAV14_ | CAM | 3 | MSLSSLLKVVTASLWLGPGIAQKITQT | 79 | MSLSSLLKVVTASL | 155 |
| | LGAGELVPMETGV | | CAM | REP | | QPGMFVQEKEAVTLDCTYDTSDQSY | | WLGPGIAQKITQT | |
| | TQTPRHLVMGMT | | REP | YYN | | GLFWYKQPSSGEMIFLIYQGSYDEQN | | QPGMFVQEKEAV | |
| | NKKSLKCEQHLGH | | YYN | QGG | | ATEGRYSLNFQKARKSANLVISASQLG | | TLDCTYDTSDQSY | |
| | NAMYWYKQSAKK | | QG | KL | | DSAMYFCAMREPYYNQGGKLIFGQG | | GLFWYKQPSSGE | |
| | PLELMFVYSLEER | | GKL | IF | | TELSVKPNIQNPDPAVYQLRDSKSSD | | MIFLIYQGSYDEQN | |
| | VENNSVPSRFSPE | | IFG_ | G | | KSVCLFTDFDSQTNVSQSKDSDVYIT | | ATEGRYSLNFQKA | |
| | CPNSSHLFLHLHT | | hTR | | | DKTVLDMRSMDFKSNSAVAWSNKSD | | RKSANLVISASQL | |
| | LQPEDSALYLCAS | | AJ23 | | | FACANAFNNSIIPEDTFFPSPESSCDV | | GDSAMYFCAMRE | |
| | SQDRLARDTQYF | | | | | KLVEKSFETDTNLNFQNLSVIGFRILL | | PYYNQGGKLIFGQ | |
| | GPGTRLTVL | | | | | LKVAGFNLLMTLRLWSS | | GTELSVKPN | |
| | MGPGLLCWVLLCL | 158 | hTRAV26- | CIL | 5 | MKLVTSITVLLSLGIMGDAKTTQPNSM | 81 | MKLVTSITVLLSLG | 157 |
| | LGAGPVDAGVTQ | | 2_C | RAV | | ESNEEEPVHLPCNHSTISGTDYIHWYR | | IMGDAKTTQPNSM | |
| | SPTHLIKTRGQQV | | ILR | YVR | | QLPSQGPEYVIHGLTSNVNNRMASLAI | | ESNEEEPVHLPCN | |
| | TLRCSPISGHKSV | | AVY | FG | | AEDRKSSTLILHRATLRDAAVYYCILRA | | HSTISGTDYIHWY | |
| | SWYQQVLGQGPQ | | VRF | | | VYVRFGAGTRLTVKPNIQNPDPAVYQ | | RQLPSQGPEYVIH | |
| | FIFQYYEKEERGR | | G_ | | | LRDSKSSDKSVCLFTDFDSQTNVSQS | | GLTSNVNNRMASL | |
| | GNFPDRFSARQFP | | hTRAJ43 | | | KDSDVYITDKTVLDMRSMDFKSNSAV | | AIAEDRKSSTLILH | |
| | NYSSELNVNALLL | | | | | AWSNKSDFACANAFNNSIIPEDTFFPS | | RATLRDAAVYYCIL | |
| | GDSALYLCASSWK | | | | | PESSCDVKLVEKSFETDTNLNFQNLS | | RAVYVRFGAGTRL | |
| | GVYNQPQHFGDG | | | | | VIGFRILLLKVAGFNLLMTLRLWSS | | TVKPN | |
| | TRLSIL | | | | | | | | |
| | MGPQLLGYVVLCL | 160 | hTRAV20_ | CAV | 7 | MEKMLECAFIVLWLQLGWLSGEDQVT | 83 | MEKMLECAFIVLW | 159 |
| | LGAGPLEAQVTQN | | CAV | QAT | | QSPEALRLQEGESSSLNCSYTVSGLR | | LQLGWLSGEDQV | |
| | PRYLITVTGKKLTV | | QAT | SGS | | GLFWYRQDPGKGPEFLFTLYSAGEEK | | TQSPEALRLQEGE | |
| | TCSQNMNHEYMS | | SG | ARQ | | EKERLKATLTKKESFLHITAPKPEDSAT | | SSSLNCSYTVSGL | |
| | WYRQDPGLGLRQI | | SAR | LTF | | YLCAVQATSGSARQLTFGSGTQLTVL | | RGLFWYRQDPGK | |
| | YYSMNVEVTDKG | | QLT | G | | PDIQNPDPAVYQLRDSKSSDKSVCLF | | GPEFLFTLYSAGE | |
| | DVPEGYKVSRKEK | | FG_ | | | TDFDSQTNVSQSKDSDVYITDKTVLD | | EKEKERLKATLTK | |
| | RNFPLILESPSPNQ | | hTRAJ22 | | | MRSMDFKSNSAVAWSNKSDFACAN | | KESFLHITAPKPED | |
| | TSLYFCASSLSKT | | | | | AFNNSIIPEDTFFPSPESSCDVKLVEK | | SATYLCAVQATSG | |
| | GRYNEQFFGPGT | | | | | SFETDTNLNFQNLSVIGFRILLLKVAG | | SARQLTFGSGTQL | |
| | RLTVL | | | | | FNLLMTLRLWSS | | TVLPD | |

| | | | | | | | | TCR | | | |
|---|---|---|---|---|---|---|---|---|---|---|---|
| | | Non | | | | | Add- | pMHC | | | |
| | | synonymus | | | | | itional | beta | SEQ | | SEQ |
| Patient | | mutations | HLA | Minimal | | TCR | Id in- | chain | ID | | ID |
| ID | Gene | SNVs | restriction | epitope | | Id | formation | CDR3 | NO | TCR pMHC beta chain | NO |
| Patient | MAGEA | | A02:05 | GLY | 232 | M1 | hTRBV05- | CAS | 10 | MGPGLLCWVLLCLLGAGPVDAGVTQSPTH | 86 |
| 2 | 10 | | | DGM | | | 5_CASS | SFF | | LIKTRGQQVTLRCSPISGHKSVSWYQQVLG | |
| | | | | EHL | | | FFP | PDS | | QGPQFIFQYYEKEERGRGNFPDRFSARQF | |
| | | | | | | | DSNQ | NQP | | PNYSSELNVNALLLGDSALYLCASSFFPDS | |
| | | | | | | | PQHF | QHF | | NQPQHFGDGTRLSILEDLNKVFPPEVAVFE | |
| | | | | | | | G_ | G | | PSEAEISHTQKATLVCLATGFFPDHVELS | |
| | | | | | | | hTRBJ01-5 | | | WWVNGKEVHSGVSTDPQPLKEQPALNDS | |
| | | | | | | | | | | RYCLSSRLRVSATFWQNPRNHFRCQVQF | |
| | | | | | | | | | | YGLSENDEWTQDRAKPVTQIVSAEAWGR | |
| | | | | | | | | | | ADCGFTSVSYQQGVLSATILYEILLGKATL | |
| | | | | | | | | | | YAVLVSALVLMAMVKRKDF | |
| | | | | | | M2 | hTRBV06- | AST | 12 | MSISLLCCAAFPLLWAGPVNAGVTQTPKFR | 88 |
| | | | | | | | 26_C | LAG | | ILKIGQSMTLQCTQDMNHNYMYWYRQDPG | |
| | | | | | | | AST | RAY | | MGLKLIYYSVGAGITDKGEVPNGYNVSRST | |
| | | | | | | | LAG | EQY | | TEDFPLRLELAAPSQTSVYFCASTLAGRAY | |
| | | | | | | | RAY | FG | | EQYFGPGTRLTVTEDLNKVFPPEVAVFEP | |
| | | | | | | | EQY | | | SEAEISHTQKATLVCLATGFFPDHVELSW | |
| | | | | | | | FG_ | | | WVNGKEVHSGVSTDPQPLKEQPALNDSR | |
| | | | | | | | hTRBJ02-7 | | | YCLSSRLRVSATFWQNPRNHFRCQVQFY | |
| | | | | | | | | | | GLSENDEWTQDRAKPVTQIVSAEAWGRA | |
| | | | | | | | | | | DCGFTSVSYQQGVLSATILYEILLGKATLY | |
| | | | | | | | | | | AVLVSALVLMAMVKRKDF | |
| | | | | | | M3 | hTRBV02_ | CA | 14 | MDTWLVCWAIFSLLKAGLTEPEVTQTPSH | 90 |
| | | | | | | | CASI | SI | | QVTQMGQEVILRCVPISNHLYFYWYRQILG | |
| | | | | | | | QG | QGT | | QKVEFLVSFYNNEISEKSEIFDDQFSVERP | |
| | | | | | | | TGL | GLA | | DGSNFTLKIRSTKLEDSAMYFCASIQGTGL | |
| | | | | | | | AYT | YTF | | AYTFGSGTRLTVVEDLNKVFPPEVAVFEPS | |
| | | | | | | | FG_ | G | | EAEISHTQKATLVCLATGFFPDHVELSWW | |
| | | | | | | | hTRBJ01-2 | | | VNGKEVHSGVSTDPQPLKEQPALNDSRY | |
| | | | | | | | | | | CLSSRLRVSATFWQNPRNHFRCQVQFYG | |
| | | | | | | | | | | LSENDEWTQDRAKPVTQIVSAEAWGRAD | |
| | | | | | | | | | | CGFTSVSYQQGVLSATILYEILLGKATLYA | |
| | | | | | | | | | | VLVSALVLMAMVKRKDF | |
| | | | | | | M4 | hTRBV02_C | CAS | 16 | MDTWLVCWAIFSLLKAGLTEPEVTQTPSH | 92 |
| | | | | | | | AS | NLG | | QVTQMGQEVILRCVPISNHLYFYWYRQILG | |
| | | | | | | | NL | QAI | | QKVEFLVSFYNNEISEKSEIFDDQFSVERP | |
| | | | | | | | GQ | KYT | | DGSNFTLKIRSTKLEDSAMYFCASNLGQAI | |
| | | | | | | | AIK | FG | | KYTFGSGTRLTVVEDLNKVFPPEVAVFEPS | |
| | | | | | | | YTF | | | EAEISHTQKATLVCLATGFFPDHVELSWW | |
| | | | | | | | G_ | | | VNGKEVHSGVSTDPQPLKEQPALNDSRY | |
| | | | | | | | hTRBJ01-2 | | | CLSSRLRVSATFWQNPRNHFRCQVQFYG | |
| | | | | | | | | | | LSENDEWTQDRAKPVTQIVSAEAWGRAD | |
| | | | | | | | | | | CGFTSVSYQQGVLSATILYEILLGKATLYA | |
| | | | | | | | | | | VLVSALVLMAMVKRKDF | |
| | | | | | | M5 | hTRBV07- | ASS | 18 | MGTRLLCWVVLGFLGTDHTGAGVSQSPRY | 94 |
| | | | | | | | 8_ | LGG | | KVAKRGQDVALRCDPISGHVSLFWYQQAL | |
| | | | | | | | CASS | SFQ | | GQGPEFLTYFQNEAQLDKSGLPSDRFFAE | |
| | | | | | | | LGGS | PQH | | RPEGSVSTLKIQRTQQEDSAVYLCASSLGG | |
| | | | | | | | FQP | FG | | SFQPQHFGDGTRLSILEDLNKVFPPEVAVF | |
| | | | | | | | QHFG_ | | | EPSEAEISHTQKATLVCLATGFFPDHVELS | |
| | | | | | | | hTRBJ01-5 | | | WWVNGKEVHSGVSTDPQPLKEQPALNDS | |
| | | | | | | | | | | RYCLSSRLRVSATFWQNPRNHFRCQVQF | |
| | | | | | | | | | | YGLSENDEWTQDRAKPVTQIVSAEAWGR | |
| | | | | | | | | | | ADCGFTSVSYQQGVLSATILYEILLGKATL | |
| | | | | | | | | | | YAVLVSALVLMAMVKRKDF | |
| | | | | | | M6 | hTRBV02_ | CAS | 20 | MDTWLVCWAIFSLLKAGLTEPEVTQTPSH | 96 |
| | | | | | | | CAS | RAN | | QVTQMGQEVILRCVPISNHLYFYWYRQILG | |
| | | | | | | | RANT | TGE | | QKVEFLVSFYNNEISEKSEIFDDQFSVERP | |
| | | | | | | | GEL | LFF | | DGSNFTLKIRSTKLEDSAMYFCASRANTGE | |
| | | | | | | | FFG_ | G | | LFFGEGSRLTVLEDLNKVFPPEVAVFEPSE | |
| | | | | | | | hTRBJ02-2 | | | AEISHTQKATLVCLATGFFPDHVELSWWV | |
| | | | | | | | | | | NGKEVHSGVSTDPQPLKEQPALNDSRYC | |
| | | | | | | | | | | LSSRLRVSATFWQNPRNHFRCQVQFYGL | |
| | | | | | | | | | | SENDEWTQDRAKPVTQIVSAEAWGRADC | |
| | | | | | | | | | | GFTSVSYQQGVLSATILYEILLGKATLYAV | |
| | | | | | | | | | | LVSALVLMAMVKRKDF | |
| | | | | | | M7 | hTRBV02_ | CA | 22 | MDTWLVCWAIFSLLKAGLTEPEVTQTPSH | 98 |
| | | | | | | | CASI | SIV | | QVTQMGQEVILRCVPISNHLYFYWYRQILG | |
| | | | | | | | VG | GQG | | QKVEFLVSFYNNEISEKSEIFDDQFSVERP | |
| | | | | | | | QG | NEQ | | DGSNFTLKIRSTKLEDSAMYFCASIVGQGN | |
| | | | | | | | NE | FFG | | EQFFGPGTRLTVLEDLNKVFPPEVAVFEPS | |
| | | | | | | | QFFG_ | | | EAEISHTQKATLVCLATGFFPDHVELSWW | |
| | | | | | | | hTRBJ02-1 | | | VNGKEVHSGVSTDPQPLKEQPALNDSRY | |
| | | | | | | | | | | CLSSRLRVSATFWQNPRNHFRCQVQFYG | |
| | | | | | | | | | | LSENDEWTQDRAKPVTQIVSAEAWGRAD | |
| | | | | | | | | | | CGFTSVSYQQGVLSATILYEILLGKATLYA | |
| | | | | | | | | | | VLVSALVLMAMVKRKDF | |
| | | | | | | M8 | hTRBV06- | CAS | 24 | MSIGLLCCVAFSLLWASPVNAGVTQTPKFQ | 100 |
| | | | | | | | 1_ | SPL | | VLKTGQSMTLQCAQDMNHNSMYWYRQDP | |
| | | | | | | | CASS | RDY | | GMGLRLIYYSASEGTTDKGEVPNGYNVSR | |
| | | | | | | | PLR | FNE | | LNKREFSLRLESAAPSQTSVYFCASSPLRD | |
| | | | | | | | DYF | QFF | | YFNEQFFGPGTRLTVLEDLNKVFPPEVAVF | |
| | | | | | | | NE | G | | EPSEAEISHTQKATLVCLATGFFPDHVELS | |
| | | | | | | | QFFG_ | | | WWVNGKEVHSGVSTDPQPLKEQPALNDS | |
| | | | | | | | hTRBJ02-1 | | | RYCLSSRLRVSATFWQNPRNHFRCQVQF | |
| | | | | | | | | | | YGLSENDEWTQDRAKPVTQIVSAEAWGR | |
| | | | | | | | | | | ADCGFTSVSYQQGVLSATILYEILLGKATL | |
| | | | | | | | | | | YAVLVSALVLMAMVKRKDF | |
| | | | | | | M9 | hTRBV09_ | CAS | 26 | MGFRLLCCVAFCLLGAGPVDSGVTQTPKH | 102 |
| | | | | | | | CASS | SLT | | LITATGQRVTLRCSPRSGDLSVYWYQQSL | |
| | | | | | | | LTG | GYE | | DQGLQFLIQYYNGEERAKGNILERFSAQQF | |
| | | | | | | | YE | QFF | | PDLHSELNLSSLELGDSALYFCASSLTGYE | |
| | | | | | | | QFFG_ | G | | QFFGPGTRLTVLEDLNKVFPPEVAVFEPSE | |
| | | | | | | | hTRBJ02-1 | | | AEISHTQKATLVCLATGFFPDHVELSWWV | |
| | | | | | | | | | | NGKEVHSGVSTDPQPLKEQPALNDSRYC | |
| | | | | | | | | | | LSSRLRVSATFWQNPRNHFRCQVQFYGL | |
| | | | | | | | | | | SENDEWTQDRAKPVTQIVSAEAWGRADC | |
| | | | | | | | | | | GFTSVSYQQGVLSATILYEILLGKATLYAV | |
| | | | | | | | | | | LVSALVLMAMVKRKDF | |
| | Melan- | A27L | A02:05 | ELA | 233 | E1 | hTRBV20_ | CSA | 28 | MLLLLLLLGPGSGLGAVVSQHPSRVICKSG | 104 |
| | A | | | GIG | | | CSAT | TEG | | TSVKIECRSLDFQATTMFWYRQFPKQSLM | |
| | | | | IL | | | EG | TPQ | | LMATSNEGSKATYEQGVEKDKFLINHASLT | |
| | | | | TV | | | TP | FFG | | LSTLTVTSAHPEDSSFYICSATEGTPQFFG | |
| | | | | | | | QFFG_ | | | PGTRLTVLEDLNKVFPPEVAVFEPSEAEIS | |
| | | | | | | | h | | | HTQKATLVCLATGFFPDHVELSWWVNGK | |
| | | | | | | | TRBJ02-1 | | | EVHSGVSTDPQPLKEQPALNDSRYCLSSR | |
| | | | | | | | | | | LRVSATFWQNPRNHFRCQVQFYGLSEND | |
| | | | | | | | | | | EWTQDRAKPVTQIVSAEAWGRADCGFTS | |
| | | | | | | | | | | VSYQQGVLSATILYEILLGKATLYAVLVSA | |
| | | | | | | | | | | LVLMAMVKRKDF | |
| | | | | | | E2 | hTRBV04- | CAS | 30 | MGCRLLCCAVLCLLGAGELVPMETGVTQT | 106 |
| | | | | | | | 3_ | SPD | | PRHLVMGMTNKKSLKCEQHLGHNAMYWY | |
| | | | | | | | CASS | LAG | | KQSAKKPLELMFVYSLEERVENNSVPSRFS | |
| | | | | | | | PDL | VNE | | PECPNSSHLFLHLHTLQPEDSALYLCASSP | |
| | | | | | | | AGVN | QFF | | DLAGVNEQFFGPGTRLTVLEDLNKVFPPE | |
| | | | | | | | EQ | G | | VAVFEPSEAEISHTQKATLVCLATGFFPDH | |
| | | | | | | | FFG_ | | | VELSWWVNGKEVHSGVSTDPQPLKEQPA | |
| | | | | | | | hTRBJ | | | LNDSRYCLSSRLRVSATFWQNPRNHFRC | |
| | | | | | | | 02-1 | | | QVQFYGLSENDEWTQDRAKPVTQIVSAEA | |
| | | | | | | | | | | WGRADCGFTSVSYQQGVLSATILYEILLG | |
| | | | | | | | | | | KATLYAVLVSALVLMAMVKRKDF | |
| | | | | | | E3 | hTRBV04- | CAS | 32 | MGCRLLCCAVLCLLGAVPMETGVTQTPRH | 108 |
| | | | | | | | 2_ | SQD | | LVMGMTNKKSLKCEQHLGHNAMYWYKQS | |
| | | | | | | | CASS | LAI | | AKKPLELMFVYNFKEQTENNSVPSRFSPE | |
| | | | | | | | QD | GEQ | | CPNSSHLFLHLHTLQPEDSALYLCASSQDL | |
| | | | | | | | LAI | YFG | | AIGEQYFGPGTRLTVTEDLNKVFPPEVAVF | |
| | | | | | | | GEQY | | | EPSEAEISHTQKATLVCLATGFFPDHVELS | |
| | | | | | | | FG | | | WWVNGKEVHSGVSTDPQPLKEQPALNDS | |
| | | | | | | | hTR | | | RYCLSSRLRVSATFWQNPRNHFRCQVQF | |
| | | | | | | | BJ02-7 | | | YGLSENDEWTQDRAKPVTQIVSAEAWGR | |
| | | | | | | | | | | ADCGFTSVSYQQGVLSATILYEILLGKATL | |
| | | | | | | | | | | YAVLVSALVLMAMVKRKDF | |
| | | | | | | E4 | hTRBV28_ | CAS | 34 | MGIRLLCRVAFCFLAVGLVDVKVTQSSRYL | 110 |
| | | | | | | | CASS | SRT | | VKRTGEKVFLECVQDMDHENMFWYRQDP | |
| | | | | | | | RTF | FRE | | GLGLRLIYFSYDVKMKEKGDIPEGYSVSRE | |
| | | | | | | | REL | LFG | | KKERFSLILESASTNQTSMYLCASSRTFRE | |
| | | | | | | | FFG_ | | | LFFGEGSRLTVLEDLNKVFPPEVAVFEPSE | |
| | | | | | | | hTRBJ02-2 | | | AEISHTQKATLVCLATGFFPDHVELSWWV | |
| | | | | | | | | | | NGKEVHSGVSTDPQPLKEQPALNDSRYC | |
| | | | | | | | | | | LSSRLRVSATFWQNPRNHFRCQVQFYGL | |
| | | | | | | | | | | SENDEWTQDRAKPVTQIVSAEAWGRADC | |
| | | | | | | | | | | GFTSVSYQQGVLSATILYEILLGKATLYAV | |
| | | | | | | | | | | LVSALVLMAMVKRKDF | |
| | | | | | | E5 | hTRBV06- | CAS | 36 | MSIGLLCCVAFSLLWASPVNAGVTQTPKFQ | 112 |
| | | | | | | | 5_ | SYS | | VLKTGQSMTLQCAQDMNHNSMYWYRQDP | |
| | | | | | | | CASS | GTS | | GMGLRLIYYSASEGTTDKGEVPNGYNVSR | |
| | | | | | | | YSG | GIY | | LNKREFSLRLESAAPSQTSVYFCASSYSGT | |
| | | | | | | | TSG | EQY | | SGIYEQYFGPGTRLTVTEDLNKVFPPEVAV | |
| | | | | | | | IYE | FG | | FEPSEAEISHTQKATLVCLATGFFPDHVEL | |
| | | | | | | | QYFG_ | | | SWWVNGKEVHSGVSTDPQPLKEQPALND | |
| | | | | | | | hTRBJ02-7 | | | SRYCLSSRLRVSATFWQNPRNHFRCQVQ | |
| | | | | | | | | | | FYGLSENDEWTQDRAKPVTQIVSAEAWG | |
| | | | | | | | | | | RADCGFTSVSYQQGVLSATILYEILLGKAT | |
| | | | | | | | | | | LYAVLVSALVLMAMVKRKDF | |
| | | | | | | E6 | hTRBV06- | CAS | 38 | MSIGLLCCVAFSLLWASPVNAGVTQTPKFQ | 114 |
| | | | | | | | 5_ | SYS | | VLKTGQSMTLQCAQDMNHNSMYWYRQDP | |
| | | | | | | | CASS | LSG | | GMGLRLIYYSASEGTTDKGEVPNGYNVSR | |
| | | | | | | | YSLSG | TSS | | LNKREFSLRLESAAPSQTSVYFCASSYSLS | |
| | | | | | | | TSS | YEQ | | GTSSYEQYFGPGTRLTVTEDLNKVFPPEV | |
| | | | | | | | YEQY | YG | | AVFEPSEAEISHTQKATLVCLATGFFPDHV | |
| | | | | | | | FG_ | | | ELSWWVNGKEVHSGVSTDPQPLKEQPAL | |
| | | | | | | | hTRBJ02-7 | | | NDSRYCLSSRLRVSATFWQNPRNHFRCQ | |
| | | | | | | | | | | VQFYGLSENDEWTQDRAKPVTQIVSAEA | |
| | | | | | | | | | | WGRADCGFTSVSYQQGVLSATILYEILLG | |
| | | | | | | | | | | KATLYAVLVSALVLMAMVKRKDF | |
| | | | | | | E7 | hTRBV04- | CAS | 40 | MGCRLLCCAVLCLLGAVPIDTEVTQTPKHL | 116 |
| | | | | | | | 1_ | SPD | | VMGMTNKKSLKCEQHMGHRAMYWYKQK | |
| | | | | | | | CASS | RSA | | AKKPPELMFVYSYEKLSINESVPSRFSPEC | |
| | | | | | | | PDRS | DTQ | | PNSSLLNLHLHALQPEDSALYLCASSPDRS | |
| | | | | | | | ADT | YFG | | ADTQYFGPGTRLTVLEDLNKVFPPEVAVFE | |
| | | | | | | | QYFG_ | | | PSEAEISHTQKATLVCLATGFFPDHVELS | |
| | | | | | | | hTRBJ02-3 | | | WWVNGKEVHSGVSTDPQPLKEQPALNDS | |
| | | | | | | | | | | RYCLSSRLRVSATFWQNPRNHFRCQVQF | |
| | | | | | | | | | | YGLSENDEWTQDRAKPVTQIVSAEAWGR | |
| | | | | | | | | | | ADCGFTSVSYQQGVLSATILYEILLGKATL | |
| | | | | | | | | | | YAVLVSALVLMAMVKRKDF | |
| | | | | | | E9 | hTRBV29_ | CSA | 42 | MLSLLLLLLGLGSVFSAVISQKPSRDICQRG | 118 |
| | | | | | | | CSAS | SR | | TSLTIQCQVDSQVTMMFWYRQQPGQSLTL | |
| | | | | | | | RDI | DID | | IATANQGSEATYESGFVIDKFPISRPNLTFS | |
| | | | | | | | DSGN | SGN | | TLTVSNMSPEDSSIYLCSASRDIDSGNTIYF | |
| | | | | | | | TIY | YFG | | IEGSWLTVVEDLNKVFPPEVAVFEPSEAEI | |
| | | | | | | | FG | | | SHTQKATLVCLATGFFPDHVELSWWVNG | |
| | | | | | | | hTRBJ01-3 | | | KEVHSGVSTDPQPLKEQPALNDSRYCLSS | |
| | | | | | | | | | | RLRVSATFWQNPRNHFRCQVQFYGLSEN | |
| | | | | | | | | | | DEWTQDRAKPVTQIVSAEAWGRADCGFT | |
| | | | | | | | | | | SVSYQQGVLSATILYEILLGKATLYAVLVS | |
| | | | | | | | | | | ALVLMAMVKRKDF | |
| | KIF1B | S918F | A02:05 | TAD | 234 | K1 | hTRBV05- | CAS | 44 | MGSRLLCWVLLCLLGAGPVKAGVTQTPRY | 120 |
| | | | | FDI | | | 1_ | KFG | | LIKTRGQQVTLSCSPISGHRSVSWYQQTP | |
| | | | | TEL | | | CASK | DTQ | | GQGLQFLFEYFSETQRNKGNFPGRFSGRQ | |
| | | | | | | | FG | YFG | | FSNSRSEMNVSTLELGDSALYLCASKFGDT | |
| | | | | | | | DT | | | QYFGPGTRLTVLEDLNKVFPPEVAVFEPSE | |
| | | | | | | | QY | | | AEISHTQKATLVCLATGFFPDHVELSWWV | |
| | | | | | | | FG_ | | | NGKEVHSGVSTDPQPLKEQPALNDSRYC | |
| | | | | | | | hTRBJ02-3 | | | LSSRLRVSATFWQNPRNHFRCQVQFYGL | |
| | | | | | | | | | | SENDEWTQDRAKPVTQIVSAEAWGRADC | |
| | | | | | | | | | | GFTSVSYQQGVLSATILYEILLGKATLYAV | |
| | | | | | | | | | | LVSALVLMAMVKRKDF | |
| | | | | | | K2 | hTRBV05- | CAS | 46 | MGSRLLCWVLLCLLGAGPVKAGVTQTPRY | 122 |
| | | | | | | | 1_ | KFG | | LIKTRGQQVTLSCSPISGHRSVSWYQQTP | |
| | | | | | | | CASK | NEL | | GQGLQFLFEYFSETQRNKGNFPGRFSGRQ | |
| | | | | | | | FG | FFG | | FSNSRSEMNVSTLELGDSALYLCASKFGN | |
| | | | | | | | NEL | | | ELFFGPGTRLTVLEDLNKVFPPEVAVFEPS | |
| | | | | | | | FFG_ | | | EAEISHTQKATLVCLATGFFPDHVELSWW | |
| | | | | | | | hTRBJ02-1 | | | VNGKEVHSGVSTDPQPLKEQPALNDSRY | |
| | | | | | | | | | | CLSSRLRVSATFWQNPRNHFRCQVQFYG | |
| | | | | | | | | | | LSENDEWTQDRAKPVTQIVSAEAWGRAD | |
| | | | | | | | | | | CGFTSVSYQQGVLSATILYEILLGKATLYA | |
| | | | | | | | | | | VLVSALVLMAMVKRKDF | |
| | | | | | | K3 | hTRBV09_ | CAS | 48 | MGFRLLCCVAFCLLGAGPVDSGVTQTPKH | 124 |
| | | | | | | | CASS | SVV | | LITATGQRVTLRCSPRSGDLSVYWYQQSL | |
| | | | | | | | VV | GTR | | DQGLQFLIQYYNGEERAKGNILERFSAQQF | |
| | | | | | | | GT | EQF | | PDLHSELNLSSLELGDSALYFCASSVVGTR | |
| | | | | | | | RE | FG | | EQFFGPGTRLTVLEDLNKVFPPEVAVFEPS | |
| | | | | | | | QFFG_ | | | EAEISHTQKATLVCLATGFFPDHVELSWW | |
| | | | | | | | hTRBJ02-1 | | | VNGKEVHSGVSTDPQPLKEQPALNDSRY | |
| | | | | | | | | | | CLSSRLRVSATFWQNPRNHFRCQVQFYG | |
| | | | | | | | | | | LSENDEWTQDRAKPVTQIVSAEAWGRAD | |
| | | | | | | | | | | CGFTSVSYQQGVLSATILYEILLGKATLYA | |
| | | | | | | | | | | VLVSALVLMAMVKRKDF | |
| | | | | | | K4 | hTRBV05- | CAS | 50 | MGSRLLCWVLLCLLGAGPVKAGVTQTPRY | 126 |
| | | | | | | | 1_ | SYG | | LIKTRGQQVTLSCSPISGHRSVSWYQQTP | |
| | | | | | | | CASS | NEQ | | GQGLQFLFEYFSETQRNKGNFPGRFSGRQ | |
| | | | | | | | YG | F | | FSNSRSEMNVSTLELGDSALYLCASSYGN | |
| | | | | | | | NE | FG | | EQFFGPGTRLTVLEDLNKVFPPEVAVFEPS | |
| | | | | | | | QFFG_ | | | EAEISHTQKATLVCLATGFFPDHVELSWW | |
| | | | | | | | hTRBJ02-1 | | | VNGKEVHSGVSTDPQPLKEQPALNDSRY | |
| | | | | | | | | | | CLSSRLRVSATFWQNPRNHFRCQVQFYG | |
| | | | | | | | | | | LSENDEWTQDRAKPVTQIVSAEAWGRAD | |
| | | | | | | | | | | CGFTSVSYQQGVLSATILYEILLGKATLYA | |
| | | | | | | | | | | VLVSALVLMAMVKRKDF | |

| | TCR pMHC beta | | | TCR | | | | TCR pMHC alpha | |
|---|---|---|---|---|---|---|---|---|---|
| | chain | | | pMHC | | | | chain | |
| | (excluding | SEQ | Additional | alpha | SEQ | | SEQ | (excluding | SEQ |
| Patient | the constant | ID | Id | chain | ID | | ID | the constant | ID |
| Id | region) | NO | information | CDR3 | NO | TCR pMHC alpha chain | NO | region) | NO |
| Patient | MGPGLLCWVLLCL | 162 | hTRAV17_ | CAT | 9 | METLLGVSLVILWLQLARVNSQQGEE | 85 | METLLGVSLVILWL | 161 |
| 2 | LGAGPVDAGVTQ | | CATV | VFY | | DPQALSIQEGENATMNCSYKTSINNLQ | | QLARVNSQQGEE | |
| | SPTHLIKTRGQQV | | FYG | GNN | | WYRQNSGRGLVHLILIRSNEREKHSG | | DPQALSIQEGENA | |
| | TLRCSPISGHKSV | | NN | RLA | | RLRVTLDTSKKSSSLLITASRAADTAS | | TMNCSYKTSINNL | |
| | SWYQQVLGQGPQ | | RLA | FG | | YFCATVFYGNNRLAFGKGNQVVVIPNI | | QWYRQNSGRGLV | |
| | FIFQYYEKEERGR | | FG_ | | | QNPDPAVYQLRDSKSSDKSVCLFTDF | | HLILIRSNEREKHS | |
| | GNFPDRFSARQFP | | hTRAJ07 | | | DSQTNVSQSKDSDVYITDKTVLDMRS | | GRLRVTLDTSKKS | |
| | NYSSELNVNALLL | | | | | MDFKSNSAVAWSNKSDFACANAFNN | | SSLLITASRAADTA | |
| | GDSALYLCASSFF | | | | | SIIPEDTFFPSPESSCDVKLVEKSFET | | SYFCATVFYGNNR | |
| | PDSNQPQHFGDG | | | | | DTNLNFQNLSVIGFRILLLKVAGFNLL | | LAFGKGNQVVVIP | |
| | TRLSIL | | | | | MTLRLWSS | | N | |
| | MSISLLCCAAFPLL | 164 | hTRAV12- | CVH | 11 | MISLRVLLVILWLQLSWVWSQRKEVE | 87 | MISLRVLLVILWLQ | 163 |
| | WAGPVNAGVTQT | | 1_ | MEY | | QDPGPFNVPEGATVAFNCTYSNSASQ | | LSWVWSQRKEVE | |
| | PKFRILKIGQSMTL | | CVH | GN | | SFFWYRQDCRKEPKLLMSVYSSGNE | | QDPGPFNVPEGAT | |
| | QCTQDMNHNYMY | | MEYG | KL | | DGRFTAQLNRASQYISLLIRDSKLSDS | | VAFNCTYSNSASQ | |
| | WYRQDPGMGLKLI | | NKL | VFG | | ATYLCVHMEYGNKLVFGAGTILRVKSY | | SFFWYRQDCRKE | |
| | YYSVGAGITDKGE | | VFG_ | | | IQNPDPAVYQLRDSKSSDKSVCLFTD | | PKLLMSVYSSGNE | |
| | VPNGYNVSRSTTE | | hTRAJ47 | | | FDSQTNVSQSKDSDVYITDKTVLDMR | | DGRFTAQLNRASQ | |
| | DFPLRLELAAPSQ | | | | | SMDFKSNSAVAWSNKSDFACANAFN | | YISLLIRDSKLSDS | |
| | TSVYFCASTLAGR | | | | | NSIIPEDTFFPSPESSCDVKLVEKSFE | | ATYLCVHMEYGNK | |
| | AYEQYFGPGTRLT | | | | | TDTNLNFQNLSVIGFRILLLKVAGFNL | | LVFGAGTILRVKSY | |
| | VT | | | | | LMTLRLWSS | | | |
| | MDTWLVCWAIFSL | 166 | hTRAV22_ | CAV | 13 | MKRILGALLGLLSAQVCCVRGIQVEQS | 89 | MKRILGALLGLLSA | 165 |
| | LKAGLTEPEVTQT | | CAV | GVL | | PPDLILQEGANSTLRCNFSDSVNNLQ | | QVCCVRGIQVEQS | |
| | PSHQVTQMGQEVI | | GVLRD | RDY | | WFHQNPWGQLINLFYIPSGTKQNGRL | | PPDLILQEGANSTL | |
| | LRCVPISNHLYFY | | YKL | KLS | | SATTVATERYSLLYISSSQTTDSGVYF | | RCNFSDSVNNLQ | |
| | WYRQILGQKVEFL | | SFG_ | FG | | CAVGVLRDYKLSFGAGTTVTVRANIQ | | WFHQNPWGQLIN | |
| | VSFYNNEISEKSEI | | hTRAJ20 | | | NPDPAVYQLRDSKSSDKSVCLFTDFD | | LFYIPSGTKQNGR | |
| | FDDQFSVERPDG | | | | | SQTNVSQSKDSDVYITDKTVLDMRSM | | LSATTVATERYSLL | |
| | SNFTLKIRSTKLED | | | | | DFKSNSAVAWSNKSDFACANAFNNSI | | YISSSQTTDSGVY | |
| | SAMYFCASIQGTG | | | | | IPEDTFFPSPESSCDVKLVEKSFETDT | | FCAVGVLRDYKLS | |
| | LAYTFGSGTRLTV | | | | | NLNFQNLSVIGFRILLLKVAGFNLLMT | | FGAGTTVTVRAN | |
| | V | | | | | LRLWSS | | | |
| | MDTWLVCWAIFSL | 168 | hTRAV21_ | CAV | 15 | METLLGLLILWLQLQWVSSKQEVTQIP | 91 | METLLGLLILWLQL | 167 |
| | LKAGLTEPEVTQT | | CAVA | AVF | | AALSVPEGENLVLNCSFTDSAIYNLQW | | QWVSSKQEVTQIP | |
| | PSHQVTQMGQEVI | | VFP | PGN | | FRQDPGKGLTSLLLIQSSQREQTSGR | | AALSVPEGENLVL | |
| | LRCVPISNHLYFY | | GN | QFY | | LNASLDKSSGRSTLYIAASQPGDSATY | | NCSFTDSAIYNLQ | |
| | WYRQILGQKVEFL | | QFY | FG | | LCAVAVFPGNQFYFGTGTSLTVIPNIQ | | WFRQDPGKGLTS | |
| | VSFYNNEISEKSEI | | FG_ | | | NPDPAVYQLRDSKSSDKSVCLFTDFD | | LLLIQSSQREQTS | |
| | FDDQFSVERPDG | | hTRAJ49 | | | SQTNVSQSKDSDVYITDKTVLDMRSM | | GRLNASLDKSSGR | |
| | SNFTLKIRSTKLED | | | | | DFKSNSAVAWSNKSDFACANAFNNSI | | STLYIAASQPGDS | |
| | SAMYFCASNLGQ | | | | | IPEDTFFPSPESSCDVKLVEKSFETDT | | ATYLCAVAVFPGN | |
| | AIKYTFGSGTRLTV | | | | | NLNFQNLSVIGFRILLLKVAGFNLLMT | | QFYFGTGTSLTVIP | |
| | V | | | | | LRLWSS | | N | |
| | MGTRLLCWVVLG | 170 | hTRAV38- | CAY | 17 | MACPGFLWALVISTCLEFSMAQTVTQ | 93 | MACPGFLWALVIS | 169 |
| | FLGTDHTGAGVSQ | | 2_ | RSA | | SQPEMSVQEAETVTLSCTYDTSESDY | | TCLEFSMAQTVTQ | |
| | SPRYKVAKRGQD | | CAYR | MYS | | YLFWYKQPPSRQMILVIRQEAYKQQN | | SQPEMSVQEAETV | |
| | VALRCDPISGHVS | | SA | GGG | | ATENRFSVNFQKAAKSFSLKISDSQLG | | TLSCTYDTSESDY | |
| | LFWYQQALGQGP | | MY | ADG | | DAAMYFCAYRSAMYSGGGADGLTFG | | YLFWYKQPPSRQ | |
| | EFLTYFQNEAQLD | | SGG | LT | | KGTHLIIQPYIQNPDPAVYQLRDSKSS | | MILVIRQEAYKQQ | |
| | KSGLPSDRFFAER | | GAD | FG | | DKSVCLFTDFDSQTNVSQSKDSDVYI | | NATENRFSVNFQK | |
| | PEGSVSTLKIQRT | | GLT | | | TDKTVLDMRSMDFKSNSAVAWSNKS | | AAKSFSLKISDSQL | |
| | QQEDSAVYLCASS | | FG_ | | | DFACANAFNNSIIPEDTFFPSPESSCD | | GDAAMYFCAYRS | |
| | LGGSFQPQHFGD | | hTRAJ45 | | | VKLVEKSFETDTNLNFQNLSVIGFRIL | | AMYSGGGADGLT | |
| | GTRLSIL | | | | | LLKVAGFNLLMTLRLWSS | | FGKGTHLIIQPY | |
| | MDTWLVCWAIFSL | 172 | hTRAV17_ | CAT | 19 | METLLGVSLVILWLQLARVNSQQGEE | 95 | METLLGVSLVILWL | 171 |
| | LKAGLTEPEVTQT | | CATD | DAY | | DPQALSIQEGENATMNCSYKTSINNLQ | | QLARVNSQQGEE | |
| | PSHQVTQMGQEVI | | AYN | NF | | WYRQNSGRGLVHLILIRSNEREKHSG | | DPQALSIQEGENA | |
| | LRCVPISNHLYFY | | FNK | NKF | | RLRVTLDTSKKSSSLLITASRAADTAS | | TMNCSYKTSINNL | |
| | WYRQILGQKVEFL | | FYF | YFG | | YFCATDAYNFNKFYFGSGTKLNVKPNI | | QWYRQNSGRGLV | |
| | VSFYNNEISEKSEI | | G_ | | | QNPDPAVYQLRDSKSSDKSVCLFTDF | | HLILIRSNEREKHS | |
| | FDDQFSVERPDG | | hTRAJ21 | | | DSQTNVSQSKDSDVYITDKTVLDMRS | | GRLRVTLDTSKKS | |
| | SNFTLKIRSTKLED | | | | | MDFKSNSAVAWSNKSDFACANAFNN | | SSLLITASRAADTA | |
| | SAMYFCASRANT | | | | | SIIPEDTFFPSPESSCDVKLVEKSFET | | SYFCATDAYNFNK | |
| | GELFFGEGSRLTV | | | | | DTNLNFQNLSVIGFRILLLKVAGFNLL | | FYFGSGTKLNVKP | |
| | L | | | | | MTLRLWSS | | N | |
| | MDTWLVCWAIFSL | 174 | hTRAV19_ | C | 21 | MLTASLLRAVIASICVVSSMAQKVTQA | 97 | MLTASLLRAVIASI | 173 |
| | LKAGLTEPEVTQT | | CALS | ALS | | QTEISVVEKEDVTLDCVYETRDTTYYL | | CVVSSMAQKVTQ | |
| | PSHQVTQMGQEVI | | ERP | ERP | | FWYKQPPSGELVFLIRRNSFDEQNEIS | | AQTEISVVEKEDVT | |
| | LRCVPISNHLYFY | | GG | GGA | | GRYSWNFQKSTSSFNFTITASQVVDS | | LDCVYETRDTTYY | |
| | WYRQILGQKVEFL | | ATN | TN | | AVYFCALSERPGGATNKLIFGTGTLLA | | LFWYKQPPSGELV | |
| | VSFYNNEISEKSEI | | KLI | KL | | VQPNIQNPDPAVYQLRDSKSSDKSVC | | FLIRRNSFDEQNEI | |
| | FDDQFSVERPDG | | FG_ | IFG | | LFTDFDSQTNVSQSKDSDVYITDKTVL | | SGRYSWNFQKST | |
| | SNFTLKIRSTKLED | | hTRAJ32 | | | DMRSMDFKSNSAVAWSNKSDFACA | | SSFNFTITASQVVD | |
| | SAMYFCASIVGQG | | | | | NAFNNSIIPEDTFFPSPESSCDVKLVE | | SAVYFCALSERPG | |
| | NEQFFGPGTRLTV | | | | | KSFETDTNLNFQNLSVIGFRILLLKVA | | GATNKLIFGTGTLL | |
| | L | | | | | GFNLLMTLRLWSS | | AVQPN | |
| | MSIGLLCCVAFSLL | 176 | hTRAV36_ | CAV | 23 | MMKCPQALLAIFWLLLSWVSSEDKVV | 99 | MMKCPQALLAIFW | 175 |
| | WASPVNAGVTQT | | CAVC | CDS | | QSPLSLVVHEGDTVTLNCSYEVTNFR | | LLLSWVSSEDKVV | |
| | PKFQVLKTGQSMT | | DS | WG | | SLLWYKQEKKAPTFLFMLTSSGIEKKS | | QSPLSLVVHEGDT | |
| | LQCAQDMNHNSM | | WG | KLQ | | GRLSSILDKKELSSILNITATQTGDSAIY | | VTLNCSYEVTNFR | |
| | YWYRQDPGMGLR | | KLQ | FG- | | LCAVCDSWGKLQFGAGTQVVVTPDIQ | | SLLWYKQEKKAPT | |
| | LIYYSASEGTTDK | | FG_ | | | NPDPAVYQLRDSKSSDKSVCLFTDFD | | FLFMLTSSGIEKKS | |
| | GEVPNGYNVSRL | | hTRAJ24 | | | SQTNVSQSKDSDVYITDKTVLDMRSM | | GRLSSILDKKELSS | |
| | NKREFSLRLESAA | | | | | DFKSNSAVAWSNKSDFACANAFNNSI | | ILNITATQTGDSAIY | |
| | PSQTSVYFCASSP | | | | | IPEDTFFPSPESSCDVKLVEKSFETDT | | LCAVCDSWGKLQ | |
| | LRDYFNEQFFGPG | | | | | NLNFQNLSVIGFRILLLKVAGFNLLMT | | FGAGTQVVVTPD | |
| | TRLTVL | | | | | LRLWSS | | | |
| | MGFRLLCCVAFCL | 178 | hTRAV12- | CAV | 25 | MMKSLRVLLVILWLQLSWVWSQQKEV | 101 | MMKSLRVLLVILW | 177 |
| | LGAGPVDSGVTQT | | 2_ | KGS | | EQNSGPLSVPEGAIASLNCTYSDRGS | | LQLSWVWSQQKE | |
| | PKHLITATGQRVTL | | CAVK | GTS | | QSFFWYRQYSGKSPELIMFIYSNGDK | | VEQNSGPLSVPEG | |
| | RCSPRSGDLSVY | | GS | YG | | EDGRFTAQLNKASQYVSLLIRDSQPS | | AIASLNCTYSDRG | |
| | WYQQSLDQGLQF | | GTS | KL | | DSATYLCAVKGSGTSYGKLTFGQGTIL | | SQSFFWYRQYSG | |
| | LIQYYNGEERAKG | | YG | TF | | TVHPNIQNPDPAVYQLRDSKSSDKSV | | KSPELIMFIYSNGD | |
| | NILERFSAQQFPD | | KLT | G | | CLFTDFDSQTNVSQSKDSDVYITDKT | | KEDGRFTAQLNKA | |
| | LHSELNLSSLELG | | FG_ | | | VLDMRSMDFKSNSAVAWSNKSDFAC | | SQYVSLLIRDSQP | |
| | DSALYFCASSLTG | | hTRAJ52 | | | ANAFNNSIIPEDTFFPSPESSCDVKLV | | SDSATYLCAVKGS | |
| | YEQFFGPGTRLTV | | | | | EKSFETDTNLNFQNLSVIGFRILLLKV | | GTSYGKLTFGQGT | |
| | L | | | | | AGFNLLMTLRLWSS | | ILTVHPN | |
| | MLLLLLLLGPGSGL | 180 | hTRAV12- | CAV | 27 | MMKSLRVLLVILWLQLSWVWSQQKEV | 103 | MMKSLRVLLVILW | 179 |
| | GAVVSQHPSRVIC | | 2_ | NSG | | EQNSGPLSVPEGAIASLNCTYSDRGS | | LQLSWVWSQQKE | |
| | KSGTSVKIECRSL | | CAVN | GGA | | QSFFWYRQYSGKSPELIMFIYSNGDK | | VEQNSGPLSVPEG | |
| | DFQATTMFWYRQ | | SGG | DG | | EDGRFTAQLNKASQYVSLLIRDSQPS | | AIASLNCTYSDRG | |
| | FPKQSLMLMATSN | | GAD | LTF | | DSATYLCAVNSGGGADGLTFGKGTHL | | SQSFFWYRQYSG | |
| | EGSKATYEQGVEK | | GLT | G | | IIQPYIQNPDPAVYQLRDSKSSDKSVC | | KSPELIMFIYSNGD | |
| | DKFLINHASLTLST | | FG_ | | | LFTDFDSQTNVSQSKDSDVYITDKTVL | | KEDGRFTAQLNKA | |
| | LTVTSAHPEDSSF | | hTRAJ45 | | | DMRSMDFKSNSAVAWSNKSDFACA | | SQYVSLLIRDSQP | |
| | YICSATEGTPQFF | | | | | NAFNNSIIPEDTFFPSPESSCDVKLVE | | SDSATYLCAVNSG | |
| | GPGTRLTVL | | | | | KSFETDTNLNFQNLSVIGFRILLLKVA | | GGADGLTFGKGT | |
| | | | | | | GFNLLMTLRLWSS | | HLIIQPY | |
| | MGCRLLCCAVLCL | 182 | hTRAV27_ | CAG | 29 | MVLKFSVSILWIQLAWVSTQLLEQSPQ | 105 | MVLKFSVSILWIQL | 181 |
| | LGAGELVPMETGV | | CAG | EAF | | FLSIQEGENLTVYCNSSSVFSSLQWY | | AWVSTQLLEQSPQ | |
| | TQTPRHLVMGMT | | EAF | GGN | | RQEPGEGPVLLVTVVTGGEVKKLKRL | | FLSIQEGENLTVYC | |
| | NKKSLKCEQHLGH | | GG | YQ | | TFQFGDARKDSSLHITAAQPGDTGLYL | | NSSSVFSSLQWYR | |
| | NAMYWYKQSAKK | | NY | LIW | | CAGEAFGGNYQLIWGAGTKLIIKPDIQ | | QEPGEGPVLLVTV | |
| | PLELMFVYSLEER | | QLI | GAG | | NPDPAVYQLRDSKSSDKSVCLFTDFD | | VTGGEVKKLKRLT | |
| | VENNSVPSRFSPE | | WGAG_ | | | SQTNVSQSKDSDVYITDKTVLDMRSM | | FQFGDARKDSSLH | |
| | CPNSSHLFLHLHT | | hTRAJ33 | | | DFKSNSAVAWSNKSDFACANAFNNSI | | ITAAQPGDTGLYL | |
| | LQPEDSALYLCAS | | | | | IPEDTFFPSPESSCDVKLVEKSFETDT | | CAGEAFGGNYQLI | |
| | SPDLAGVNEQFFG | | | | | NLNFQNLSVIGFRILLLKVAGFNLLMT | | WGAGTKLIIKPD | |
| | PGTRLTVL | | | | | LRLWSS | | | |
| | MGCRLLCCAVLCL | 184 | hTRAV35_ | CAG | 31 | MLLEHLLIILWMQLTWVSGQQLNQSP | 107 | MLLEHLLIILWMQL | 183 |
| | LGAVPMETGVTQT | | CAG | PNA | | QSMFIQEGEDVSMNCTSSSIFNTWLW | | TWVSGQQLNQSP | |
| | PRHLVMGMTNKK | | PNA | GGT | | YKQEPGEGPVLLIALYKAGELTSNGRL | | QSMFIQEGEDVSM | |
| | SLKCEQHLGHNA | | GG | SYG | | TAQFGITRKDSFLNISASIPSDVGIYFC | | NCTSSSIFNTWLW | |
| | MYWYKQSAKKPL | | TSY | KL | | AGPNAGGTSYGKLTFGQGTILTVHPNI | | YKQEPGEGPVLLI | |
| | ELMFVYNFKEQTE | | GKL | TF | | QNPDPAVYQLRDSKSSDKSVCLFTDF | | ALYKAGELTSNGR | |
| | NNSVPSRFSPECP | | TFG_ | G | | DSQTNVSQSKDSDVYITDKTVLDMRS | | LTAQFGITRKDSFL | |
| | NSSHLFLHLHTLQ | | hTRAJ52 | | | MDFKSNSAVAWSNKSDFACANAFNN | | NISASIPSDVGIYF | |
| | PEDSALYLCASSQ | | | | | SIIPEDTFFPSPESSCDVKLVEKSFET | | CAGPNAGGTSYG | |
| | DLAIGEQYFGPGT | | | | | DTNLNFQNLSVIGFRILLLKVAGFNLL | | KLTFGQGTILTVHP | |
| | RLTVT | | | | | MTLRLWSS | | N | |
| | MGIRLLCRVAFCF | 186 | hTRAV26- | CIV | 33 | MRLVARVTVFLTFGTIIDAKTTQPPSM | 109 | MRLVARVTVFLTF | 185 |
| | LAVGLVDVKVTQS | | 1_ | RVP | | DCAEGRAANLPCNHSTISGNEYVYWY | | GTIIDAKTTQPPSM | |
| | SRYLVKRTGEKVF | | CIVR | SDN | | RQIHSQGPQYIIHGLKNNETNEMASLII | | DCAEGRAANLPCN | |
| | LECVQDMDHENM | | VPS | FNK | | TEDRKSSTLILPHATLRDTAVYYCIVRV | | HSTISGNEYVYWY | |
| | FWYRQDPGLGLR | | DNF | FYF | | PSDNFNKFYFGSGTKLNVKPNIQNPD | | RQIHSQGPQYIIHG | |
| | LIYFSYDVKMKEK | | NKF | G | | PAVYQLRDSKSSDKSVCLFTDFDSQT | | LKNNETNEMASLII | |
| | GDIPEGYSVSREK | | YFG_ | | | NVSQSKDSDVYITDKTVLDMRSMDFK | | TEDRKSSTLILPHA | |
| | KERFSLILESASTN | | hTRAJ21 | | | SNSAVAWSNKSDFACANAFNNSIIPE | | TLRDTAVYYCIVRV | |
| | QTSMYLCASSRTF | | | | | DTFFPSPESSCDVKLVEKSFETDTNL | | PSDNFNKFYFGSG | |
| | RELFFGEGSRLTV | | | | | NFQNLSVIGFRILLLKVAGFNLLMTLR | | TKLNVKPN | |
| | L | | | | | LWSS | | | |
| | MSIGLLCCVAFSLL | 188 | hTRAV19_ | C | 35 | MLTASLLRAVIASICVVSSMAQKVTQA | 111 | MLTASLLRAVIASI | 187 |
| | WASPVNAGVTQT | | CALS | ALS | | QTEISVVEKEDVTLDCVYETRDTTYYL | | CVVSSMAQKVTQ | |
| | PKFQVLKTGQSMT | | EAR | EAR | | FWYKQPPSGELVFLIRRNSFDEQNEIS | | AQTEISVVEKEDVT | |
| | LQCAQDMNHNSM | | GG | GGA | | GRYSWNFQKSTSSFNFTITASQVVDS | | LDCVYETRDTTYY | |
| | YWYRQDPGMGLR | | AD | DG | | AVYFCALSEARGGADGLTFGKGTHLII | | LFWYKQPPSGELV | |
| | LIYYSASEGTTDK | | GLT | LTF | | QPYIQNPDPAVYQLRDSKSSDKSVCL | | FLIRRNSFDEQNEI | |
| | GEVPNGYNVSRL | | FG_ | G | | FTDFDSQTNVSQSKDSDVYITDKTVL | | SGRYSWNFQKST | |
| | NKREFSLRLESAA | | hTRAJ45 | | | DMRSMDFKSNSAVAWSNKSDFACA | | SSFNFTITASQVVD | |
| | PSQTSVYFCASSY | | | | | NAFNNSIIPEDTFFPSPESSCDVKLVE | | SAVYFCALSEARG | |
| | SGTSGIYEQYFGP | | | | | KSFETDTNLNFQNLSVIGFRILLLKVA | | GADGLTFGKGTHL | |
| | GTRLTVT | | | | | GFNLLMTLRLWSS | | IIQPY | |
| | MSIGLLCCVAFSLL | 190 | hTRAV12- | CVV | 37 | MISLRVLLVILWLQLSWVWSQRKEVE | 113 | MISLRVLLVILWLQ | 189 |
| | WASPVNAGVTQT | | 1_ | NGG | | QDPGPFNVPEGATVAFNCTYSNSASQ | | LSWVWSQRKEVE | |
| | PKFQVLKTGQSMT | | CVVN | YNN | | SFFWYRQDCRKEPKLLMSVYSSGNE | | QDPGPFNVPEGAT | |
| | LQCAQDMNHNSM | | GG | NDM | | DGRFTAQLNRASQYISLLIRDSKLSDS | | VAFNCTYSNSASQ | |
| | YWYRQDPGMGLR | | YN | R | | ATYLCVVNGGYNNNDMRFGAGTRLT | | SFFWYRQDCRKE | |
| | LIYYSASEGTTDK | | NN | | | VKPNIQNPDPAVYQLRDSKSSDKSVC | | PKLLMSVYSSGNE | |
| | GEVPNGYNVSRL | | DM | | | LFTDFDSQTNVSQSKDSDVYITDKTVL | | DGRFTAQLNRASQ | |
| | NKREFSLRLESAA | | RFG_ | | | DMRSMDFKSNSAVAWSNKSDFACA | | YISLLIRDSKLSDS | |
| | PSQTSVYFCASSY | | hTRAJ43 | | | NAFNNSIIPEDTFFPSPESSCDVKLVE | | ATYLCVVNGGYNN | |
| | SLSGTSSYEQYFG | | | | | KSFETDTNLNFQNLSVIGFRILLLKVA | | NDMRFGAGTRLTV | |
| | PGTRLTVT | | | | | GFNLLMTLRLWSS | | KPN | |
| | MGCRLLCCAVLCL | 192 | hTRAV12- | CAV | 39 | MMKSLRVLLVILWLQLSWVWSQQKEV | 115 | MMKSLRVLLVILW | 191 |
| | LGAVPIDTEVTQTP | | 2_ | NAG | | EQNSGPLSVPEGAIASLNCTYSDRGS | | LQLSWVWSQQKE | |
| | KHLVMGMTNKKSL | | CAVN | NQF | | QSFFWYRQYSGKSPELIMFIYSNGDK | | VEQNSGPLSVPEG | |
| | KCEQHMGHRAMY | | AG | YFG | | EDGRFTAQLNKASQYVSLLIRDSQPS | | AIASLNCTYSDRG | |
| | WYKQKAKKPPEL | | NQ | | | DSATYLCAVNAGNQFYFGTGTSLTVIP | | SQSFFWYRQYSG | |
| | MFVYSYEKLSINE | | FYF | | | NIQNPDPAVYQLRDSKSSDKSVCLFT | | KSPELIMFIYSNGD | |
| | SVPSRFSPECPNS | | G_ | | | DFDSQTNVSQSKDSDVYITDKTVLDM | | KEDGRFTAQLNKA | |
| | SLLNLHLHALQPE | | hTRAJ49 | | | RSMDFKSNSAVAWSNKSDFACANAF | | SQYVSLLIRDSQP | |
| | DSALYLCASSPDR | | | | | NNSIIPEDTFFPSPESSCDVKLVEKSF | | SDSATYLCAVNAG | |
| | SADTQYFGPGTRL | | | | | ETDTNLNFQNLSVIGFRILLLKVAGFN | | NQFYFGTGTSLTVI | |
| | TVL | | | | | LLMTLRLWSS | | PN | |
| | MLSLLLLLLGLGSV | 194 | hTRAV12- | CAV | 41 | MMKSLRVLLVILWLQLSWVWSQQKEV | 117 | MMKSLRVLLVILW | 193 |
| | FSAVISQKPSRDIC | | 2_ | GDY | | EQNSGPLSVPEGAIASLNCTYSDRGS | | LQLSWVWSQQKE | |
| | QRGTSLTIQCQVD | | CAV | KLS | | QSFFWYRQYSGKSPELIMFIYSNGDK | | VEQNSGPLSVPEG | |
| | SQVTMMFWYRQQ | | GD | FG | | EDGRFTAQLNKASQYVSLLIRDSQPS | | AIASLNCTYSDRG | |
| | PGQSLTLIATANQ | | YKL | | | DSATYLCAVGDYKLSFGAGTTVTVRA | | SQSFFWYRQYSG | |
| | GSEATYESGFVID | | SFG_ | | | NIQNPDPAVYQLRDSKSSDKSVCLFT | | KSPELIMFIYSNGD | |
| | KFPISRPNLTFSTL | | hTRAJ20 | | | DFDSQTNVSQSKDSDVYITDKTVLDM | | KEDGRFTAQLNKA | |
| | TVSNMSPEDSSIY | | | | | RSMDFKSNSAVAWSNKSDFACANAF | | SQYVSLLIRDSQP | |
| | LCSASRDIDSGNTI | | | | | NNSIIPEDTFFPSPESSCDVKLVEKSF | | SDSATYLCAVGDY | |
| | YFGEGSWLTVV | | | | | ETDTNLNFQNLSVIGFRILLLKVAGFN | | KLSFGAGTTVTVR | |
| | | | | | | LLMTLRLWSS | | AN | |
| | MGSRLLCWVLLCL | 196 | hTRAV20_ | CAV | 43 | MEKMLECAFIVLWLQLGWLSGEDQVT | 119 | MEKMLECAFIVLW | 195 |
| | LGAGPVKAGVTQT | | CAV | QAP | | QSPEALRLQEGESSSLNCSYTVSGLR | | LQLGWLSGEDQV | |
| | PRYLIKTRGQQVT | | QA | YSG | | GLFWYRQDPGKGPEFLFTLYSAGEEK | | TQSPEALRLQEGE | |
| | LSCSPISGHRSVS | | PYS | AGS | | EKERLKATLTKKESFLHITAPKPEDSAT | | SSSLNCSYTVSGL | |
| | WYQQTPGQGLQF | | GA | YQ | | YLCAVQAPYSGAGSYQLTFGKGTKLS | | RGLFWYRQDPGK | |
| | LFEYFSETQRNKG | | GS | LT | | VIPNIQNPDPAVYQLRDSKSSDKSVC | | GPEFLFTLYSAGE | |
| | NFPGRFSGRQFS | | YQL | FG | | LFTDFDSQTNVSQSKDSDVYITDKTVL | | EKEKERLKATLTK | |
| | NSRSEMNVSTLEL | | TFG_ | | | DMRSMDFKSNSAVAWSNKSDFACA | | KESFLHITAPKPED | |
| | GDSALYLCASKFG | | hTRAJ28 | | | NAFNNSIIPEDTFFPSPESSCDVKLVE | | SATYLCAVQAPYS | |
| | DTQYFGPGTRLTV | | | | | KSFETDTNLNFQNLSVIGFRILLLKVA | | GAGSYQLTFGKGT | |
| | L | | | | | GFNLLMTLRLWSS | | KLSVIPN | |
| | MGSRLLCWVLLCL | 198 | hTRAV01- | CA | 45 | MWGVFLLYVSMKMGGTTGQNIDQPT | 121 | MWGVFLLYVSMK | 197 |
| | LGAGPVKAGVTQT | | 2_ | VIR | | EMTATEGAIVQINCTYQTSGFNGLFW | | MGGTTGQNIDQPT | |
| | PRYLIKTRGQQVT | | CAVI | GYS | | YQQHAGEAPTFLSYNVLDGLEEKGRF | | EMTATEGAIVQINC | |
| | LSCSPISGHRSVS | | RG | GAG | | SSFLSRSKGYSYLLLKELQMKDSASYL | | TYQTSGFNGLFWY | |
| | WYQQTPGQGLQF | | YS | SYQ | | CAVIRGYSGAGSYQLTFGKGTKLSVIP | | QQHAGEAPTFLSY | |
| | LFEYFSETQRNKG | | GA | LTF | | NIQNPDPAVYQLRDSKSSDKSVCLFT | | NVLDGLEEKGRFS | |
| | NFPGRFSGRQFS | | GS | G | | DFDSQTNVSQSKDSDVYITDKTVLDM | | SFLSRSKGYSYLLL | |
| | NSRSEMNVSTLEL | | YQL | | | RSMDFKSNSAVAWSNKSDFACANAF | | KELQMKDSASYLC | |
| | GDSALYLCASKFG | | TFG_ | | | NNSIIPEDTFFPSPESSCDVKLVEKSF | | AVIRGYSGAGSYQ | |
| | NELFFGPGTRLTV | | hTRAJ28 | | | ETDTNLNFQNLSVIGFRILLLKVAGEN | | LTFGKGTKLSVIPN | |
| | L | | | | | LLMTLRLWSS | | | |
| | MGFRLLCCVAFCL | 200 | hTRAV26- | CIV | 47 | MRLVARVTVFLTFGTIIDAKTTQPPSM | 123 | MRLVARVTVFLTF | 199 |
| | LGAGPVDSGVTQT | | 1_ | RVP | | DCAEGRAANLPCNHSTISGNEYVYWY | | GTIIDAKTTQPPSM | |
| | PKHLITATGQRVTL | | CIVR | SGA | | RQIHSQGPQYIIHGLKNNETNEMASLII | | DCAEGRAANLPCN | |
| | RCSPRSGDLSVY | | VPS | GSY | | TEDRKSSTLILPHATLRDTAVYYCIVRV | | HSTISGNEYVYWY | |
| | WYQQSLDQGLQF | | GA | QLT | | PSGAGSYQLTFGKGTKLSVIPNIQNPD | | RQIHSQGPQYIIHG | |
| | LIQYYNGEERAKG | | GS | FG | | PAVYQLRDSKSSDKSVCLFTDFDSQT | | LKNNETNEMASLII | |
| | NILERFSAQQFPD | | YQL | | | NVSQSKDSDVYITDKTVLDMRSMDFK | | TEDRKSSTLILPHA | |
| | LHSELNLSSLELG | | TFG_ | | | SNSAVAWSNKSDFACANAFNNSIIPE | | TLRDTAVYYCIVRV | |
| | DSALYFCASSVVG | | hTRAJ28 | | | DTFFPSPESSCDVKLVEKSFETDTNL | | PSGAGSYQLTFGK | |
| | TREQFFGPGTRLT | | | | | NFQNLSVIGFRILLLKVAGFNLLMTLR | | GTKLSVIPN | |
| | VL | | | | | LWSS | | | |
| | MGSRLLCWVLLCL | 202 | hTRAV01- | CAV | 49 | MWGVFLLYVSMKMGGTTGQNIDQPT | 125 | MWGVFLLYVSMK | 201 |
| | LGAGPVKAGVTQT | | 2_ | RTG | | EMTATEGAIVQINCTYQTSGFNGLFW | | MGGTTGQNIDQPT | |
| | PRYLIKTRGQQVT | | CAVR | YSG | | YQQHAGEAPTFLSYNVLDGLEEKGRF | | EMTATEGAIVQINC | |
| | LSCSPISGHRSVS | | TGY | AGS | | SSFLSRSKGYSYLLLKELQMKDSASYL | | TYQTSGFNGLFWY | |
| | WYQQTPGQGLQF | | SG | YQ | | CAVRTGYSGAGSYQLTFGKGTKLSVI | | QQHAGEAPTFLSY | |
| | LFEYFSETQRNKG | | AG | LT | | PNIQNPDPAVYQLRDSKSSDKSVCLF | | NVLDGLEEKGRFS | |
| | NFPGRFSGRQFS | | SY | FG | | TDFDSQTNVSQSKDSDVYITDKTVLD | | SFLSRSKGYSYLLL | |
| | NSRSEMNVSTLEL | | QLT | | | MRSMDFKSNSAVAWSNKSDFACAN | | KELQMKDSASYLC | |
| | GDSALYLCASSYG | | FG_ | | | AFNNSIIPEDTFFPSPESSCDVKLVEK | | AVRTGYSGAGSY | |
| | NEQFFGPGTRLTV | | hTRAJ28 | | | SFETDTNLNFQNLSVIGFRILLLKVAG | | QLTFGKGTKLSVIP | |
| | L | | | | | FNLLMTLRLWSS | | N | |

| | | | | | | | | TCR | | | |
|---|---|---|---|---|---|---|---|---|---|---|---|
| | | Non | | | | | | pMHC | | | |
| | | synonymus | HLA | | | | Additional | beta | SEQ | | SEQ |
| Patient | | mutations | re- | Minimal | | TCR | Id | chain | ID | | ID |
| ID | Gene | SNVs | striction | epitope | | Id | information | CDR3 | NO | TCR pMHC beta chain | NO |
| Patient | Tyrosinase | | B08:01* | LPE | 235 | A | hTRBV04- | CAS | 52 | MGCRLLCCAVLCLLGAVPIDTEVTQTPKHL | 128 |
| 3 | (508-514) | | | EKQ | | | 1_ | SQK | | VMGMTNKKSLKCEQHMGHRAMYWYKQK | |
| | | | | P | | | CASS | QAY | | AKKPPELMFVYSYEKLSINESVPSRESPEC | |
| | | | | | | | QK | GYT | | PNSSLLNLHLHALQPEDSALYLCASSQKQA | |
| | | | | | | | QAYG | FG | | YGYTFGSGTRLTVVEDLNKVFPPEVAVFE | |
| | | | | | | | YTFG_ | | | PSEAEISHTQKATLVCLATGFFPDHVELS | |
| | | | | | | | hTRBJ01-2 | | | WWVNGKEVHSGVSTDPQPLKEQPALNDS | |
| | | | | | | | | | | RYCLSSRLRVSATFWQNPRNHFRCQVQF | |
| | | | | | | | | | | YGLSENDEWTQDRAKPVTQIVSAEAWGR | |
| | | | | | | | | | | ADCGFTSVSYQQGVLSATILYEILLGKATL | |
| | | | | | | | | | | YAVLVSALVLMAMVKRKDF | |
| | CES2 | P126S | B15:01 | VQT | 236 | B | hTRBV27_ | CAS | 54 | MGPQLLGYVVLCLLGAGPLEAQVTQNPRY | 130 |
| | | | | FLG | | | CASS | SFR | | LITVTGKKLTVTCSQNMNHEYMSWYRQDP | |
| | | | | ISF | | | FRA | AEA | | GLGLRQIYYSMNVEVTDKGDVPEGYKVSR | |
| | | | | | | | EAY | YEQ | | KEKRNFPLILESPSPNQTSLYFCASSFRAE | |
| | | | | | | | EQ | YFG | | AYEQYFGPGTRLTVTEDLNKVFPPEVAVF | |
| | | | | | | | YFG_ | | | EPSEAEISHTQKATLVCLATGFFPDHVELS | |
| | | | | | | | hTRBJ02-7 | | | WWVNGKEVHSGVSTDPQPLKEQPALNDS | |
| | | | | | | | | | | RYCLSSRLRVSATFWQNPRNHFRCQVQF | |
| | | | | | | | | | | YGLSENDEWTQDRAKPVTQIVSAEAWGR | |
| | | | | | | | | | | ADCGFTSVSYQQGVLSATILYEILLGKATL | |
| | | | | | | | | | | YAVLVSALVLMAMVKRKDF | |
| | Tyrosinase | | B15:01 | RLP | 237 | C | hTRBV07- | CAS | 56 | MGTSLLCWMALCLLGADHADTGVSQNPR | 132 |
| | (310-320) | | | SSA | | | 9_ | SLG | | HKITKRGQNVTFRCDPISEHNRLYWYRQTL | |
| | | | | DVE | | | CASS | KGR | | GQGPEFLTYFQNEAQLEKSRLLSDRFSAE | |
| | | | | F | | | LGK | NTE | | RPKGSFSTLEIQRTEQGDSAMYLCASSLGK | |
| | | | | | | | GR | A | | GRNTEAFFGQGTRLTVVEDLNKVFPPEVA | |
| | | | | | | | NTE | | | VFEPSEAEISHTQKATLVCLATGFFPDHVE | |
| | | | | | | | AFFG_ | | | LSWWVNGKEVHSGVSTDPQPLKEQPALN | |
| | | | | | | | hTRBJ01-1 | | | DSRYCLSSRLRVSATFWQNPRNHFRCQV | |
| | | | | | | | | | | QFYGLSENDEWTQDRAKPVTQIVSAEAW | |
| | | | | | | | | | | GRADCGFTSVSYQQGVLSATILYEILLGKA | |
| | | | | | | | | | | TLYAVLVSALVLMAMVKRKDF | |

| | TCR pMHC beta | | | TCR | | | | TCR pMHC alpha | |
|---|---|---|---|---|---|---|---|---|---|
| | chain | | | pMHC | | | | chain | |
| | (excluding | SEQ | Additional | alpha | SEQ | | SEQ | (excluding | SEQ |
| Patient | the constant | ID | Id | chain | ID | | ID | the constant | ID |
| ID | region) | NO | information | CDR3 | NO | TCR pMHC alpha chain | NO | region) | NO |
| Patient | MGCRLLCCAVLCL | 204 | hTRAV21_ | CAV | 51 | METLLGLLILWLQLQWVSSKQEVTQIP | 127 | METLLGLLILWLQL | 203 |
| 3 | LGAVPIDTEVTQTP | | CAVS | SPN | | AALSVPEGENLVLNCSFTDSAIYNLQW | | QWVSSKQEVTQIP | |
| | KHLVMGMTNKKSL | | PNY | YGQ | | FRQDPGKGLTSLLLIQSSQREQTSGR | | AALSVPEGENLVL | |
| | KCEQHMGHRAMY | | GQ | NFV | | LNASLDKSSGRSTLYIAASQPGDSATY | | NCSFTDSAIYNLQ | |
| | WYKQKAKKPPEL | | NFV | FG | | LCAVSPNYGQNFVFGPGTRLSVLPYI | | WFRQDPGKGLTS | |
| | MFVYSYEKLSINE | | FG_ | | | QNPDPAVYQLRDSKSSDKSVCLFTDF | | LLLIQSSQREQTS | |
| | SVPSRFSPECPNS | | hTRAJ26 | | | DSQTNVSQSKDSDVYITDKTVLDMRS | | GRLNASLDKSSGR | |
| | SLLNLHLHALQPE | | | | | MDFKSNSAVAWSNKSDFACANAFNN | | STLYIAASQPGDS | |
| | DSALYLCASSQKQ | | | | | SIIPEDTFFPSPESSCDVKLVEKSFET | | ATYLCAVSPNYGQ | |
| | AYGYTFGSGTRLT | | | | | DTNLNFQNLSVIGFRILLLKVAGFNLL | | NFVFGPGTRLSVL | |
| | VV | | | | | MTLRLWSS | | PY | |
| | MGPQLLGYVVLCL | 206 | hTRAV26- | CI | 53 | MKLVTSITVLLSLGIMGDAKTTQPNSM | 129 | MKLVTSITVLLSLGI | 205 |
| | LGAGPLEAQVTQN | | 2_ | TTN | | ESNEEEPVHLPCNHSTISGTDYIHWYR | | MGDAKTTQPNSM | |
| | PRYLITVTGKKLTV | | CITT | AGK | | QLPSQGPEYVIHGLTSNVNNRMASLAI | | ESNEEEPVHLPCN | |
| | TCSQNMNHEYMS | | NA | STF | | AEDRKSSTLILHRATLRDAAVYYCITTN | | HSTISGTDYIHWY | |
| | WYRQDPGLGLRQI | | GK | G | | AGKSTFGDGTTLTVKPNIQNPDPAVY | | RQLPSQGPEYVIH | |
| | YYSMNVEVTDKG | | STFG_ | | | QLRDSKSSDKSVCLFTDFDSQTNVSQ | | GLTSNVNNRMASL | |
| | DVPEGYKVSRKEK | | hTRAJ27 | | | SKDSDVYITDKTVLDMRSMDFKSNSA | | AIAEDRKSSTLILH | |
| | RNFPLILESPSPNQ | | | | | VAWSNKSDFACANAFNNSIIPEDTFFP | | RATLRDAAVYYCIT | |
| | TSLYFCASSFRAE | | | | | SPESSCDVKLVEKSFETDTNLNFQNL | | TNAGKSTFGDGTT | |
| | AYEQYFGPGTRLT | | | | | SVIGFRILLLKVAGFNLLMTLRLWSS | | LTVKPN | |
| | VT | | | | | | | | |
| | MGTSLLCWMALC | 208 | hTRAV01- | CAV | 55 | MWGVFLLYVSMKMGGTTGQNIDQPT | 131 | MWGVFLLYVSMK | 207 |
| | LLGADHADTGVSQ | | 2_ | RDN | | EMTATEGAIVQINCTYQTSGFNGLFW | | MGGTTGQNIDQPT | |
| | NPRHKITKRGQNV | | CAVR | NND | | YQQHAGEAPTFLSYNVLDGLEEKGRF | | EMTATEGAIVQINC | |
| | TFRCDPISEHNRL | | DN | MRF | | SSFLSRSKGYSYLLLKELQMKDSASYL | | TYQTSGFNGLFWY | |
| | YWYRQTLGQGPE | | NN | G | | CAVRDNNNDMRFGAGTRLTVKPNIQN | | QQHAGEAPTFLSY | |
| | FLTYFQNEAQLEK | | DM | | | PDPAVYQLRDSKSSDKSVCLFTDFDS | | NVLDGLEEKGRFS | |
| | SRLLSDRFSAERP | | RFG_ | | | QTNVSQSKDSDVYITDKTVLDMRSMD | | SFLSRSKGYSYLLL | |
| | KGSFSTLEIQRTE | | hTRAJ43 | | | FKSNSAVAWSNKSDFACANAFNNSII | | KELQMKDSASYLC | |
| | QGDSAMYLCASSL | | | | | PEDTFFPSPESSCDVKLVEKSFETDT | | AVRDNNNDMRFG | |
| | GKGRNTEAFFGQ | | | | | NLNFQNLSVIGFRILLLKVAGFNLLMT | | AGTRLTVKPN | |
| | GTRLTVV | | | | | LRLWSS | | | |

| | | | | | | | | TCR | | | |
|---|---|---|---|---|---|---|---|---|---|---|---|
| | | Non | | | | | | pMHC | | | |
| | | synonymus | | | | | Additional | beta | SEQ | | SEQ |
| Patient | | mutations | HLA | Minimal | | TCR | Id | chain | ID | | ID |
| ID | Gene | SNVs | restriction | epitope | | Id | information | CDR3 | NO | TCR pMHC beta chain | NO |
| Patient | APOO | P107L | A02:01 | ALP | 238 | A1 | hTRBV20_ | CSA | 58 | MLLLLLLLGPGSGLGAVVSQHPSRVICKSG | 134 |
| 5 | | | | GFF | | | CSAP | PGL | | TSVKIECRSLDFQATTMFWYRQFPKQSLM | |
| | | | | PRL | | | GLA | AGD | | LMATSNEGSKATYEQGVEKDKFLINHASLT | |
| | | | | | | | GD | PYN | | LSTLTVTSAHPEDSSFYICSAPGLAGDPYN | |
| | | | | | | | PY | EQF | | EQFFGPGTRLTVLEDLNKVFPPEVAVFEPS | |
| | | | | | | | NE | G | | EAEISHTQKATLVCLATGFFPDHVELSWW | |
| | | | | | | | QFFG_ | | | VNGKEVHSGVSTDPQPLKEQPALNDSRY | |
| | | | | | | | hTRBJ02-1 | | | CLSSRLRVSATFWQNPRNHFRCQVQFYG | |
| | | | | | | | | | | LSENDEWTQDRAKPVTQIVSAEAWGRAD | |
| | | | | | | | | | | CGFTSVSYQQGVLSATILYEILLGKATLYA | |
| | | | | | | | | | | VLVSALVLMAMVKRKDF | |
| | | | | | | A2 | hTRBV24_ | CAT | 60 | MASLLFFCGAFYLLGTGSMDADVTQTPRN | 136 |
| | | | | | | | CATS | SGA | | RITKTGKRIMLECSQTKGHDRMYWYRQDP | |
| | | | | | | | GA | EN | | GLGLRLIYYSFDVKDINKGEISDGYSVSRQA | |
| | | | | | | | ENT | TGE | | QAKFSLSLESAIPNQTALYFCATSGAENTG | |
| | | | | | | | GEL | LFFG | | ELFFGEGSRLTVLEDLNKVFPPEVAVFEPS | |
| | | | | | | | FFG_ | | | EAEISHTQKATLVCLATGFFPDHVELSWW | |
| | | | | | | | hTRBJ02-2 | | | VNGKEVHSGVSTDPQPLKEQPALNDSRY | |
| | | | | | | | | | | CLSSRLRVSATFWQNPRNHFRCQVQFYG | |
| | | | | | | | | | | LSENDEWTQDRAKPVTQIVSAEAWGRAD | |
| | | | | | | | | | | CGFTSVSYQQGVLSATILYEILLGKATLYA | |
| | | | | | | | | | | VLVSALVLMAMVKRKDF | |
| | | | | | | A3 | hTRBV13_ | CAS | 62 | MLSPDLPDSAWNTRLLCHVMLCLLGAVSV | 138 |
| | | | | | | | CASS | SGQ | | AAGVIQSPRHLIKEKRETATLKCYPIPRHDT | |
| | | | | | | | GQ | GGS | | VYWYQQGPGQDPQFLISFYEKMQSDKGSI | |
| | | | | | | | GG | DT | | PDRFSAQQFSDYHSELNMSSLELGDSALY | |
| | | | | | | | SDT | QYF | | FCASSGQGGSDTQYFGPGTRLTVLEDLNK | |
| | | | | | | | QY | G | | VFPPEVAVFEPSEAEISHTQKATLVCLATG | |
| | | | | | | | FG_ | | | FFPDHVELSWWVNGKEVHSGVSTDPQPL | |
| | | | | | | | hTRBJ02-3 | | | KEQPALNDSRYCLSSRLRVSATFWQNPR | |
| | | | | | | | | | | NHFRCQVQFYGLSENDEWTQDRAKPVTQI | |
| | | | | | | | | | | VSAEAWGRADCGFTSVSYQQGVLSATILY | |
| | | | | | | | | | | EILLGKATLYAVLVSALVLMAMVKRKDF | |
| | | | | | | A5 | hTRBV12- | CAS | 64 | MDSWTFCCVSLCILVAKHTDAGVIQSPRHE | 140 |
| | | | | | | | 3_ | SSS | | VTEMGQEVTLRCKPISGHNSLFWYRQTMM | |
| | | | | | | | CASS | ETG | | RGLELLIYFNNNVPIDDSGMPEDRFSAKMP | |
| | | | | | | | SSE | ELF | | NASFSTLKIQPSEPRDSAVYFCASSSSETG | |
| | | | | | | | TG | FG | | ELFFGEGSRLTVLEDLNKVFPPEVAVFEPS | |
| | | | | | | | ELF | | | EAEISHTQKATLVCLATGFFPDHVELSWW | |
| | | | | | | | FG_ | | | VNGKEVHSGVSTDPQPLKEQPALNDSRY | |
| | | | | | | | hTRBJ02-2 | | | CLSSRLRVSATFWQNPRNHFRCQVQFYG | |
| | | | | | | | | | | LSENDEWTQDRAKPVTQIVSAEAWGRAD | |
| | | | | | | | | | | CGFTSVSYQQGVLSATILYEILLGKATLYA | |
| | | | | | | | | | | VLVSALVLMAMVKRKDF | |

| | TCR pMHC beta | | | TCR | | | | TCR pMHC alpha | |
|---|---|---|---|---|---|---|---|---|---|
| | chain | | | pMHC | | | | chain | |
| | (excluding | SEQ | Additional | alpha | SEQ | | SEQ | (excluding | SEQ |
| Patient | the constant | ID | Id | chain | ID | | ID | the constant | ID |
| ID | region) | NO | information | CDR3 | NO | TCR pMHC alpha chain | NO | region) | NO |
| Patient | MLLLLLLLGPGSGL | 210 | hTRAV12- | CAV | 57 | MMKSLRVLLVILWLQLSWVWSQQKEV | 133 | MMKSLRVLLVILW | 209 |
| 5 | GAVVSQHPSRVIC | | 2_ | GGS | | EQNSGPLSVPEGAIASLNCTYSDRGS | | LQLSWVWSQQKE | |
| | KSGTSVKIECRSL | | CAV | ARQ | | QSFFWYRQYSGKSPELIMFIYSNGDK | | VEQNSGPLSVPEG | |
| | DFQATTMFWYRQ | | GG | LTF | | EDGRFTAQLNKASQYVSLLIRDSQPS | | AIASLNCTYSDRG | |
| | FPKQSLMLMATSN | | SAR | G | | DSATYLCAVGGSARQLTFGSGTQLTV | | SQSFFWYRQYSG | |
| | EGSKATYEQGVEK | | QLT | | | LPDIQNPDPAVYQLRDSKSSDKSVCL | | KSPELIMFIYSNGD | |
| | DKFLINHASLTLST | | FG_ | | | FTDFDSQTNVSQSKDSDVYITDKTVL | | KEDGRFTAQLNKA | |
| | LTVTSAHPEDSSF | | hTRAJ22 | | | DMRSMDFKSNSAVAWSNKSDFACA | | SQYVSLLIRDSQP | |
| | YICSAPGLAGDPY | | | | | NAFNNSIIPEDTFFPSPESSCDVKLVE | | SDSATYLCAVGGS | |
| | NEQFFGPGTRLTV | | | | | KSFETDTNLNFQNLSVIGFRILLLKVA | | ARQLTFGSGTQLT | |
| | L | | | | | GFNLLMTLRLWSS | | VLPD | |
| | MASLLFFCGAFYL | 212 | hTRAV12- | CAV | 59 | MMKSLRVLLVILWLQLSWVWSQQKEV | 135 | MMKSLRVLLVILW | 211 |
| | LGTGSMDADVTQ | | 2_ | NPL | | EQNSGPLSVPEGAIASLNCTYSDRGS | | LQLSWVWSQQKE | |
| | TPRNRITKTGKRIM | | CAVN | NNF | | QSFFWYRQYSGKSPELIMFIYSNGDK | | VEQNSGPLSVPEG | |
| | LECSQTKGHDRM | | PLN | NKF | | EDGRFTAQLNKASQYVSLLIRDSQPS | | AIASLNCTYSDRG | |
| | YWYRQDPGLGLR | | NFN | YFG | | DSATYLCAVNPLNNFNKFYFGSGTKL | | SQSFFWYRQYSG | |
| | LIYYSFDVKDINKG | | KFY | | | NVKPNIQNPDPAVYQLRDSKSSDKSV | | KSPELIMFIYSNGD | |
| | EISDGYSVSRQAQ | | FG_ | | | CLFTDFDSQTNVSQSKDSDVYITDKT | | KEDGRFTAQLNKA | |
| | AKFSLSLESAIPNQ | | hTRAJ21 | | | VLDMRSMDFKSNSAVAWSNKSDFAC | | SQYVSLLIRDSQP | |
| | TALYFCATSGAEN | | | | | ANAFNNSIIPEDTFFPSPESSCDVKLV | | SDSATYLCAVNPL | |
| | TGELFFGEGSRLT | | | | | EKSFETDTNLNFQNLSVIGFRILLLKV | | NNFNKFYFGSGTK | |
| | VL | | | | | AGFNLLMTLRLWSS | | LNVKPN | |
| | MLSPDLPDSAWN | 214 | hTRAV20_ | CAV | 61 | MEKMLECAFIVLWLQLGWLSGEDQVT | 137 | MEKMLECAFIVLW | 213 |
| | TRLLCHVMLCLLG | | CAVR | RVA | | QSPEALRLQEGESSSLNCSYTVSGLR | | LQLGWLSGEDQV | |
| | AVSVAAGVIQSPR | | VA | GGT | | GLFWYRQDPGKGPEFLFTLYSAGEEK | | TQSPEALRLQEGE | |
| | HLIKEKRETATLKC | | GG | SYG | | EKERLKATLTKKESFLHITAPKPEDSAT | | SSSLNCSYTVSGL | |
| | YPIPRHDTVYWYQ | | TSY | KL | | YLCAVRVAGGTSYGKLTFGQGTILTVH | | RGLFWYRQDPGK | |
| | QGPGQDPQFLISF | | GKL | TE | | PNIQNPDPAVYQLRDSKSSDKSVCLF | | GPEFLFTLYSAGE | |
| | YEKMQSDKGSIPD | | TFG_ | | | TDFDSQTNVSQSKDSDVYITDKTVLD | | EKEKERLKATLTK | |
| | RFSAQQFSDYHSE | | hTRAJ52 | | | MRSMDFKSNSAVAWSNKSDFACAN | | KESFLHITAPKPED | |
| | LNMSSLELGDSAL | | | | | AFNNSIIPEDTFFPSPESSCDVKLVEK | | SATYLCAVRVAGG | |
| | YFCASSGQGGSD | | | | | SFETDTNLNFQNLSVIGFRILLLKVAG | | TSYGKLTFGQGTIL | |
| | TQYFGPGTRLTVL | | | | | FNLLMTLRLWSS | | TVHPN | |
| | MDSWTFCCVSLCI | 216 | hTRAV20_ | CAV | 63 | MEKMLECAFIVLWLQLGWLSGEDQVT | 139 | MEKMLECAFIVLW | 215 |
| | LVAKHTDAGVIQS | | CAV | QGG | | QSPEALRLQEGESSSLNCSYTVSGLR | | LQLGWLSGEDQV | |
| | PRHEVTEMGQEV | | QG | SNY | | GLFWYRQDPGKGPEFLFTLYSAGEEK | | TQSPEALRLQEGE | |
| | TLRCKPISGHNSLF | | GS | KL | | EKERLKATLTKKESFLHITAPKPEDSAT | | SSSLNCSYTVSGL | |
| | WYRQTMMRGLEL | | NYK | TFG | | YLCAVQGGSNYKLTFGKGTLLTVNPNI | | RGLFWYRQDPGK | |
| | LIYFNNNVPIDDSG | | LTFG_ | | | QNPDPAVYQLRDSKSSDKSVCLFTDF | | GPEFLFTLYSAGE | |
| | MPEDRFSAKMPN | | hTRAJ53 | | | DSQTNVSQSKDSDVYITDKTVLDMRS | | EKEKERLKATLTK | |
| | ASFSTLKIQPSEPR | | | | | MDFKSNSAVAWSNKSDFACANAFNN | | KESFLHITAPKPED | |
| | DSAVYFCASSSSE | | | | | SIIPEDTFFPSPESSCDVKLVEKSFET | | SATYLCAVQGGSN | |
| | TGELFFGEGSRLT | | | | | DTNLNFQNLSVIGFRILLLKVAGFNLL | | YKLTFGKGTLLTV | |
| | VL | | | | | MTLRLWSS | | NPN | |

| | | | | | | | | TCR | | | |
|---|---|---|---|---|---|---|---|---|---|---|---|
| | | Non | | | | | | pMHC | | | |
| | | synonymus | | | | | Additional | beta | SEQ | | SEQ |
| Patient | | mutations | HLA | Minimal | | TCR | Id | chain | ID | | ID |
| ID | Gene | SNVs | restriction | epitope | | Id | information | CDR3 | NO | TCR pMHC beta chain | NO |
| Patient | NUP205 | Q471H | B35:03 | EPL | 239 | B | hTRBV05- | CAS | 66 | MGSRLLCWVLLCLLGAGPVKAGVTQTPRY | 142 |
| 6 | | | | HTP | | | 1_ | SLV | | LIKTRGQQVTLSCSPISGHRSVSWYQQTP | |
| | | | | TM | | | CASS | TGG | | GQGLQFLFEYFSETQRNKGNFPGRFSGRQ | |
| | | | | | | | LVT | LVD | | FSNSRSEMNVSTLELGDSALYLCASSLVTG | |
| | | | | | | | GGL | YEQ | | GLVDYEQYFGPGTRLTVTEDLNKVFPPEV | |
| | | | | | | | VDY | YFG | | AVFEPSEAEISHTQKATLVCLATGFFPDHV | |
| | | | | | | | EQY | | | ELSWWVNGKEVHSGVSTDPQPLKEQPAL | |
| | | | | | | | FG_ | | | NDSRYCLSSRLRVSATFWQNPRNHFRCQ | |
| | | | | | | | hTRBJ0 | | | VQFYGLSENDEWTQDRAKPVTQIVSAEA | |
| | | | | | | | 2-7 | | | WGRADCGFTSVSYQQGVLSATILYEILLG | |
| | | | | | | | | | | KATLYAVLVSALVLMAMVKRKDF | |
| | | | | | | C | hTRBV07- | CAS | 68 | MGTSLLCWVVLGFLGTDHTGAGVSQSPRY | 144 |
| | | | | | | | 6_ | SLL | | KVTKRGQDVALRCDPISGHVSLYWYRQAL | |
| | | | | | | | CASS | TGT | | GQGPEFLTYFNYEAQQDKSGLPNDRFSAE | |
| | | | | | | | LLT | GEL | | RPEGSISTLTIQRTEQRDSAMYRCASSLLT | |
| | | | | | | | GT | FFG | | GTGELFFGEGSRLTVLEDLNKVFPPEVAVF | |
| | | | | | | | GEL | | | EPSEAEISHTQKATLVCLATGFFPDHVELS | |
| | | | | | | | FFG_ | | | WWVNGKEVHSGVSTDPQPLKEQPALNDS | |
| | | | | | | | hTRBJ02-2 | | | RYCLSSRLRVSATFWQNPRNHFRCQVQF | |
| | | | | | | | | | | YGLSENDEWTQDRAKPVTQIVSAEAWGR | |
| | | | | | | | | | | ADCGFTSVSYQQGVLSATILYEILLGKATL | |
| | | | | | | | | | | YAVLVSALVLMAMVKRKDF | |
| | K1T | D165N | B35:03 | PNP | 240 | G | hTRBV02_ | CAS | 70 | MDTWLVCWAIFSLLKAGLTEPEVTQTPSH | 146 |
| | | | | KAG | | | CASE | EGL | | QVTQMGQEVILRCVPISNHLYFYWYRQILG | |
| | | | | M | | | GLA | AGA | | QKVEFLVSFYNNEISEKSEIFDDQFSVERP | |
| | | | | | | | GA | EQY | | DGSNFTLKIRSTKLEDSAMYFCASEGLAGA | |
| | | | | | | | EQ | FG | | EQYFGPGTRLTVTEDLNKVFPPEVAVFEP | |
| | | | | | | | YFG_ | | | SEAEISHTQKATLVCLATGFFPDHVELSW | |
| | | | | | | | hTRBJ02-7 | | | WVNGKEVHSGVSTDPQPLKEQPALNDSR | |
| | | | | | | | | | | YCLSSRLRVSATFWQNPRNHFRCQVQFY | |
| | | | | | | | | | | GLSENDEWTQDRAKPVTQIVSAEAWGRA | |
| | | | | | | | | | | DCGFTSVSYQQGVLSATILYEILLGKATLY | |
| | | | | | | | | | | AVLVSALVLMAMVKRKDF | |

| | TCR pMHC beta | | | TCR | | | | TCR pMHC alpha | |
|---|---|---|---|---|---|---|---|---|---|
| | chain | | | pMHC | | | | chain | |
| | (excluding | SEQ | Additional | alpha | SEQ | | SEQ | (excluding | SEQ |
| Patient | the constant | ID | Id | chain | ID | | ID | the constant | ID |
| ID | region) | NO | information | CDR3 | NO | TCR pMHC alpha chain | NO | region) | NO |
| Patient | MGSRLLCWVLLCL | 218 | hTRAV27_ | CAG | 65 | MVLKFSVSILWIQLAWVSTQLLEQSPQ | 141 | MVLKFSVSILWIQL | 217 |
| 6 | LGAGPVKAGVTQT | | CAG | AGS | | FLSIQEGENLTVYCNSSSVFSSLQWY | | AWVSTQLLEQSPQ | |
| | PRYLIKTRGQQVT | | AG | NSG | | RQEPGEGPVLLVTVVTGGEVKKLKRL | | FLSIQEGENLTVYC | |
| | LSCSPISGHRSVS | | SNS | YAL | | TFQFGDARKDSSLHITAAQPGDTGLYL | | NSSSVFSSLQWYR | |
| | WYQQTPGQGLQF | | GY | NFG | | CAGAGSNSGYALNFGKGTSLLVTPHI | | QEPGEGPVLLVTV | |
| | LFEYFSETQRNKG | | ALN | | | QNPDPAVYQLRDSKSSDKSVCLFTDF | | VTGGEVKKLKRLT | |
| | NFPGRFSGRQFS | | FG_ | | | DSQTNVSQSKDSDVYITDKTVLDMRS | | FQFGDARKDSSLH | |
| | NSRSEMNVSTLEL | | hTRAJ41 | | | MDFKSNSAVAWSNKSDFACANAFNN | | ITAAQPGDTGLYL | |
| | GDSALYLCASSLV | | | | | SIIPEDTFFPSPESSCDVKLVEKSFET | | CAGAGSNSGYAL | |
| | TGGLVDYEQYFGP | | | | | DTNLNFQNLSVIGFRILLLKVAGFNLL | | NFGKGTSLLVTPH | |
| | GTRLTVT | | | | | MTLRLWSS | | | |
| | MGTSLLCWVVLGF | 220 | hTRAV12- | CVV | 67 | MISLRVLLVILWLQLSWVWSQRKEVE | 143 | MISLRVLLVILWLQ | 219 |
| | LGTDHTGAGVSQ | | 1_ | NEG | | QDPGPFNVPEGATVAFNCTYSNSASQ | | LSWVWSQRKEVE | |
| | SPRYKVTKRGQD | | CVVN | MRF | | SFFWYRQDCRKEPKLLMSVYSSGNE | | QDPGPFNVPEGAT | |
| | VALRCDPISGHVS | | EG | G | | DGRFTAQLNRASQYISLLIRDSKLSDS | | VAFNCTYSNSASQ | |
| | LYWYRQALGQGP | | MR | | | ATYLCVVNEGMRFGAGTRLTVKPNIQ | | SFFWYRQDCRKE | |
| | EFLTYFNYEAQQD | | FG_ | | | NPDPAVYQLRDSKSSDKSVCLFTDFD | | PKLLMSVYSSGNE | |
| | KSGLPNDRFSAER | | hTRAJ43 | | | SQTNVSQSKDSDVYITDKTVLDMRSM | | DGRFTAQLNRASQ | |
| | PEGSISTLTIQRTE | | | | | DFKSNSAVAWSNKSDFACANAFNNSI | | YISLLIRDSKLSDS | |
| | QRDSAMYRCASS | | | | | IPEDTFFPSPESSCDVKLVEKSFETDT | | ATYLCVVNEGMRF | |
| | LLTGTGELFFGEG | | | | | NLNFQNLSVIGFRILLLKVAGFNLLMT | | GAGTRLTVKPN | |
| | SRLTVL | | | | | LRLWSS | | | |
| | MDTWLVCWAIFSL | 222 | hTRAV08- | CAV | 69 | MLLLLIPVLGMIFALRDARAQSVSQHN | 145 | MLLLLIPVLGMIFAL | 221 |
| | LKAGLTEPEVTQT | | 1_ | NSG | | HHVILSEAASLELGCNYSYGGTVNLF | | RDARAQSVSQHN | |
| | PSHQVTQMGQEVI | | CAVN | GGA | | WYVQYPGQHLQLLLKYFSGDPLVKGI | | HHVILSEAASLELG | |
| | LRCVPISNHLYFY | | SG | DG | | KGFEAEFIKSKFSFNLRKPSVQWSDT | | CNYSYGGTVNLF | |
| | WYRQILGQKVEFL | | GG | LT | | AEYFCAVNSGGGADGLTFGKGTHLIIQ | | WYVQYPGQHLQL | |
| | VSFYNNEISEKSEI | | AD | FG | | PYIQNPDPAVYQLRDSKSSDKSVCLF | | LLKYFSGDPLVKGI | |
| | FDDQFSVERPDG | | GLT | | | TDFDSQTNVSQSKDSDVYITDKTVLD | | KGFEAEFIKSKFSF | |
| | SNFTLKIRSTKLED | | FG_ | | | MRSMDFKSNSAVAWSNKSDFACAN | | NLRKPSVQWSDT | |
| | SAMYFCASEGLAG | | hTRAJ45 | | | AFNNSIIPEDTFFPSPESSCDVKLVEK | | AEYFCAVNSGGG | |
| | AEQYFGPGTRLTV | | | | | SFETDTNLNFQNLSVIGFRILLLKVAG | | ADGLTFGKGTHLII | |
| | T | | | | | FNLLMTLRLWSS | | QPY | |

| | | | | | | | | TCR | | | |
|---|---|---|---|---|---|---|---|---|---|---|---|
| | | Non | | | | | | pMHC | | | |
| | | synonymus | | | | | Additional | beta | SEQ | | SEQ |
| Patient | | mutations | HLA | Minimal | | TCR | Id | chain | ID | | ID |
| ID | Gene | SNVs | restriction | epitope | | Id | information | CDR3 | NO | TCR pMHC beta chain | NO |
| Patient | PHLPP2 | N1186Y | A01:01 | QSD | 241 | A | hTRBV10- | C | 72 | MGTRLFFYVALCLLWTGHMDAGITQSPRH | 148 |
| 7 | | | | NGL | | | 3_ | AIS | | KVTETGTPVTLRCHQTENHRYMYWYRQD | |
| | | | | DSD | | | CAIS | GGS | | PGHGLRLIHYSYGVKDTDKGEVSDGYSVS | |
| | | | | Y | | | GG | VGE | | RSKTEDFLLTLESATSSQTSVYFCAISGGS | |
| | | | | | | | SV | QYF | | VGEQYFGPGTRLTVTEDLNKVFPPEVAVF | |
| | | | | | | | GE | G | | EPSEAEISHTQKATLVCLATGFFPDHVELS | |
| | | | | | | | QY | | | WWVNGKEVHSGVSTDPQPLKEQPALNDS | |
| | | | | | | | FG_ | | | RYCLSSRLRVSATFWQNPRNHFRCQVQF | |
| | | | | | | | hTRBJ02-7 | | | YGLSENDEWTQDRAKPVTQIVSAEAWGR | |
| | | | | | | | | | | ADCGFTSVSYQQGVLSATILYEILLGKATL | |
| | | | | | | | | | | YAVLVSALVLMAMVKRKDF | |
| | | | | | | B | hTRBV05- | CAS | 74 | MGPGLLCWVLLCLLGAGSVETGVTQSPTH | 150 |
| | | | | | | | 4_ | TLS | | LIKTRGQQVTLRCSSQSGHNTVSWYQQAL | |
| | | | | | | | CAST | TGQ | | GQGPQFIFQYYREEENGRGNFPPRFSGLQ | |
| | | | | | | | LST | GYG | | FPNYSSELNVNALELDDSALYLCASTLSTG | |
| | | | | | | | GQ | YTF | | QGIYGYTFGSGTRLTVVEDLNKVFPPEVAV | |
| | | | | | | | GIY | G | | FEPSEAEISHTQKATLVCLATGFFPDHVEL | |
| | | | | | | | GY | | | SWWVNGKEVHSGVSTDPQPLKEQPALND | |
| | | | | | | | TFG_ | | | SRYCLSSRLRVSATFWQNPRNHFRCQVQ | |
| | | | | | | | hTRBJ | | | FYGLSENDEWTQDRAKPVTQIVSAEAWG | |
| | | | | | | | 01-2 | | | RADCGFTSVSYQQGVLSATILYEILLGKAT | |
| | | | | | | | | | | LYAVLVSALVLMAMVKRKDF | |
| | | | | | | C | hTRBV05- | CAS | 76 | MGPGLLCWVLLCLLGAGSVETGVTQSPTH | 152 |
| | | | | | | | 4_ | SPT | | LIKTRGQQVTLRCSSQSGHNTVSWYQQAL | |
| | | | | | | | CASS | TSG | | GQGPQFIFQYYREEENGRGNFPPRFSGLQ | |
| | | | | | | | PTT | RIG | | FPNYSSELNVNALELDDSALYLCASSPTTS | |
| | | | | | | | SG | ELF | | GRIGELFFGEGSRLTVLEDLNKVFPPEVAV | |
| | | | | | | | RIG | FG | | FEPSEAEISHTQKATLVCLATGFFPDHVEL | |
| | | | | | | | ELF | | | SWWVNGKEVHSGVSTDPQPLKEQPALND | |
| | | | | | | | FG_ | | | SRYCLSSRLRVSATFWQNPRNHFRCQVQ | |
| | | | | | | | hTRBJ02-2 | | | FYGLSENDEWTQDRAKPVTQIVSAEAWG | |
| | | | | | | | | | | RADCGFTSVSYQQGVLSATILYEILLGKAT | |
| | | | | | | | | | | LYAVLVSALVLMAMVKRKDF | |

| | TCR pMHC beta | | | TCR | | | | TCR pMHC alpha | |
|---|---|---|---|---|---|---|---|---|---|
| | chain | | | pMHC | | | | chain | |
| | (excluding | SEQ | Additional | alpha | SEQ | | SEQ | (excluding | SEQ |
| Patient | the constant | ID | Id | chain | ID | | ID | the constant | ID |
| ID | region) | NO | information | CDR3 | NO | TCR pMHC alpha chain | NO | region) | NO |
| Patient | MGTRLFFYVALCL | 224 | hTRAV23_ | CAA | 71 | MDKILGASFLVLWLQLCWVSGQQKEK | 147 | MDKILGASFLVLW | 223 |
| 7 | LWTGHMDAGITQS | | CAAP | PMP | | SDQQQVKQSPQSLIVQKGGISIINCAY | | LQLCWVSGQQKE | |
| | PRHKVTETGTPVT | | MP | MDT | | ENTAFDYFPWYQQFPGKGPALLIAIRP | | KSDQQQVKQSPQ | |
| | LRCHQTENHRYM | | MD | GRR | | DVSEKKEGRFTISFNKSAKQFSLHIMD | | SLIVQKGGISIINCA | |
| | YWYRQDPGHGLR | | TG | AL | | SQPGDSATYFCAAPMPMDTGRRALT | | YENTAFDYFPWYQ | |
| | LIHYSYGVKDTDK | | RR | TFG | | FGSGTRLQVQPNIQNPDPAVYQLRDS | | QFPGKGPALLIAIR | |
| | GEVSDGYSVSRS | | ALT | | | KSSDKSVCLFTDFDSQTNVSQSKDSD | | PDVSEKKEGRFTI | |
| | KTEDFLLTLESATS | | FG_ | | | VYITDKTVLDMRSMDFKSNSAVAWSN | | SFNKSAKQFSLHI | |
| | SQTSVYFCAISGG | | hTRAJ05 | | | KSDFACANAFNNSIIPEDTFFPSPESS | | MDSQPGDSATYE | |
| | SVGEQYFGPGTRL | | | | | CDVKLVEKSFETDTNLNFQNLSVIGFR | | CAAPMPMDTGRR | |
| | TVT | | | | | ILLLKVAGFNLLMTLRLWSS | | ALTFGSGTRLQVQ | |
| | | | | | | | | PN | |
| | MGPGLLCWVLLCL | 226 | hTRAV21_ | CAV | 73 | METLLGLLILWLQLQWVSSKQEVTQIP | 149 | METLLGLLILWLQL | 225 |
| | LGAGSVETGVTQS | | CAVS | SSG | | AALSVPEGENLVLNCSFTDSAIYNLQW | | QWVSSKQEVTQIP | |
| | PTHLIKTRGQQVT | | SG | SAR | | FRQDPGKGLTSLLLIQSSQREQTSGR | | AALSVPEGENLVL | |
| | LRCSSQSGHNTVS | | SAR | QLT | | LNASLDKSSGRSTLYIAASQPGDSATY | | NCSFTDSAIYNLQ | |
| | WYQQALGQGPQF | | QLT | FG | | LCAVSSGSARQLTFGSGTQLTVLPDIQ | | WFRQDPGKGLTS | |
| | IFQYYREEENGRG | | FG_ | | | NPDPAVYQLRDSKSSDKSVCLFTDFD | | LLLIQSSQREQTS | |
| | NFPPRFSGLQFPN | | hTRAJ22 | | | SQTNVSQSKDSDVYITDKTVLDMRSM | | GRLNASLDKSSGR | |
| | YSSELNVNALELD | | | | | DFKSNSAVAWSNKSDFACANAFNNSI | | STLYIAASQPGDS | |
| | DSALYLCASTLST | | | | | IPEDTFFPSPESSCDVKLVEKSFETDT | | ATYLCAVSSGSAR | |
| | GQGIYGYTFGSGT | | | | | NLNFQNLSVIGFRILLLKVAGFNLLMT | | QLTFGSGTQLTVL | |
| | RLTVV | | | | | LRLWSS | | PD | |
| | MGPGLLCWVLLCL | 228 | hTRAV21_ | CAV | 75 | METLLGLLILWLQLQWVSSKQEVTQIP | 151 | METLLGLLILWLQL | 227 |
| | LGAGSVETGVTQS | | CAV | GGS | | AALSVPEGENLVLNCSFTDSAIYNLQW | | QWVSSKQEVTQIP | |
| | PTHLIKTRGQQVT | | GG | GSA | | FRQDPGKGLTSLLLIQSSQREQTSGR | | AALSVPEGENLVL | |
| | LRCSSQSGHNTVS | | SG | RQ | | LNASLDKSSGRSTLYIAASQPGDSATY | | NCSFTDSAIYNLQ | |
| | WYQQALGQGPQF | | SAR | LTF | | LCAVGGSGSARQLTFGSGTQLTVLPD | | WFRQDPGKGLTS | |
| | IFQYYREEENGRG | | QLT | G | | IQNPDPAVYQLRDSKSSDKSVCLFTD | | LLLIQSSQREQTS | |
| | NFPPRFSGLQFPN | | FG_ | | | FDSQTNVSQSKDSDVYITDKTVLDMR | | GRLNASLDKSSGR | |
| | YSSELNVNALELD | | hTRAJ22 | | | SMDFKSNSAVAWSNKSDFACANAFN | | STLYIAASQPGDS | |
| | DSALYLCASSPTT | | | | | NSIIPEDTFFPSPESSCDVKLVEKSFE | | ATYLCAVGGSGSA | |
| | SGRIGELFFGEGS | | | | | TDTNLNFQNLSVIGFRILLLKVAGFNL | | RQLTFGSGTQLTV | |
| | RLTVL | | | | | LMTLRLWSS | | LPD | |

Note:
Underline: variable region; Italic: CDR3; Bolded: constant region. Patient identification number (Id); gene; single nucleotide variants (SNVs); HLA restriction (predicted restrictions not confirmed by pMHC multimers are shown with an *); peptide sequence; TCR Id; validated TCRα and TCRß chains when TCRαß pair was validated by TCR cloning as described in the Methods section (NA: not applicable when the TCRα chain was not identified by TCR cloning); Percentage of 4-1BB expression after tumor challenge of transfected T cells, after subtraction of the 4-1BB background obtained with transfected T cells alone (NA: when autologous tumor cell lines were not available).

TABLE 6
Protein Data Bank entries used to model the 3D structures of the PHLPP2_{N1186Y}-
specific TCRs A, B, and C.

| | | Non | | | | | | |
|---|---|---|---|---|---|---|---|---|
| | | synonymous | | | | | | Templates TCRα\|\|TCRβ\|\|a:b |
| Patient | | mutations | HLA | Minimal | TCR | | | orientation\|\|peptide MHC\|\| |
| ID | Gene | SNVs | restriction | epitope | Id | TCR pMHC beta chain | TCR pMHC alpha chain | TCR:p:MHC orientation |
| Patient | PHLPP2 | N1186Y | AD1:01 | QSDNGLD | A | hTRBV10-3_CAISGGSVGEQYFG_ | hTRAV23_CAAPMPMDTGRRALTFG_ | 3mff, 3c6\|, 3vxm, 1uh3\|\| |
| 7 | | | | SDY | | hTRBJ02-7 | hTRAJ05 | 3qeq,3vxm\|\|1u3h:1u3h\|\| |
| | | | | | | | | 5brs, 5bs0, 6at9\|\|5brs, 5bs0 |
| | | | | | B | hTRBV05-4_CCASTLSTGQGIYGYTFG_ | hTRAV21_CAVSSGSARQLTFG_ | 6eh4, 4eup\|\|6bj2, 4h\|\|\|6eh4: |
| | | | | | | hTRBJ01-2 | ATRAJ22 | 6eh4\|\|5brs, 5bs0, 6at9\|\|5brs, |
| | | | | | | | | 5bs0 |
| | | | | | C | hTRBV05-4_CASSPTTSGRIGELFFG_ | hTRAV21_CAVGGSGSARQLTFG_ | 6eh4, 4h1\|\|\|6bj2, 4h1\|\|\|6eh4: |
| | | | | | | hTRBJ02-2 | hTRAJ22 | 6eh4\|\|5brs,5bs0, 6at9\|\| |
| | | | | | | | | 5brs, 5hs0 |

Patient identification number (Id); gene; single nucleotide variants (SNVs); HLA restriction; peptide sequence; TCR Id; validated TCRα and TCRβ chains; Modeled structures of three PHLPP2_{N1186Y}-specific TCRs; Protein Data Bank entries used as templates to model the 3D structures are displayed on FIGS. 2D and 10.

1. A T cell receptor (TCR) or antigen-binding fragment thereof that binds specifically to a tumor-associated antigen, wherein the TCR or antigen-binding fragment thereof comprises an amino acid sequence having at least 90% sequence identity to an amino acid sequence of SEQ ID NOs: 1-228 or comprises an amino acid sequence of SEQ ID NOs: 1-228.

2. The TCR or antigen-binding fragment thereof of claim 1, comprising an α chain amino acid sequence having at least 90% sequence identity to an amino acid sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, or 227; or comprises an α chain amino acid sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, or 227.

3. The TCR or antigen-binding fragment thereof of claim 1, comprising a β chain amino acid sequence having at least 90% sequence identity to an amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, or 228; or comprises a β chain amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, or 228.

4. The TCR or antigen-binding fragment thereof of claim 1, comprising the α chain amino acid sequence and the β chain amino acid sequence having respective amino acid sequences of SEQ ID NOs: 1-2, 3-4, 5-6, 7-8, 9-10, 11-12, 13-14, 15-16, 17-18, 19-20, 21-22, 23-24, 25-26, 27-28, 29-30, 31-32, 33-34, 35-36, 37-38, 39-40, 41-42, 43-44, 45-46, 47-48, 49-50, 51-52, 53-54, 55-56, 57-58, 59-60, 61-62, 63-64, 65-66, 67-68, 69-70, 71-72, 73-74, 75-76, 77-78, 79-80, 81-82, 83-84, 85-86, 87-88, 89-90, 91-92, 93-94, 95-96, 97-98, 99-100, 101-102, 103-104, 105-106, 107-108, 109-110, 111-112, 113-114, 115-116, 117-118, 119-120, 121-122, 123-124, 125-126, 127-128, 129-130, 131-132, 133-134, 135-136, 137-138, 139-140, 141-142, 143-144, 145-146, 147-148, 149-150, 151-152, 153-154, 155-156, 157-158, 159-160, 161-162, 163-164, 165-166, 167-168, 169-170, 171-172, 173-174, 175-176, 177-178, 179-180, 181-182, 183-184, 185-186, 187-188, 189-190, 191-192, 193-194, 195-196, 197-198, 199-200, 201-202, 203-204, 205-206, 207-208, 209-210, 211-212, 213-214, 215-216, 217-218, 219-220, 221-222, 223-224, 225-226, or 227-228.

5. The TCR or antigen-binding fragment thereof of claim 1, wherein the TCR is an αβ heterodimeric TCR.

6. The TCR or antigen-binding fragment thereof of claim 1, wherein the TCR is an αβ single chain TCR.

7. The TCR or antigen-binding fragment thereof of claim 1, wherein the tumor-associated antigen is selected from the group consisting of: 707-AP, a biotinylated molecule, a-Actinin-4, abl-bcr alb-b3 (b2a2), abl-bcr alb-b4 (b3a2), adipophilin, AFP, AIM-2, Annexin II, ART-4, BAGE, BCMA, b-Catenin, bcr-abl, bcr-abl p190 (e1a2), bcr-abl p210 (b2a2), bcr-abl p210 (b3a2), BING-4, CA-125, CAG-3, CAIX, CAMEL, Caspase-8, CD171, CD19, CD20, CD22, CD4, CD23, CD24, CD30, CD33, CD38, CD44v7/8, CD70, CD123, CD133, CDC27, CDK-4, CEA, CLCA2, CLL-1, CTAG1B, Cyp-B, DAM-10, DAM-6, DEK-CAN, DLL3, EGFR, EGFRvIII, EGP-2, EGP-40, ELF2, Ep-CAM, EphA2, EphA3, erb-B2, erb-B3, erb-B4, ES-ESO-1a, ETV6/AML, FAP, FBP, fetal acetylcholine receptor, FGF-5, FN, FR-α, G250, GAGE-1, GAGE-2, GAGE-3, GAGE-4, GAGE-5, GAGE-6, GAGE-7B, GAGE-8, GD2, GD3, GnT-V, Gp100, gp75, GPC3, GPC-2, Her-2, HLA-A*0201-R170I, HMW-MAA, HSP70-2 M, HST-2 (FGF6), HST-2/neu, hTERT, iCE, IL-11Rα, IL-13Rα2, KDR, KIAA0205, K-RAS, L1-cell adhesion molecule, LAGE-1, LDLR/FUT, Lewis Y, L1-CAM, MAGE-1, MAGE-10, MAGE-12, MAGE-2, MAGE-3, MAGE-4, MAGE-6, MAGE-A1, MAGE-A2, MAGE-A3, MAGE-A6, MAGE-B1, MAGE-B2, Malic enzyme, Mammaglobin-A, MART-1/Melan-A, MART-2, MC1R, M-CSF, mesothelin, MUC1, MUC16, MUC2, MUM-1, MUM-2, MUM-3, Myosin, NA88-A, Neo-PAP, NKG2D, NPM/ALK, N-RAS, NY-ESO-1, OA1, OGT, oncofetal antigen (h5T4), OS-9, P polypeptide, P15, P53, PRAME, PSA, PSCA, PSMA, PTPRK, RAGE, ROR1, RU1, RU2, SART-1, SART-2, SART-3, SOX10, SSX-2, Survivin, Survivin-2B, SYT/SSX, TAG-72, TEL/AML1, TGFαRII, TGFβRII, TP1, TRAG-3, TRG, TRP-1, TRP-2, TRP-2/INT2, TRP-2-6b, Tyrosinase, VEGF-R2, WT1, α-folate receptor, and κ-light chain.

8. The TCR or antigen-binding fragment thereof of claim 1, wherein the tumor-associated antigen comprises an amino acid sequence of SEQ ID NOs: 229-268.

9. A polypeptide comprising an amino acid sequence having at least 90% sequence identity to an amino acid sequence of SEQ ID NOs: 1-228 or comprising an amino acid sequence of SEQ ID NOs: 1-228.

10. The polypeptide of claim 9, comprising a first polypeptide chain that comprises an amino acid sequence having at least 90% sequence identity to an amino acid sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, or 227; or comprises an amino acid sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, or 227.

11. The polypeptide of claim 9, comprising a second polypeptide chain that comprises an amino acid sequence having at least 90% sequence identity to an amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, or 228; or comprises an amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, or 228.

12. The polypeptide of claim 9, comprising the first polypeptide chain and the second polypeptide chain having respective amino acid sequences of SEQ ID NOs: 1-2, 3-4, 5-6, 7-8, 9-10, 11-12, 13-14, 15-16, 17-18, 19-20, 21-22, 23-24, 25-26, 27-28, 29-30, 31-32, 33-34, 35-36, 37-38, 39-40, 41-42, 43-44, 45-46, 47-48, 49-50, 51-52, 53-54, 55-56, 57-58, 59-60, 61-62, 63-64, 65-66, 67-68, 69-70, 71-72, 73-74, 75-76, 77-78, 79-80, 81-82, 83-84, 85-86, 87-88, 89-90, 91-92, 93-94, 95-96, 97-98, 99-100, 101-102, 103-104, 105-106, 107-108, 109-110, 111-112, 113-114, 115-116, 117-118, 119-120, 121-122, 123-124, 125-126, 127-128, 129-130, 131-132, 133-134, 135-136, 137-138, 139-140, 141-142, 143-144, 145-146, 147-148, 149-150, 151-152, 153-154, 155-156, 157-158, 159-160, 161-162, 163-164, 165-166, 167-168, 169-170, 171-172, 173-174, 175-176, 177-178, 179-180, 181-182, 183-184, 185-186, 187-188, 189-190, 191-192, 193-194, 195-196, 197-198, 199-200, 201-202, 203-204, 205-206, 207-208, 209-210, 211-212, 213-214, 215-216, 217-218, 219-220, 221-222, 223-224, 225-226, or 227-228.

13. A bifunctional molecule comprising the polypeptide of claim 9 and a second polypeptide that specifically binds to a cell surface protein on a T cell.

14. The bifunctional molecule of claim 13, wherein the second polypeptide comprises an immune effector polypeptide.

15. The bifunctional molecule of claim 13, wherein the cell surface protein is selected from the group consisting of CD2, CD3, CD4, CD8, CD44, CD45RA, CD45RB, CD45RO, CD49a, CD49b, CD49c, CD49d, CD49e, CD49f, CD16, CD28, and IL-2R.

16. The bifunctional molecule of claim 15, wherein the cell surface protein is CD3.

17. The bifunctional molecule of claim claim 14, wherein the immune effector polypeptide comprises IL-1, IL-1α, IL-3, IL-4, IL-5, IL-6, IL-7, IL-10, IL-11, IL-12, IL-13, IL-15, IL-21, IL-23, TGF-β, IFN-γ, TNFα, an anti-CD2 antibody, an anti-CD3 antibody, an anti-CD4 antibody, an anti-CD8 antibody, an anti-CD44 antibody, an anti-CD45RA antibody, an anti-CD45RB antibody, an anti-CD45RO antibody, an anti-CD49a antibody, an anti-CD49b antibody, an anti-CD49c antibody, an anti-CD49d antibody, an anti-CD49e antibody, an anti-CD49f antibody, an anti-CD16 antibody, an anti-CD28 antibody, or an anti-IL-2R antibody.

18. The bifunctional molecule of claim 17, wherein the anti-CD3 antibody is selected from the group consisting of OKT3, UCHT-1, BMA031, 12F6, and an scFv derived therefrom.

19. The bifunctional molecule of claim 13, wherein the polypeptide is linked to the second polypeptide directly or via a linker.

20. A nucleic acid comprising a polynucleotide sequence that encodes the bifunctional molecule of claim 13.

21. A vector comprising the nucleic acid of claim 20.

22. The vector of claim 21, wherein the vector comprises a retroviral vector or a lentiviral vector.

23. A cell comprising the vector of claim 21.

24. The cell of claim 23, wherein the cell comprises an immune cell.

25. The cell of claim 24, wherein the immune cell comprises a lymphocyte.

26. The cell of claim 25, wherein the lymphocyte comprises a T cell or a natural killer (NK) cell.

27. The cell of claim 26, wherein the T cell comprises a CD8+ T cell or a CD4+ T cell.

28. The cell of claim 26, wherein the T cell comprises a human T cell.

29. A composition comprising the the cell claim 23.

30. The composition of claim 29, further comprising a therapeutic agent.

31. The composition of claim 29, wherein the therapeutic agent comprises an anti-tumor or anti-cancer agent.

32. The composition of claim 31, wherein the anti-tumor or anti-cancer agent comprises any one of taxotere, carboplatin, trastuzumab, epirubicin, cyclophosphamide, cisplatin, docetaxel, doxorubicin, etoposide, 5-FU, gemcitabine, methotrexate, and paclitaxel, mitoxantrone, epothilone B, epidermal-growth factor receptor (EGFR)-targeting monoclonal antibody 7A7.27, vorinostat, romidepsin, docosahexaenoic acid, bortezomib, shikonin, an oncolytic virus, and combinations thereof.

33. The composition of claim 30, wherein the therapeutic agent comprises a chemotherapeutic agent selected from the group consisting of asparaginase, busulfan, carboplatin, cisplatin, daunorubicin, doxorubicin, fluorouracil, gemcitabine, hydroxyurea, methotrexate, paclitaxel, rituximab, vinblastine, and vincristine.

34. A kit comprising the the composition of claim 29.

35. A method of preparing a population of cells expressing a TCR specific for a target cell in a patient, comprising:
isolating a plurality of cells from a subject;
transfecting the plurality of cells with the vector of claim 21; and
optionally expanding the transfected cells.

36. The method of claim 35, wherein the subject is the patient.

37. The method of claim 35, wherein the subject is a healthy donor.

38. The method of claim 35, wherein the target cell comprises a tumor cell.

39. A method of directing immune cells to a target cell in a patient, comprising administering to the patient the composition of claim 29.

40. A method for an adoptive T cell therapy in a patient, comprising administering to the patient a therapeutically effective amount of the composition of claim 29.

41. A method for stimulating or enhancing an immune response in a subject in need thereof, comprising administering to the subject the composition of claim 29.

42. A method of preventing or treating cancer in a patient in need thereof, comprising administering to the patient a therapeutically effective amount of the composition of claim 29.

43. The method of claim 42, wherein the cancer is selected from adrenal gland tumors, biliary cancer, bladder cancer, brain cancer, breast cancer, carcinoma, central or peripheral nervous system tissue cancer, cervical cancer, colon cancer, endocrine or neuroendocrine cancer or hematopoietic cancer, esophageal cancer, fibroma, gastrointestinal cancer, glioma, head and neck cancer, Li-Fraumeni tumors, liver cancer, lung cancer, lymphoma, melanoma, meningioma, multiple neuroendocrine type I and type II tumors, nasopharyngeal cancer, oral cancer, oropharyngeal cancer, osteogenic sarcoma tumors, ovarian cancer, pancreatic cancer, pancreatic islet cell cancer, parathyroid cancer, pheochromocytoma, pituitary tumors, prostate cancer, rectal cancer, renal cancer, respiratory cancer, sarcoma, skin cancer, stomach cancer, testicular cancer, thyroid cancer, tracheal cancer, urogenital cancer, and uterine cancer.

44. The method of claim 42, wherein the cancer comprises a solid tumor.

45. The method of claim 42, further comprising administering to the patient a second agent or therapy.

46. The method of claim 45, wherein the second agent comprises an anti-tumor or anti-cancer agent.

47. The method of claim 45, wherein the second agent or therapy is administered before or after the composition.

48. The method of claim 45, wherein the second agent or therapy is administered concurrently with the composition.

49. The method of claim 39, wherein the composition is administered by intravenous infusion.

50. A method of detecting cancer in a biological sample, comprising: a) contacting the biological sample with the TCR or antigen-binding fragment thereof of claim 1, and b) detecting binding of the TCR or antigen-binding fragment thereof to the biological sample.

51. The method of claim 50, wherein the TCR or antigen-binding fragment thereof comprises a detectable label.

52. The method of claim 51, wherein the detectable label is selected from the group consisting of a radionuclide, a fluorophore, and biotin.
